(19) United States
(12) Patent Application Publication
(10) Pub. No.: US 2026/0008762 A1
(43) Pub. Date: Jan. 8, 2026

(54) CANNABINOID ACIDS CRYSTALLINE FORMS, METHODS OF PRODUCING, AND USES THEREOF

(71) Applicants: ARIZONA BOARD OF REGENTS ON BEHALF OF ARIZONA STATE UNIVERSITY, Scottsdale, AZ (US); VEXT SCIENCE, INC., Phoenix, AZ (US); BIOSCITECH LLC, Gilbert, AZ (US)

(72) Inventors: Jeffery L. YARGER, Gilbert, AZ (US); Stephen DAVIDOWSKI, Chandler, AZ (US); Michael SETH, Peroria, AZ (US)

(21) Appl. No.: 18/867,051

(22) PCT Filed: May 22, 2023
(86) PCT No.: PCT/US2023/023137

§ 371 Date: Nov. 19, 2024

Related U.S. Application Data
(60) Provisional application No. 63/344,230, filed on May 20, 2022.

(51) Int. Cl. C07D 311/80

(52) U.S. Cl. CPC C07D 311/80

(57) ABSTRACT

This invention is directed to crystalline forms of cannabinoid acids, producing crystalline forms, and methods of use thereof.

This application claims the benefit of and priority to U.S. Provisional Application No. 63/344,230, filed on May 20, 2022, the entire content of which is incorporated herein by reference in its entirety.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed to crystalline forms of cannabinoid acids, producing crystalline forms, and methods of use thereof.

### BACKGROUND OF THE INVENTION

Cannabinoid acids are a class of natural products most commonly isolated from the plant C. sativa. Purified forms of cannabinoids extracted from herbal cannabis or synthetically produced cannabinoids are both potentially useful as active pharmaceutical agents. For example, THC is FDA approved for the treatment of anorexia associated with weight loss in AIDS patients and shows potential pharmacological activity in the treatment of nausea, anxiety, glaucoma and migraines. However, cannabinoid acids tend to degrade over time leading to shelf-life stability concerns. This degradation changes their structure in a way that affects their binding affinity to receptors and in turn their efficacy for a desired application.

### SUMMARY OF THE INVENTION

Aspects of the inventions are drawn towards crystalline forms of cannabinoid acids. Aspects of the invention are drawn towards a crystalline form of tetrahydrocannabinolic acid (THCa) comprising the structure

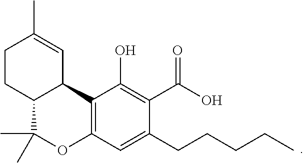 [SMILES: CCCCCc1cc2c(c(O)c1C(=O)O)[C@@H]1C=C(C)CC[C@H]1C(C)(C)O2]

In embodiments the crystalline form comprises a single component crystalline form or a multicomponent crystalline form. In embodiments the multicomponent crystalline form comprises a co-crystal or a salt.

In embodiments, the single component crystalline form comprises at least one of the following features (i) to (ix): (i) a powder x-ray diffraction (PXRD) pattern comprising at least one characteristic peak at 2θ angles of about 6.5, 8.8, 9.6, 10.1, 12.7, 13.1, 14.1, 15.1, 16.4, 17.0, 18.7, 19.4, 19.8, 20.3, 21.3, 21.7, 22.7, 23.6, 25.3, 27.1, and 29.4; (ii) a single crystal x-ray diffraction (SCXRD) pattern comprising at least one characteristic peak at 2θ angles described herein; (iii) a Fourier transform infrared spectroscopy spectrum comprising at least one characteristic peak at about 742, 820, 887, 973, 1029, 1070, 1107, 1167, 1234, 1360, 1428, 1547, 1644, 1700, 2860, 2926, 2957 cm⁻¹; (iv) a liquid state 1H nuclear magnetic resonance (NMR) spectrum comprising at least one characteristic peak at about 0.903, 1.11, 1.35 (m), 1.44, 1.58 (m), 1.68, 1.92 (m), 2.17, 2.79 (m), 2.95 (m), 3.23, 6.26, 6.39, 11.70, 12.16 ppm in CDCl₃; (v) a liquid state ¹³C (1H decoupled) nuclear magnetic resonance (NMR) spectrum comprising at least one characteristic peak at about 14.1, 19.5, 22.5, 23.4, 25.0, 27.4, 31.2, 31.3, 32.0, 33.5, 36.5, 45.6, 78.9, 102.3, 109.9, 112.7, 123.6, 133.9, 147.0, 159.8, 164.7, 176.5 ppm in CDCl3; (vi) a solid state ¹H NMR spectrum comprising at least one characteristic peak at about 1.5, 6.2, 11.2, 11.8, 12.7 ppm; (vii) a solid state ¹H-¹³C cross polarization (CP) magic angle spinning (MAS) NMR spectrum comprising at least one characteristic peak at about 13.8, 15.4, 17.9, 19.3, 20.7, 23.6, 24.2, 24.6, 25.1, 25.9, 26.6, 27.5, 28.5, 31.0, 32.3, 32.9, 35.3, 37.6, 44.1, 45.9, 77.8, 78.9, 101.5, 102.6, 109.6, 110.1, 102.6, 109.6, 110.1, 113.3, 123.1, 124.0, 132.3, 133.2, 146.6, 161.0, 162.0, 165.0, 165.6, 178.1, and 178.5 ppm; (viii) a thermogravimetric analysis (TGA) thermogram comprising a 20% w/w loss from about 125° C. to about 165° C.; and 70% w/w loss from about 165° C. to 200° C., and/or (ix) a differential scanning calorimetry (DSC) thermogram comprising an endothermic onset at about 149° C.

In embodiments, a single crystal belongs to the orthorhombic system, P2₁2₁2₁ space group, and comprises the following single-crystal lattice parameters: a=11.4635(8)Å, b=18.0506(13)A, c=19.2347(14)Å, α=90 degrees, β=90 degrees, and γ=90 degrees.

In embodiments, the single crystal x-ray diffraction (SCXRD) pattern is characterized by an Oak Ridge Thermal Ellipsoid Plot substantially as shown in FIG. 4. the powder x-ray diffraction (PXRD) pattern comprises further comprises at least one peak at 2θ angles described herein. In embodiments, the crystalline form is characterized by a powder x-ray diffraction (PXRD) pattern substantially as shown in FIG. 5. In embodiments, the crystalline form is characterized by a single crystal x-ray diffraction (SCXRD) pattern substantially as shown in FIGS. 44 and 45. In embodiments, the crystalline form is characterized by the Fourier transform infrared spectroscopy spectrum substantially as shown in FIG. 8. In embodiments, the crystalline form is characterized by the liquid state ¹H nuclear magnetic resonance (NMR) spectrum substantially as shown in FIG. 2. In embodiments, the crystalline form is characterized by the ¹H-¹³C cross polarization (CP) magic-angle spinning (MAS) NMR spectrum substantially as shown in FIG. 7. In embodiments, the crystalline form is characterized by the solid state ¹H NMR spectrum substantially as shown in FIG. 6. In embodiments, the crystalline form is characterized by the liquid state ¹³C (¹H decoupled) nuclear magnetic resonance (NMR) spectrum substantially as shown in FIG. 3. In embodiments, the TGA thermogram comprises a degradation onset of about 125° C. In embodiments, the crystalline form is characterized by the thermogravimetric analysis (TGA) thermogram substantially as shown in FIG. 9. In embodiments, the crystalline form is characterized by a differential scanning calorimetry thermogram comprising an endothermic event in the range of about 140° C. to 149° C. In embodiments, the crystalline form is characterized by the differential scanning calorimetry (DSC) thermogram substantially as shown in FIG. 10.

Aspects of the invention are drawn towards a method of preparing a single component crystalline form or a multicomponent crystalline form of tetrahydrocannabinolic acid (THCa) comprising: suspending a plant extract comprising THCa in minimum amount (10-50% w/w) of a solvent at a temperature of about 25 to about 80; storing the suspension in at about −15° C. for about 6 to 48 hrs; and repeating the process until obtaining a purified crystal. In embodiments, the solvent comprises heptane, hexane, pentane, acetonitrile, and 40-80% methanol in water.

Aspects of the invention are drawn towards a method of preparing a single component or multicomponent crystalline form of tetrahydrocannabinolic acid (THCa) comprising: suspending a plant extract comprising THCa in a solvent at a temperature of about −15° C. to about 25° C. and allowing the solvent to evaporate.

Aspects of the invention are drawn towards a crystalline form of cannabigerolic acid (CBGa) comprising the structure

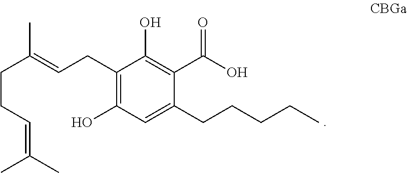 [SMILES: CCCCCc1cc(O)c(C/C=C(\C)CCC=C(C)C)c(O)c1C(=O)O]

In embodiments, the crystalline form comprises a single component crystalline form or a multicomponent crystalline form. In embodiments, the multicomponent crystalline form comprises a co-crystal or a salt.

In embodiments, the single component crystalline form comprises at least one of the following features (i) to (ix): (i) a powder x-ray diffraction (PXRD) pattern comprising at least one characteristic peak at 2θ angles of about 4.6, 8.0, 9.3, 10.8, 12.9, 13.9, 14.8, 16.1, 18.7, 19.3, 21.0, 21.8, 26.1, 27.0 and 28.7; (ii) a single crystal x-ray diffraction (SCXRD) pattern comprising at least one characteristic peak at 2θ angles described herein; (iii) a Fourier transform infrared spectroscopy spectrum comprising at least one characteristic peak at about 675, 734, 753, 809, 831, 880, 924, 973, 1044, 1066, 1085, 1107, 1167, 1245, 1271, 1379, 1413, 1457, 1498, 1580, 1610, 1636, 2851, 2911, 2960, 3399 cm⁻¹; (iv) a liquid state ¹H nuclear magnetic resonance (NMR) spectrum comprising at least one characteristic peak at about 0.90, 1.35, 1.59, 1.67, 1.82, 2.08(m), 2.89, 3.44, 5.06, 5.28, 5.93, 11.90 ppm in CDCl₃; (v) a liquid state ¹³C (¹H decoupled) nuclear magnetic resonance (NMR) spectrum comprising at least one characteristic peak at about 14.1, 16.2, 17.7, 22.1, 22.5, 25.7, 26.4, 31.4, 32.0, 36.6, 39.7, 103.1, 111.4, 111.6, 121.3, 123.8, 132.1, 139.2, 147.5, 160.6, 163.6, 176.1 ppm in CDCl₃; (vi) a solid state ¹H NMR spectrum comprising at least one characteristic peak at about 1.5, 5.2, 11.5 ppm; (vii) a solid state ¹H-¹³C cross polarization (CP) magic angle spinning (MAS) NMR spectrum comprising at least one characteristic peak at about 9.8, 11.3, 13.5, 16.7, 18.8, 21.8, 23.6, 26.3, 28.3, 33.8, 37.7, 98.9, 107.3, 109.0, 117.9, 120.9, 126.0, 132.3, 142.5, 153.5, 159.7, 171.8 ppm; (vii) a thermogravimetric analysis (TGA) thermogram comprising a 13% w/w loss from about 115° C. to about 155° C.; and a 87% w/w loss from about 155 C to 250 C, and/or (ix) a differential scanning calorimetry (DSC) thermogram comprising an endothermic onset at about 115 C.

In embodiments, a single crystal belongs to the triclinic system, P−1 space group, and comprises the following sing-crystal lattice parameters: a=4.6536(7)Å, b=12.1645(17)Å, c=18.693(3)Å, α=98.955(7) degrees, β=92.461(7) degrees, and γ=100.497(&) degrees. In embodiments, wherein the single crystal x-ray diffraction (SCXRD) data allows the visualization of the molecular confirmation using an Oak Ridge Thermal Ellipsoid Plot substantially as shown in FIG. 49.

In embodiments, the crystalline form is characterized by a powder x-ray diffraction (PXRD) pattern substantially as shown in FIG. 15. In embodiments, the crystalline form is characterized by a single crystal x-ray diffraction (SCXRD) pattern substantially as shown in FIG. 49. In embodiments, the crystalline form is characterized by the Fourier transform infrared spectroscopy spectrum substantially as shown in FIG. 17. In embodiments, the crystalline form is characterized by the liquid state ¹H nuclear magnetic resonance (NMR) spectrum substantially as shown in FIG. 31. In embodiments, the crystalline form is characterized by the ¹H-¹³C cross polarization (CP) magic-angle spinning (MAS) NMR spectrum substantially as shown in FIG. 35. In embodiments, the crystalline form is characterized by the solid state ¹H NMR spectrum substantially as shown in FIG. 30. In embodiments, the crystalline form is characterized by the liquid state ¹³C (¹H decoupled) nuclear magnetic resonance (NMR) spectrum substantially as shown in FIG. 41. In embodiments, the crystalline form is characterized by a TGA thermogram comprising a degradation onset of about 115° C. In embodiments, the crystalline form is characterized by the thermogravimetric analysis (TGA) thermogram substantially as shown in FIG. 33. In embodiments, the crystalline form is further characterized by an exothermic peak at about 61° C. In embodiments the crystalline form is characterized the differential scanning calorimetry (DSC) thermogram substantially as shown in FIG. 32.

Aspects of the invention are drawn towards a method of preparing a single component crystalline form of cannabigerolic acid (CBGa) comprising: extracting CBGa hemp with about −15° C. solvent; agitating the CBGa hemp and solvent mixture; vacuum filtrating the mixture; and drying the vacuum filtrate to produce crystals. In embodiments, the solvent comprises acetonitrile or heptane. In further embodiments, the method comprises recrystallizing the crystals. In embodiments, recrystallizing the crystals comprises dissolving the crystals in about 40-80° C. solvent and allowing to cool to about 0° C. In embodiments, recrystallizing the crystals comprises dissolving the crystals in and allowing the solvent to evaporate. In embodiments, the method is repeated three times.

Aspects of the invention are drawn towards a crystalline form of cannabidiolic acid (CBDa) comprising the structure

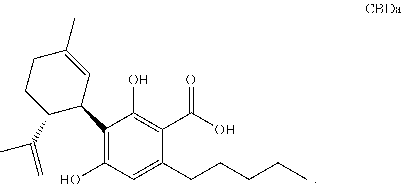 [SMILES: C=C(C)[C@@H]1CCC(C)=C[C@H]1c1c(O)cc(CCCCC)c(C(=O)O)c1O]

In embodiments, the crystalline form comprises a single component crystalline form or a multicomponent crystalline form. In embodiments, the multicomponent crystalline form comprises a co-crystal or a salt.

In embodiments, the single component crystalline form comprises at least one of the following features (i) to (ix): (i) a room temperature powder x-ray diffraction (pXRD) pattern using a copper (Cu) K-alpha1 (wavelength of 1.541 Angstroms) and K-Alpha2 (wavelength of 1.544 Angstroms) anode with a ratio K-Alpha2/K-Alpha1=0.5 comprising at least one characteristic peak at 2θ angles of about 8.1, 9.2, 9.9, 10.9, 12.1, 12.9, 13.3, 14.1, 15.1, 16.9, 17.7, 18.1, 18.7, 19.1, 19.8, 20.2, 20.6, 21.1, 21.8, 22.4, 23.6, 24.2, 24.8, 25.6, 26, 26.9, 27.5, 28.5, 28.7, 30.6, 33.1, 33.3, 34.3, 38.3, 39.1, 39.7 degrees; (ii) a single crystal x-ray diffraction (SCXRD) pattern comprising at least one characteristic peak at 2θ angles described herein; (iii) a Fourier transform infrared spectroscopy spectrum comprising at least one characteristic peak at about 727, 757, 783, 809, 854, 891, 962, 1014, 1032, 1092, 1111, 1144, 1182, 1237, 1278, 1334, 1357, 1375, 1409, 1446, 1495, 1573, 1610, 2855, 2926, 3388 cm⁻¹; (iv) a liquid state ¹H nuclear magnetic resonance (NMR) spectrum comprising at least one characteristic peak at about 0.90, 1.33, 1.58(m), 1.72, 1.80, 2.12, 2.22, 2.38, 2.82(m), 2.93(m), 4.40, 4.55, 5.57, 6.26, 6.64, 11.84 ppm in CDCl₃ relative to TMS at 0 ppm; (v) a liquid state ¹³C (H decoupled) nuclear magnetic resonance (NMR) spectrum comprising at least one characteristic peak at about 14.1, 18.9, 22,5, 23.7, 30.2, 31.2, 32.0, 35.4, 36.6, 46.6, 102.5, 111.4, 112.0, 114.5, 123.9, 140.5, 147.6, 161.0, 164.2, 176.0 ppm in CDCl₃ relative to TMS at 0 ppm; (vi) a solid state ¹H NMR spectrum comprising at least one characteristic peak at about 1.2, 4.3, 5.3, 6.5, 11.2, 12.6 ppm; (vii) a solid state ¹H-¹³C cross polarization (CP) magic angle spinning (MAS) NMR spectrum comprising at least one characteristic peak at about 16.2, 19.0, 22.8, 24.2, 30.5, 31.0, 33.8, 34.94, 35.43, 38.6, 47.5, 102.0, 113.5, 115.3, 115.8, 127.8, 138.0, 146.5, 147.5, 162.8, 164.8, 178.1 ppm; (viii) a thermogravimetric analysis (TGA) thermogram comprising a 100% w/w loss from about 150° C. to about 200° C.; and/or (ix) a differential scanning calorimetry (DSC) thermogram comprising an endothermic onset at about 85.3 C.

In embodiments, a single crystal belongs to the tetragonal system, P4₁2₁2 space group, and comprises the following sing-crystal lattice parameters: a=9.791(3)Å, b=9.791(3)Å, c=43.170(13)Å, α=90 degrees, β=90 degrees, and γ=90 degrees. In embodiments, the single crystal x-ray diffraction (SCXRD) data allows the visualization of the molecular confirmation using an Oak Ridge Thermal Ellipsoid Plot (ORTEP) substantially as shown in FIGS. 50-52.

In embodiments, the crystalline form is characterized by a powder x-ray diffraction (PXRD) pattern substantially as shown in FIG. 40. In embodiments, the crystalline form is characterized by a single crystal x-ray diffraction (SCXRD) pattern substantially as shown in FIGS. 50-52. In embodiments, the crystalline form is characterized by the Fourier transform infrared spectroscopy spectrum substantially as shown in FIG. 23. In embodiments, the crystalline form is characterized by the liquid state ¹H nuclear magnetic resonance (NMR) spectrum substantially as shown in FIG. 22. In embodiments, the crystalline form is characterized by the ¹H-¹³C cross polarization (CP) magic-angle spinning (MAS) NMR spectrum substantially as shown in FIG. 28. In embodiments, the crystalline form is characterized by the solid state ¹H NMR spectrum substantially as shown in FIG. 54. In embodiments, the crystalline form is characterized by the liquid state ¹³C (¹H decoupled) nuclear magnetic resonance (NMR) spectrum substantially as shown in FIG. 29. In embodiments, crystalline form is characterized by a TGA thermogram comprising a degradation onset of about 150° C. In embodiments, the crystalline form is characterized by the thermogravimetric analysis (TGA) thermogram substantially as shown in FIG. 26. In embodiments, the crystalline form is characterized by the differential scanning calorimetry (DSC) thermogram substantially as shown in FIG. 27.

Aspects of the invention are drawn towards a method of preparing a single component crystalline form of cannabidiolic acid (CBDa) comprising: dissolving CBDa hemp with cold acetonitrile; shaking the CBDa hemp and acetonitrile mixture; collecting the vacuum filtrate of the CBDa hemp acetonitrile mixture; concentrating the acetonitrile sample; and subjecting the sample to a temperature of about 5 to −15° C. until crystals form, 1 to 30 days.

Aspects of the invention are drawn towards a method described herein wherein the crystalline form further comprises a pharmaceutically acceptable carrier or an editable carrier.

Aspects of the invention are drawn towards a method described herein, wherein the crystalline form can further be decarboxylated prior to administering to a subject.

Other objects and advantages of this invention will become readily apparent from the ensuing description.

### BRIEF DESCRIPTION OF THE FIGURES

FIG. 1 shows the known molecular bonding (structure) in the cannabinoid acids cannabigerolic acid (CBGa), cannabidiolic acid (CBDa), and tetrahydrocannabinolic acid (THCa).

FIG. 2 shows a liquid state ¹H nuclear magnetic resonance (NMR) of pure crystalline THCa dissolved in deuterated chloroform (CDCl₃) with 0.05% v/v tetramethylsilane (TMS) as the chemical shift reference.

FIG. 3 shows a liquid state ¹³C (1H decoupled) nuclear magnetic resonance (NMR) of pure crystalline THCa dissolved in deuterated chloroform (CDCl₃) with 0.05% v/v tetramethylsilane (TMS) as the chemical shift reference.

FIG. 4 shows the Oak Ridge Thermal Ellipsoid Plot (ORTEP) ball-and-stick type illustration of the primary intermolecular hydrogen bonding across the carboxylic acids in the single crystal x-ray diffraction determined structure of THCa. The crystal structure reveals molecular dimerization across the carboxylic acids as an important structural motif in THCa. This motif is also found in other crystalline cannabinoid acids (e.g., CBDa, CBGa).

FIG. 5 shows a powder x-ray diffraction of polycrystalline THCa. The pXRD pattern was acquired using a Copper (Cu) K-alpha1/K-alpha2 source.

FIG. 6 shows a solid-state ¹H magic angle spinning (MAS) nuclear magnetic resonance (NMR) spectrum for purified polycrystalline form THCa with a spinning speed of 40 kHz.

FIG. 7 shows a Solid state ¹H-¹³C cross polarization (CP) magic angle spinning (MAS) nuclear magnetic resonance (NMR) spectrum for crystalline THCa with a spinning speed of 40 kHz.

FIG. 8 shows the Fourier transform infrared (FT-IR) spectrum of polycrystalline THCa.

FIG. 9 shows thermogravimetric analysis (TGA) of crystalline THCa, at 1° C. per minute.

FIG. 10 shows differential scanning calorimetry (DSC) thermogram of crystalline THCa with a heating rate of 20° C. per minute. The experimentally determined melting point (Tm) for this crystalline polymorph of THCa was found to be 146° C. and enthalpy of fusion (heat of melting) was determined to be 16.3 kJ/mol.

FIG. 11 shows a FT-IR spectrum of amorphous THCa powder.

FIG. 12 shows a DSC thermogram of amorphous THCa with a heating rate of 20° C. per minute. The glass transition temperature (Tg) was determined to be 49° C.

FIG. 13 shows a liquid state ¹H-¹³C heteronuclear single quantum coherence (HSQC) NMR spectrum of THCa dissolved in deuterated chloroform (CDCl₃) with TMS as a chemical shift standard.

FIG. 14 shows a FT-IR spectrum of crystalline CBDa.

FIG. 15 shows a powder x-ray diffraction (pXRD) pattern of crystalline CBGa. The pXRD pattern was acquired using a Copper (Cu) K-alpha1/K-alpha2 source.

FIG. 16 shows a TGA thermogram of crystalline CBGa.

FIG. 17 shows a FT-IR spectrum of crystalline CBGa.

FIG. 18 shows a FT-IR spectrum of tetrahydrocannabinolic acid dicyclohexylamine (THCa:DCHA) cocrystal.

FIG. 19 shows a pXRD pattern of cannabigerolic acid:caffeine (CBGa:Caff) cocrystal. The pXRD pattern was acquired using a Copper (Cu) K-alpha1/K-alpha2 source.

FIG. 20 shows a TGA thermogram of CBGa:Caffeine.

FIG. 21 shows data from single crystal XRD of a THCa:DCHA cocrystal.

FIG. 22 shows a ¹H NMR spectrum of crystalline CBDa dissolved in CDCl₃.

FIG. 23 shows a FT-IR spectrum of crystalline CBDa.

FIG. 24 shows a ¹H NMR of crystalline CBDa dissolved in perdeuterated methanol (d4-MeOD).

FIG. 25 shows DSC thermogram of crystalline CBDa with a heating rate of 10° C. per minute. The experimentally determined melting point (Tm) for this crystalline polymorph of CBDa was found to be 85.3° C. and enthalpy of fusion (heat of melting) was determined to be 16.5 kJ/mol.

FIG. 26 shows TGA thermogram of crystalline CBDa.

FIG. 27 shows a DSC thermogram of amorphous CBDa. The glass transition temperature (Tg) was determined to be 28.0° C.

FIG. 28 shows a ¹H-¹³C cross polarization (CP) MAS solid-state NMR spectrum of crystalline CBDa (MAS spinning speed of 40 kHz).

FIG. 29 shows a ¹³C (¹H decoupled) NMR spectrum of crystalline CBDa dissolved in CDCl₃.

FIG. 30 shows a ¹H MAS ssNMR spectrum of crystalline CBGa with a spinning speed of 40 kHz.

FIG. 31 shows a ¹H NMR spectrum of crystalline CBGa dissolved in CDCl₃.

FIG. 32 shows a DSC thermogram of crystalline CBGa with a heating rate of 10° C. per minute. The experimentally determined melting point (Tm) for this crystalline form of CBGa was found to be 115° C. and enthalpy of fusion (heat of melting) was determined to be 34.7 kJ/mol.

FIG. 33 shows a TGA thermogram of crystalline CBGa.

FIG. 34 shows a powder x-ray diffraction pattern of crystalline CBGa (using a Cu Kα₁/Kα₂ source).

FIG. 35 shows a ¹H-¹³C ssNMR CP-MAS of crystalline CBGa.

FIG. 36 shows a FT-IR spectrum of a 1:1 CBGa:caffeine cocrystal.

FIG. 37 shows a dissolution profile for crystalline CBGa vs. the CBGa Caffeine Cocrystal.

FIG. 38 shows a ¹H-¹³C ssNMR CP-MAS of a 1:1 CBGa:caffeine cocrystal.

FIG. 39 shows a FT-IR spectrum of crystalline sodium THCa salt (Na-THCa).

FIG. 40 shows a pXRD pattern of polycrystalline CBDa. The pXRD pattern was acquired using a Copper (Cu) K-alpha1/K-alpha2 source.

FIG. 41 shows a ¹³C (¹H decoupled) liquid-state NMR of crystalline CBGa dissolved in CDCl₃.

FIG. 42 shows the room temperature single crystal x-ray diffraction data for a 1:1 CBGa:caffeine cocrystal.

FIG. 43 shows the low temperature (100 Kelvin) single crystal x-ray diffraction data for a 1:1 CBGa:caffeine cocrystal.

FIG. 44 shows single crystal x-ray diffraction data (at a temperature of 100 K) for crystalline THCa.

FIG. 45 shows crystal structural data for crystalline THCa.

FIG. 46 shows a ball and stick illustration of a 1:1 THCa:caffeine cocrystal with that associated intermolecular hydrogen bonding.

FIG. 47 shows single crystal x-ray diffraction (at a temperature of 100 K) of a 1:1 THCa:caffeine cocrystal.

FIG. 48 shows single crystal x-ray diffraction (at a temperature of 100 K) of a 1:1 THCa:dicyclohexylamine cocrystal.

FIG. 49 shows single crystal x-ray diffraction (at a temperature of 100 K) of the crystalline solid form of CBGa.

FIG. 50 shows the room temperature (300 K) single crystal x-ray diffraction for the crystalline solid form of CBDa.

FIG. 51 shows single crystal data for crystalline CBDa.

FIG. 52 shows standard low temperature (100 K) single crystal x-ray diffraction for the crystalline solid form of CBDa.

FIG. 53 shows crystal structure data for CBD.

FIG. 54 shows a ¹H MAS ssNMR spectrum of crystalline CBDa.

FIG. 55 shows cannabinoid acid-DCHA cocrystal data.

FIG. 56 shows CBGa-DCHA cocrystal data.

FIG. 57 shows cannabinoid acid-caffeine cocrystal data.

FIG. 58 shows CBDa-Caffeine cocrystal data.

FIG. 59 shows a FTIR spectrum of the CBDa Caffeine Cocrystal.

FIG. 60 shows a FTIR spectrum for the CBDa:DCHA cocrystal.

FIG. 61 shows a FTIR spectrum for the CBGa:DCHA cocrystal.

FIG. 62 shows a FTIR spectrum for the potassium THCa salt (K-THCa).

FIG. 63 shows a FTIR spectrum for the THCa:Proline cocrystal.

FIG. 64 shows a pXRD pattern of crystalline Na-THCa. The pXRD pattern was acquired using a Copper (Cu) K-alpha1/K-alpha2 source.

FIG. 65 shows a pXRD pattern of crystalline K-THCa. The pXRD pattern was acquired using a Copper (Cu) K-alpha1/K-alpha2 source.

FIG. 66 shows a pXRD pattern of a 1:1 THCa:DHCA cocrystal. The pXRD pattern was acquired using a Copper (Cu) K-alpha1/K-alpha2 source.

FIG. 67 shows a pXRD pattern of a 1:1 CBGa:DHCA cocrystal. The pXRD pattern was acquired using a Copper (Cu) K-alpha1/K-alpha2 source.

FIG. 68 shows a pXRD pattern of a 1:1 CBDa:DHCA cocrystal. The pXRD pattern was acquired using a Copper (Cu) K-alpha1/K-alpha2 source.

FIG. 69 shows a pXRD pattern of a 1:1 CBDa:Caffeine cocrystal. The pXRD pattern was acquired using a Copper (Cu) K-alpha1/K-alpha2 source.

FIG. 70 shows a TGA thermogram for the Na-THCa salt. Collected with a heating rate of 1 C per minute.

FIG. 71 shows a TGA thermogram for a 1:1 CBDa-caffeine cocrystal. Collected with a heating rate of 1 C per minute.

FIG. 72 shows a TGA thermogram for amorphous THCa (a-THCa) and crystalline THCa (x-THCa) on the same plot. Collected with a heating rate of 1 C per minute.

FIG. 73 shows an isothermal TGA thermogram for amorphous THCa (a-THCa) and crystalline THCa (x-THCa) on the same plot.

FIG. 74 shows a TGA thermogram for a 1:1 CBDa-caffeine cocrystal. Collected with a heating rate of 1 C per minute.

FIG. 75 shows dissolution profiles for the Na-THCa salt and the crystalline THCa.

FIG. 76 shows a pXRD pattern of a 1:1 THCa:Proline cocrystal. The pXRD pattern was acquired using a Copper (Cu) K-alpha1/K-alpha2 source.

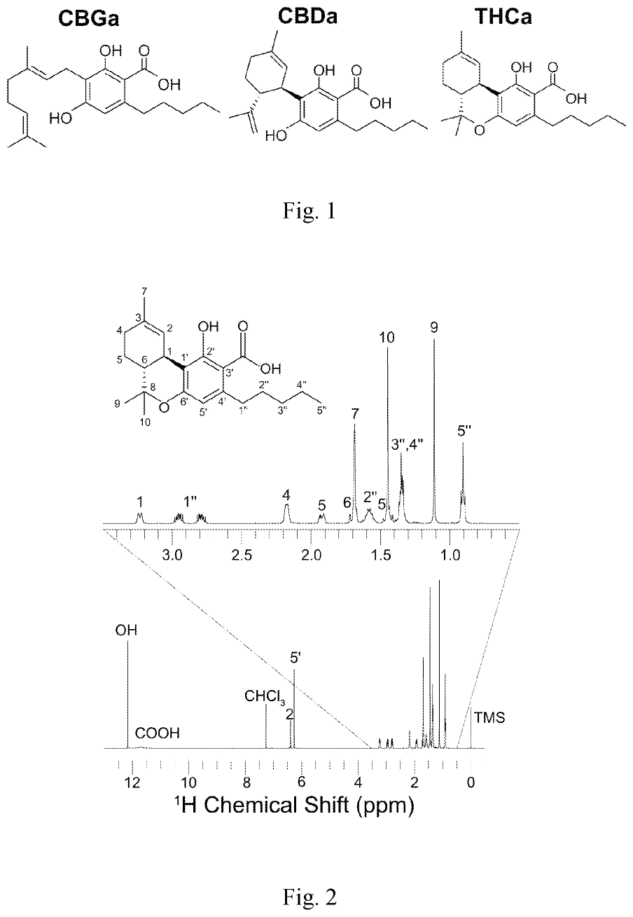

### DETAILED DESCRIPTION OF THE INVENTION

Aspects described herein provide crystalline solid forms of cannabinoid acids that can be applicable to a wide range of products, including pharmacological active ingredients. For example, the molecular structural of these crystalline states of the cannabinoid acids produced herein have improved stability and resistance to chemical reaction and degradation compared to their non-crystalline or amorphous forms that are naturally occurring. The compositions and methods described herein provide increased shelf life and can reduce the cost that can be caused by cannabinoid acid active pharmaceutical ingredient (API) degradation.

Detailed descriptions of one or more preferred embodiments are provided herein. It is to be understood, however, that the present invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein, the term “about” can refer to approximately, roughly, around, or in the region of When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

As used herein, the term “substantially the same” or “substantially” can refer to variability typical for a particular method is taken into account. For example, with reference to X-ray diffraction peak positions, the term “substantially” can refer to typical variability in peak position and intensity are taken into account. One skilled in the art will appreciate that the peak positions (2θ) can show some variability, for example about ±0.2° 2θ. Further, one skilled in the art will appreciate that relative peak intensities will show inter-apparatus variability as well as variability due to degree of crystallinity, preferred orientation, prepared sample surface, and other factors known to those skilled in the art, and are to be taken as qualitative measures only. Similarly, ¹³C and ¹⁹F solid state NMR spectrum (ppm) show variability, for example about ±0.2 ppm.

As used here the term “crystalline” as used herein can refer to having a regularly repeating arrangement of molecules or external face planes. Crystalline forms can differ with respect to thermodynamic stability, physical parameters, x-ray structure and preparation processes.

As used herein, the term “single component crystalline form” can refer to a crystal containing one chemical compound.

As used herein, the term “crystal” can refer to a solid in which the constituent atoms, molecules or ions are arranged in an ordered repeating three-dimensional pattern.

As used herein the term “polymorph” can refer to a specific crystal form of a material for which multiple crystal forms have been identified.

As used herein, the term “amorphous” can refer to a disordered solid state.

As used herein, the term “multicomponent crystalline form” can refer to a crystalline form containing at least two or more distinct, non-covalently bonded compounds. In embodiments, multicomponent crystalline forms can comprise cocrystals, salts, and solvates.

As used herein, the term “solvate” can refer to having, in a lattice, a stoichiometric or non-stoichiometric amount of a solvent such as water, acetic add, methanol, etc., or mixtures thereof, bound by non-covalent intermolecular forces.

As used herein, the term “cocrystal” can refer to a crystalline form of a material containing at least 2 or more molecular or ionic atoms or compounds.

As used herein, the term “salt” can refer to a crystalline form of a material containing at least 2 or more ionic atoms or compounds.

Aspects of the invention are drawn towards crystalline forms of cannabinoids. As used herein, the term “cannabinoid” can refer to compound form a class of molecules which can be found in plants of the genus cannabis and their derivatives thereof.

In embodiments, aspects of the invention comprise new crystalline forms of cannabinoids. In embodiments, these crystalline forms provide increased stability over there non-crystalline forms. As used herein, the term “stability” can refer to chemical stability and/or solid-state stability. In embodiments, the increased the crystalline compounds with increased chemical and/or solid-state stability can be stored in an isolated form, or in the form of a formulation in which it is provided in admixture with pharmaceutically acceptable carriers, diluents or adjuvants (e.g., in an oral dosage form, such as tablet, capsule etc.), under normal storage conditions, with a limited degree of chemical degradation or decomposition.

Aspects of the invention are drawn towards crystalline forms of tetrahydrocannabinolic acid (THCa), wherein THCa can comprise the structure:

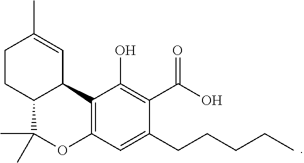 [SMILES: CCCCCc1cc2c(c(O)c1C(=O)O)[C@@H]1C=C(C)CC[C@H]1C(C)(C)O2]

Aspects of the invention are drawn towards crystalline forms of cannabigerolic acid (CBGa), wherein CBGa can comprise the structure:

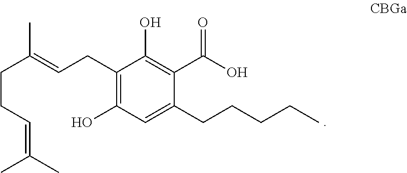 [SMILES: CCCCCc1cc(O)c(C/C=C(\C)CCC=C(C)C)c(O)c1C(=O)O]

Aspects of the invention are drawn towards crystalline forms of cannabidiolic acid (CBDa) comprising the structure

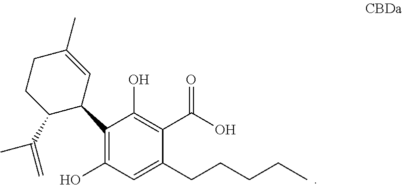 [SMILES: C=C(C)[C@@H]1CCC(C)=C[C@H]1c1c(O)cc(CCCCC)c(C(=O)O)c1O]

In embodiments, crystalline forms described herein can be characterized by powder x-ray diffraction (PXRD), single crystal x-ray diffraction (SCXRD), Fourier transform infrared spectroscopy, liquid and solid state nuclear magnetic resonance NMR (¹H and ¹³C) thermogravimetric analysis (TGA), and differential scanning calorimetry (DSC).

In embodiments, X-ray powder diffraction can be used to characterize crystalline materials. In embodiments X-ray powder diffraction can provide information on unit cell dimensions. As used herein, the terms “X-ray powder diffraction” (XRPD or XPRD) and “powder X-ray diffraction” (PXRD or pXRD) can be used interchangeably.

The term “powder X-ray diffraction pattern” or “PXRD-pattern” can refer to an experimentally observed diffractogram or to parameters derived from it. In embodiments, powder X-ray diffraction patterns are characterized by the position of the peak (abscissa) and the intensity of the peak (ordinates). The term “peak intensity” can refer to the relative intensity of a signal in a given X-ray diffraction pattern. The factors influencing the relative intensity of the peak can be (1) the thickness of the sample and (2) the preferred orientation (i.e., the effect arising from non-random orientation of crystalline particles). The term “peak position”, used herein, can refer to the position of the x-ray reflex measured and observed in powder diffraction experiments. The peaks positions can be used to determine the dimensions of the unit cell.

It is known in the art that an X-ray powder diffraction pattern can be obtained which has one or more measurement errors depending on measurement conditions (such as equipment, sample preparation or machine used). In particular, it is generally known that intensities in an X-ray powder diffraction pattern can fluctuate depending on measurement conditions and sample preparation. For example, persons skilled in the art of X-ray powder diffraction will realize that the relative intensity of peaks can be affected by, for example, grains above 30 microns in size and non-unitary aspect ratios, which can affect analysis of samples. The skilled person will also realize that the position of reflections can be affected by the precise height at which the sample sits in the diffractometer and the zero calibration of the diffractometer. The surface planarity of the sample can also have a small effect. Hence a person skilled in the art will appreciate that the diffraction pattern data presented herein is not to be construed as absolute (for further information see, e.g., Pecharsky, V. K., &amp; Zavalij, P. Y. (2009). Fundamentals of powder diffraction and structural characterization of material. Springer and F., D. W. I. (2006). Structure determination from Powder Diffraction Data. Oxford Univ. Press.). Thus, the crystalline forms of the cannabinoids of the present invention are not limited to the crystals that provide X-ray powder diffraction patterns identical to the X-ray powder diffraction patterns shown in the accompanying Figures and any crystals providing X-ray powder diffraction patterns substantially the same as that shown in Figures fall within the scope of the present invention. A person skilled in the art of X-ray powder diffraction is able to judge the substantial identity of X-ray powder diffraction patterns.

The “2-theta value” or “2θ” can refer to the peak position (in degrees) which is derived from the experimental X-ray diffraction data and can be the abscissa measurement unit in powder X-ray diffraction. In general, in an X-ray diffraction experiment, an incident beam falls on a sample with an angle of θ (to the normal) and reflects with an angle of 2θ (to the incident beam). In embodiments, references to specific values of 2θ for crystalline forms described herein can be measured using a diffractometer of equivalent quality and under the experimental conditions outlined herein. With our diffractometers and the outlined conditions, 2θ precision comprises about ±0.1-0.2 degrees.

In embodiments, characteristic peaks can be used to identify compounds. As used herein, the term “characteristic peak” can refer to a peak which one of skill in the art can identify a product.

### Powder Crystallography Sample Preparation and Conditions

Powder x-ray diffraction was performed on a Malvern PANalytical Aeris benchtop XRD system. The system uses a copper (Cu) k-alpha1/2 (k_{α2}/k_{α1}=12) source with PIXcel detector and samples were measured in a standard reflection setup with the ability to scan the angle (2*theta) between 0-120 degrees. Samples were dispersed on a zero-diffraction silicon plate with sample cavity (Sil'tronix). Cerium oxide, quartz, alumina and silver behenate were used as XRD standards.

In embodiments the crystalline form of tetrahydrocannabinolic acid (THCa) can comprise a powder X-ray diffraction spectrum characterized by diffraction angle 2θ comprising at least one characteristic peak at about 6.5, 8.8, 9.6, 10.1, 12.7, 13.1, 14.1, 15.1, 16.4, 17.0, 18.7, 19.4, 19.8, 20.3, 21.3, 21.7, 22.7, 23.6, 25.3, 27.1, and 29.4. In embodiments, the crystalline form of tetrahydrocannabinolic acid (THCa) can comprise a powder X-ray diffraction spectrum characterized by diffraction angle 2θ comprising the spectrum substantially as show in FIG. 5. In embodiments, the crystalline form of tetrahydrocannabinolic acid (THCa) can comprise a powder X-ray diffraction spectrum characterized by Table 2.

TABLE 2
THCa pXRD result summary

| Peak | Pos. | d-spacing | |
|---|---|---|---|
| No. | [°2θ] | [Å] | I/Io |
| 1 | 1.97 | 44.87 | 100 |
| 2 | 6.55 | 13.49 | 10.9 |
| 3 | 8.86 | 9.98 | 91.6 |
| 4 | 9.66 | 9.16 | 11.3 |
| 5 | 10.15 | 8.71 | 15.8 |
| 6 | 12.69 | 6.98 | 11.1 |
| 7 | 13.16 | 6.73 | 3.1 |
| 8 | 14.13 | 6.27 | 10.2 |
| 9 | 15.18 | 5.84 | 4.9 |
| 10 | 16.33 | 5.43 | 5.2 |
| 11 | 17.08 | 5.19 | 5.7 |
| 12 | 18.73 | 4.74 | 8.1 |
| 13 | 19.90 | 4.46 | 20.0 |
| 14 | 20.39 | 4.35 | 4.1 |
| 15 | 21.37 | 4.16 | 25.6 |
| 16 | 21.65 | 4.10 | 5.1 |
| 17 | 22.74 | 3.91 | 7.8 |
| 18 | 23.59 | 3.77 | 8.7 |
| 19 | 25.33 | 3.52 | 3.1 |
| 20 | 27.09 | 3.29 | 3.3 |

In embodiments the crystalline form of cannabigerolic acid (CBGa) can comprise a powder X-ray diffraction spectrum characterized by diffraction angle 2θ comprising at least one characteristic peak at about 0.6, 8.0, 9.3, 10.8, 12.9, 13.9, 14.8, 16.1, 18.7, 19.3, 21.0, 21.8, 26.1, 27.0 and 28.7. In embodiments, the crystalline form of cannabigerolic acid (CBGa) can comprise a powder X-ray diffraction spectrum characterized by diffraction angle 2θ comprising the spectrum substantially as show in FIG. 15. In embodiments, the crystalline form of cannabigerolic acid (CBGa) can comprise a powder X-ray diffraction spectrum characterized by Table 3.

TABLE 3
CBGa pXRD result summary

| peak | Pos. | d-spacing | |
|---|---|---|---|
| No. | [°2θ] | [Å] | I/Io |
| 1 | 1.1 | 82.01 | 0.66 |
| 2 | 4.7 | 18.91 | 100 |
| 3 | 5.9 | 15.11 | 0.38 |
| 4 | 6.4 | 13.84 | 0.28 |
| 5 | 7.5 | 11.78 | 0.46 |
| 6 | 8.1 | 10.91 | 1.10 |
| 7 | 9.3 | 9.47 | 12.0 |
| 8 | 10.2 | 8.71 | 0.24 |
| 9 | 10.9 | 8.12 | 2.80 |
| 10 | 13.0 | 6.82 | 0.32 |
| 11 | 14.0 | 6.31 | 1.84 |
| 12 | 14.9 | 5.94 | 1.71 |
| 13 | 16.2 | 5.46 | 3.31 |
| 14 | 18.7 | 4.74 | 2.37 |
| 15 | 20.9 | 4.25 | 0.34 |
| 16 | 21.9 | 4.07 | 0.39 |
| 17 | 23.5 | 3.79 | 0.43 |
| 18 | 24.4 | 3.64 | 0.21 |
| 19 | 26.0 | 3.43 | 0.46 |
| 20 | 27.0 | 3.30 | 0.27 |

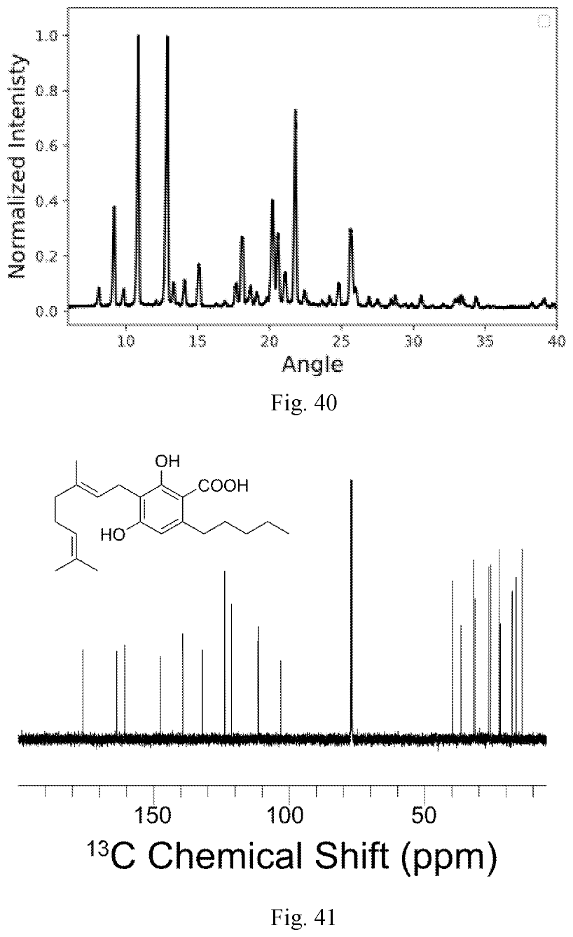

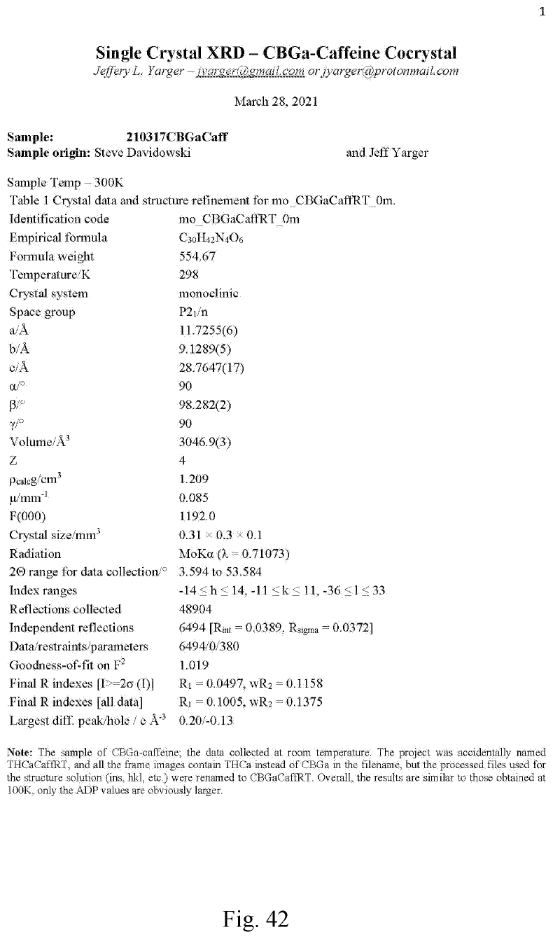

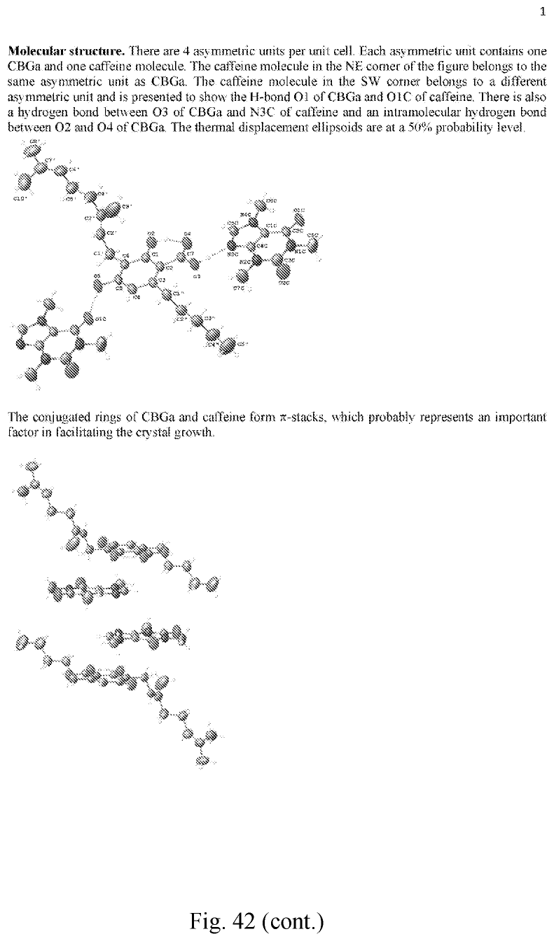

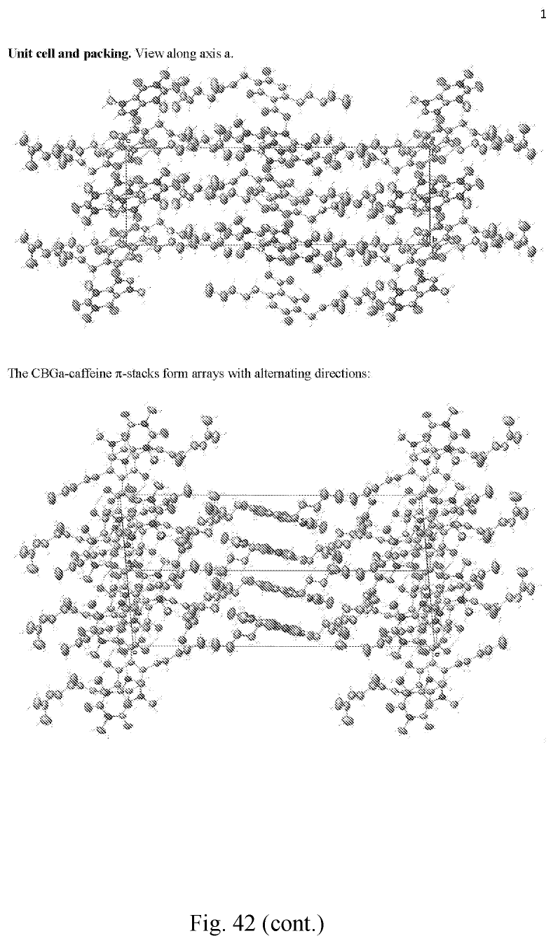

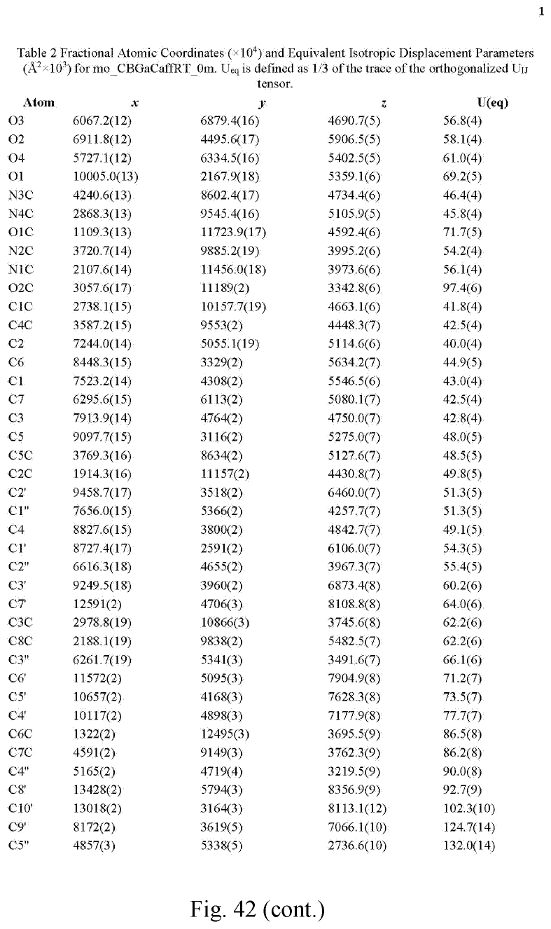

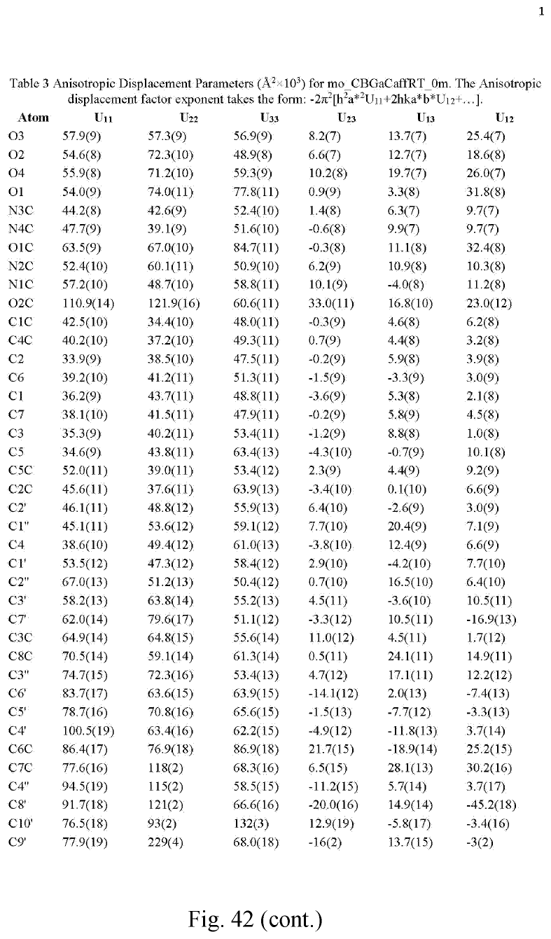

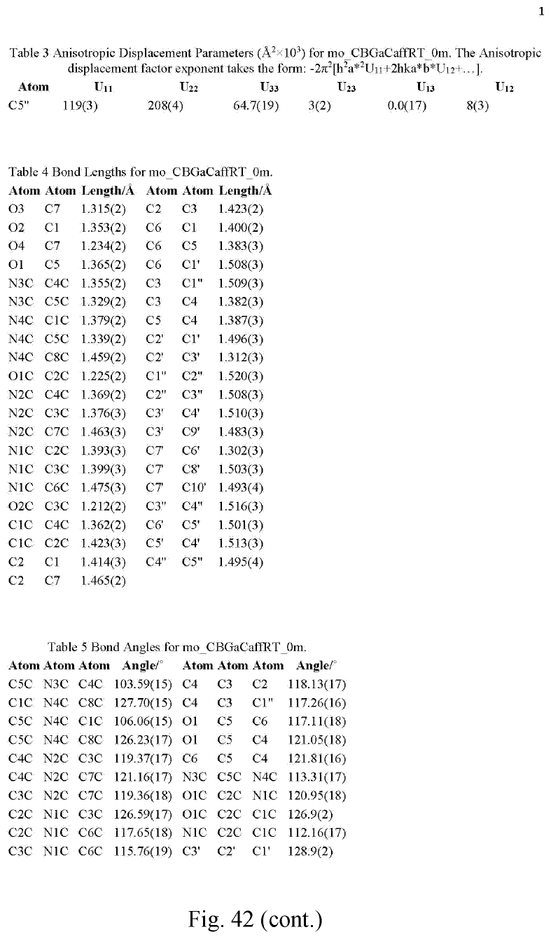

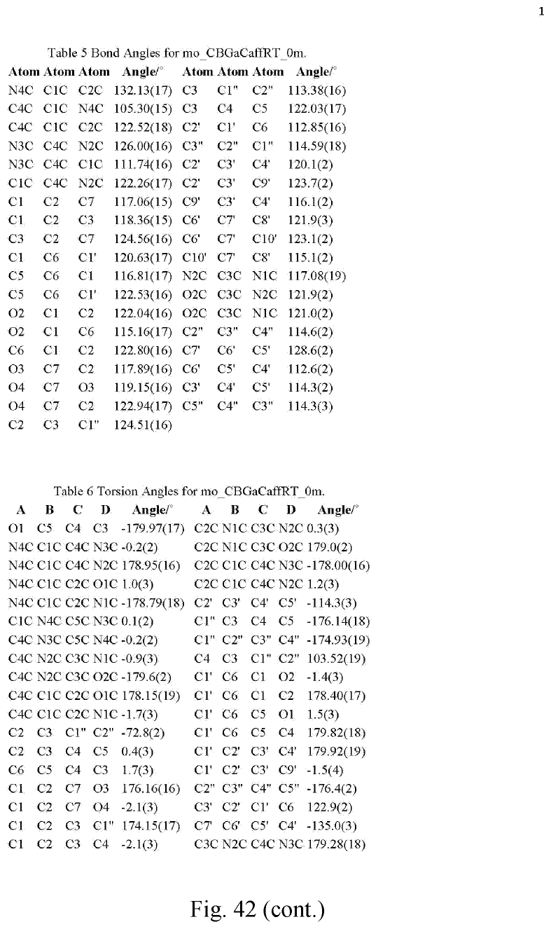

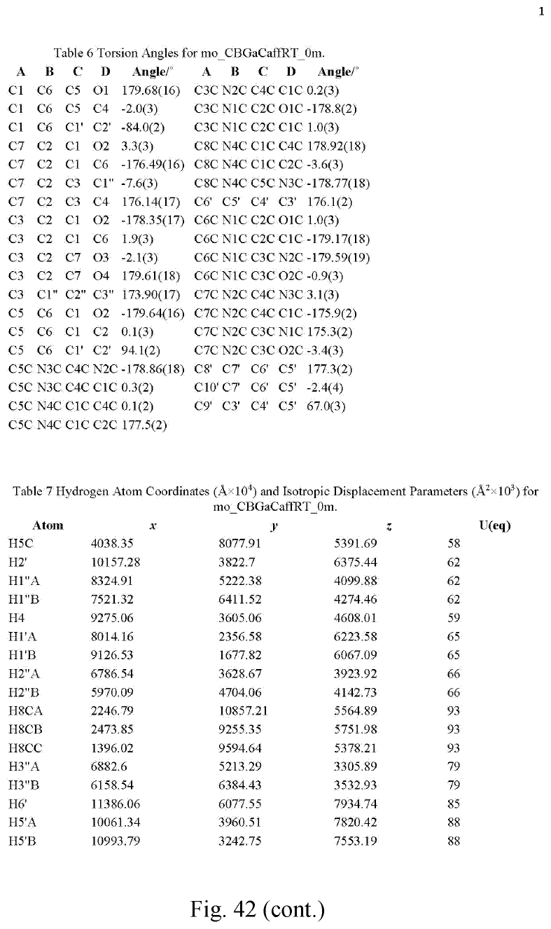

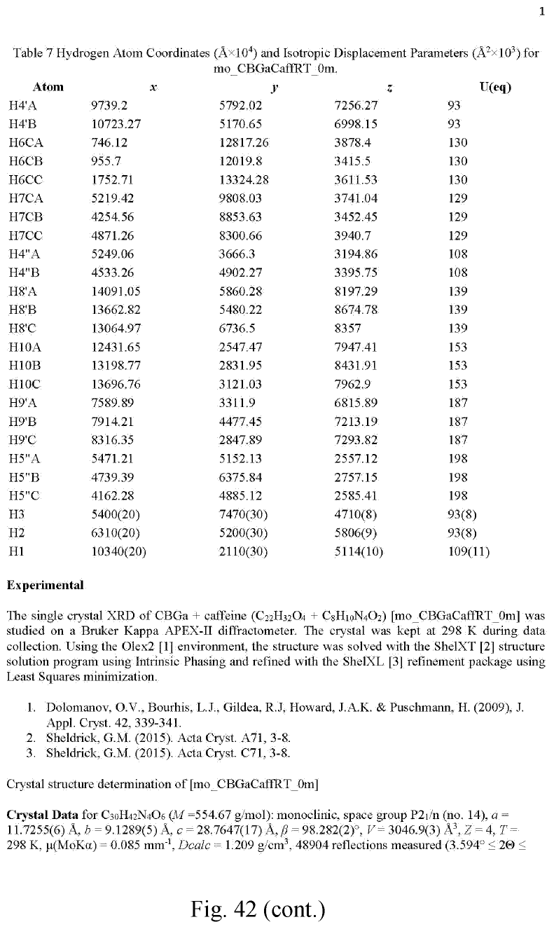

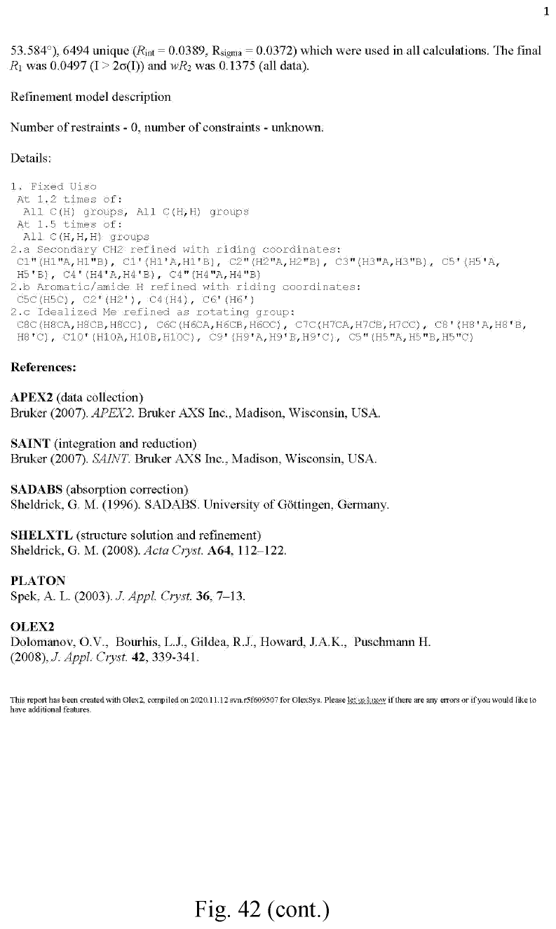

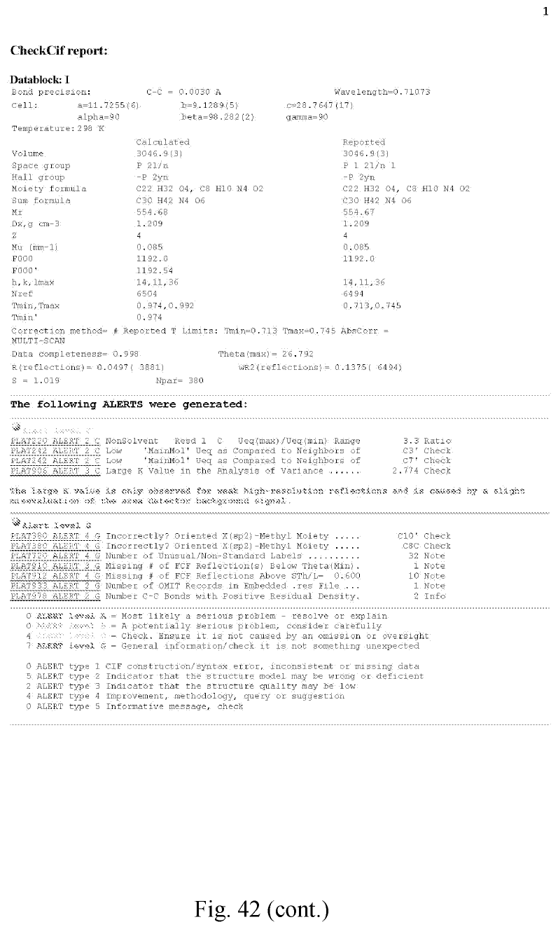

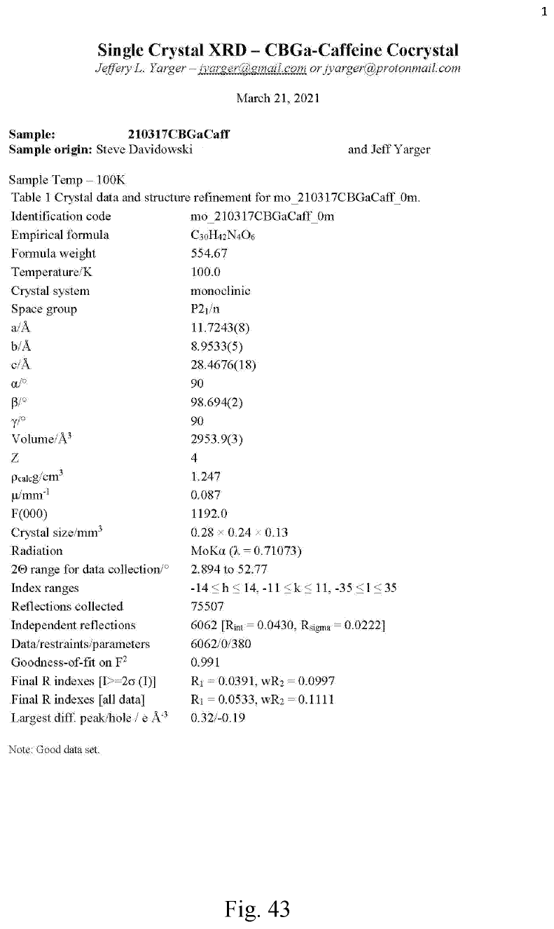

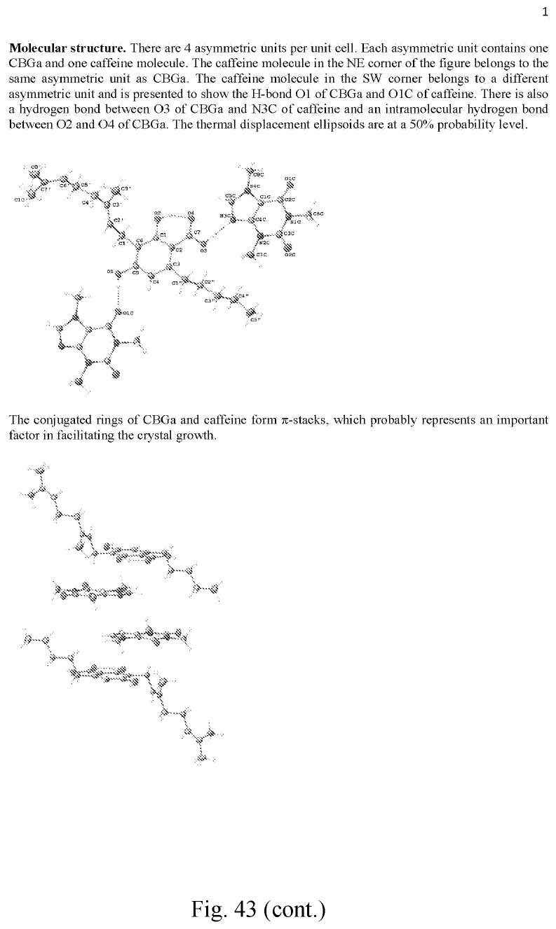

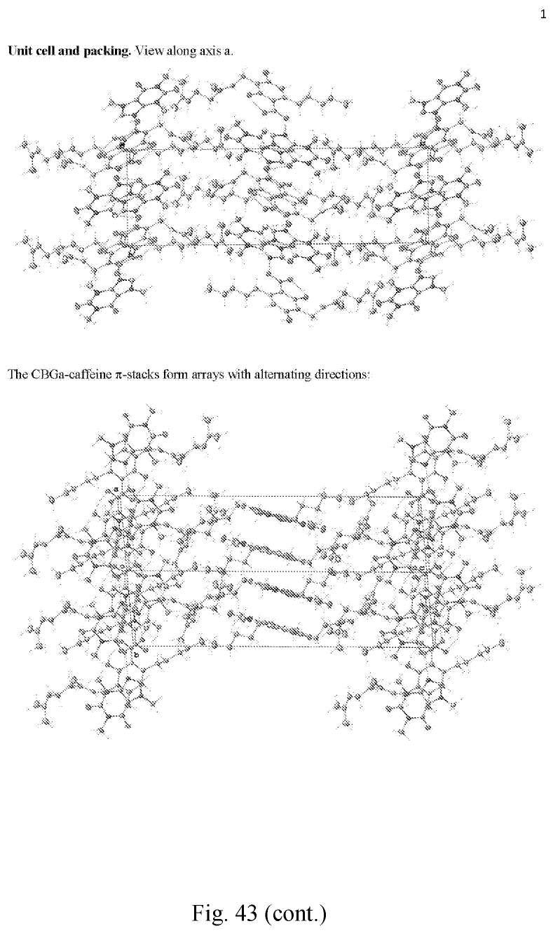

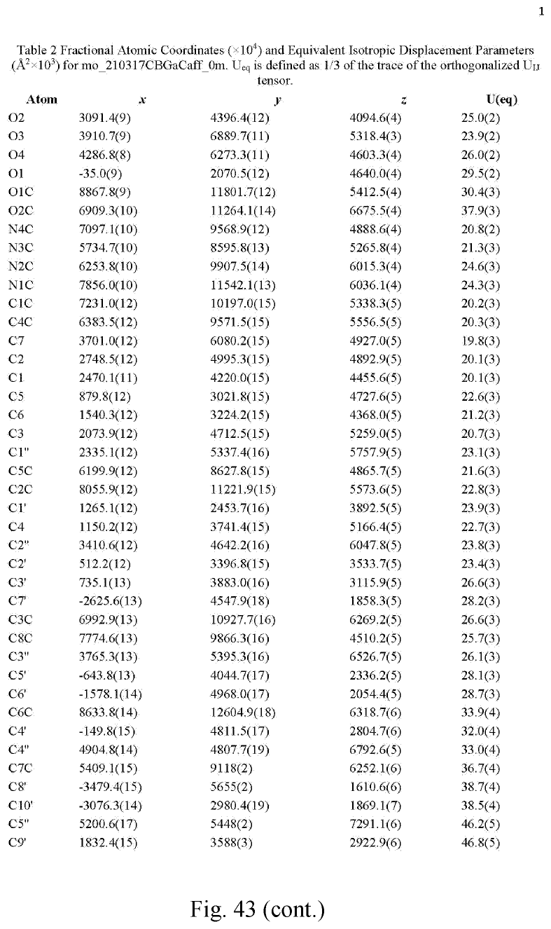

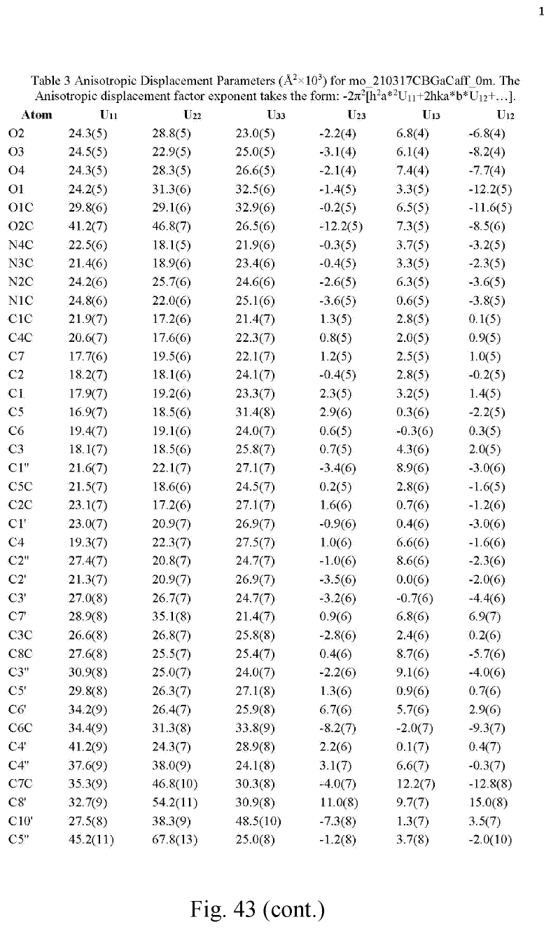

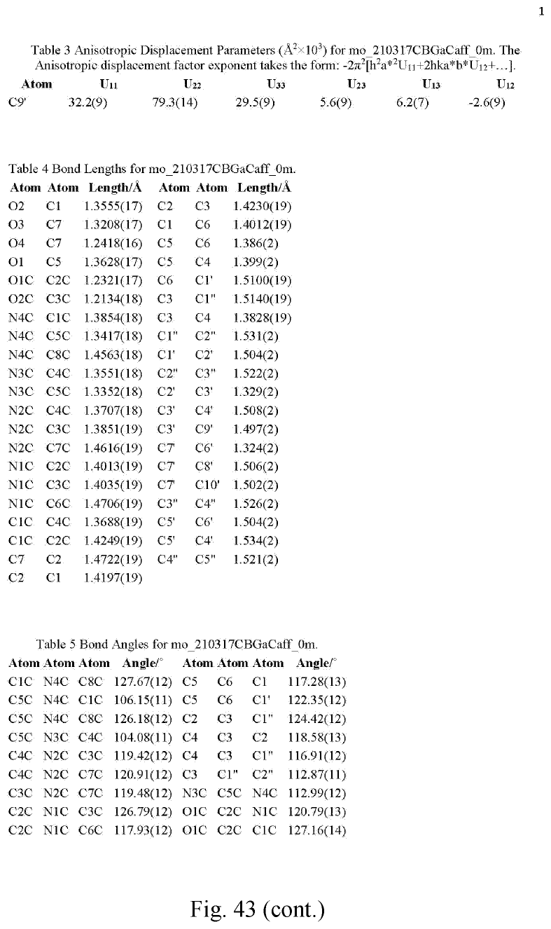

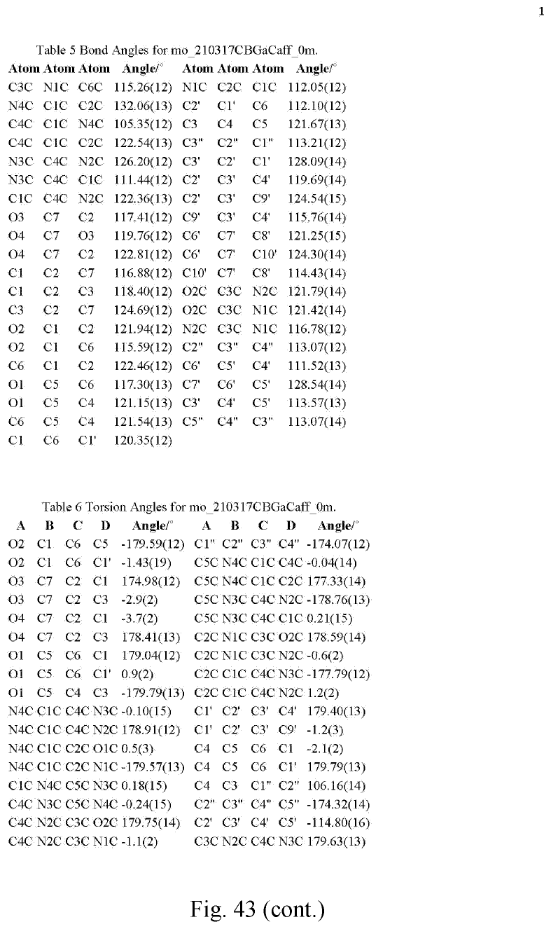

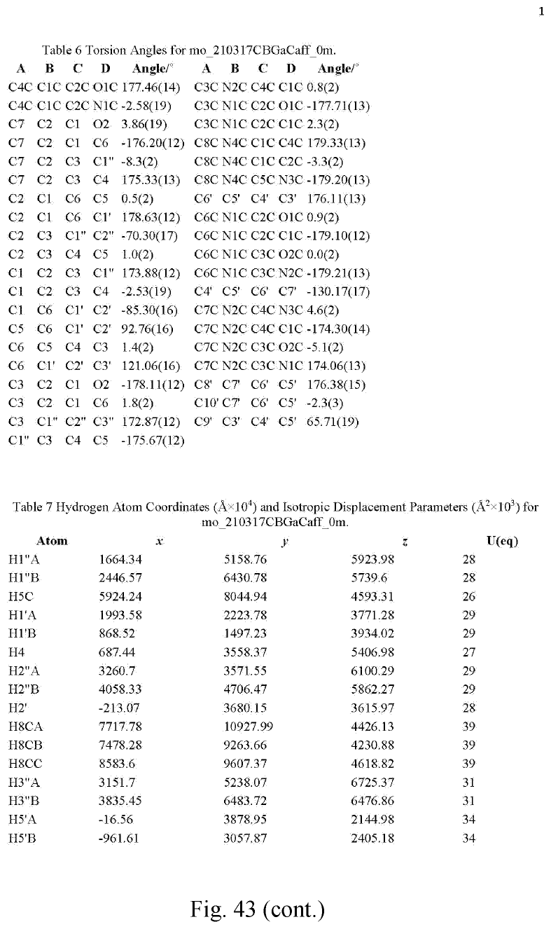

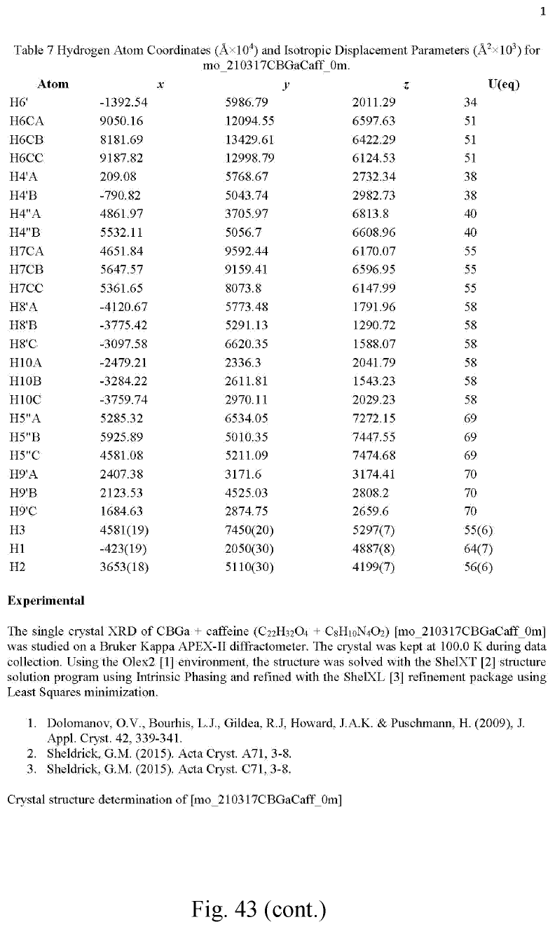

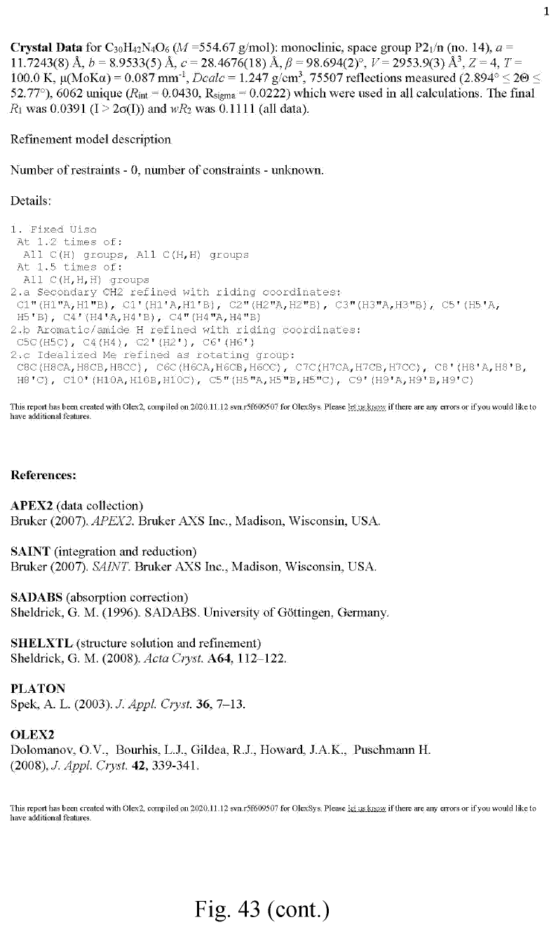

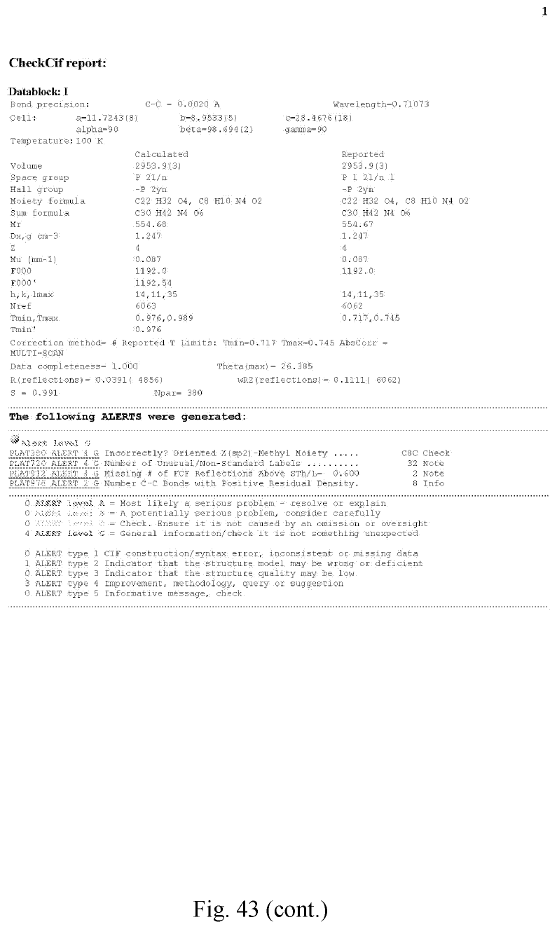

In embodiments the crystalline form of cannabidiolic acid (CBDa) can comprise a powder X-ray diffraction spectrum characterized by diffraction angle 2θ comprising at least one characteristic peak at about 8.1, 9.2, 9.9, 10.9, 12.1, 12.9, 13.3, 14.1, 15.1, 16.9, 17.7, 18.1, 18.7, 19.1, 19.8, 20.2, 20.6, 21.1, 21.8, 22.4, 23.6, 24.2, 24.8, 25.6, 26, 26.9, 27.5, 28.5, 28.7, 30.6, 33.1, 33.3, 34.3, 38.3, 39.1, and 39.7. In embodiments, the crystalline form of cannabidiolic acid (CBDa) can comprise a powder X-ray diffraction spectrum characterized by diffraction angle 2θ comprising the spectrum substantially as show in FIG. 40. In embodiments, the crystalline form of cannabidiolic acid (CBDa) can comprise a powder X-ray diffraction spectrum characterized by Table 4.

TABLE 4
CBDa pXRD result summary

| | Pos. | d-spacing | |
|---|---|---|---|
| No. | [°2θ] | [Å] | I/Io |
| 1 | 8.12 | 10.89 | 6.8 |
| 2 | 9.20 | 9.61 | 36.7 |
| 3 | 9.86 | 8.97 | 6.5 |
| 4 | 10.89 | 8.13 | 100 |
| 5 | 12.87 | 6.88 | 96.7 |
| 6 | 13.35 | 6.63 | 8.6 |
| 7 | 14.10 | 6.28 | 9.8 |
| 8 | 15.17 | 5.84 | 13.0 |
| 9 | 17.72 | 5.01 | 7.8 |
| 10 | 18.03 | 4.92 | 23.4 |
| 11 | 18.72 | 4.74 | 7.5 |
| 12 | 19.13 | 4.64 | 4.7 |
| 13 | 20.18 | 4.40 | 38.0 |
| 14 | 20.61 | 4.31 | 26.4 |
| 15 | 21.14 | 4.20 | 11.5 |
| 16 | 21.81 | 4.07 | 70.7 |
| 17 | 22.43 | 3.96 | 5.9 |
| 18 | 24.85 | 3.58 | 8.4 |
| 19 | 25.59 | 3.48 | 24.4 |
| 20 | 26.06 | 3.42 | 5.9 |

### Single Crystal X-Ray Diffraction Preparation and Experimental Conditions

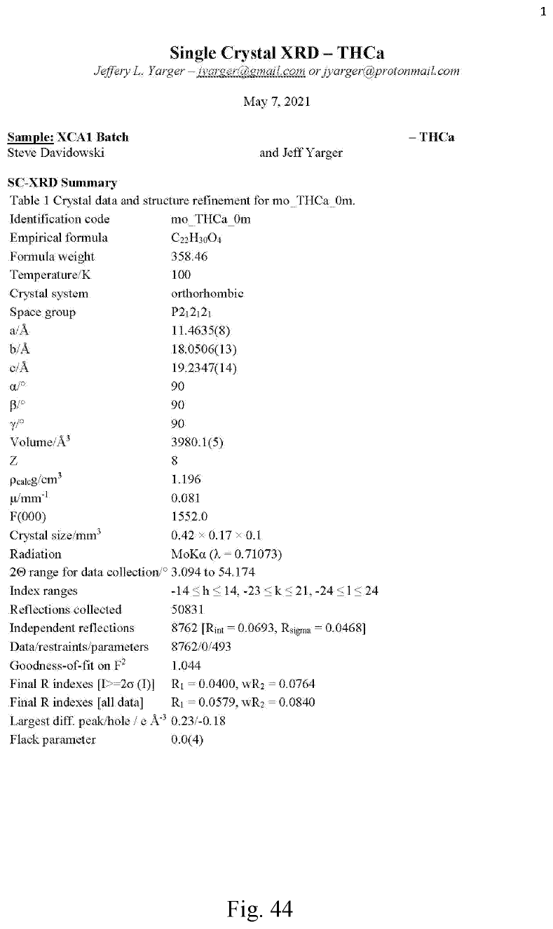

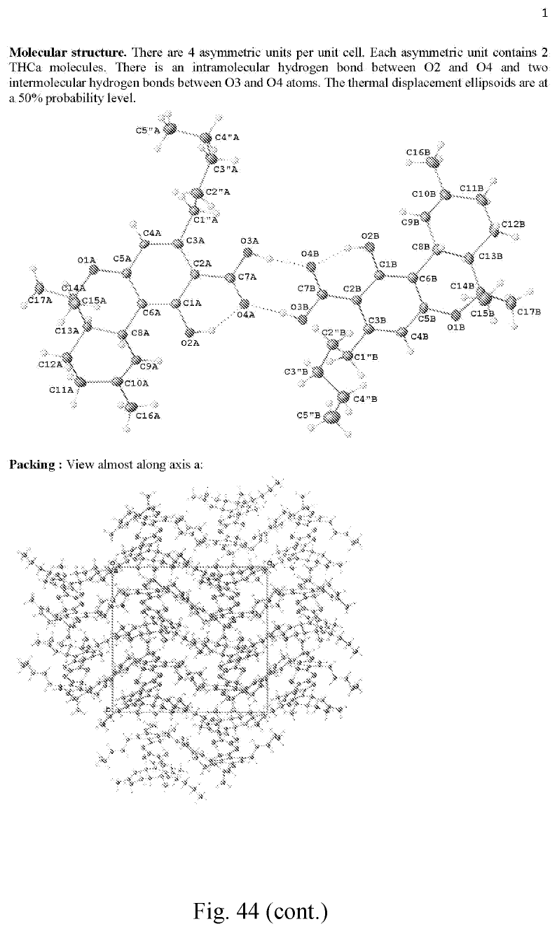

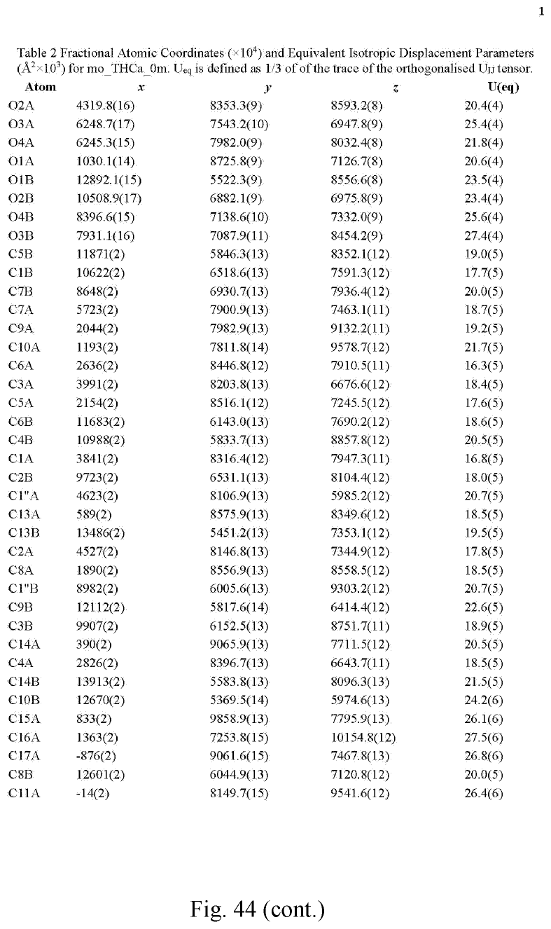

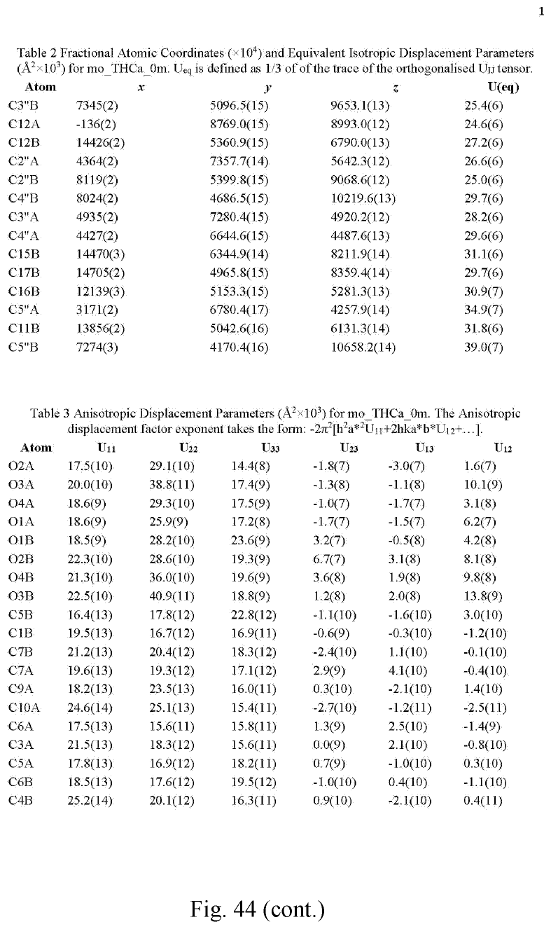

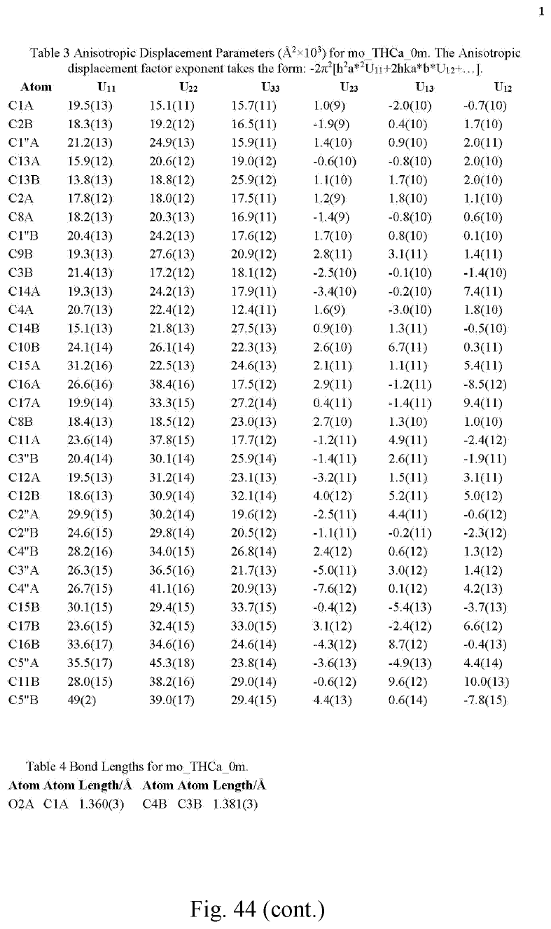

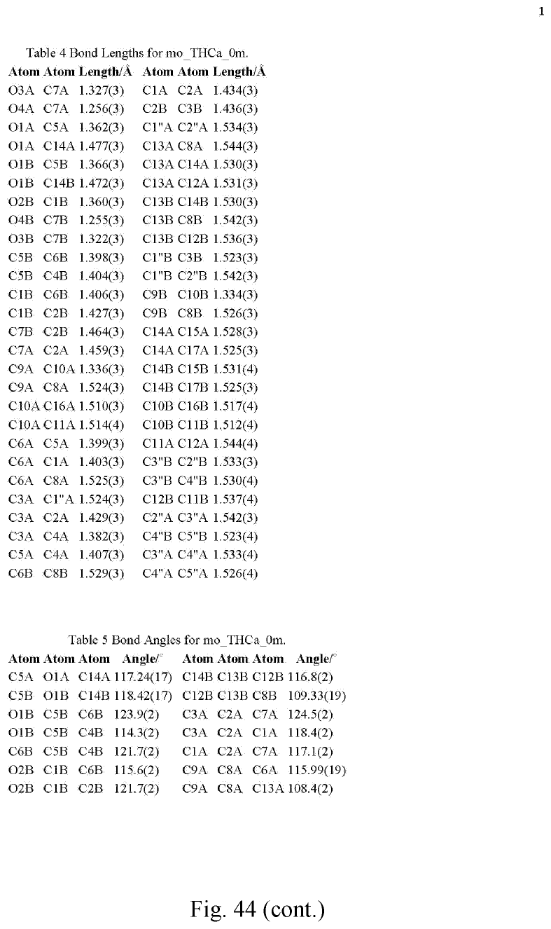

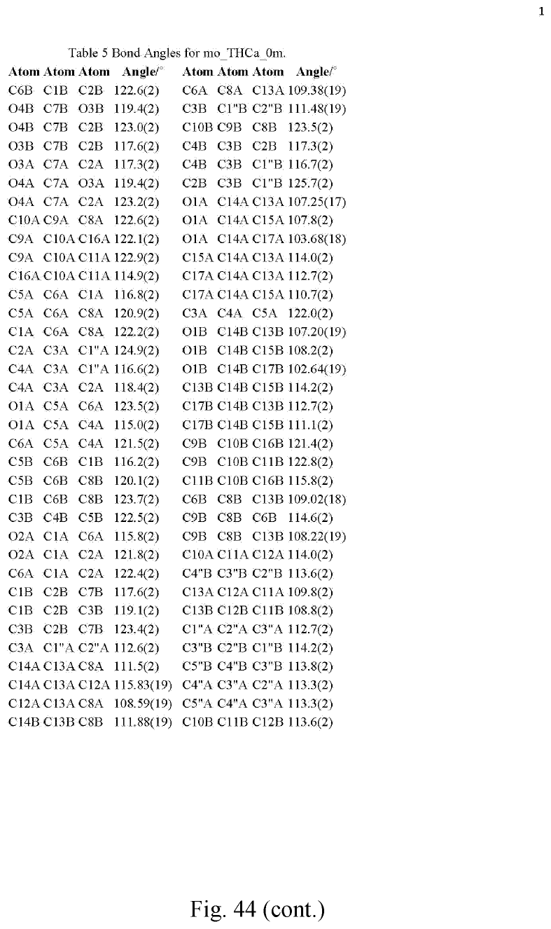

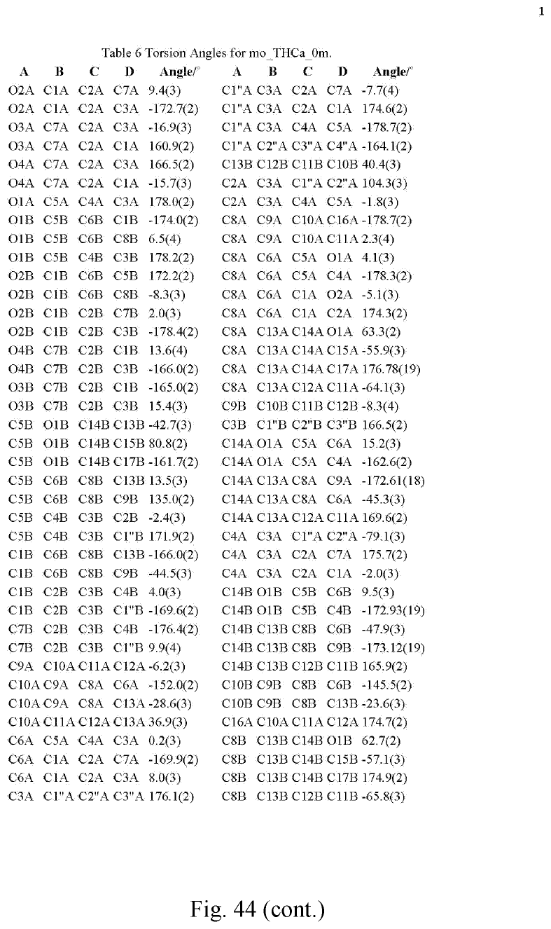

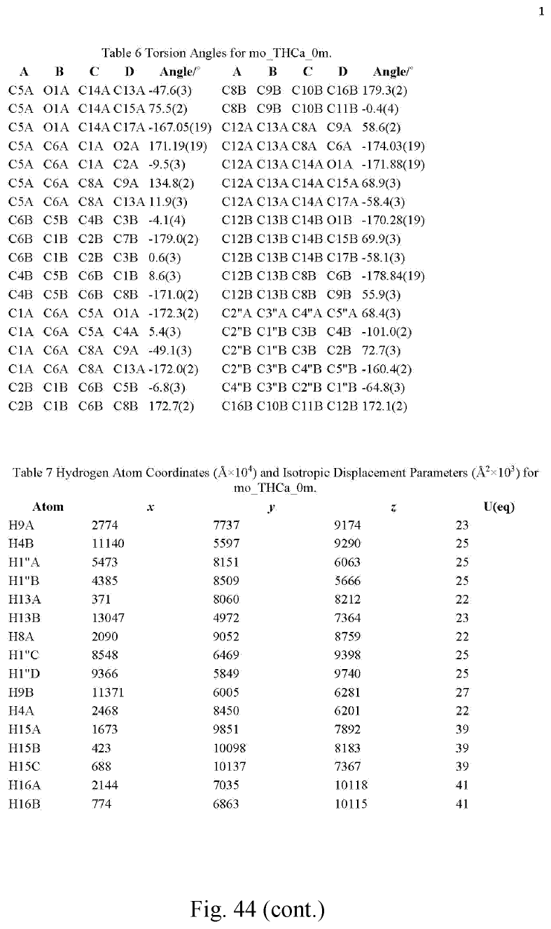

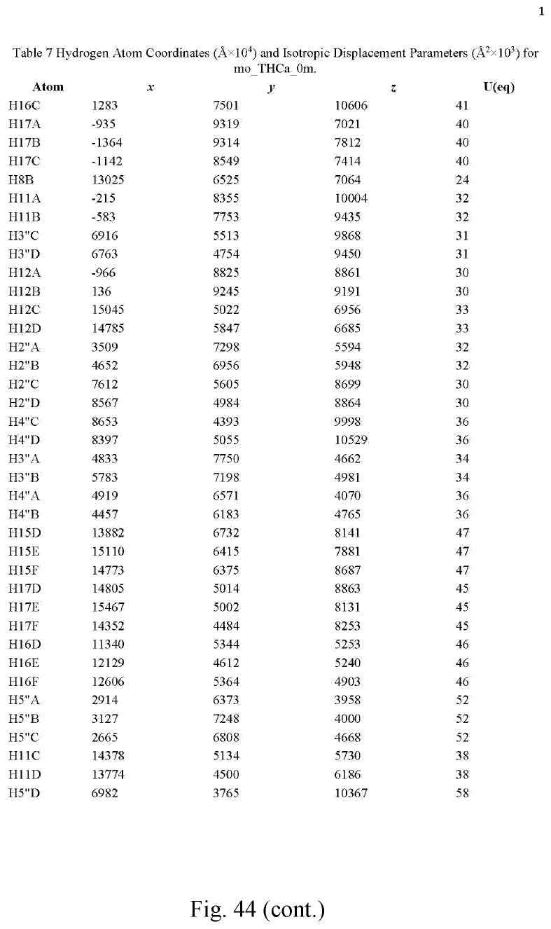

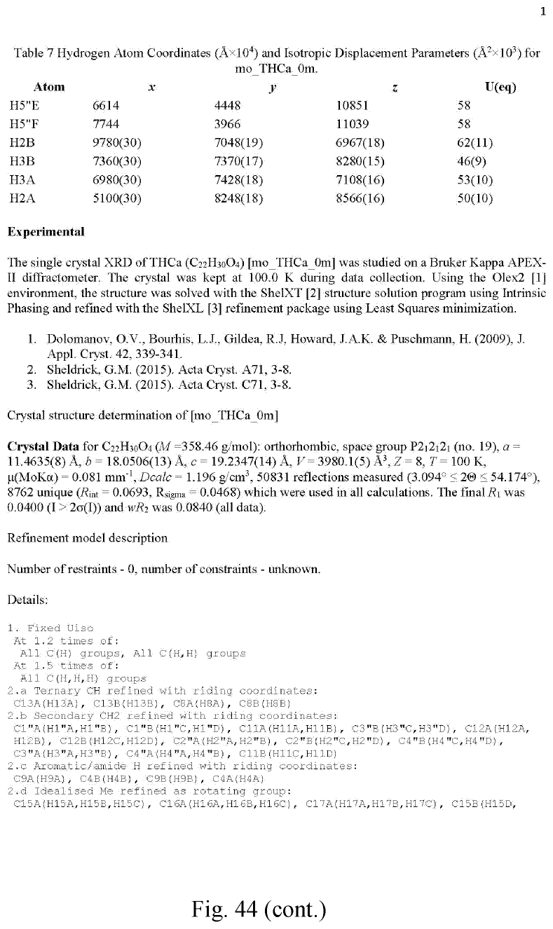

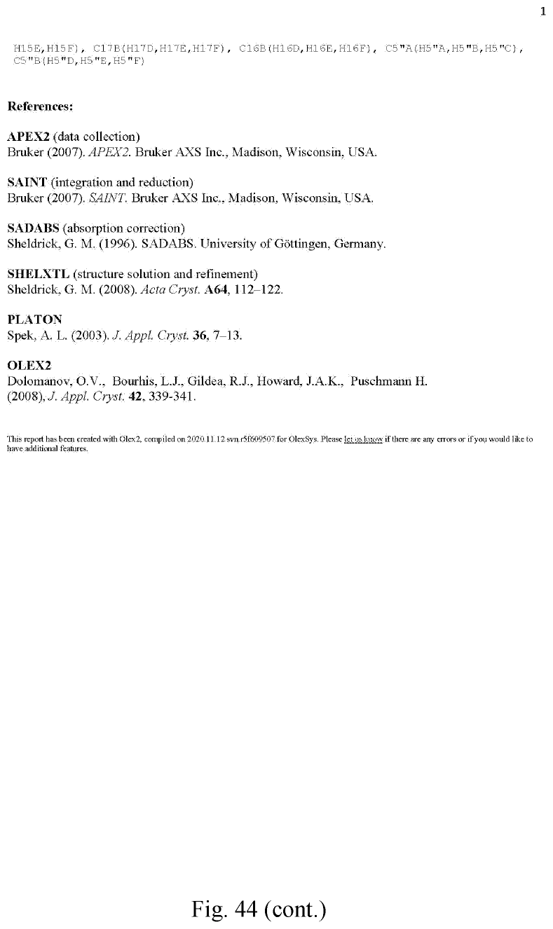

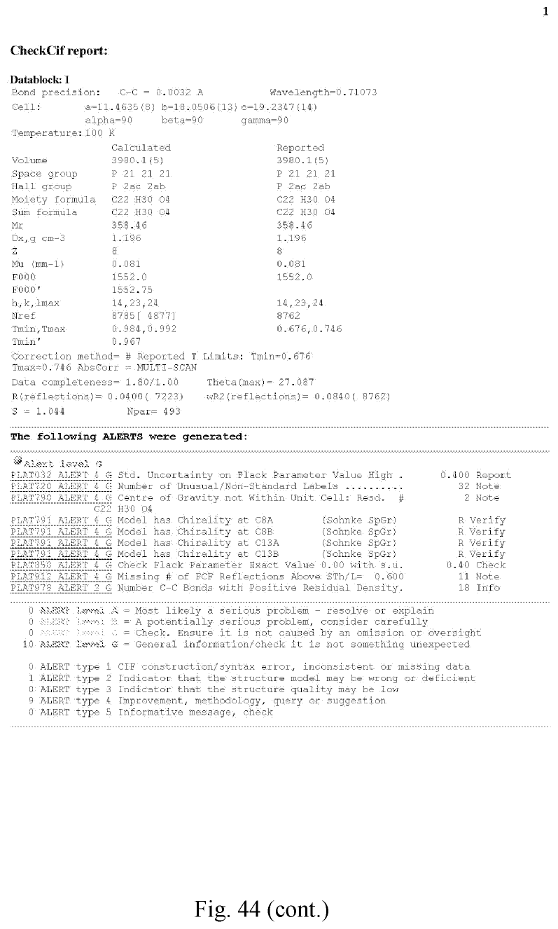

In embodiments the crystalline form of tetrahydrocannabinolic acid (THCa) can comprise a single crystal X-ray diffraction (SRD) spectrum characterized by diffraction angle 2θ comprising at least one characteristic peak as described herein. In embodiments, the crystalline form of tetrahydrocannabinolic acid (THCa) can comprise a single crystal X-ray diffraction spectrum characterized by diffraction angle 2θ comprising the spectrum substantially as show in FIG. 44.

In embodiments the single crystal XRD can be used to determine crystal data and crystal structure.

In embodiments, the crystalline form of THCa can be characterized by the data substantially as show in Table 1.

### Crystalline Form THCa

TABLE 1
Crystal data and XRD structure refinement for THCa.

| Empirical formula | C₂₂H₃₀O₄ | |
|---|---|---|
| Formula weight | 358.46 | |
| Temperature/K | 100 | |
| Crystal system | orthorhombic | |
| Space group | P2₁2₁2₁ | |
| a/Å | 11.4635 | (8) |
| b/Å | 18.0506 | (13) |
| c/Å | 19.2347 | (14) |
| α/° | 90 | |
| β/° | 90 | |
| γ/° | 90 | |
| Volume/Å³ | 3980.1 | (5) |
| Z | 8 | |
| ρ_{calc}g/cm³ | 1.196 | |
| μ/mm⁻¹ | 0.081 | |
| F(000) | 1552.0 | |
| Crystal size/mm³ | 0.42 × 0.17 × 0.1 | |
| Radiation | MoKα (λ = 0.71073) | |
| 2Θ range for data collection/° | 3.094 to 54.174 | |
| Index ranges | −l4 ≤ h ≤ l4, −23 ≤ | |
| | k ≤ 2l, −2 ≤ 24 | |
| Reflections collected | 50831 | |
| Independent reflections | 8762 [Rᵢₙₜ = 0.0693, R_{sigma} = 0.0468] | |
| Data/restraints/parameters | 8762/0/493 | |
| Goodness-of-fit on F² | 1.044 | |
| Final R indexes [I >= 2σ (I)] | R₁ = 0.0400, wR₂ = 0.0764 | |
| Final R indexes [all data] | R₁ = 0.0579, wR₂ = 0.0840 | |
| Largest diff. peak/hole/e Å⁻³ | 0.23/−0.18 | |
| Flack parameter | 0.0 | (4) |

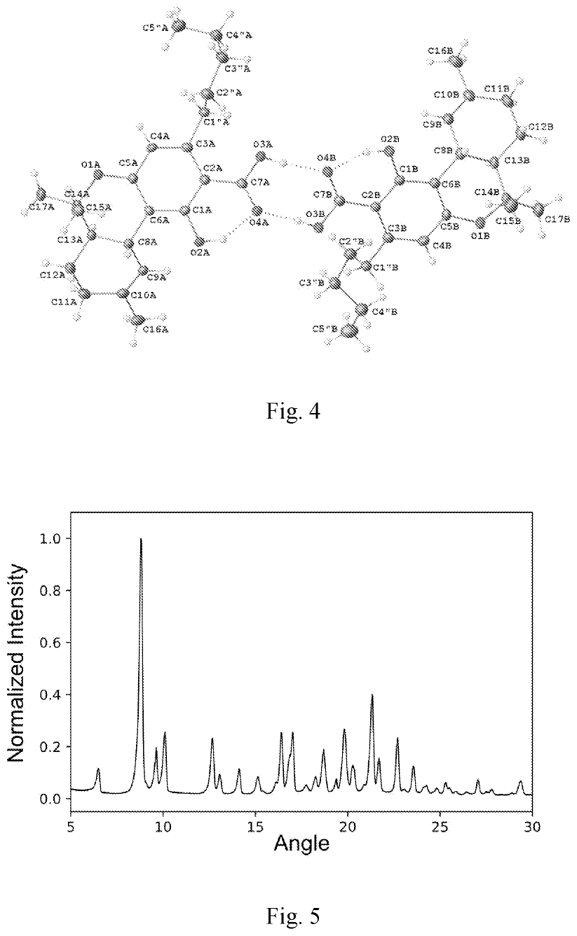

In embodiments, an Oak Ridge Thermal Ellipsoid Plot (ORTEP) ball-and-stick type illustration can be determined by SCXRD. In embodiments, the crystalline form of THCa can be characterized by the ORTEP illustration substantially as show in FIG. 4.

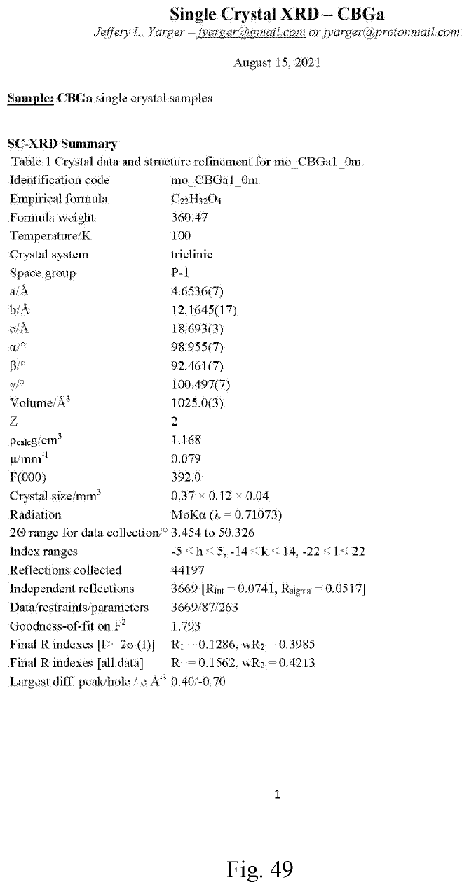

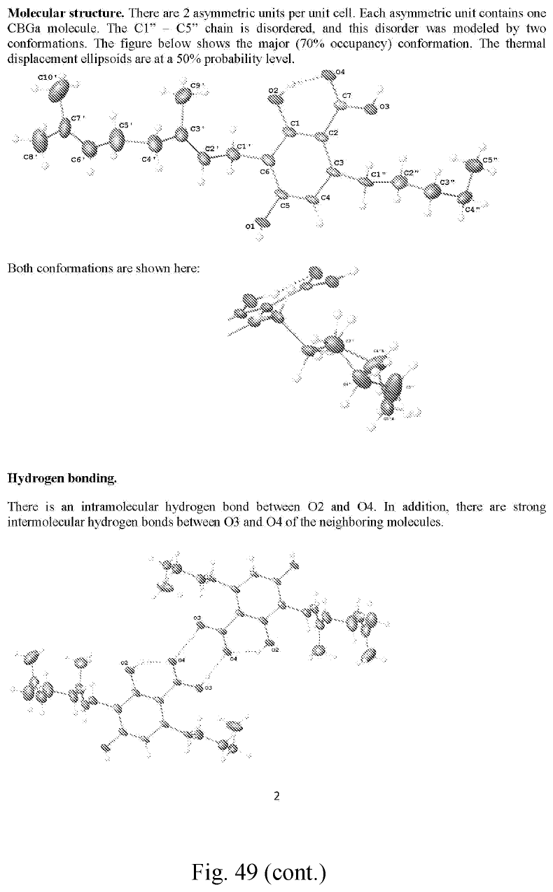

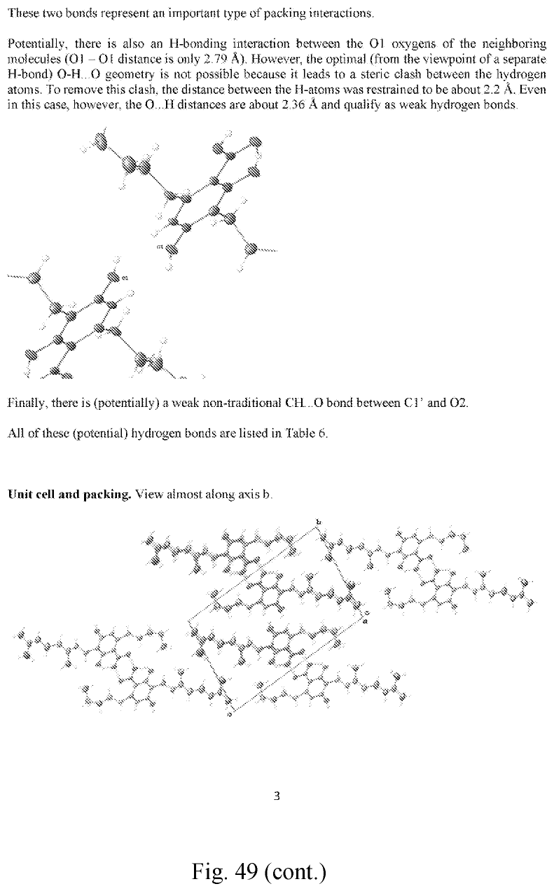

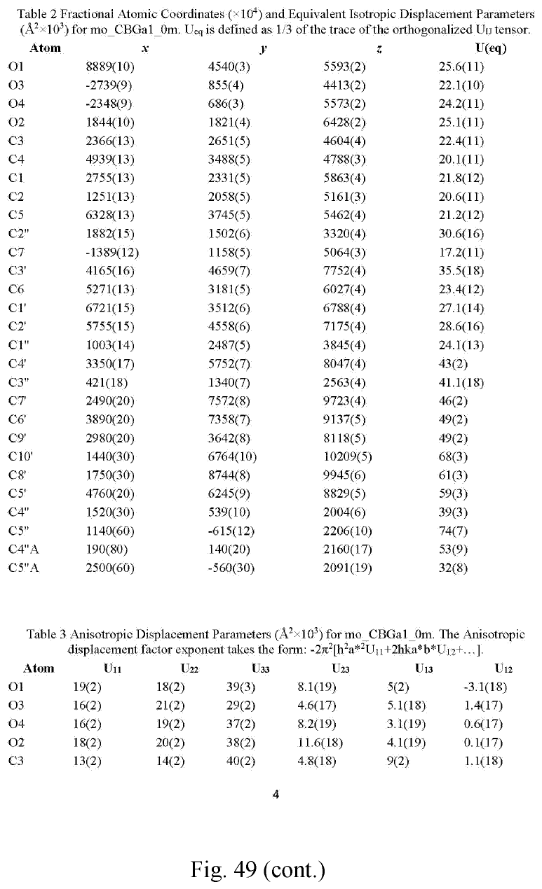

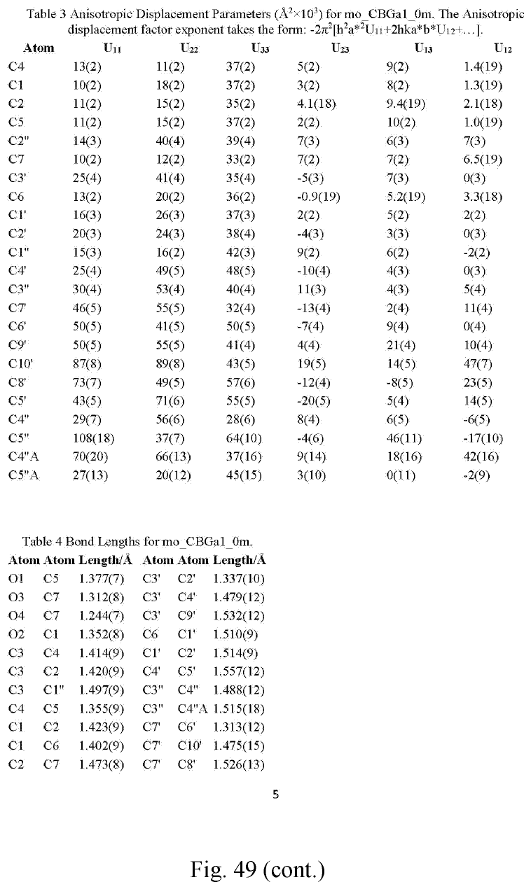

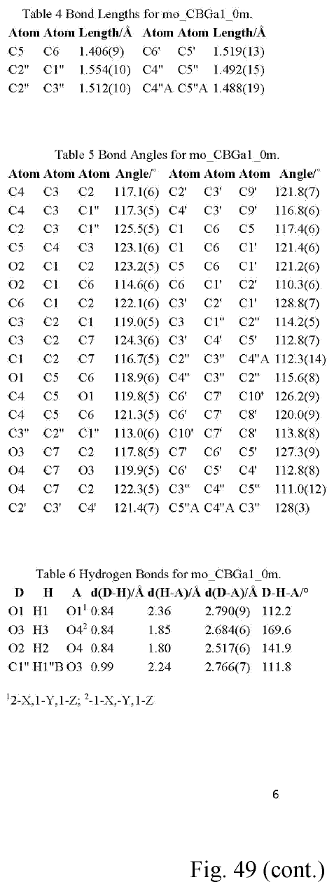

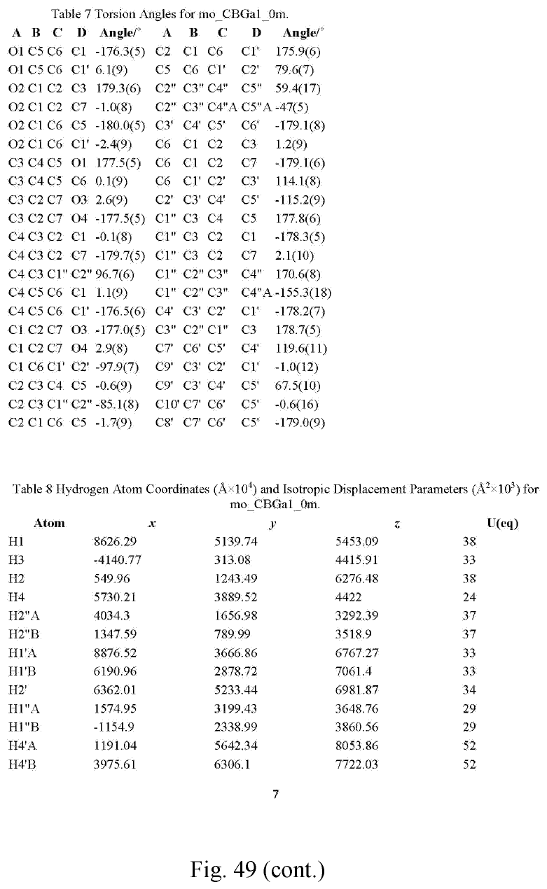

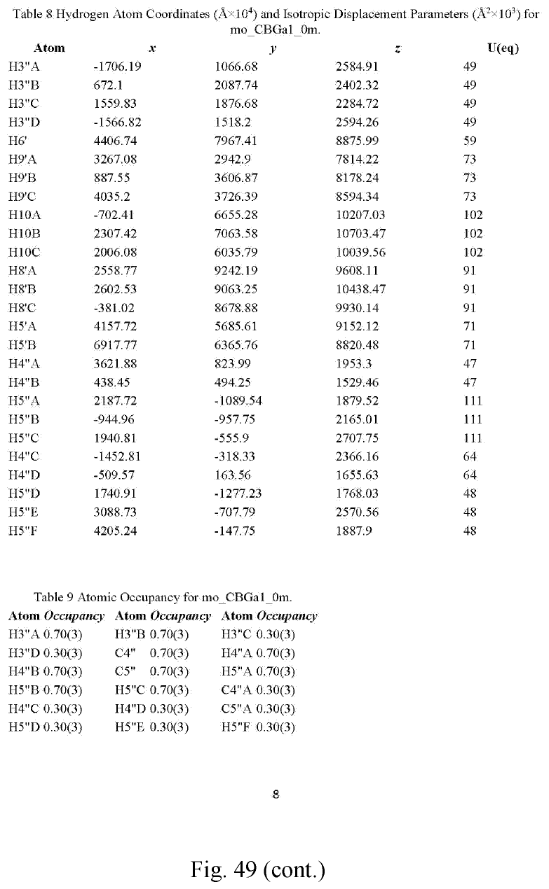

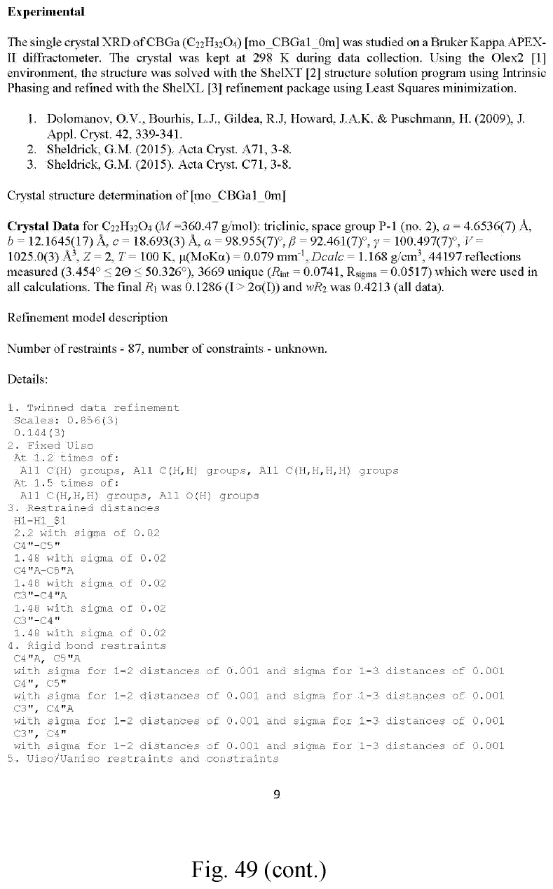

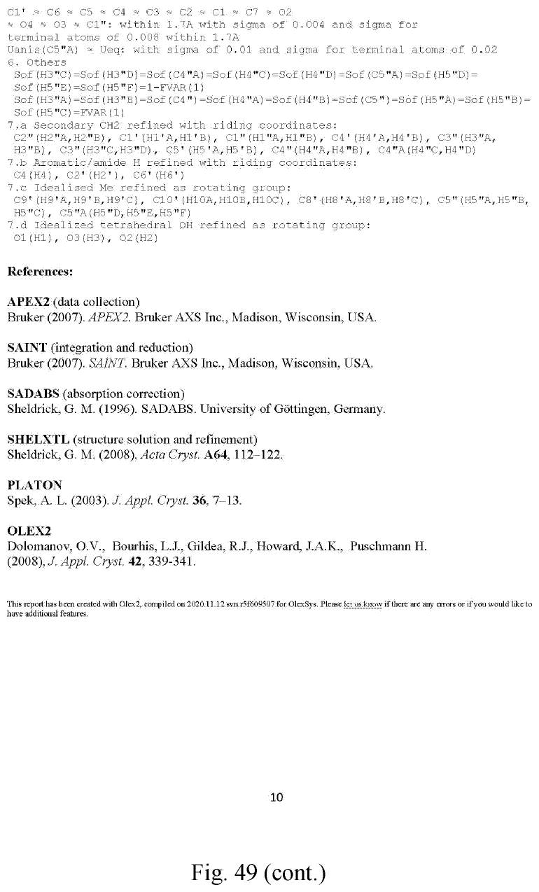

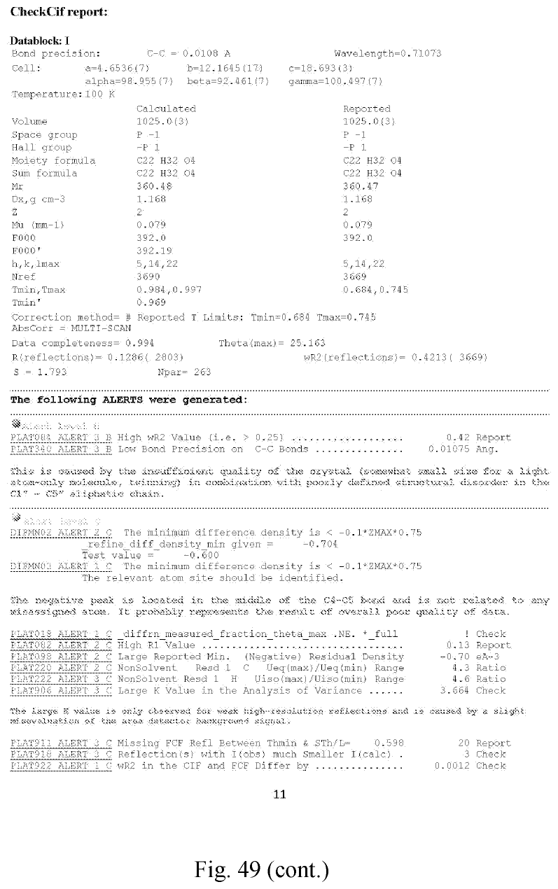

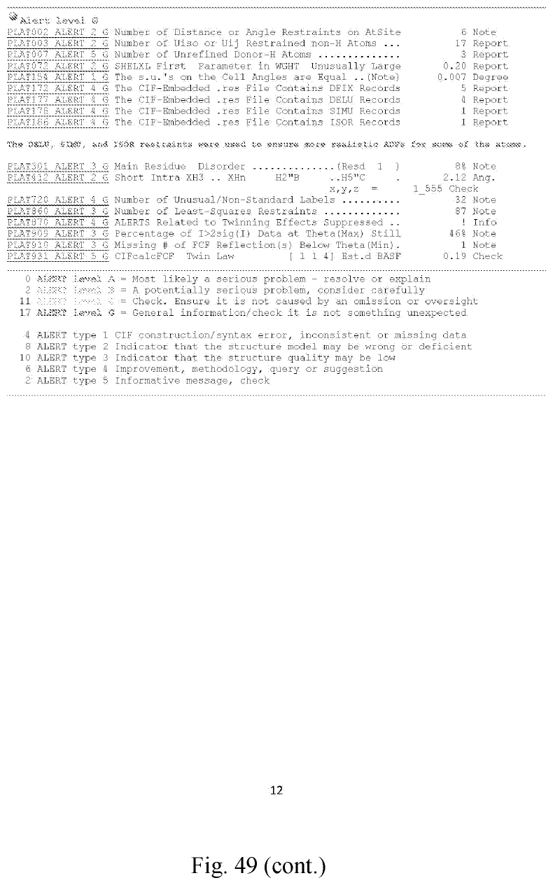

In embodiments the crystalline form of cannabigerolic acid (CBGa) can comprise a single crystal X-ray diffraction spectrum characterized by diffraction angle 2θ comprising at least one characteristic peak as described herein. In embodiments, the crystalline form of cannabigerolic acid (CBGa) can comprise a single crystal X-ray diffraction spectrum characterized by diffraction angle 2θ comprising the spectrum substantially as show in FIG. 49.

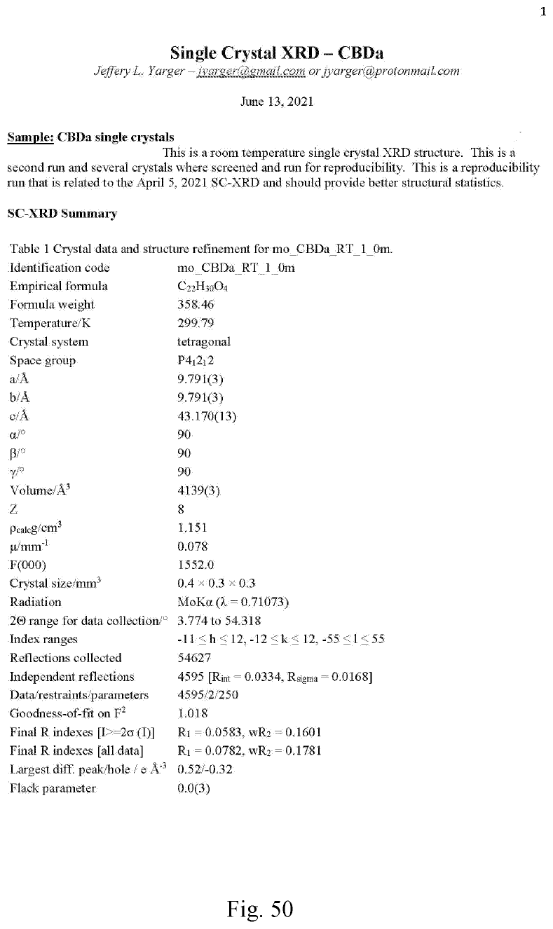

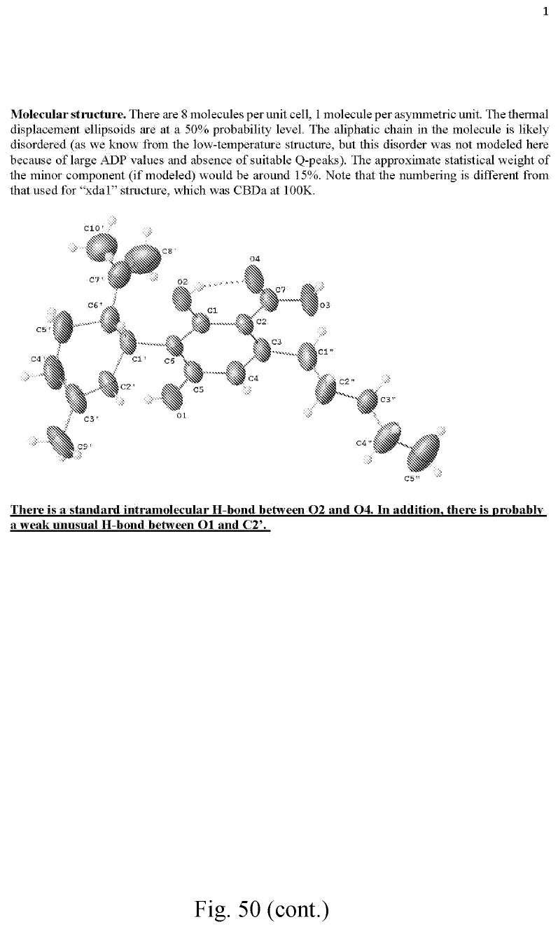

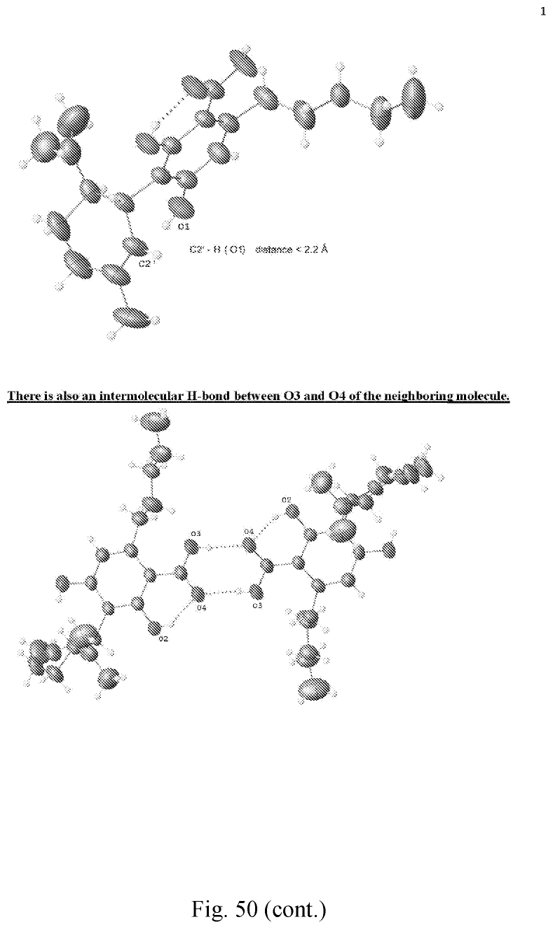

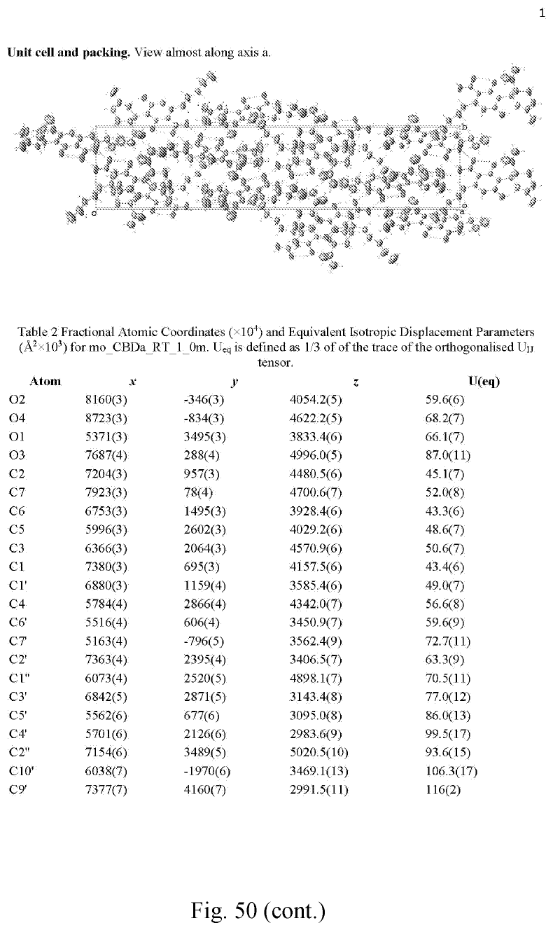

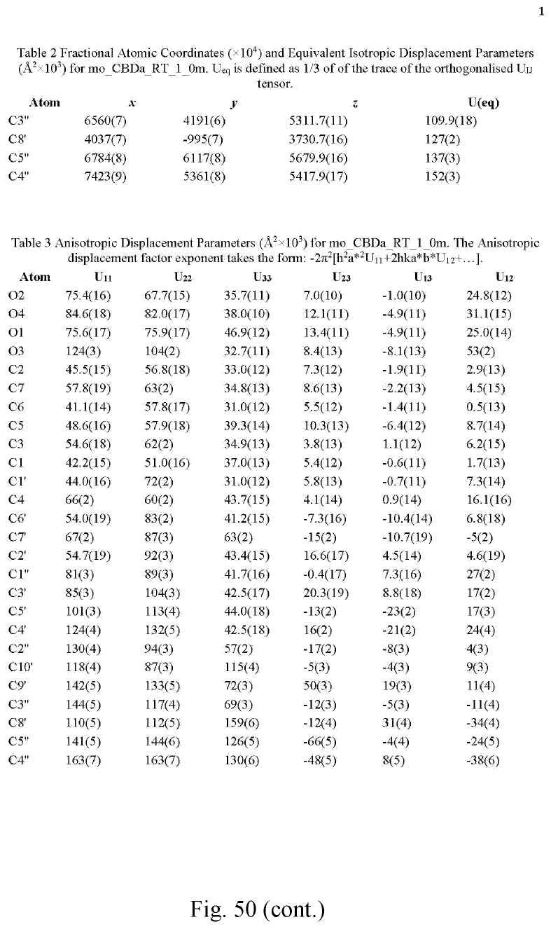

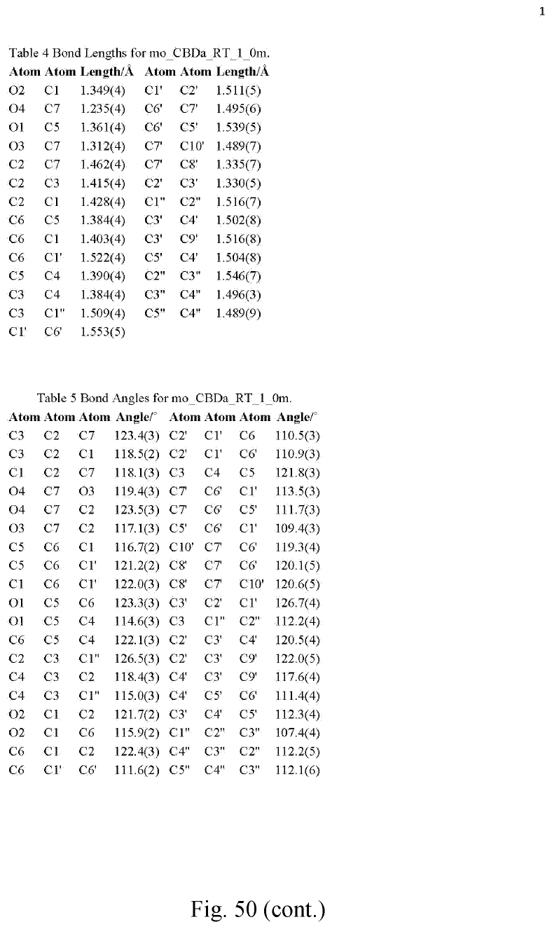

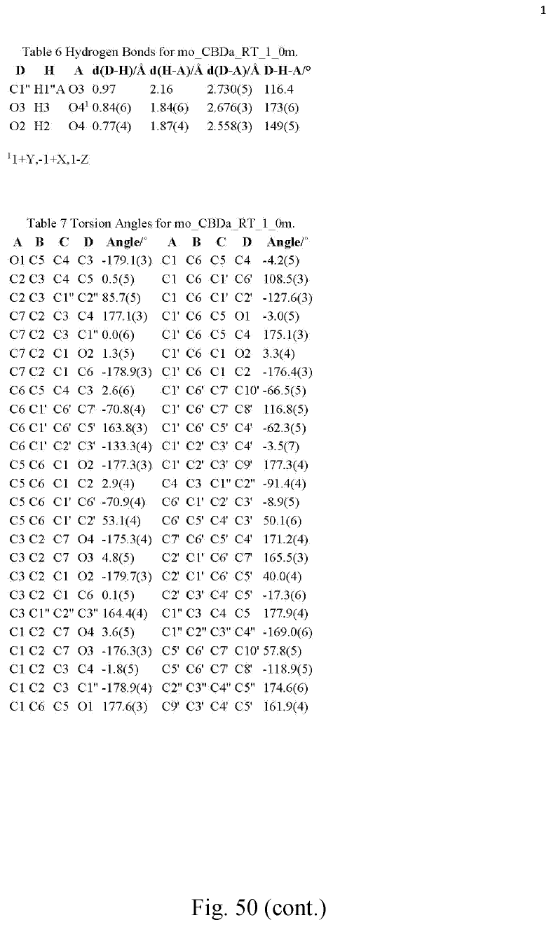

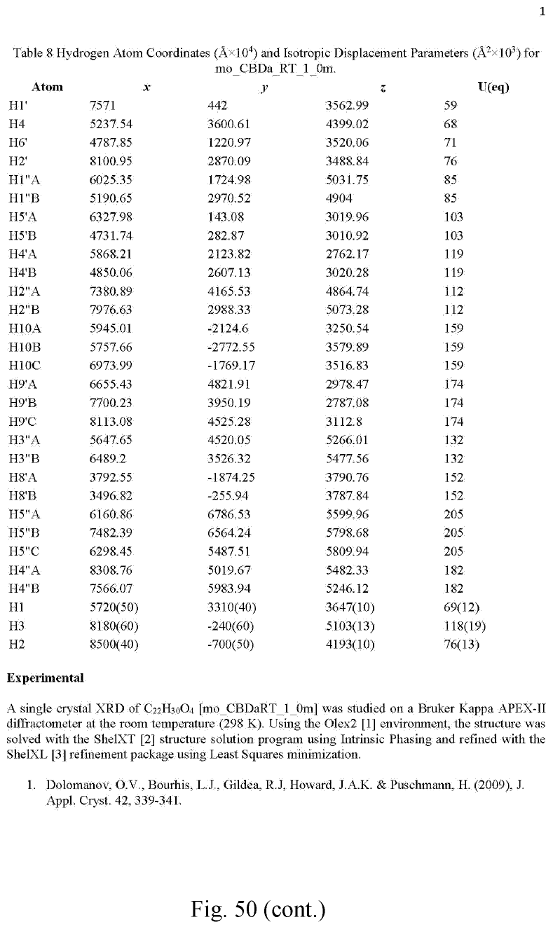

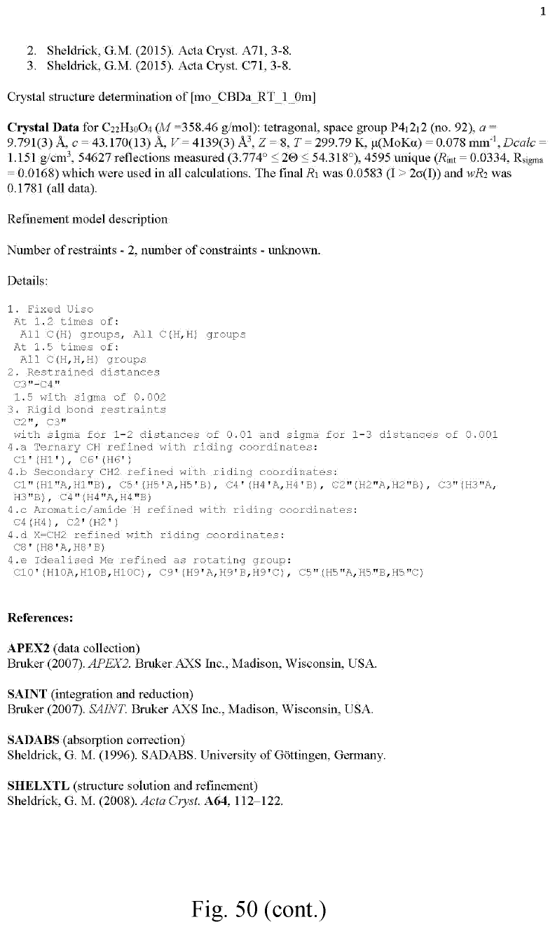

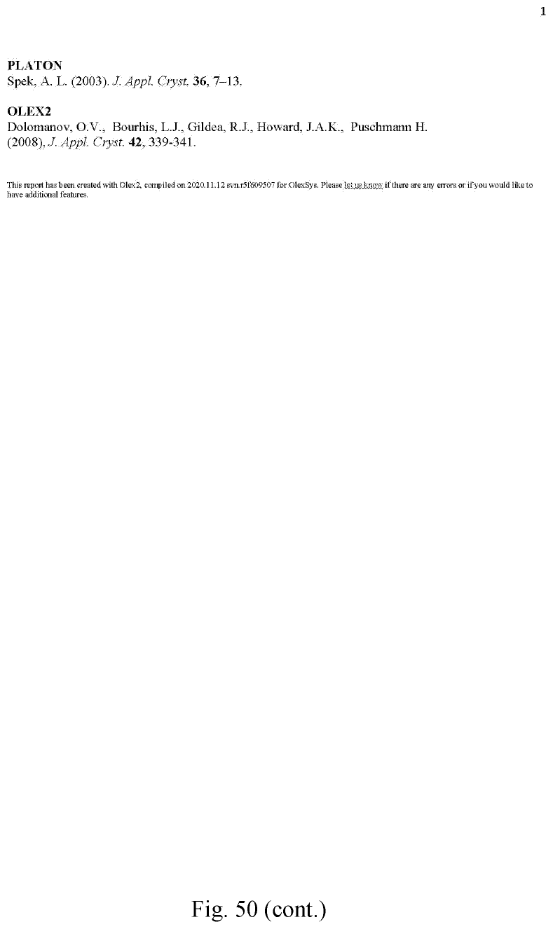

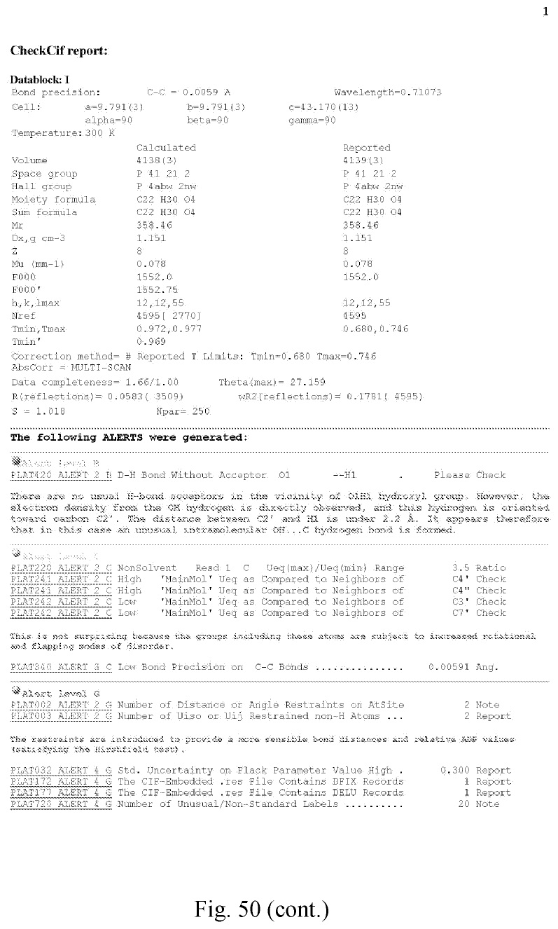

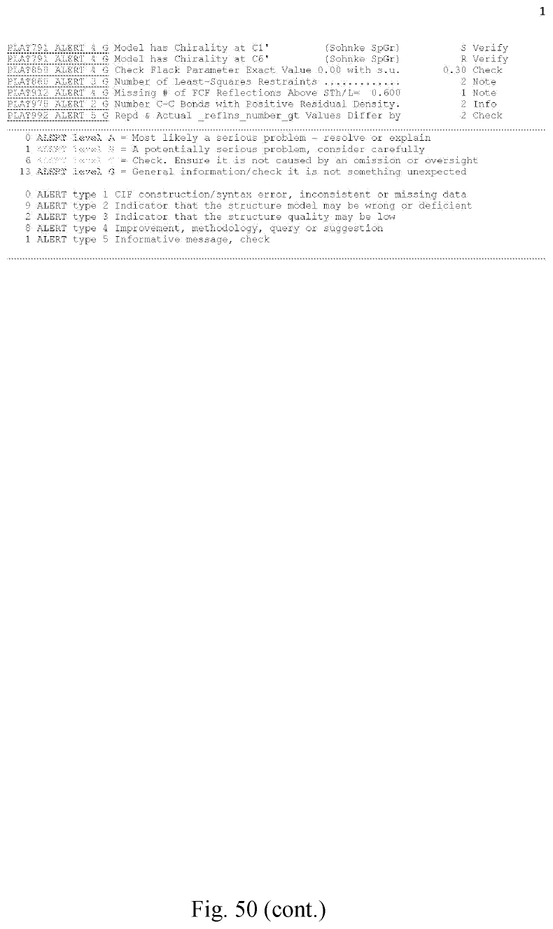

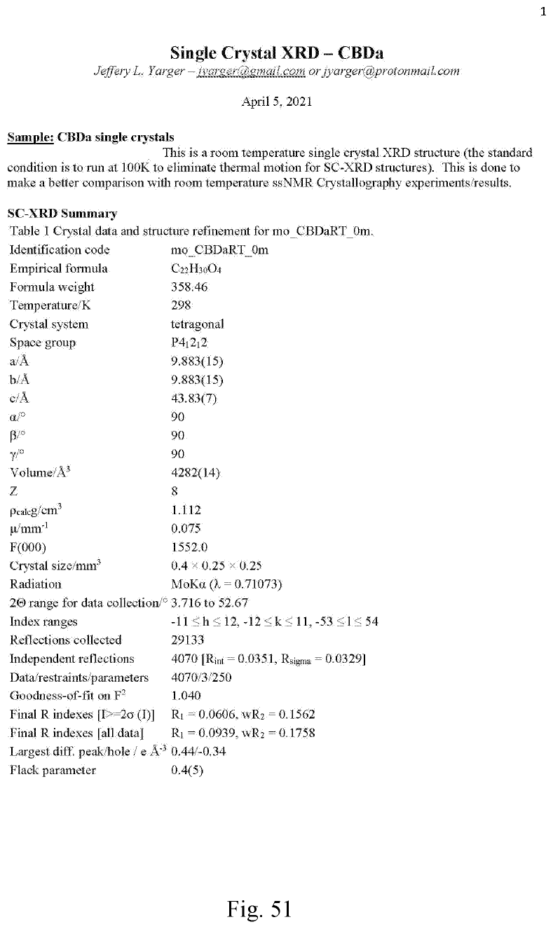

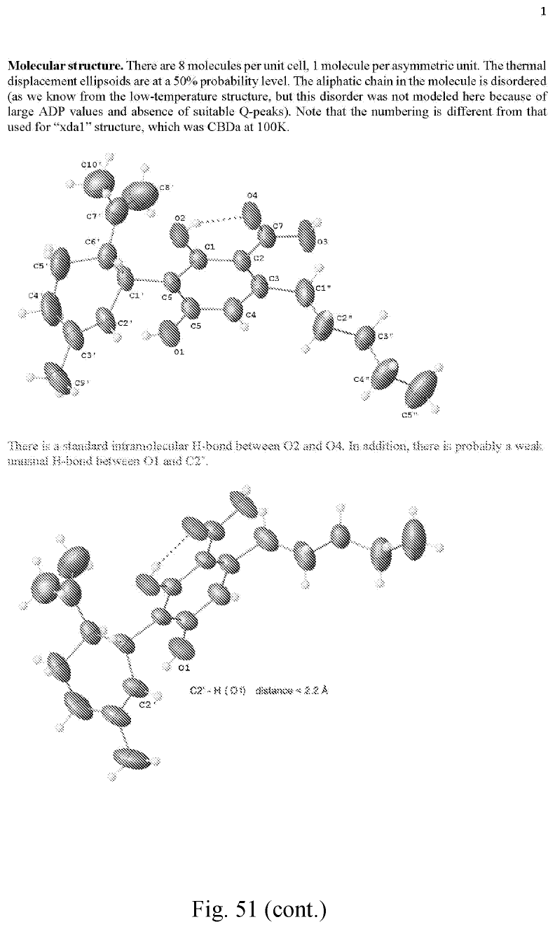

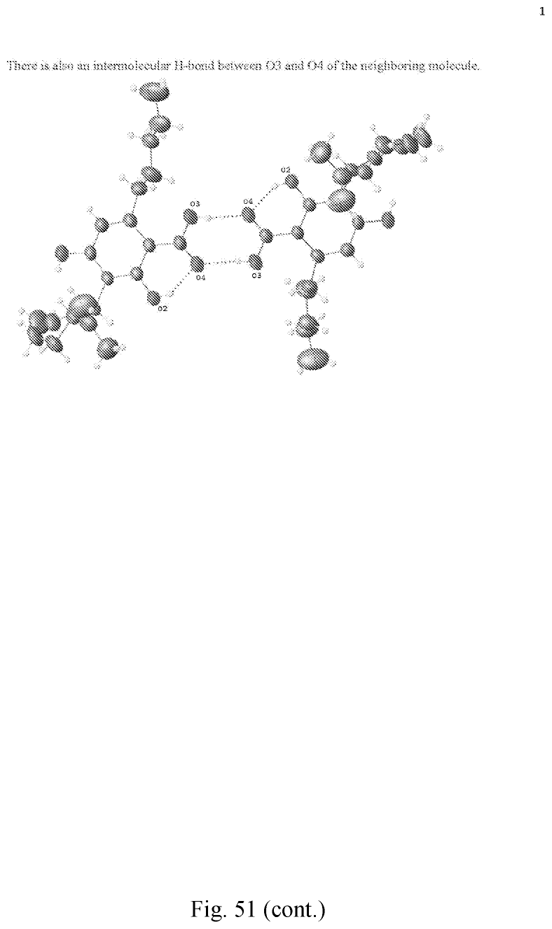

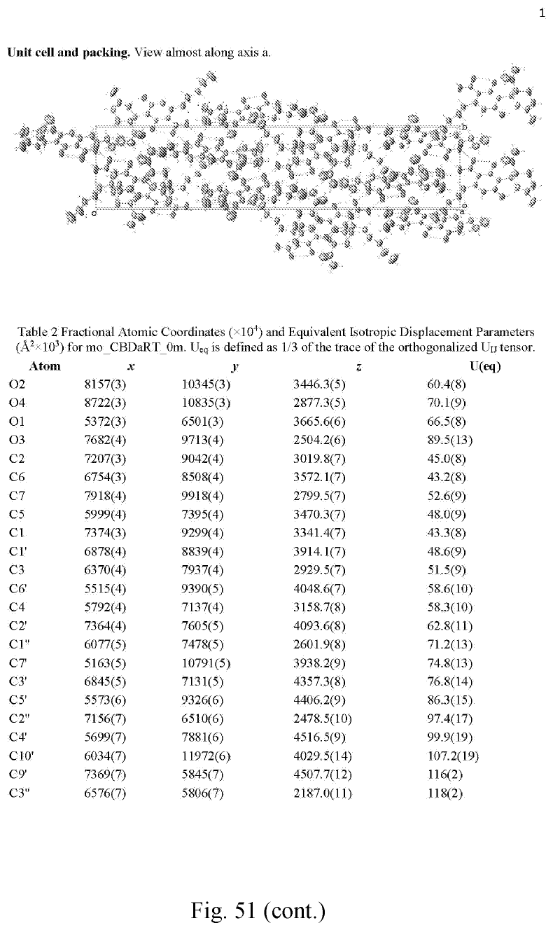

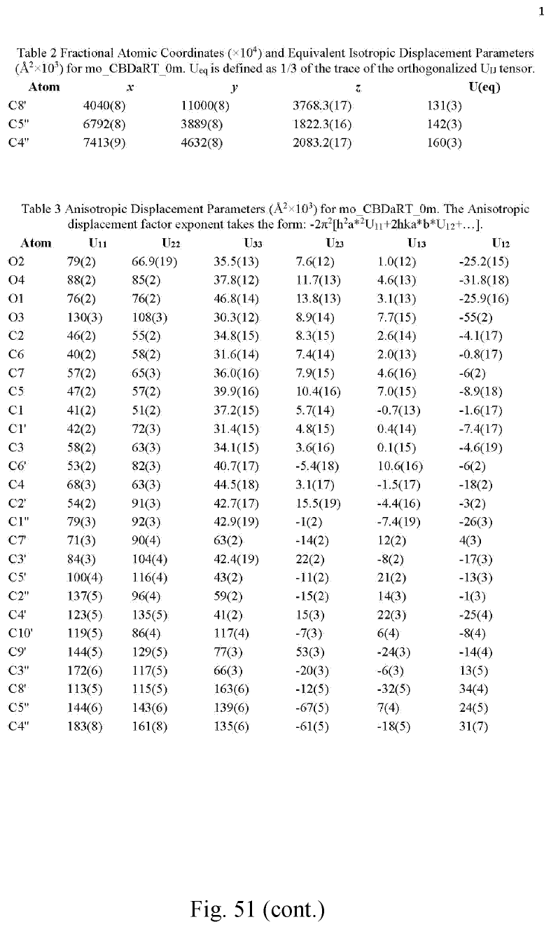

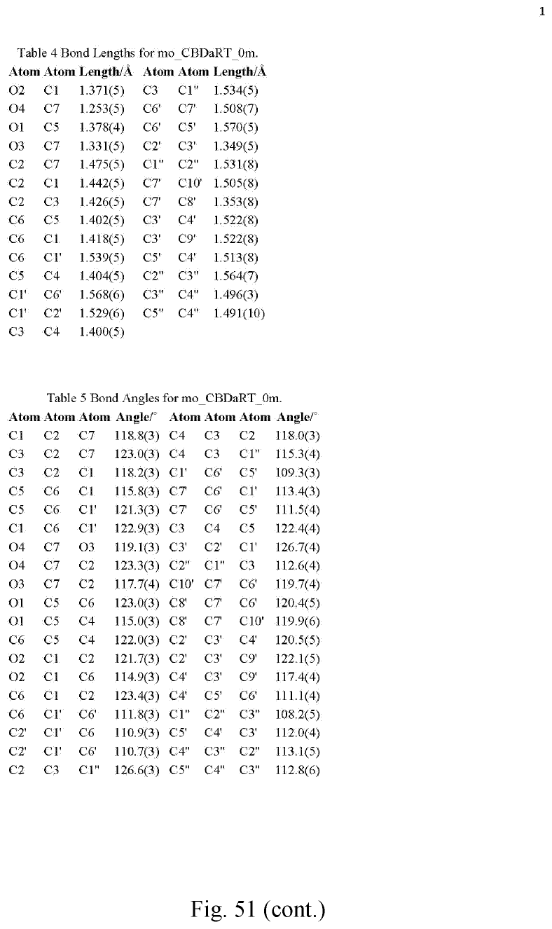

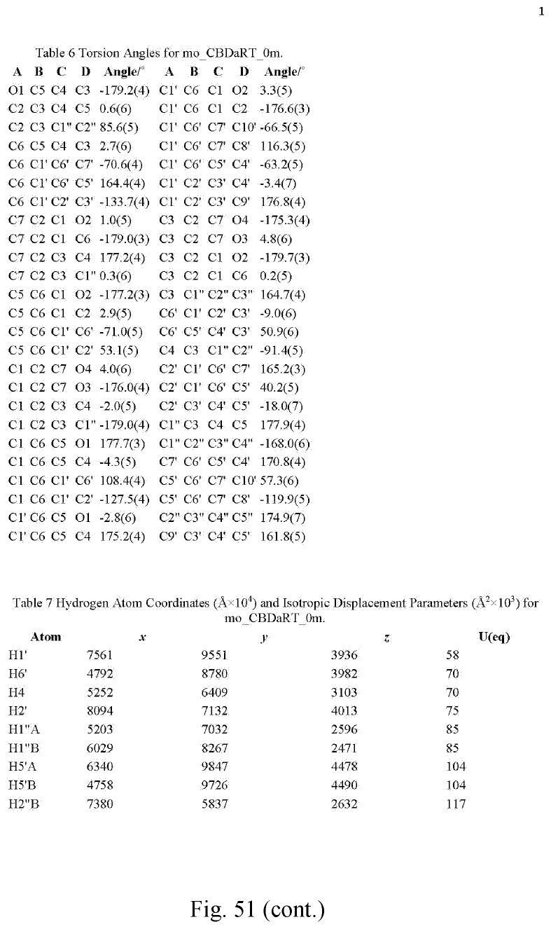

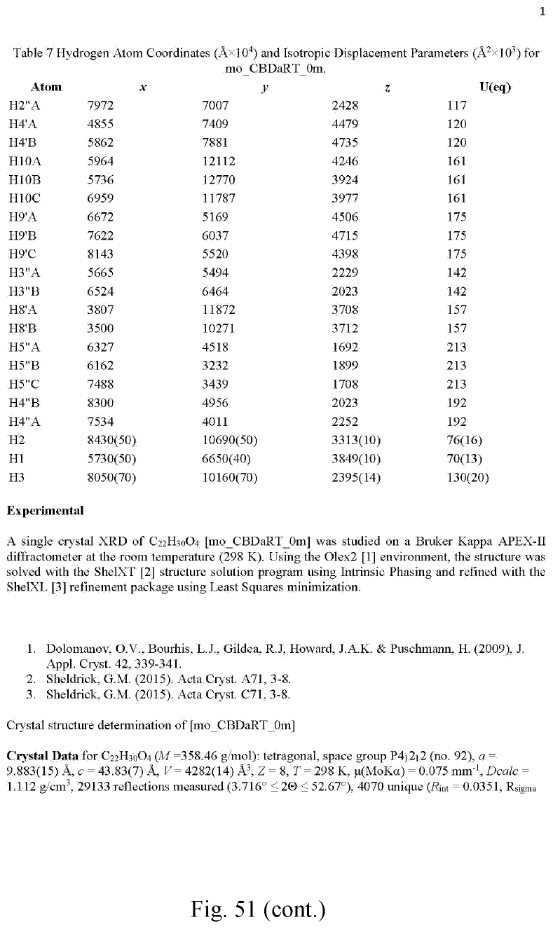

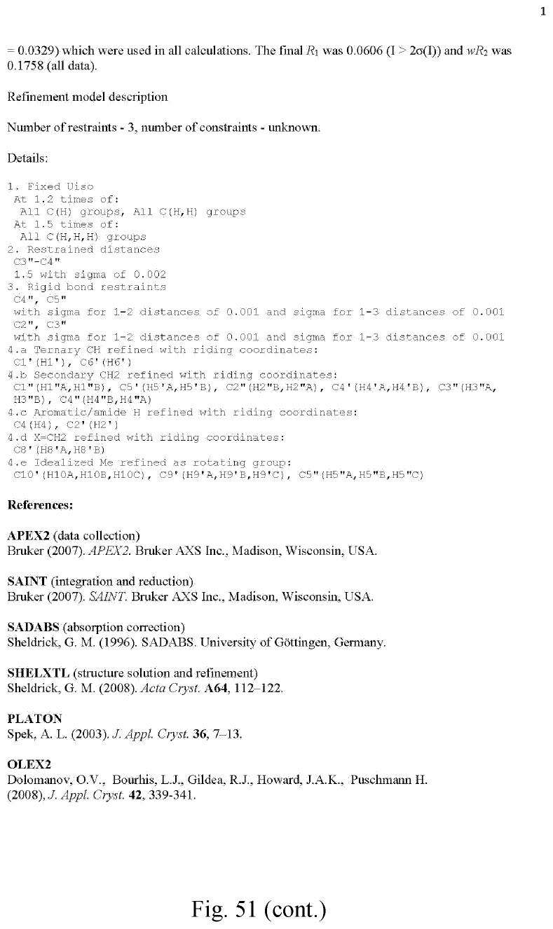

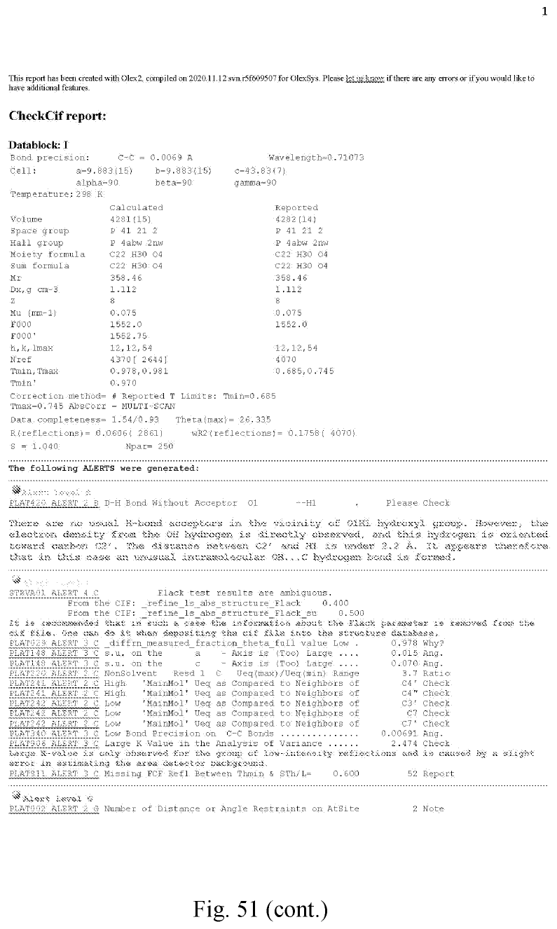

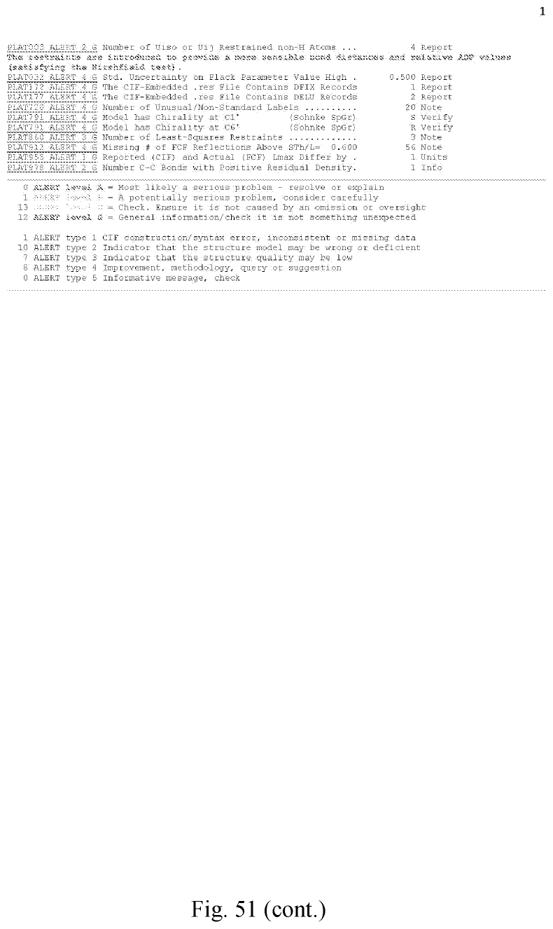

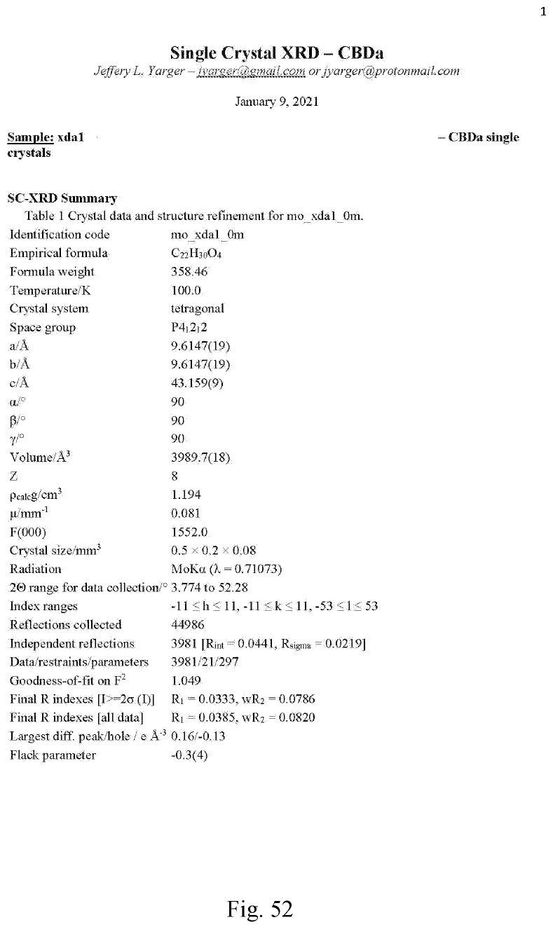

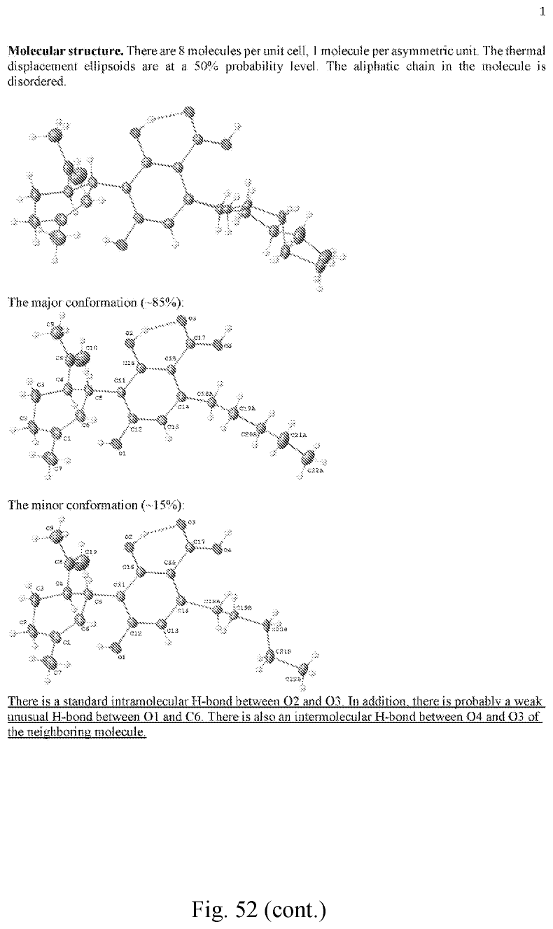

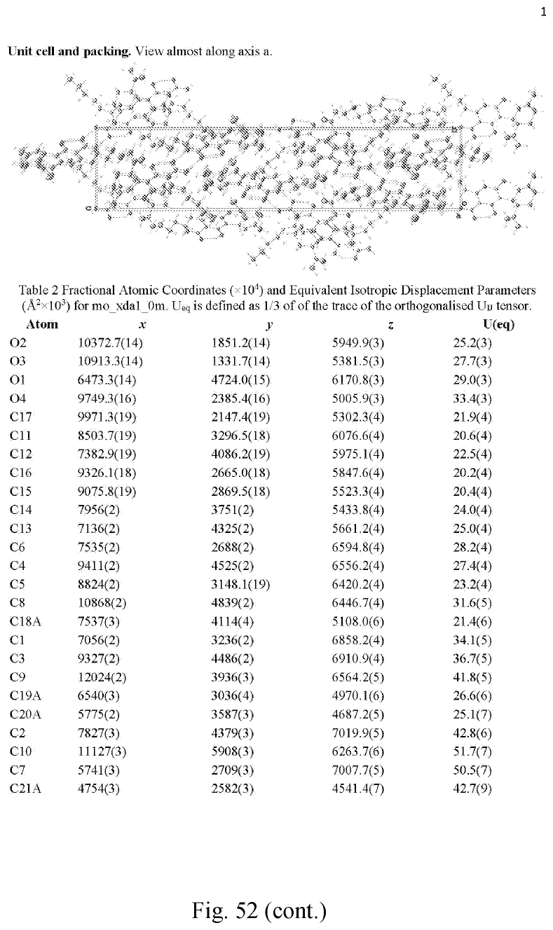

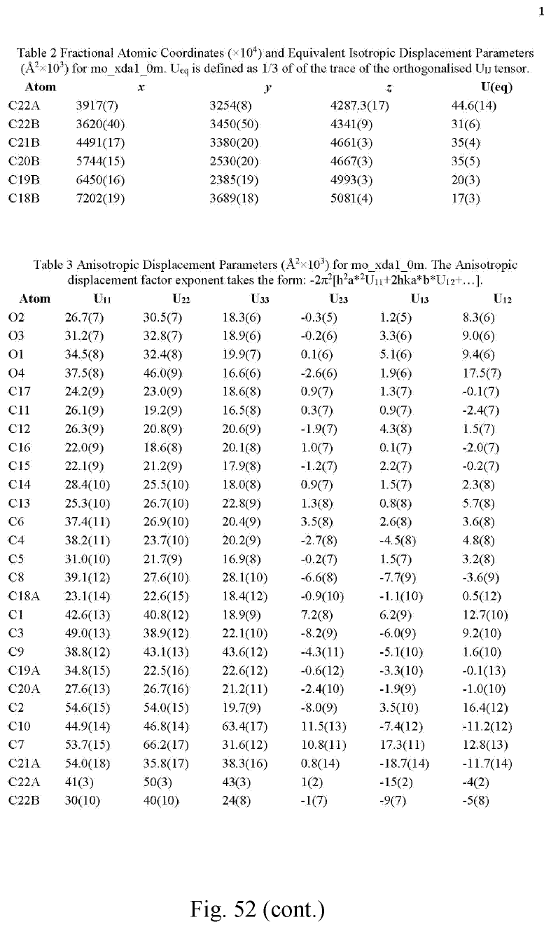

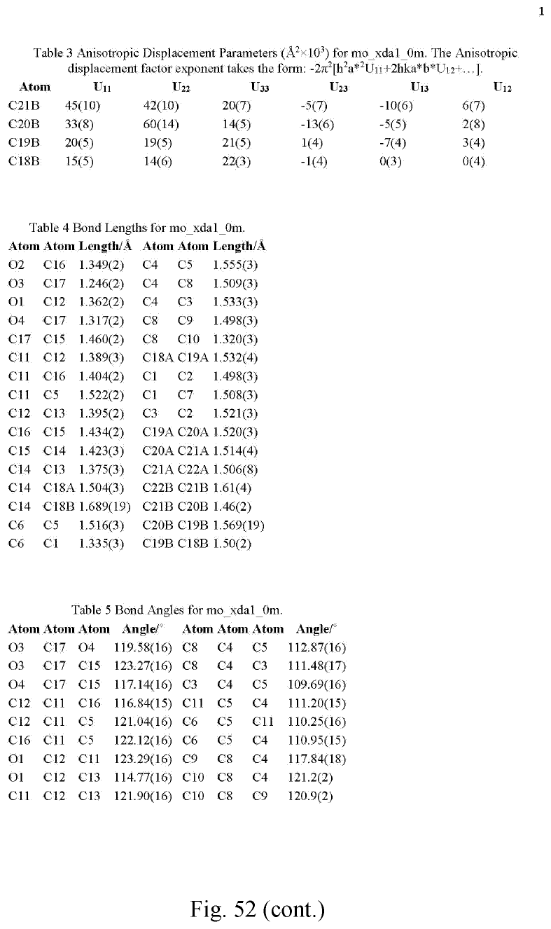

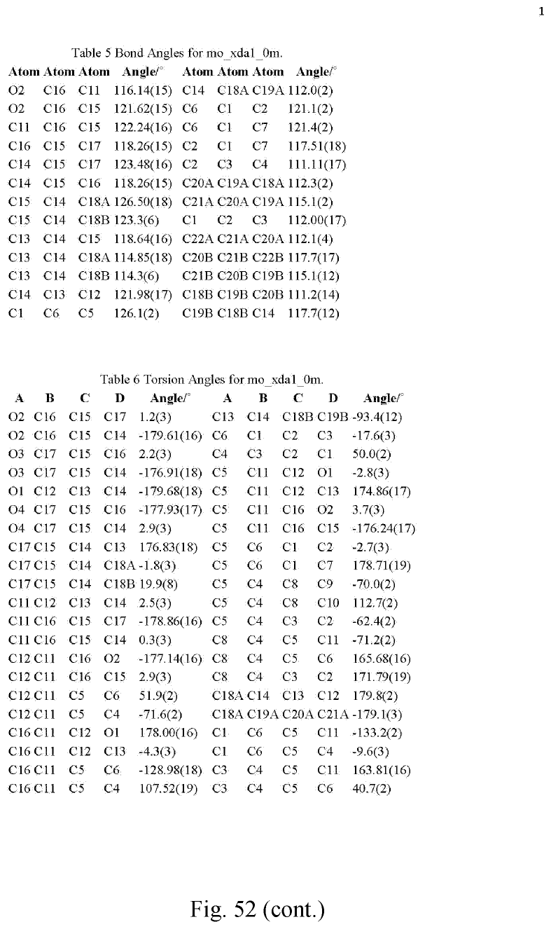

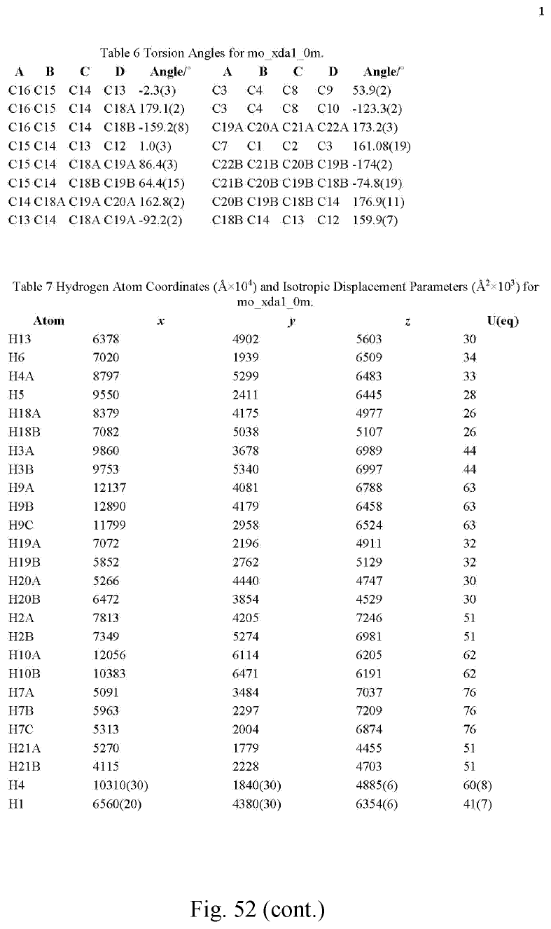

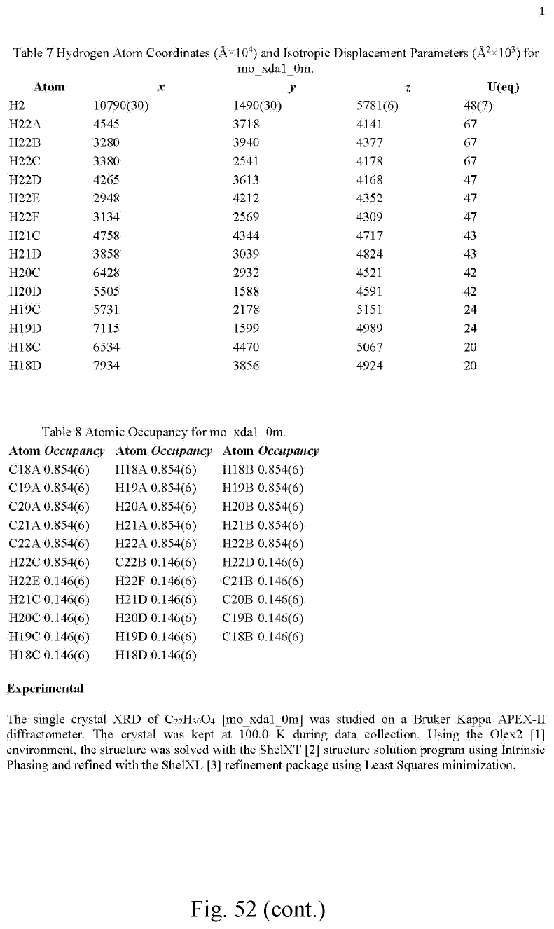

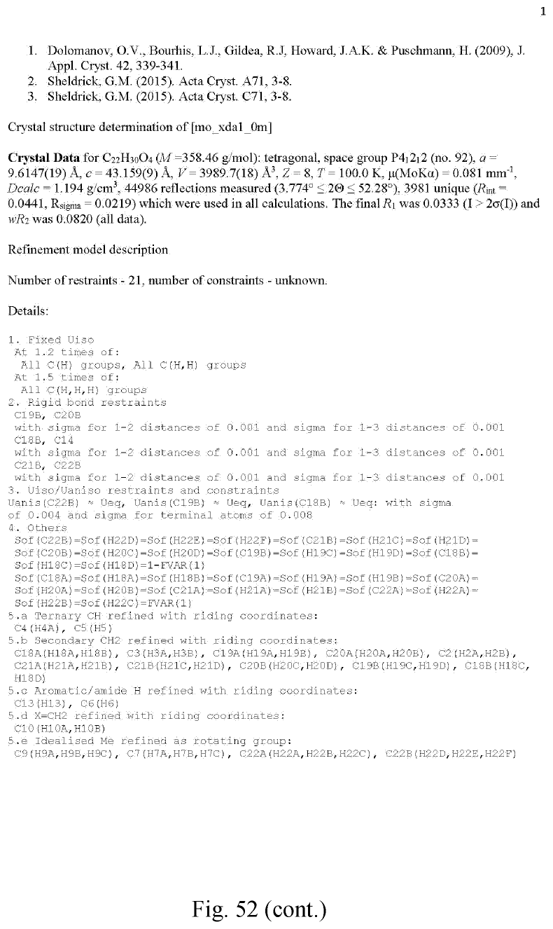

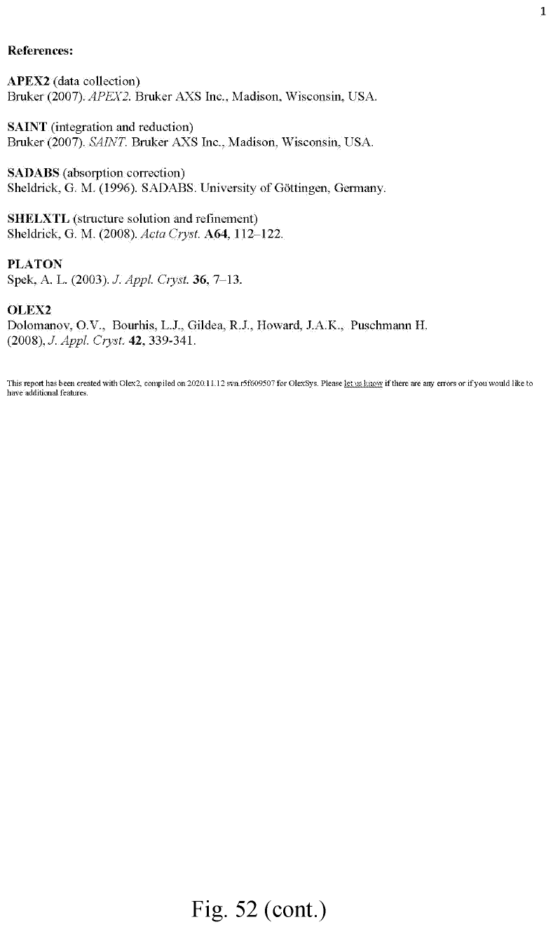

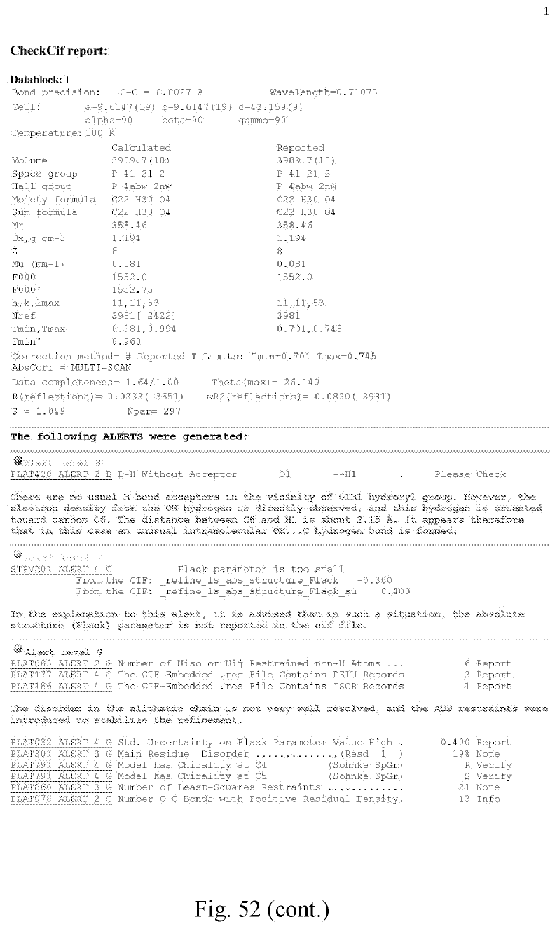

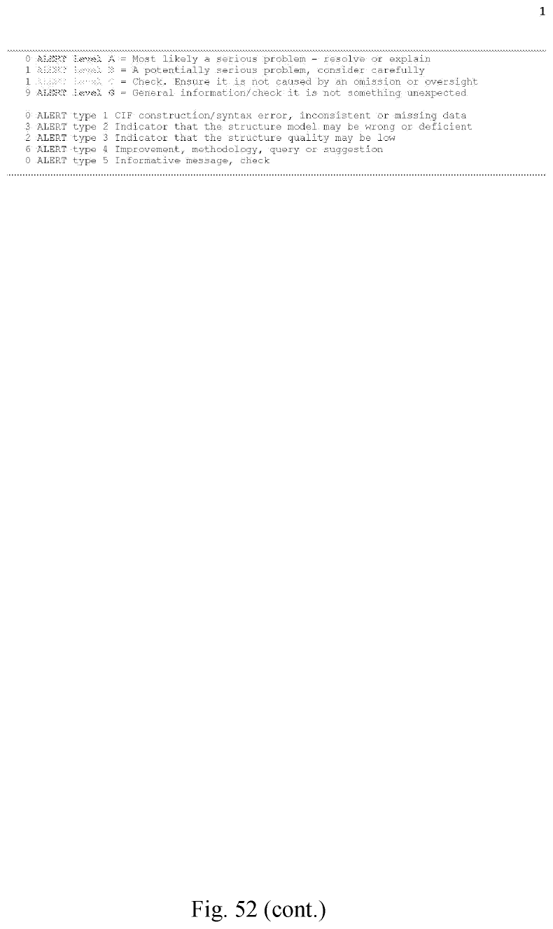

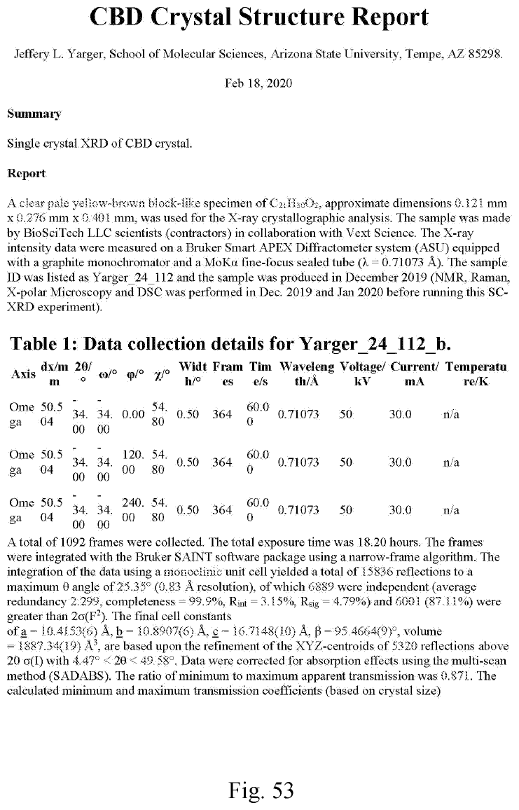

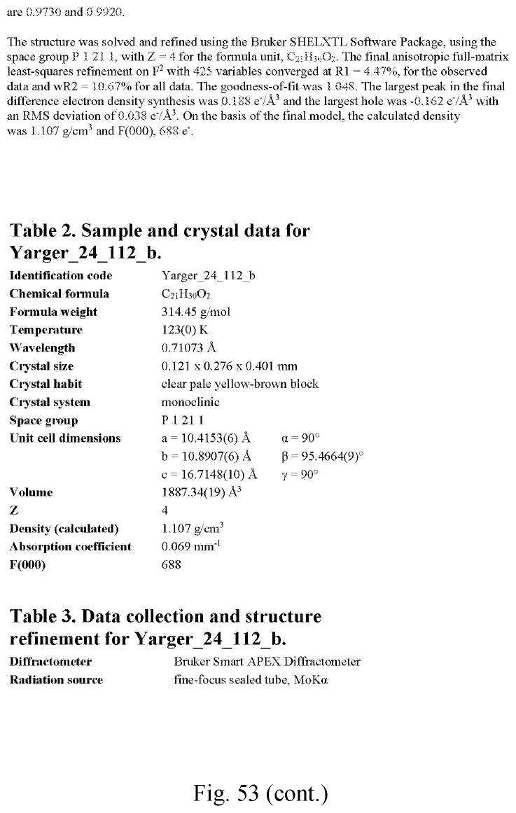

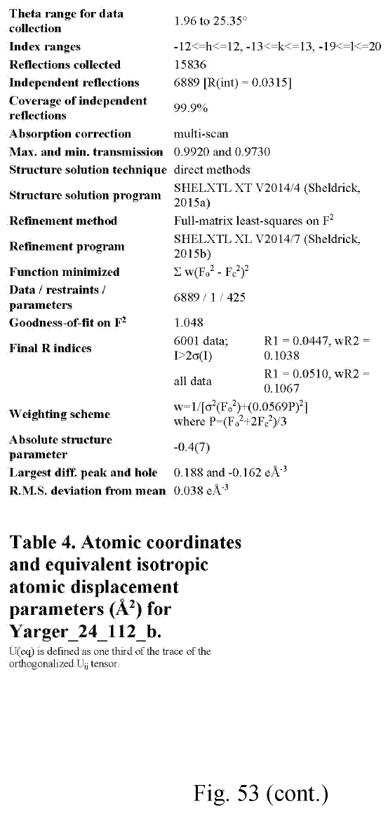

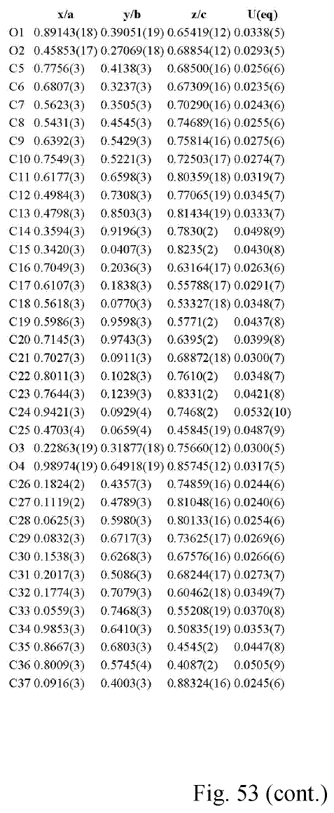

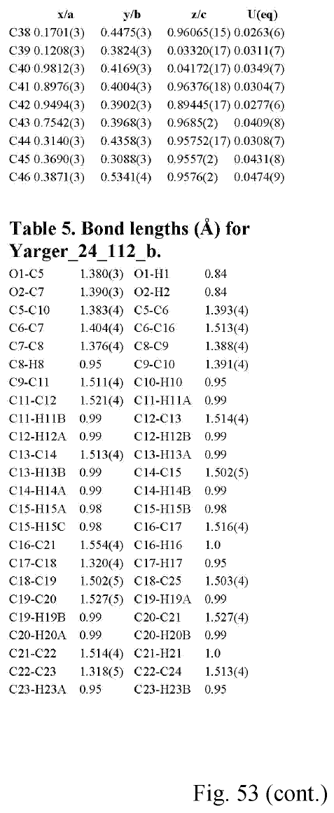

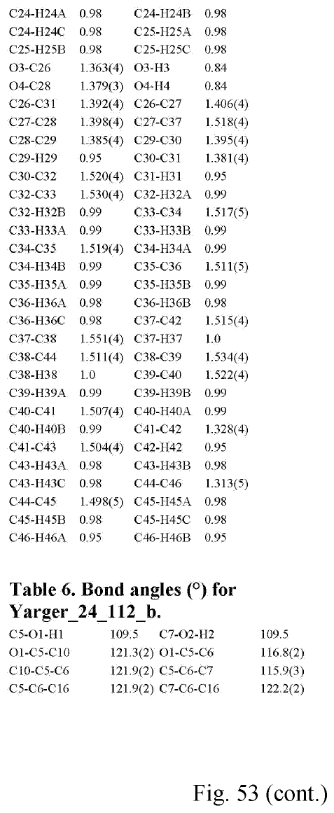

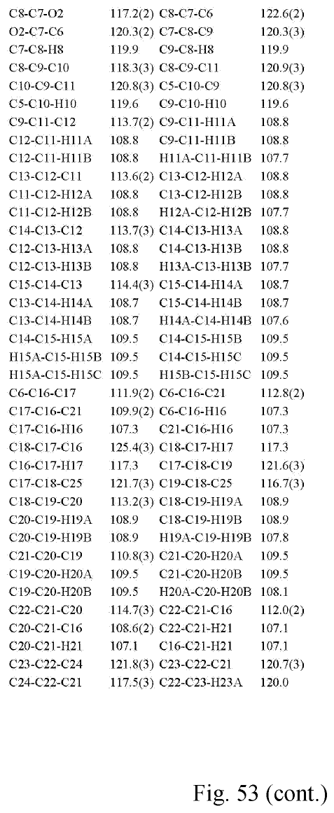

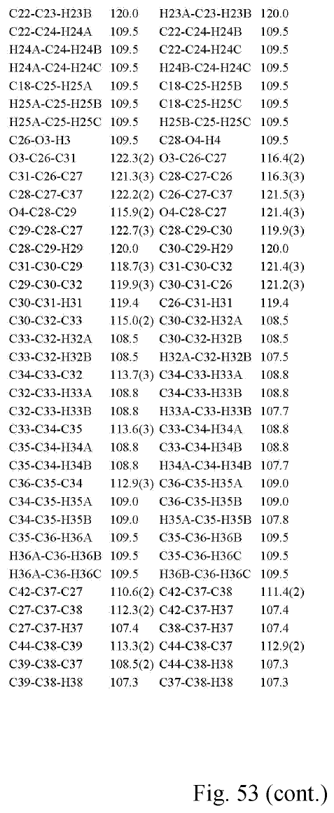

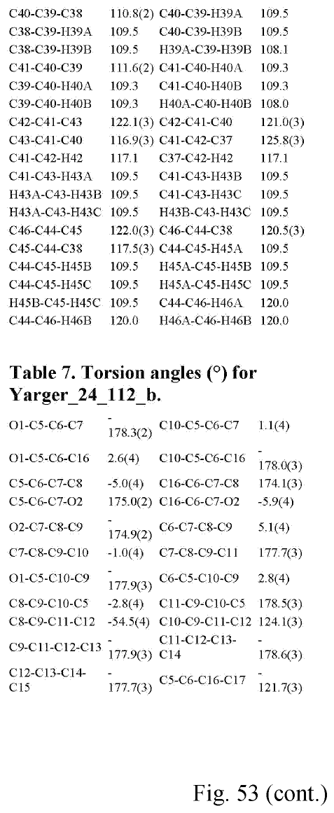

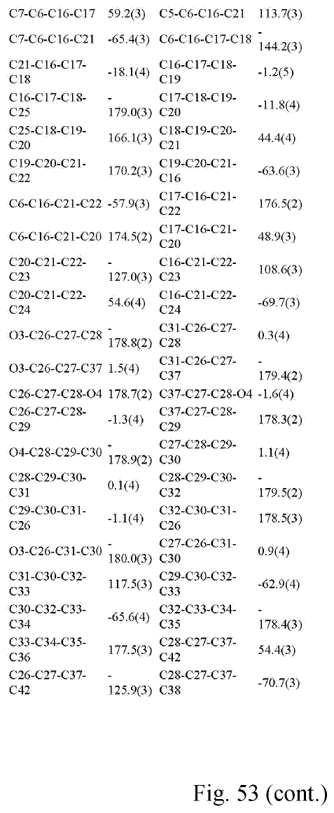

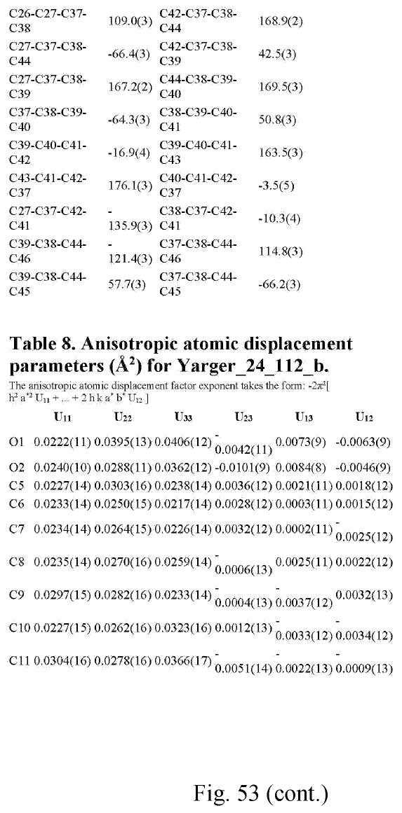

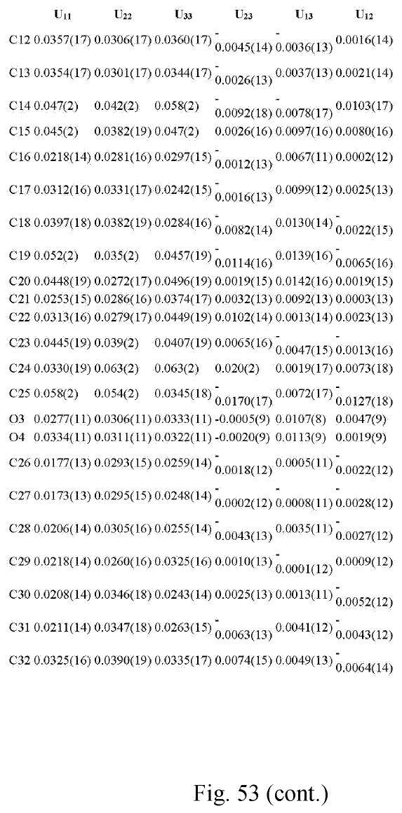

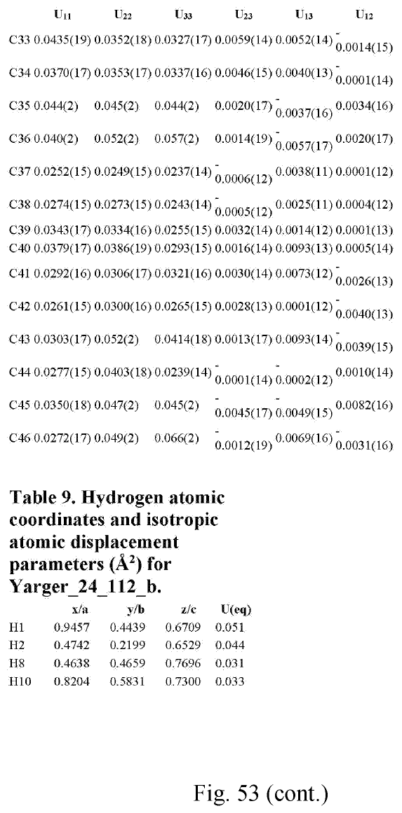

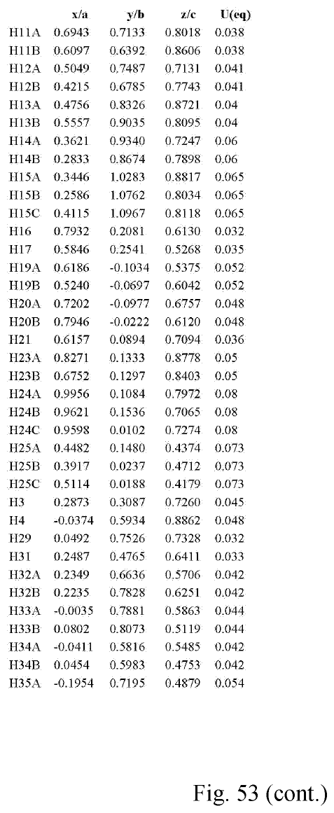

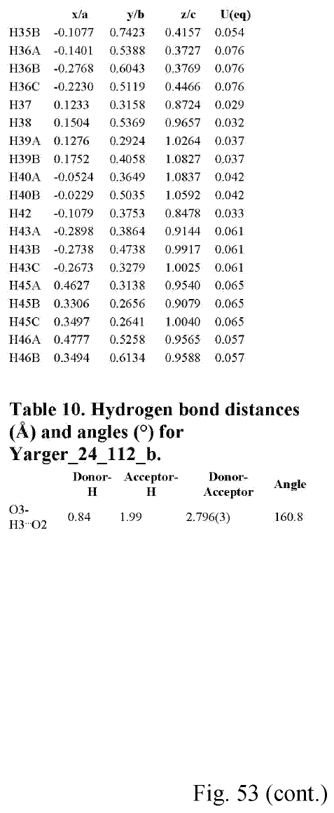

In embodiments the crystalline form of cannabidiolic acid (CBDa) can comprise a single crystal X-ray diffraction spectrum characterized by diffraction angle 2θ comprising at least one characteristic peak as described herein. In embodiments, the crystalline form of cannabidiolic acid (CBDa) can comprise a single crystal X-ray diffraction spectrum characterized by diffraction angle 2θ comprising the spectrum substantially as show in FIGS. 50-52.

### Fourier Transform Infrared (FTIR) Spectroscopy Preparation and Parameters

Fourier transform infrared (FT-IR) spectroscopy data were collected using an Agilent Cary 630 FT-IR spectrometer equipped with a diamond attenuated total reflectance (ATR) sample stage. Prior to analysis of the sample a background was collected with identical experimental conditions such that it can be subtracted from the sample spectrum by the expertlabs software. The solid powder (approximately 20 mg) was compressed onto the diamond ATR using the sample piston prior to data collection. The spectrum and background were both collected with 128 scans, and spectral range of 600 to 4000 cm⁻¹.

In embodiments, the crystalline forms described herein can be characterized by FTIR. In embodiments, spectral characteristics can be used to determine crystallinity. For example, these spectral characteristics can comprise frequencies, relative intensities, band contours, numbers of bands, and changes thereof. One of skill in the art will recognize that the FTIR peaks at specified wavenumbers are not absolute and can vary with by about 5 cm⁻¹.

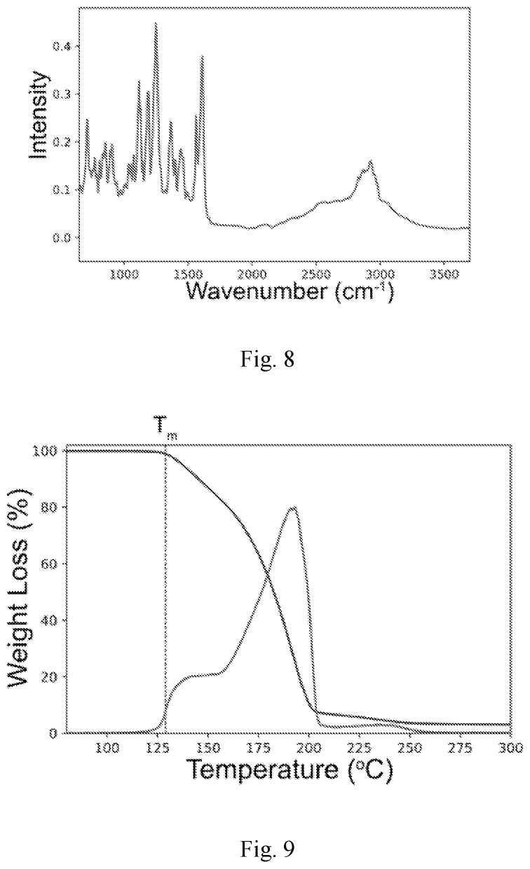

In embodiments the crystalline form of tetrahydrocannabinolic acid (THCa) can comprise an FTIR spectrum with at least one characteristic peak at about 742, 820, 887, 973, 1029, 1070, 1107, 1167, 1234, 1360, 1428, 1547, 1644, 1700, 2860, 2926, 2957 cm⁻¹. In embodiments, the crystalline form of tetrahydrocannabinolic acid (THCa) can comprise an FTIR spectrum substantially as show in FIG. 8.

In embodiments the crystalline form of cannabigerolic acid (CBGa) can comprise an FTIR spectrum with at least one characteristic peak at about 675, 734, 753, 809, 831, 880, 924, 973, 1044, 1066, 1085, 1107, 1167, 1245, 1271, 1379, 1413, 1457, 1498, 1580, 1610, 1636, 2851, 2911, 2960, 3399 cm⁻¹. In embodiments, the crystalline form of cannabigerolic acid (CBGa) can comprise an FTIR spectrum substantially as show in 17.

In embodiments the crystalline form of cannabidiolic acid (CBDa) can comprise an FTIR spectrum with at least one characteristic peak at about 727, 757, 783, 809, 854, 891, 962, 1014, 1032, 1092, 1111, 1144, 1182, 1237, 1278, 1334, 1357, 1375, 1409, 1446, 1495, 1573, 1610, 2855, 2926, 3388 cm⁻¹. In embodiments, the crystalline form of cannabidiolic acid (CBDa) can comprise an FTIR spectrum substantially as show in FIG. 23.

### Liquid State ¹H NMR Sample Preparation and Experimental Conditions

Liquid-state NMR data were collected using a 500 MHz Bruker AVANCE III spectrometer equipped with a 5 mm broad-band H-X probe tuned for ¹H-¹³C operation. The ¹H NMR spectrum of the samples were collected by dissolving approximately 10-20 mg of solid material in 750 μL of CDCl₃ which contained 0.1% TMS (v/v) as an internal chemical shift standard for ¹H and ¹³C. The ¹H NMR spectrum of THCa was collected on a sample which was prepared by dissolving approximately 10 mg of THCa in 750 μL of CDCl3 which contained 0.1% TMS (v/v) as an internal chemical shift standard for ¹H and ¹³C.

In embodiments ¹H NMR spectrum can be used to characterize crystalline forms of the compositions described herein. One of skill in the art will appreciate that peak shifts are not absolute and can vary based experimental conditions, e.g., solvent used. In embodiments, one of skill in the art will recognize that peak shifts can vary by about +/−0.2 ppm. In embodiments, an ¹H NMR spectrum of a crystalline form can possess narrower, more well-defined peaks when compared to a non-crystalline form ¹H NMR spectrum.

In embodiments the crystalline form of tetrahydrocannabinolic acid (THCa) can comprise an ¹H NMR spectrum with at least one characteristic peak at about 0.903, 1.11, 1.35(m), 1.44, 1.58(m), 1.68, 1.92(m), 2.17, 2.79(m), 2.95(m), 3.23, 6.26, 6.39, 11.70, 12.16 ppm in CDCl₃. In embodiments, the crystalline form of tetrahydrocannabinolic acid (THCa) can comprise an ¹H NMR spectrum substantially as show in FIG. 2.

In embodiments the crystalline form of cannabigerolic acid (CBGa) can comprise an ¹H NMR spectrum with at least one characteristic peak at about 0.90, 1.35, 1.59, 1.67, 1.82, 2.08(m), 2.89, 3.44, 5.06, 5.28, 5.93, 11.90 ppm in CDCl₃. In embodiments, the crystalline form of cannabigerolic acid (CBGa) can comprise an ¹H NMR spectrum substantially as show in FIG. 31.

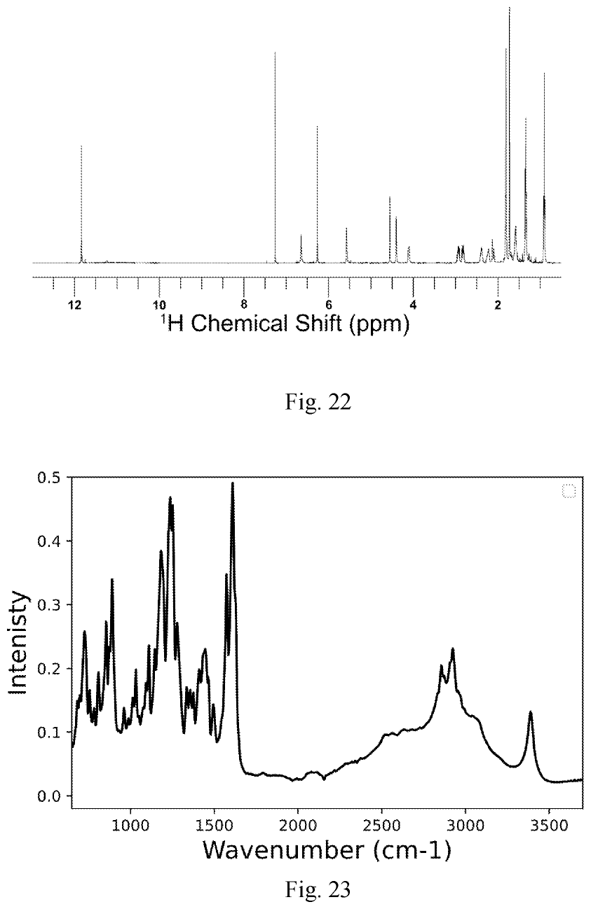

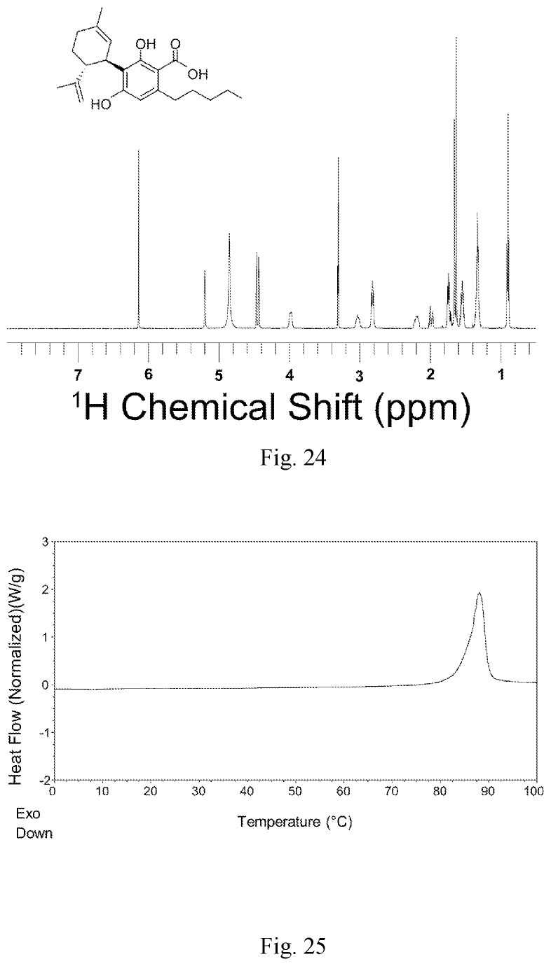

In embodiments the crystalline form of cannabidiolic acid (CBDa) can comprise an ¹H NMR spectrum with at least one characteristic peak at about 0.90, 1.33, 1.58(m), 1.72, 1.80, 2.12, 2.22, 2.38, 2.82(m), 2.93(m), 4.40, 4.55, 5.57, 6.26, 6.64, 11.84, ppm in CDCl₃. In embodiments, the crystalline form cannabidiolic acid (CBDa) can comprise an ¹H NMR spectrum substantially as show in FIG. 22.

### Liquid State ¹³C NMR Sample Preparation and Experimental Conditions

Liquid-state NMR data were collected using a 500 MHz Bruker AVANCE III spectrometer equipped with a 5 mm broad-band H-X probe tuned for ¹H-¹³C operation. The ¹³C NMR spectrum of samples were collected by dissolving approximately 10-20 mg of the sample in 750 μL of CDCl₃ which contained 0.1% TMS (v/v) as an internal chemical shift standard for ¹H and ¹³C.

In embodiments liquid ¹³C NMR spectrum can be used to characterize crystalline forms of the compositions described herein. One of skill in the art will appreciate that peak shifts are not absolute and can vary based experimental conditions, e.g., solvent used. In embodiments, one of skill in the art will recognize that peak shifts can vary by about +/−0.2 ppm.

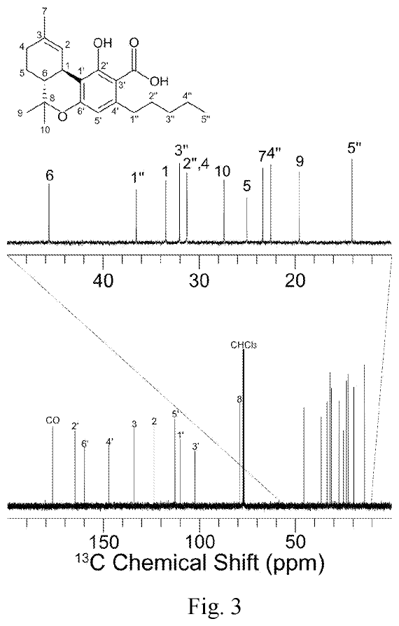

In embodiments the crystalline form of tetrahydrocannabinolic acid (THCa) can comprise a state ¹³C (¹H decoupled) nuclear magnetic resonance (NMR) spectrum comprising at least one characteristic peak at about 14.1, 19.5, 22.5, 23.4, 25.0, 27.4, 31.2, 31.3, 32.0, 33.5, 36.5, 45.6, 78.9, 102.3, 109.9, 112.7, 123.6, 133.9, 147.0, 159.8, 164.7, 176.5 ppm in CDCl₃. In embodiments, the crystalline form of tetrahydrocannabinolic acid (THCa) can comprise a ¹³C NMR spectrum substantially as show in FIG. 3.

In embodiments the crystalline form of cannabigerolic acid (CBGa) can comprise a ¹³C NMR spectrum with characteristic peaks at about 14.1, 16.2, 17.7, 22.1, 22.5, 25.7, 26.4, 31.4, 32.0, 36.6, 39.7, 103.1, 111.4, 111.6, 121.3, 123.8, 132.1, 139.2, 147.5, 160.6, 163.6, 176.1 ppm. In embodiments, the crystalline form of cannabigerolic acid (CBGa) can comprise an ¹³C NMR spectrum substantially as show in FIG. 41.

In embodiments the crystalline form of cannabidiolic acid (CBDa) can comprise liquid state ¹³C (¹H decoupled) nuclear magnetic resonance (NMR) spectrum comprising at least one characteristic peak at about 14.1, 18.9, 22,5, 23.7, 30.2, 31.2, 32.0, 35.4, 36.6, 46.6, 102.5, 111.4, 112.0, 114.5, 123.9, 140.5, 147.6, 161.0, 164.2, 176.0 ppm in CDCl₃. In embodiments, the crystalline form cannabidiolic acid (CBDa) can comprise a 13C NMR spectrum substantially as show in FIG. 29.

### Solid State (Ss) NMR Preparation and Procedure

The ssNMR data were collected using an 800 MHz Varian VNMRS system equipped with a 1.6 mm Varian triple resonance broadband probe operating in double resonance mode at 799.84 and 201.14 for ¹H and ¹³C respectively. The ¹H→¹³C CP-MAS spectrum was collected with a recycle delay of 10 seconds, 8 k scans, and a sweep width of 100 kHz. The ¹H→¹³C CP-MAS NMR spectra were collected using a 2.5 μs 1H 90° degree pulse, a 1 ms ramped (˜10%)¹H spin-lock pulse and a ¹³C contact pulse with a radio frequency field (rf) strength of 100 kHz, and 40 kHz MAS. The rf field strength of the ¹H channel during the contact time was matched to the +1 spinning side band of the ¹H→¹³C Hartmann-Hahn profile (140 kHz). During the acquisition 100 kHz two pulse phase modulated (TPPM) proton decoupling was applied to increase the spectral resolution. The ¹H NMR spectrum was collected with the DEPTH pulse sequence to eliminate background signals from outside the coil using a recycle delay of 10 seconds, 16 scans and a spinning speed of 40 kHz. All ¹H and ¹³C chemical shifts were indirectly referenced to TMS using adamantane set to 1.8 and 38.48 ppm respectively.

### Solid State ¹H Magic Angle Spinning (MAS) NMR

In embodiments solid state ¹H Magic Angle Spinning (MAS) NMR spectrum can be used to characterize crystalline forms of the compositions described herein. One of skill in the art will appreciate that peak shifts are not absolute and can vary based experimental conditions. In embodiments, one of skill in the art will recognize that peak shifts can vary by about +/−0.5 ppm.

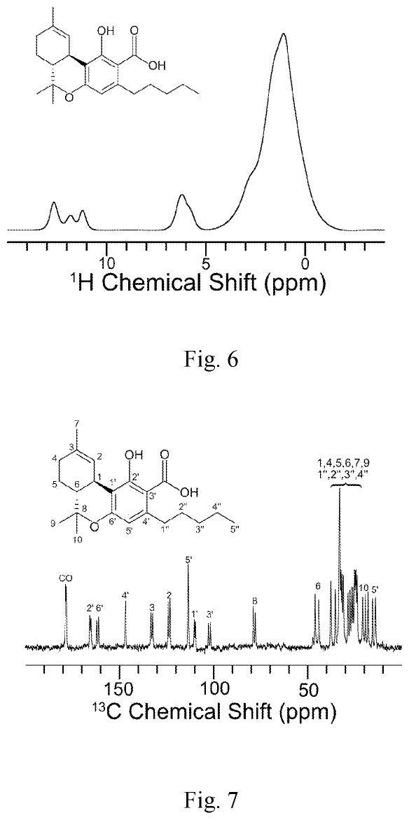

In embodiments the crystalline form of tetrahydrocannabinolic acid (THCa) can comprise an ¹H MAS NMR spectrum with at least one characteristic peak at about 1.5, 6.2, 11.2, 11.8, 12.7 ppm. In embodiments, the crystalline form of tetrahydrocannabinolic acid (THCa) can comprise an ¹H MAS NMR spectrum substantially as show in FIG. 6.

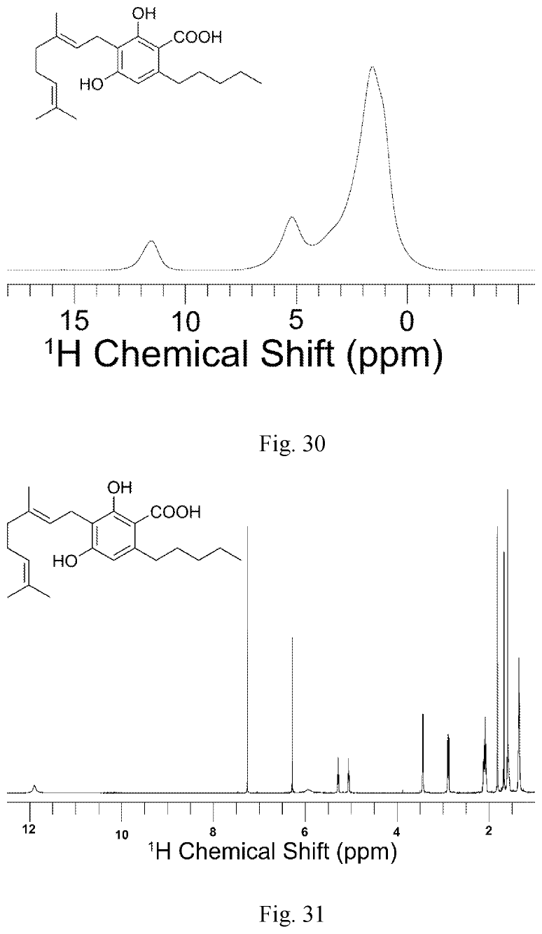

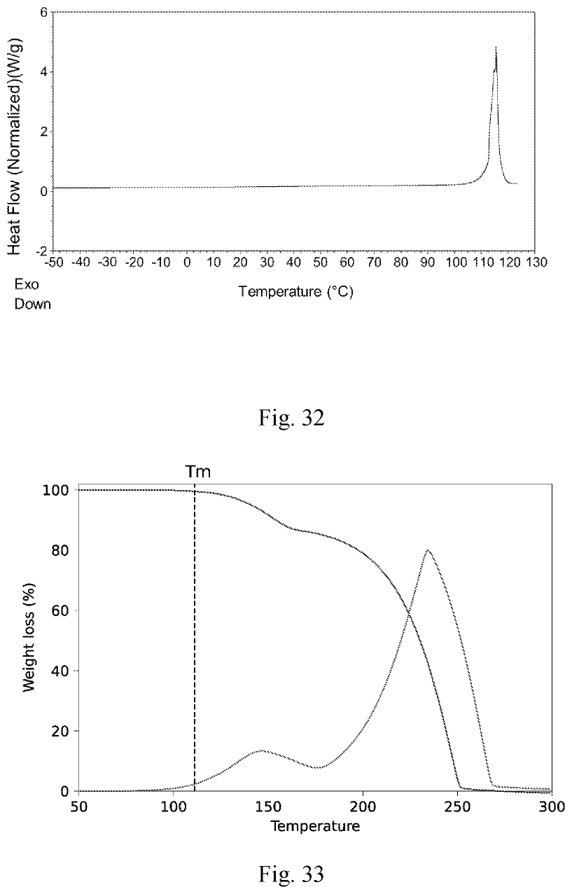

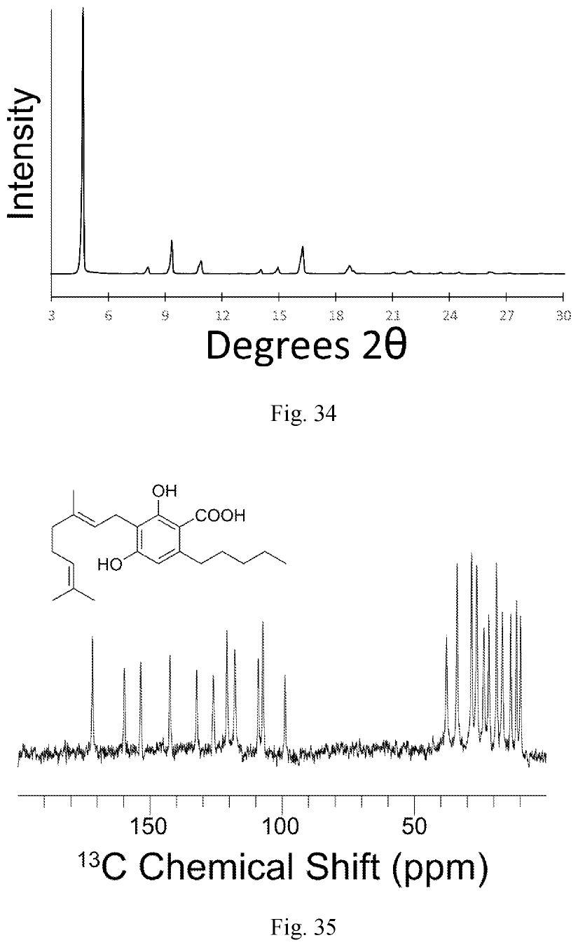

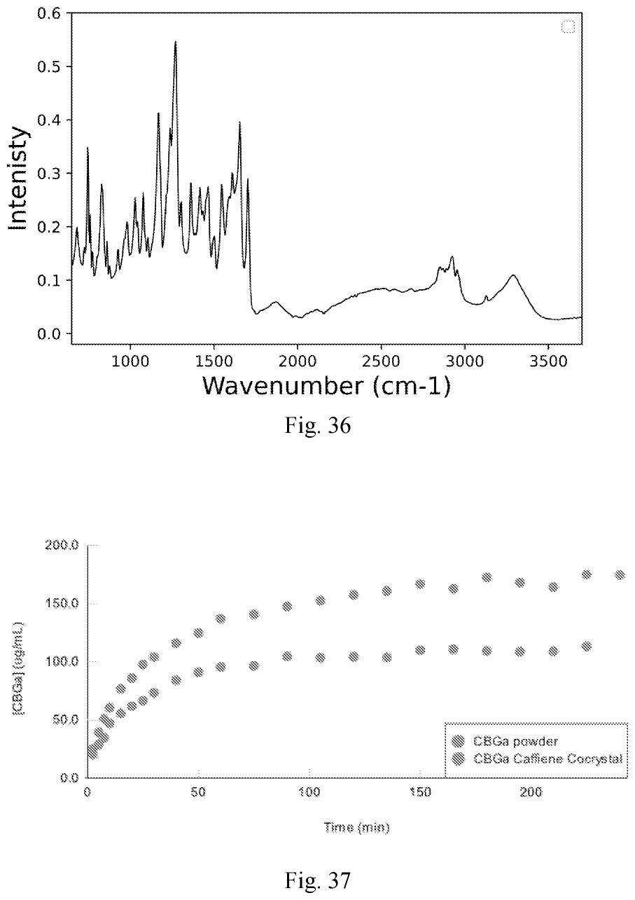

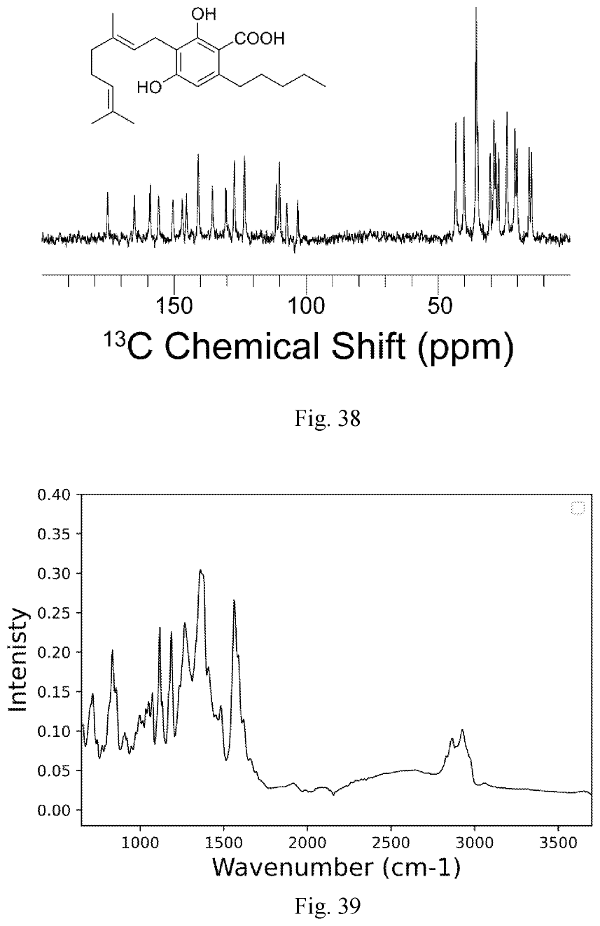

In embodiments the crystalline form of cannabigerolic acid (CBGa) can comprise an ¹H MAS NMR spectrum with at least one characteristic peak at about 1.5, 5.2, 11.5 ppm. In embodiments, the crystalline form of cannabigerolic acid (CBGa) can comprise an ¹H MAS NMR spectrum substantially as show in FIG. 30.

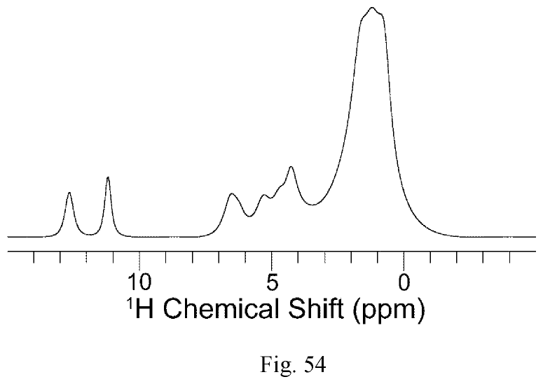

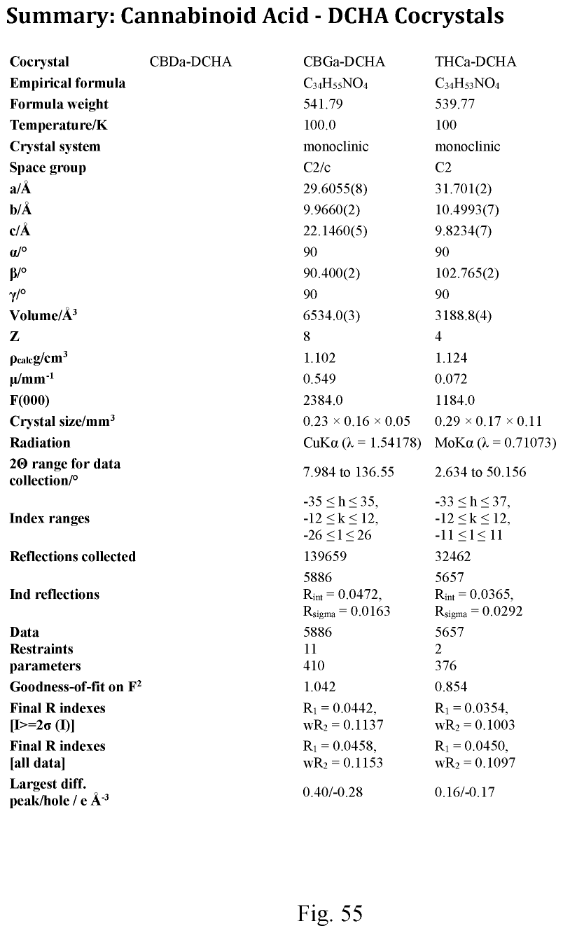

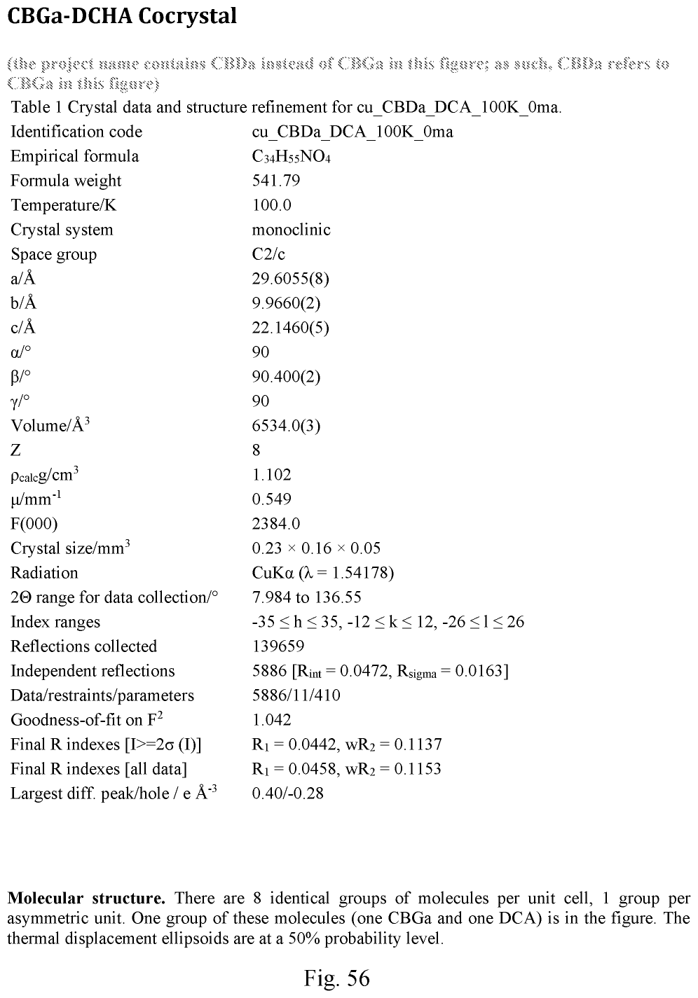

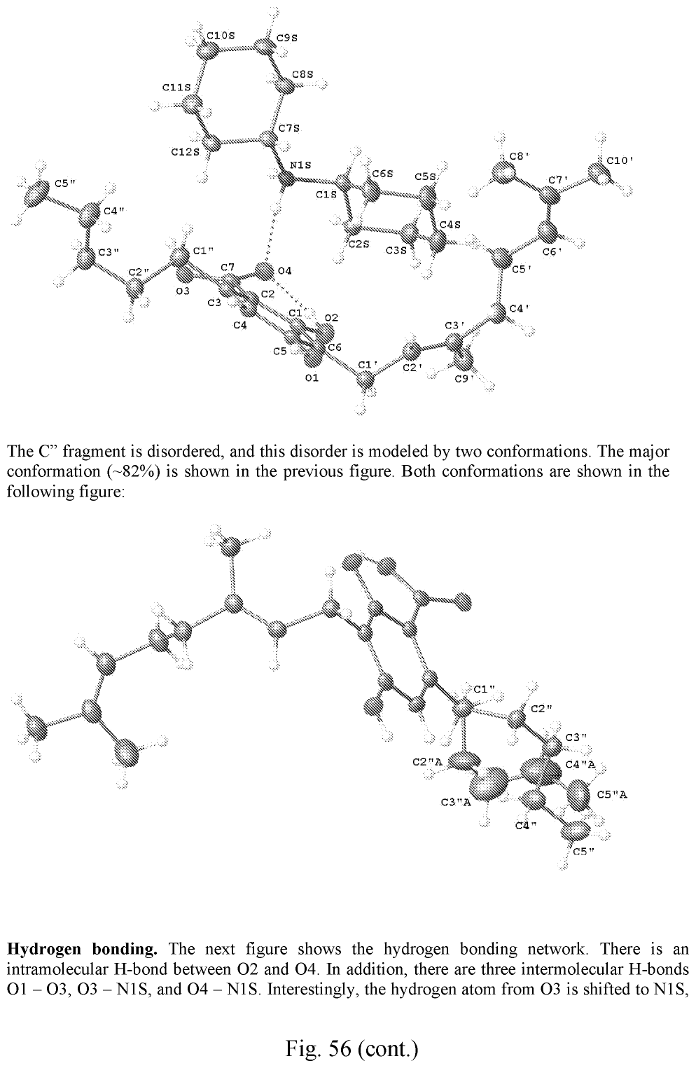

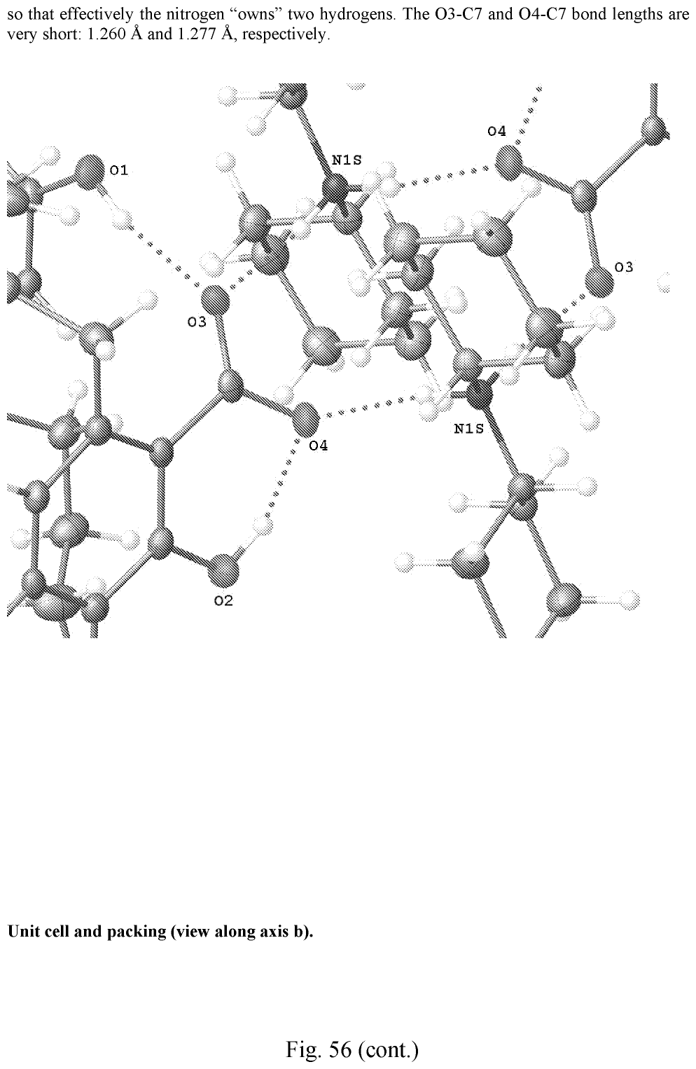

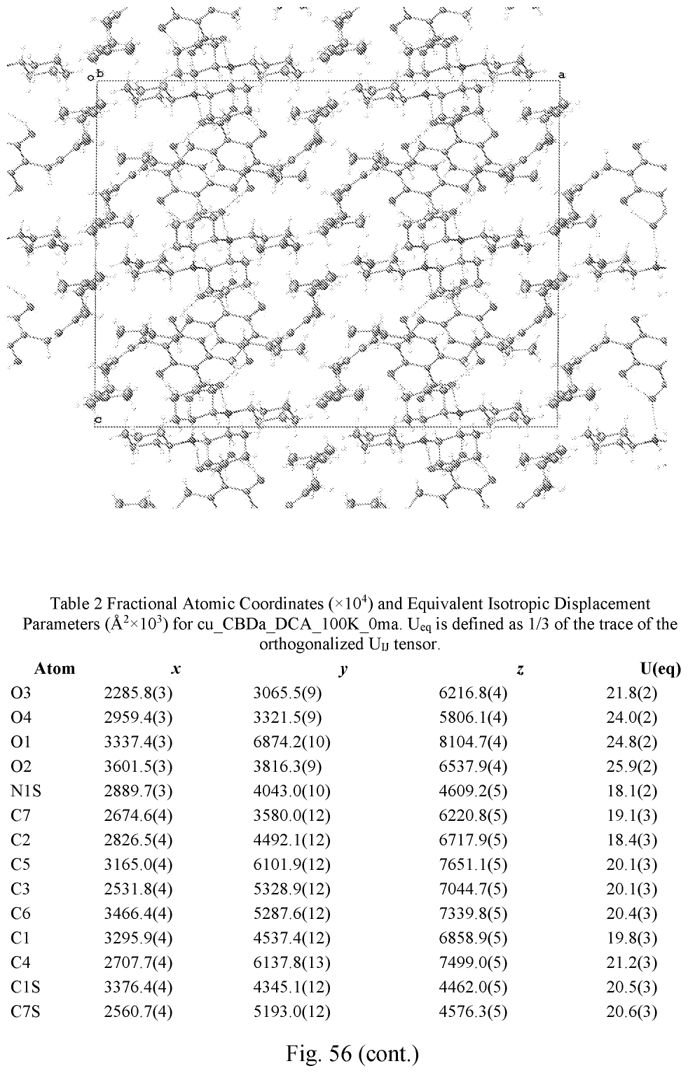

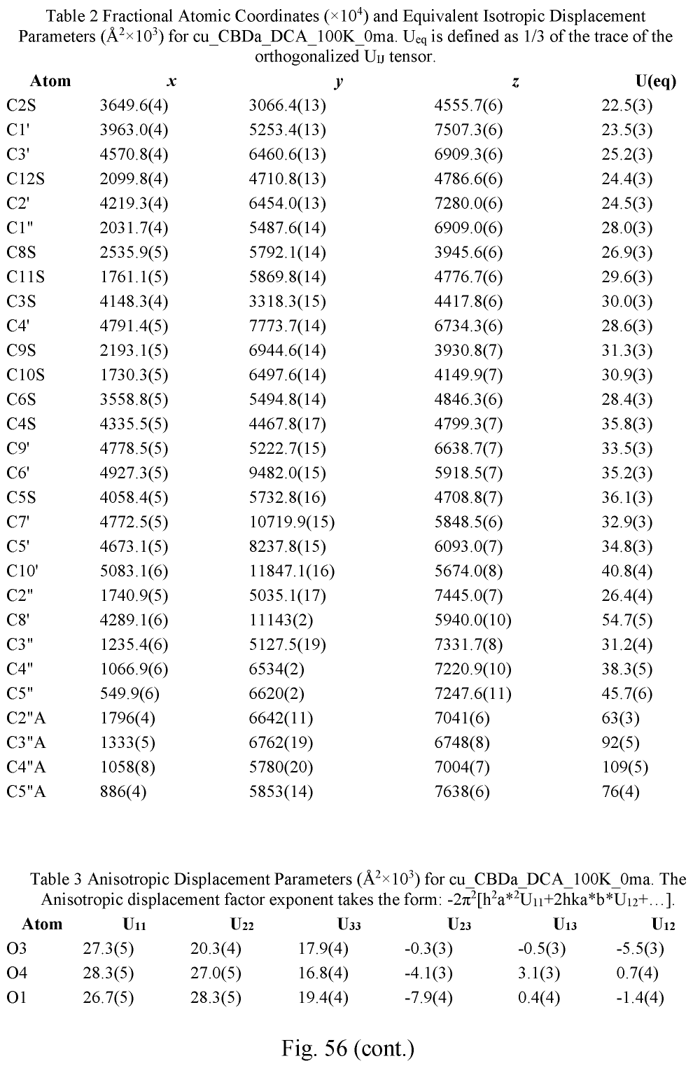

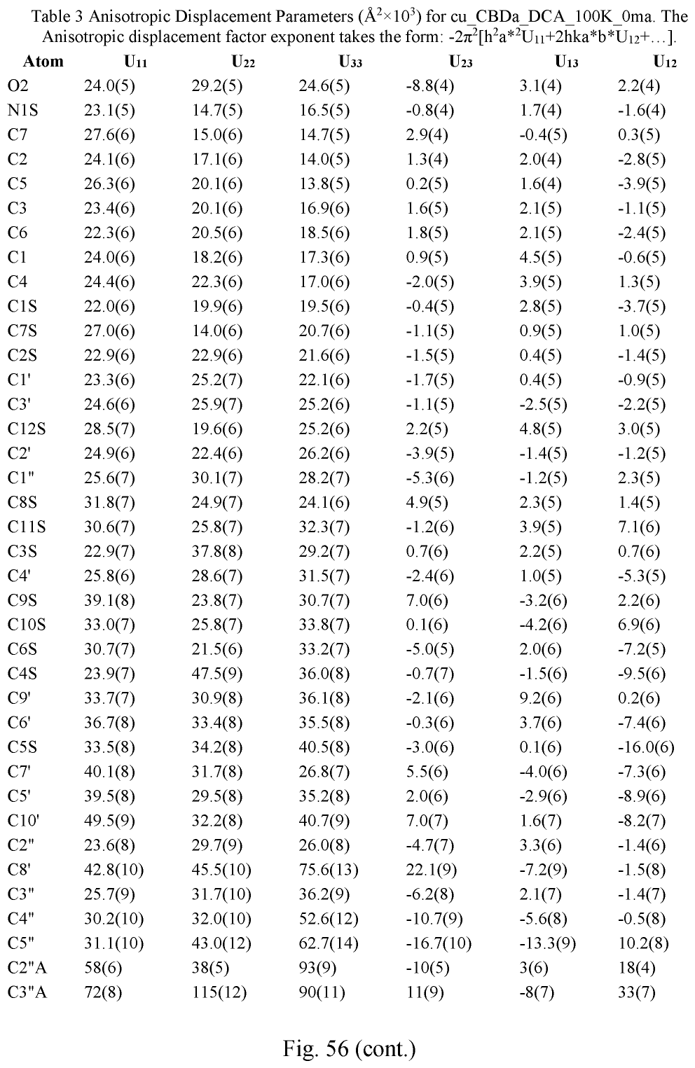

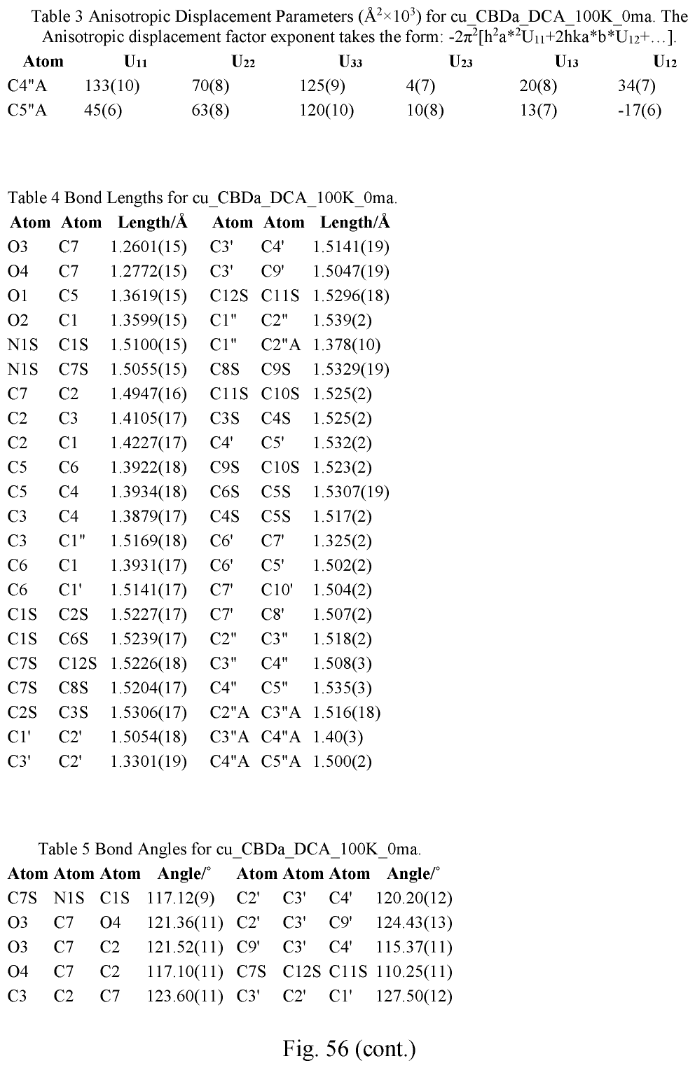

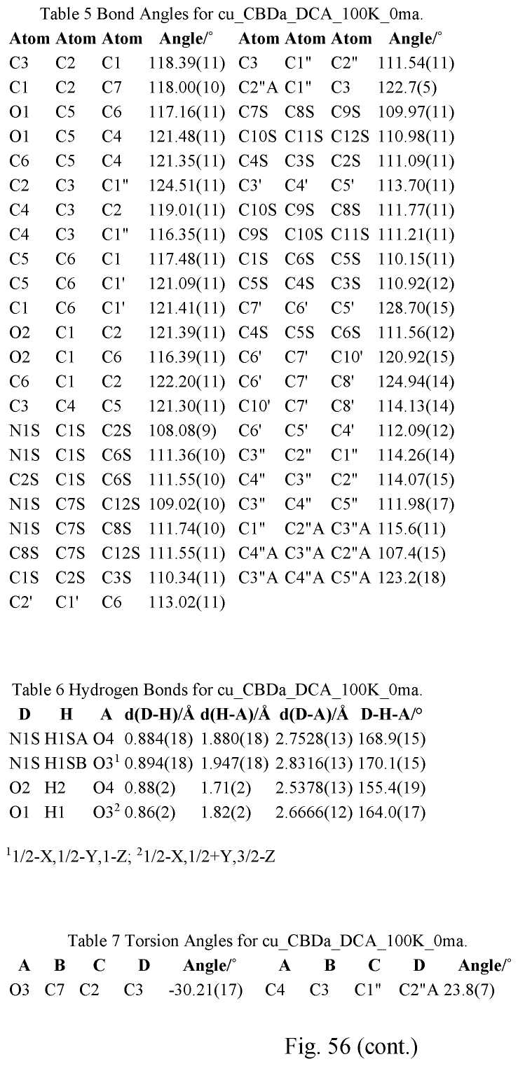

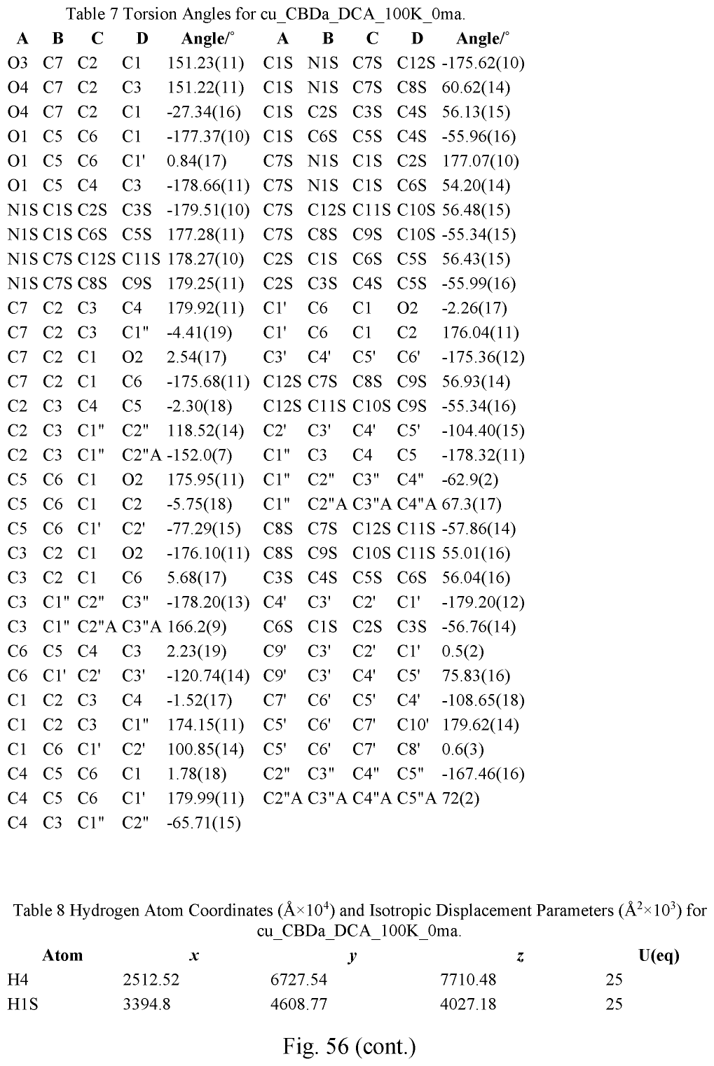

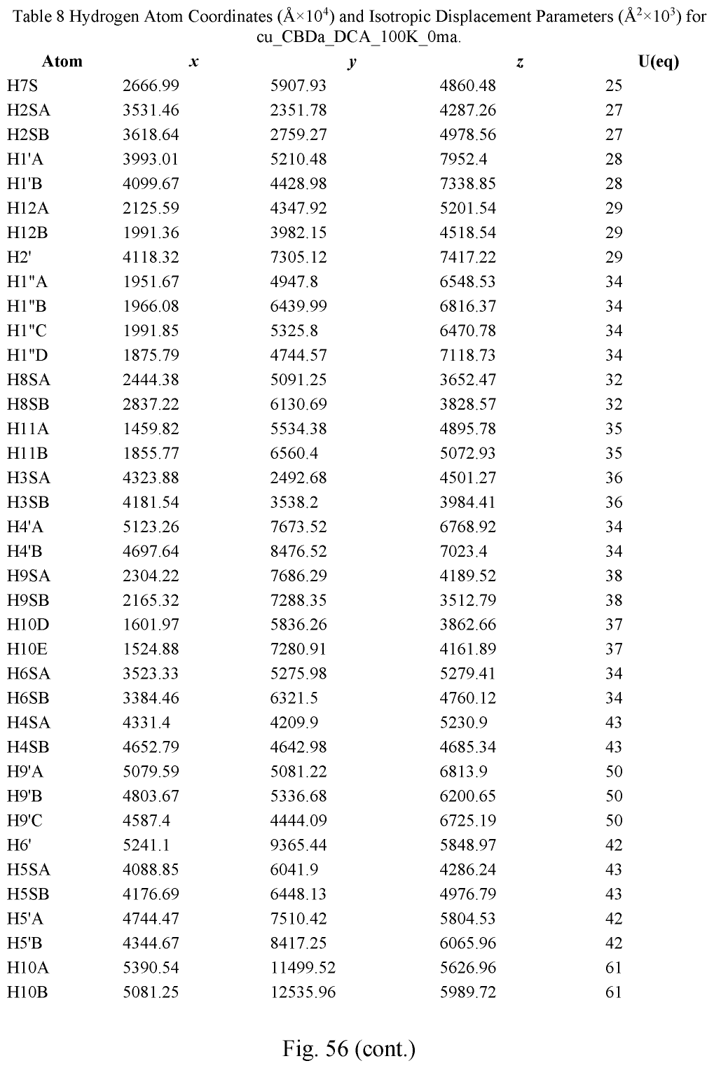

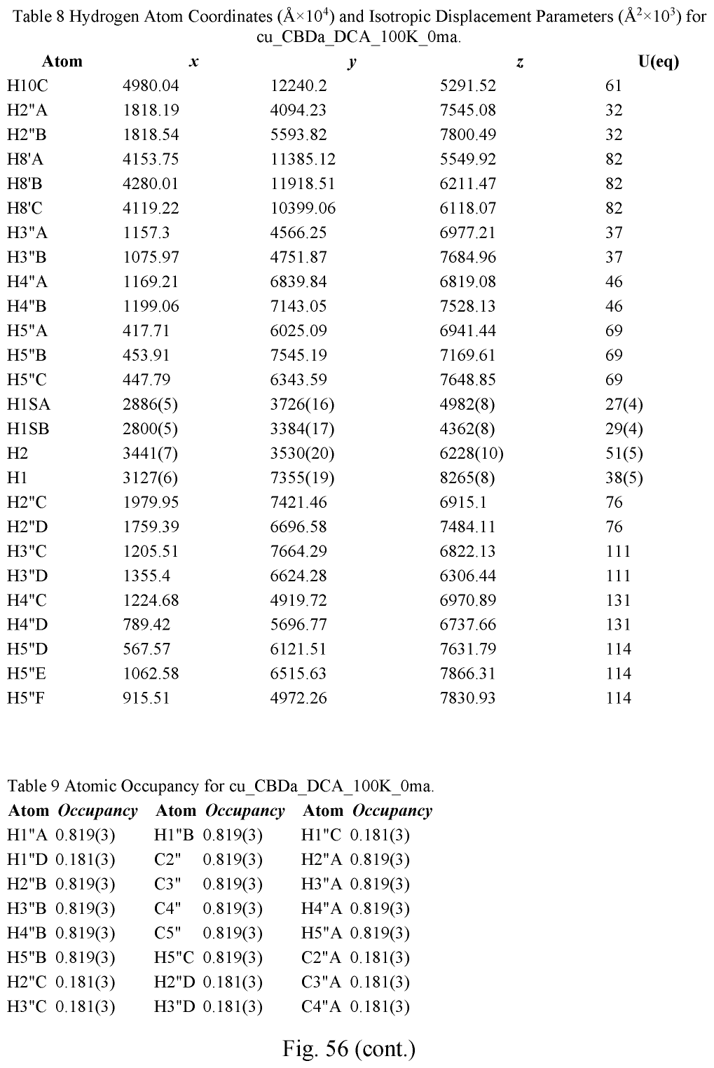

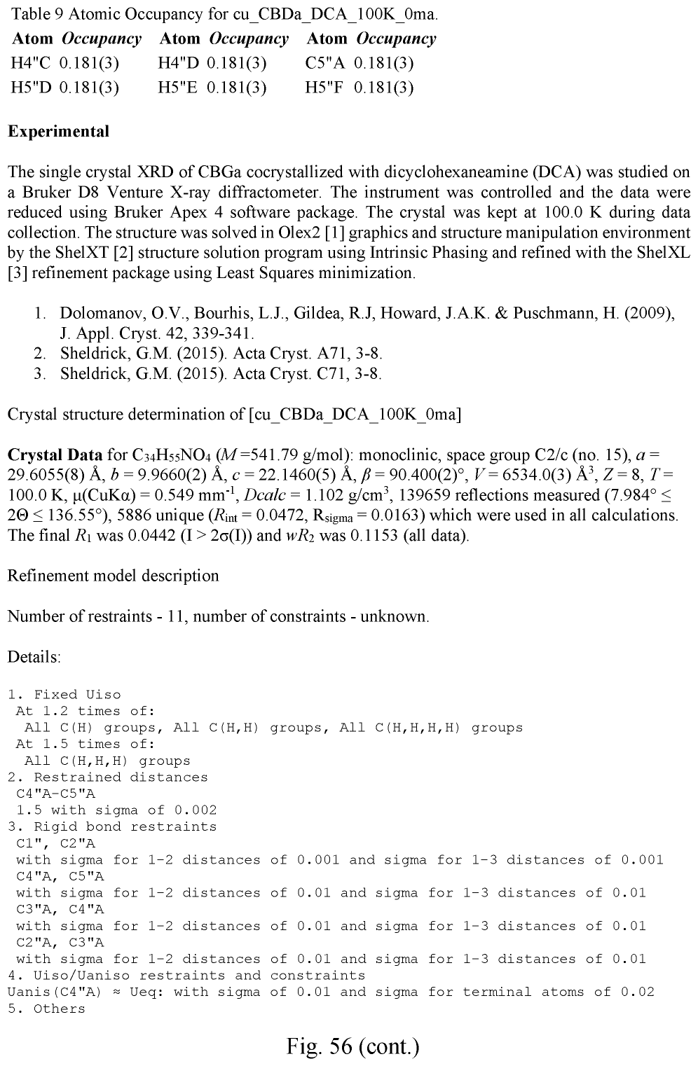

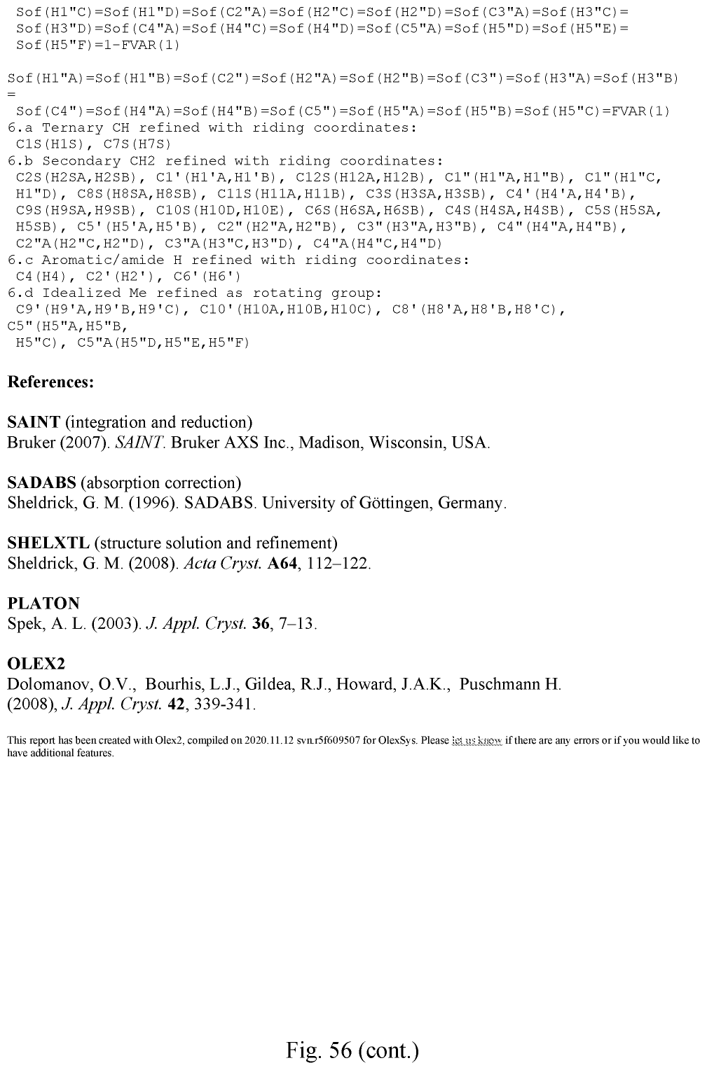

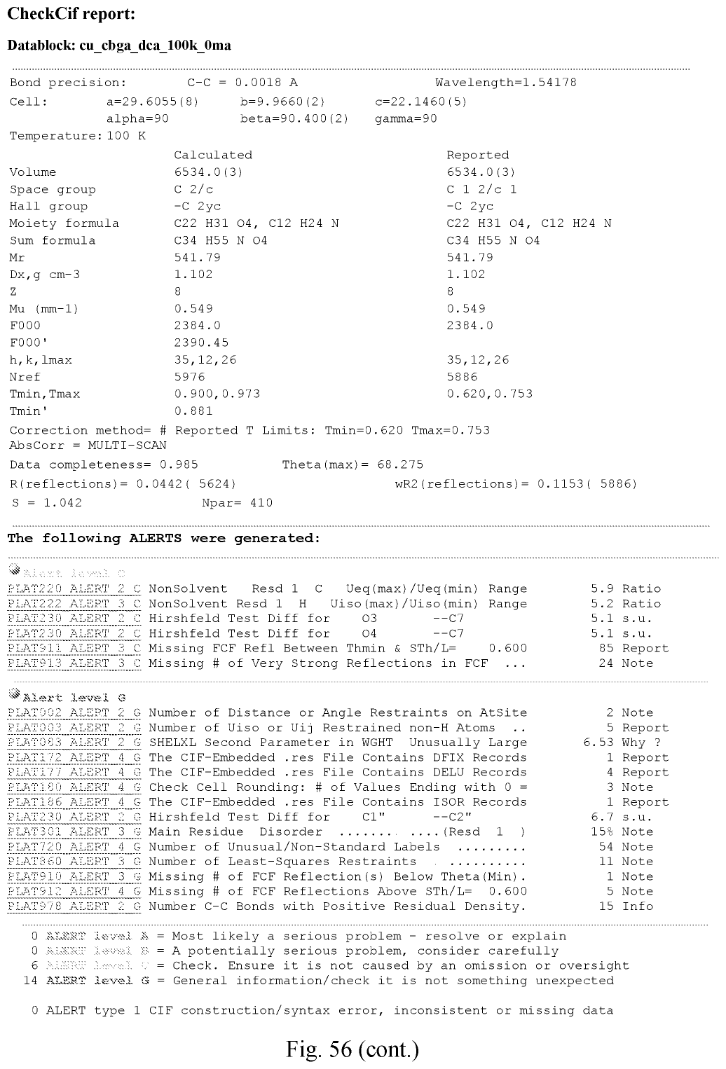

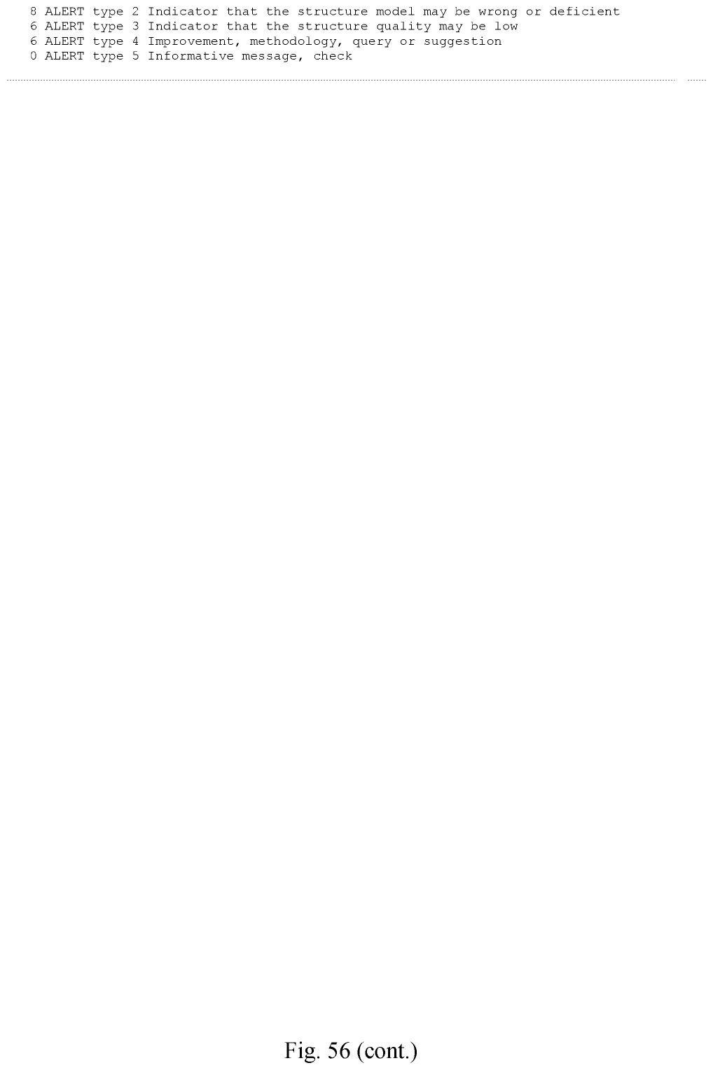

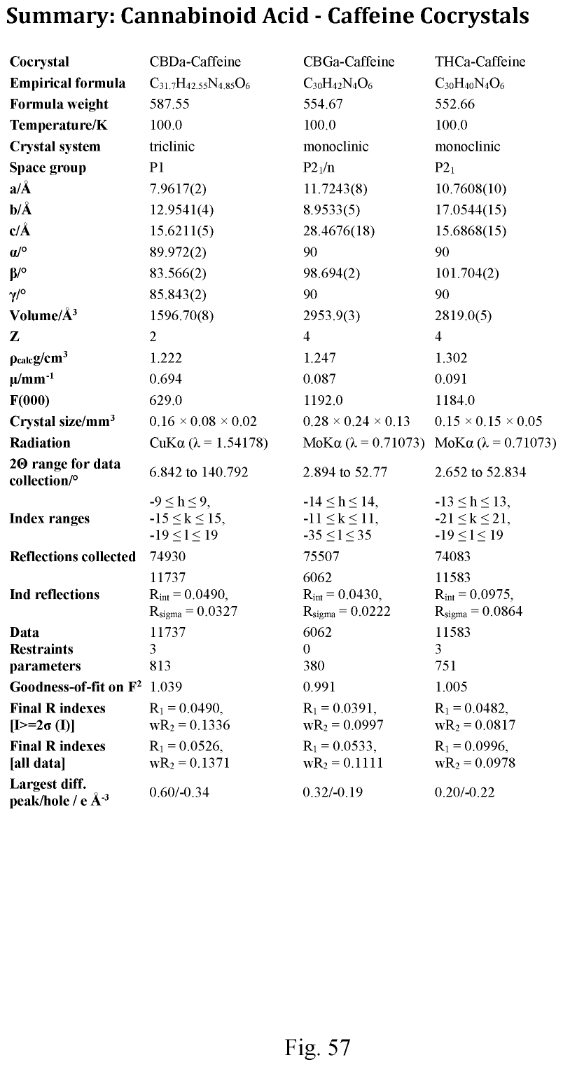

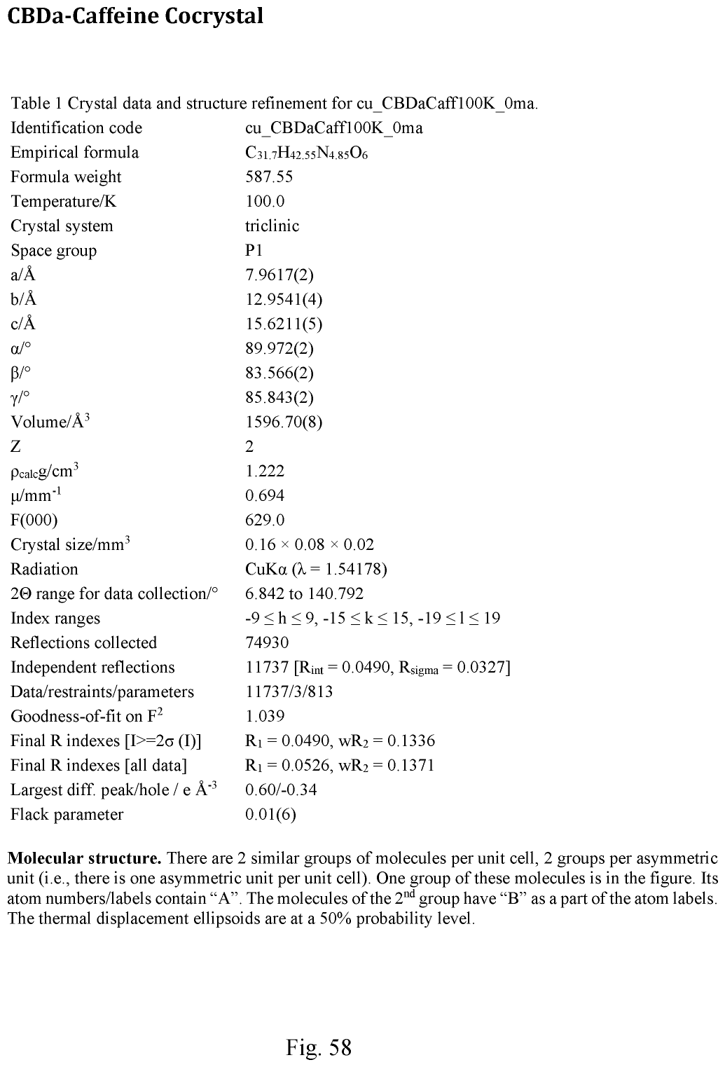

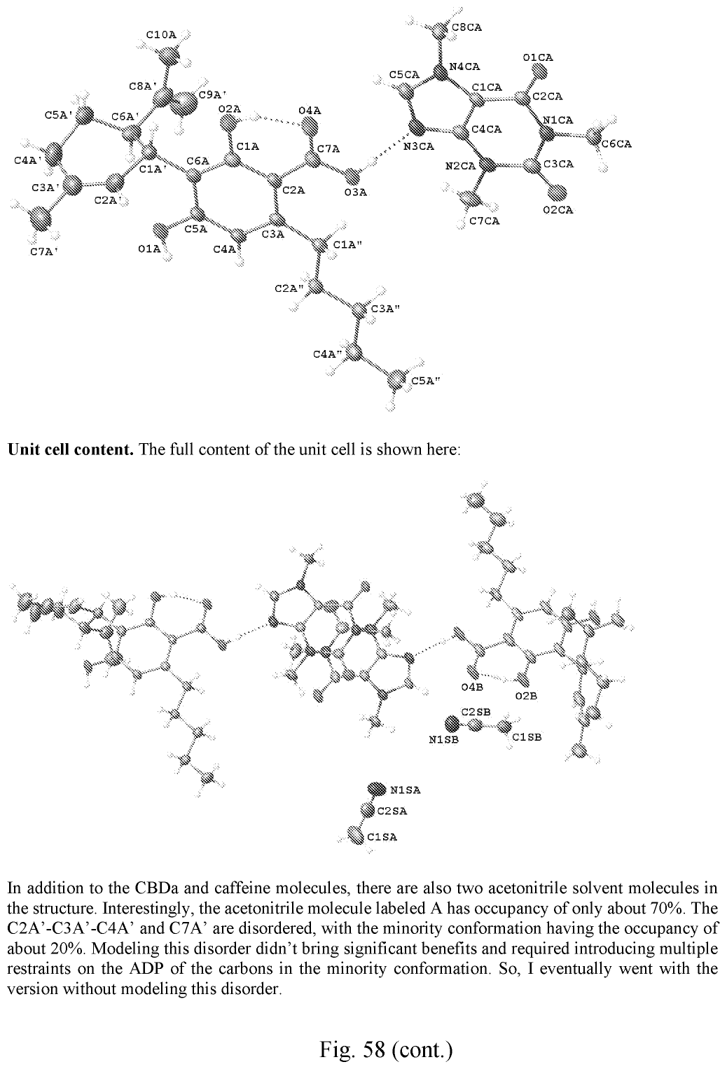

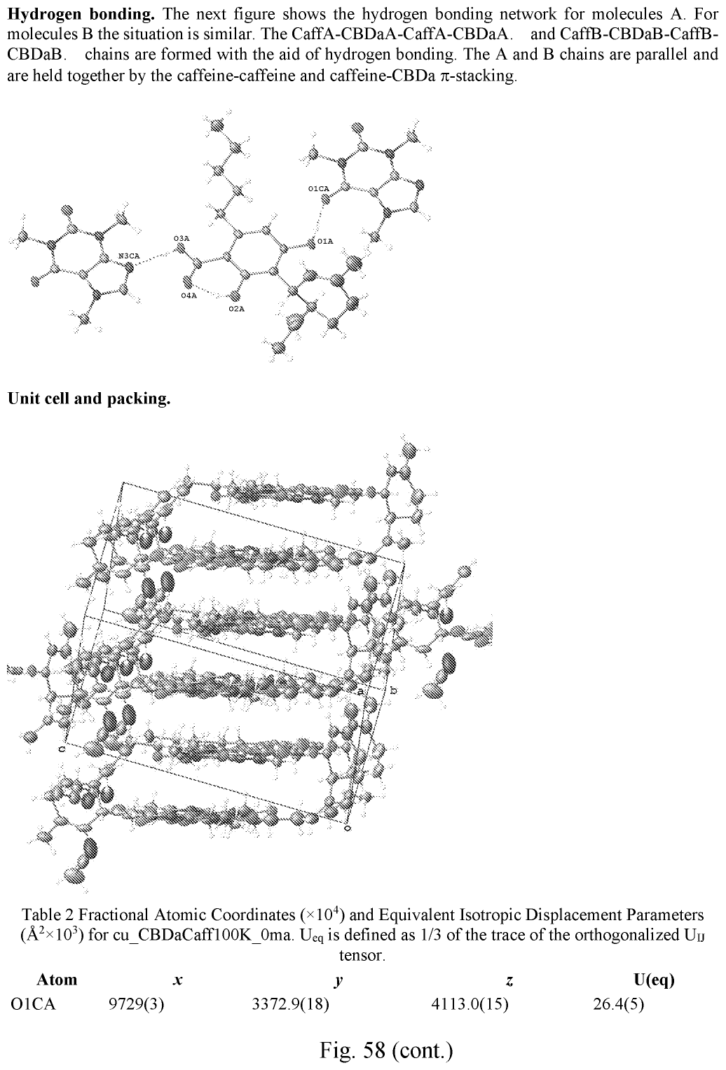

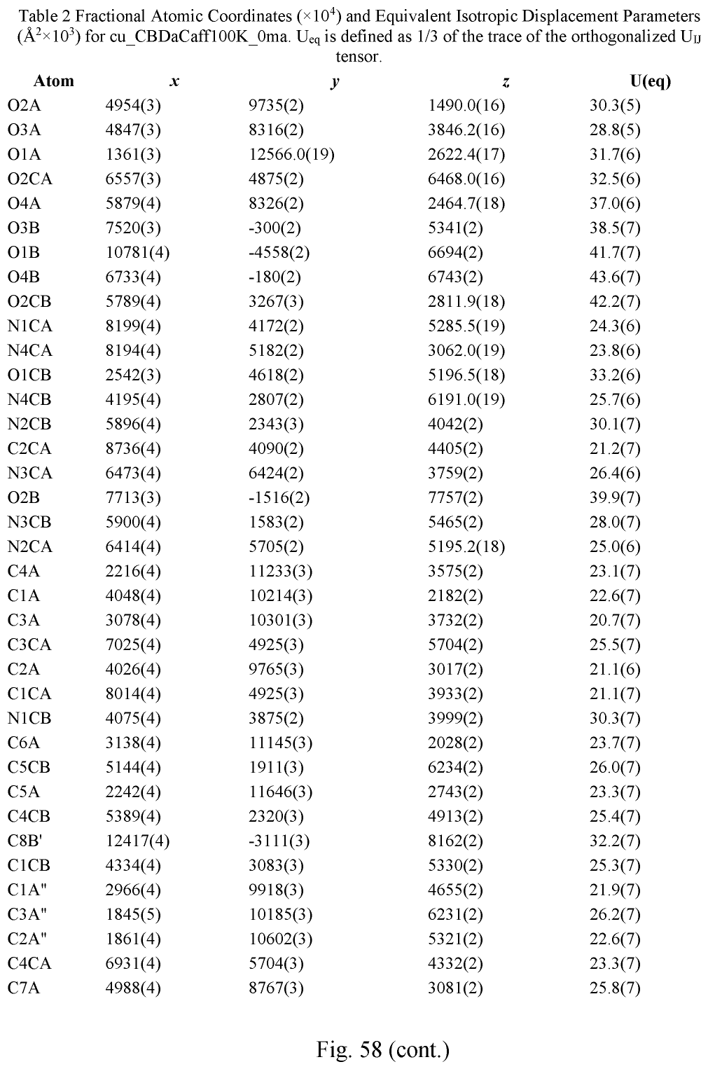

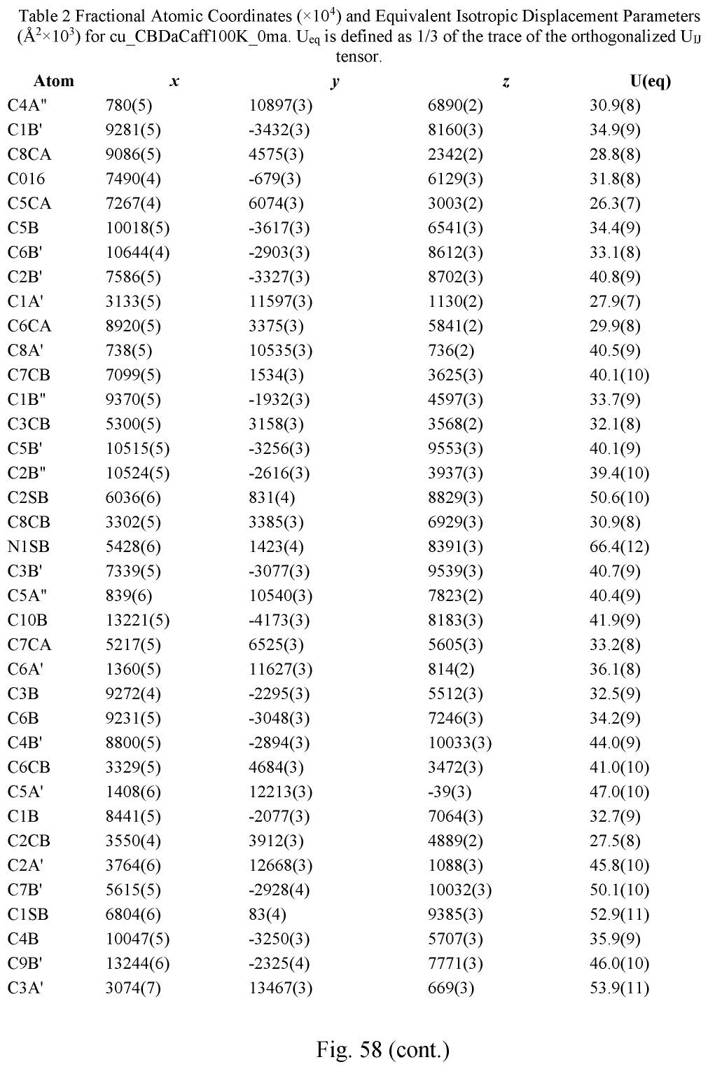

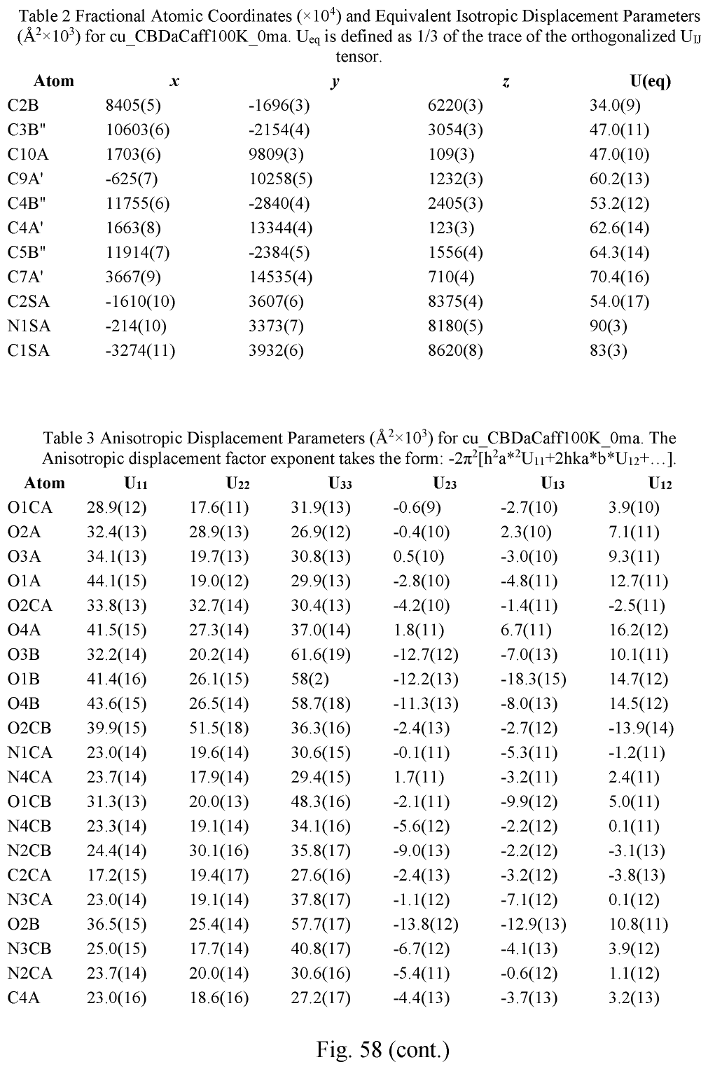

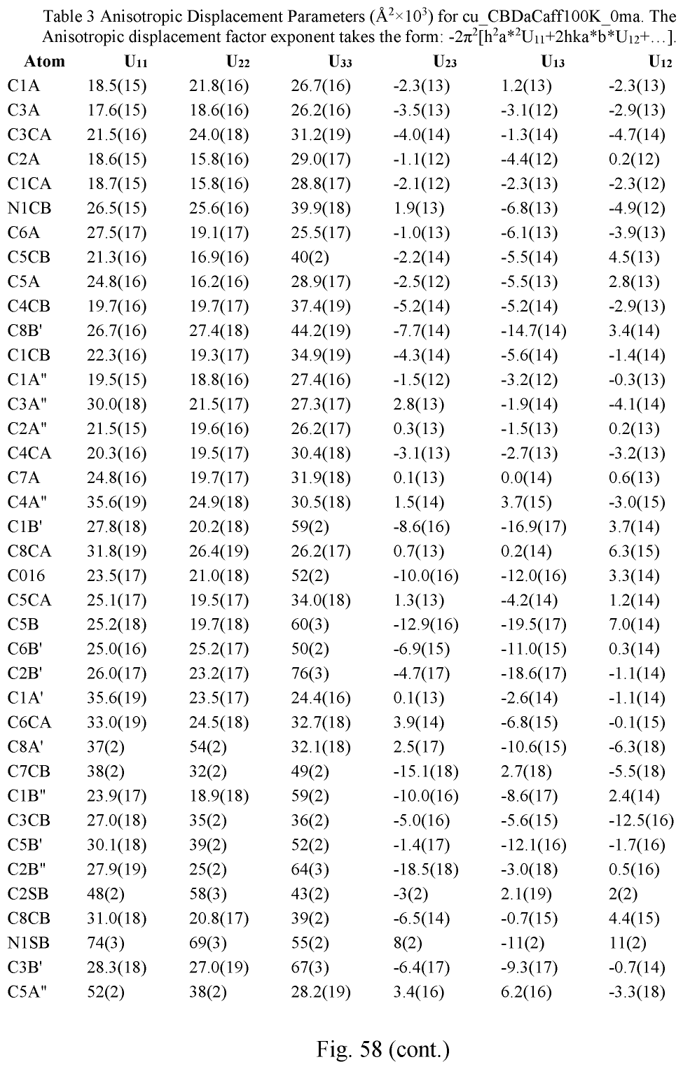

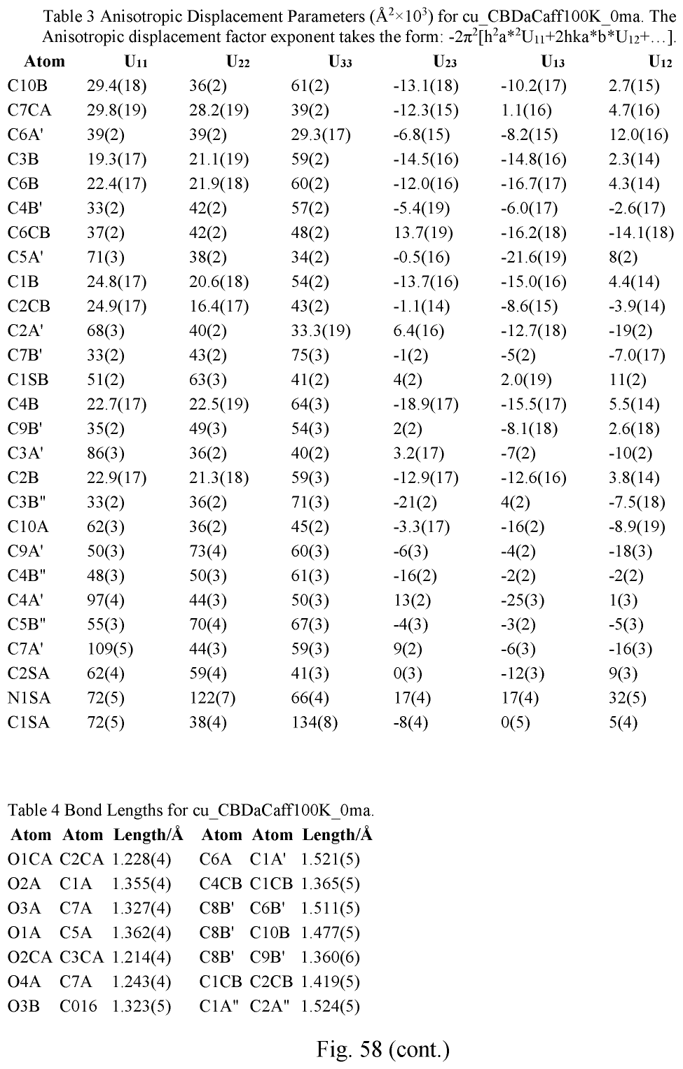

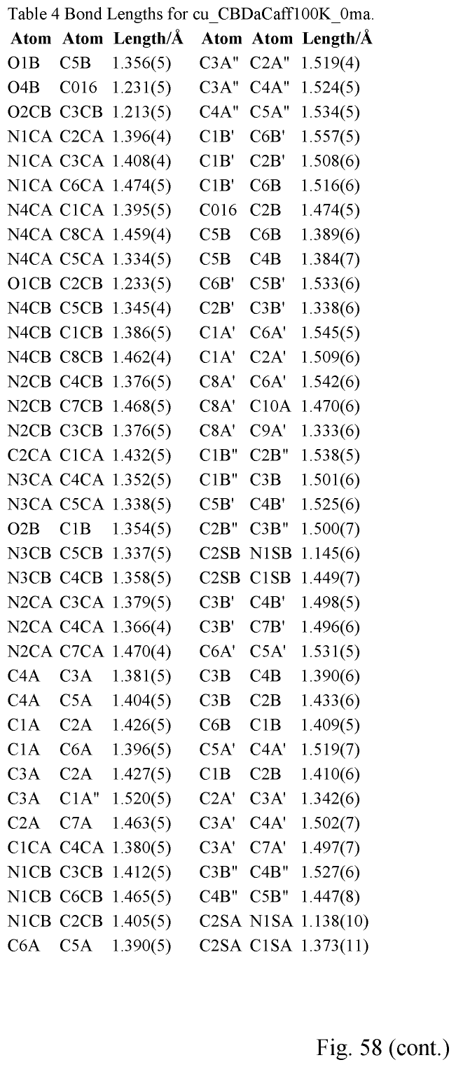

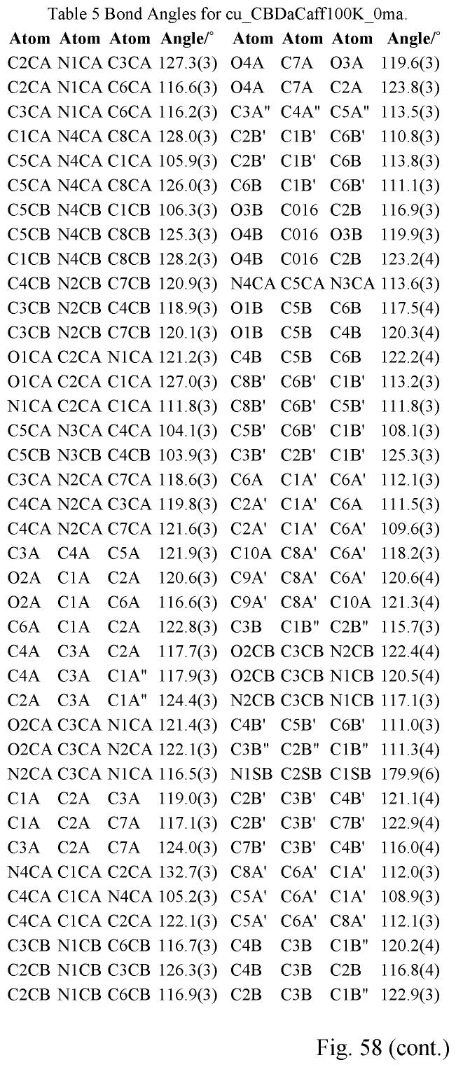

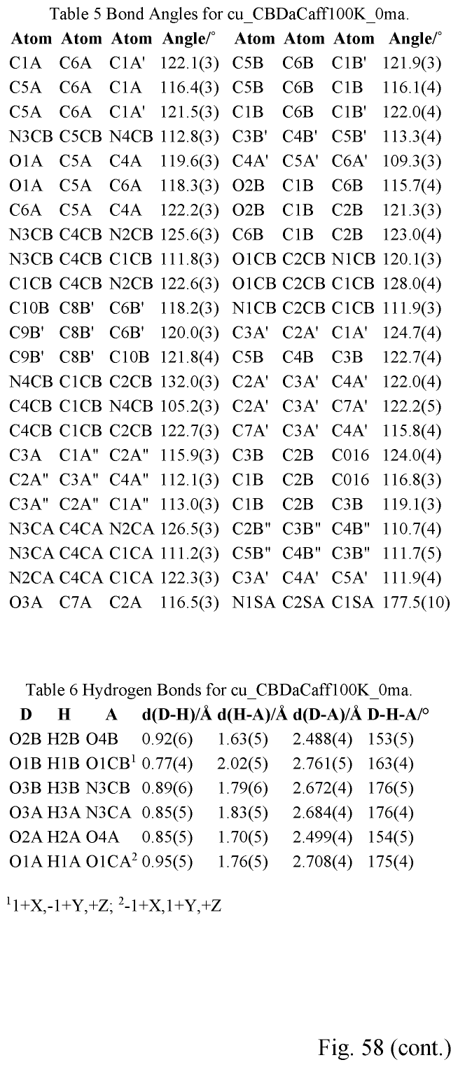

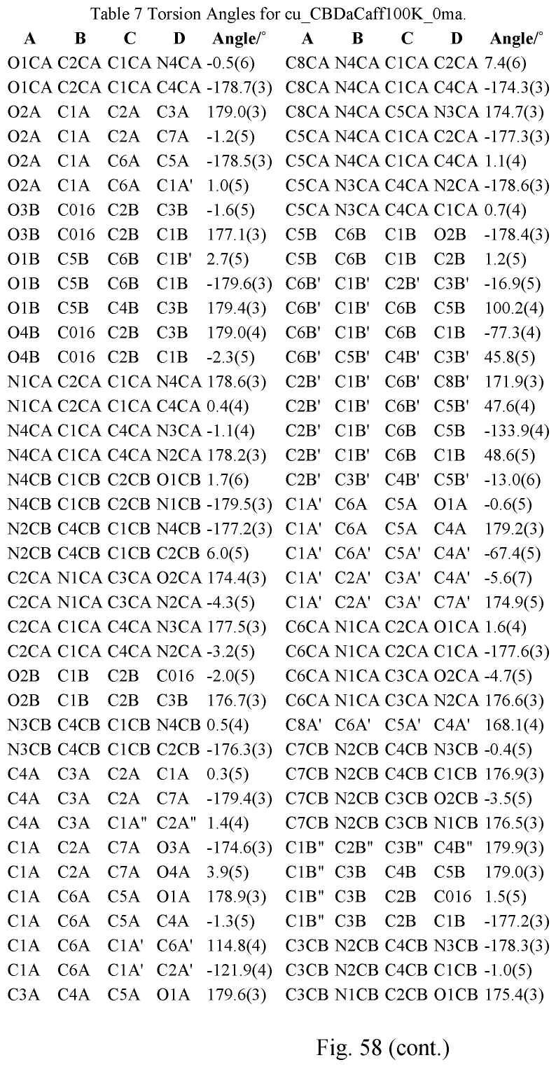

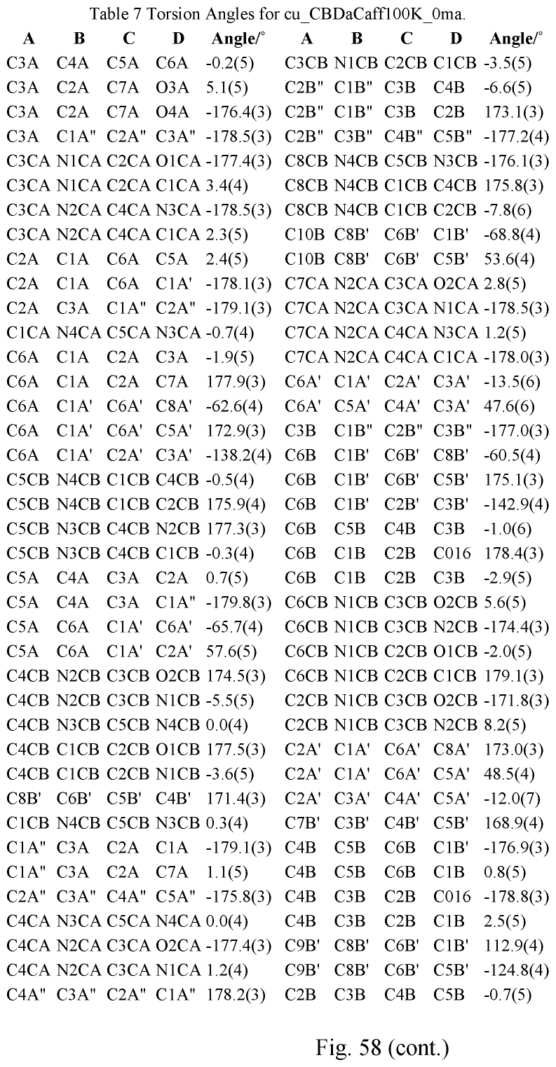

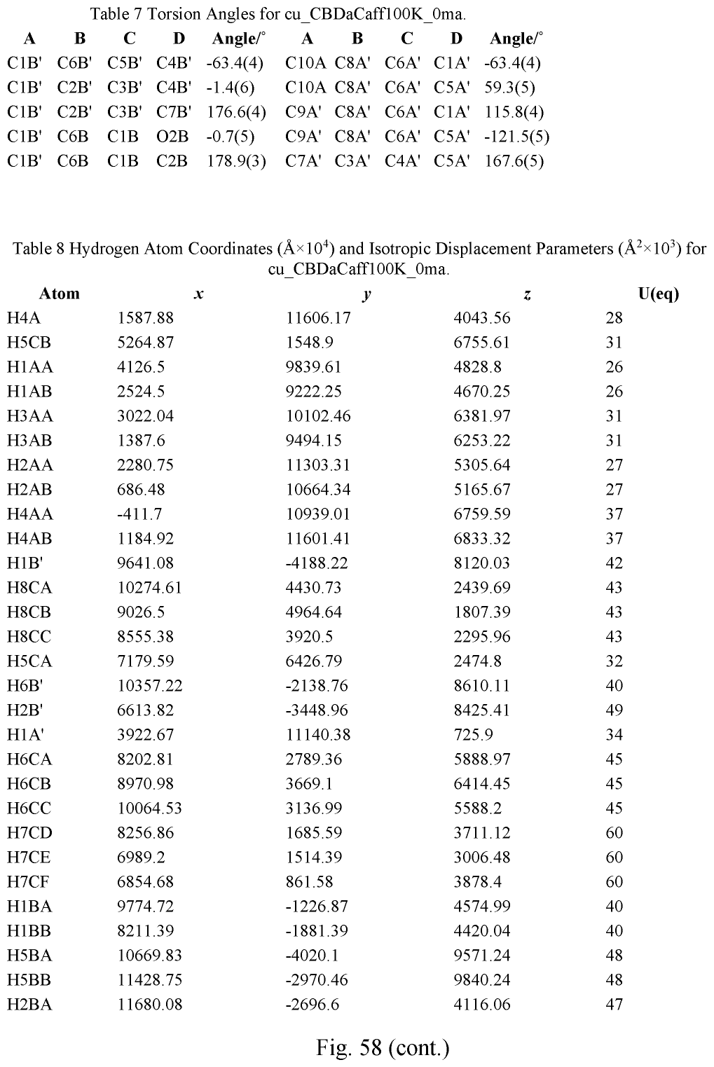

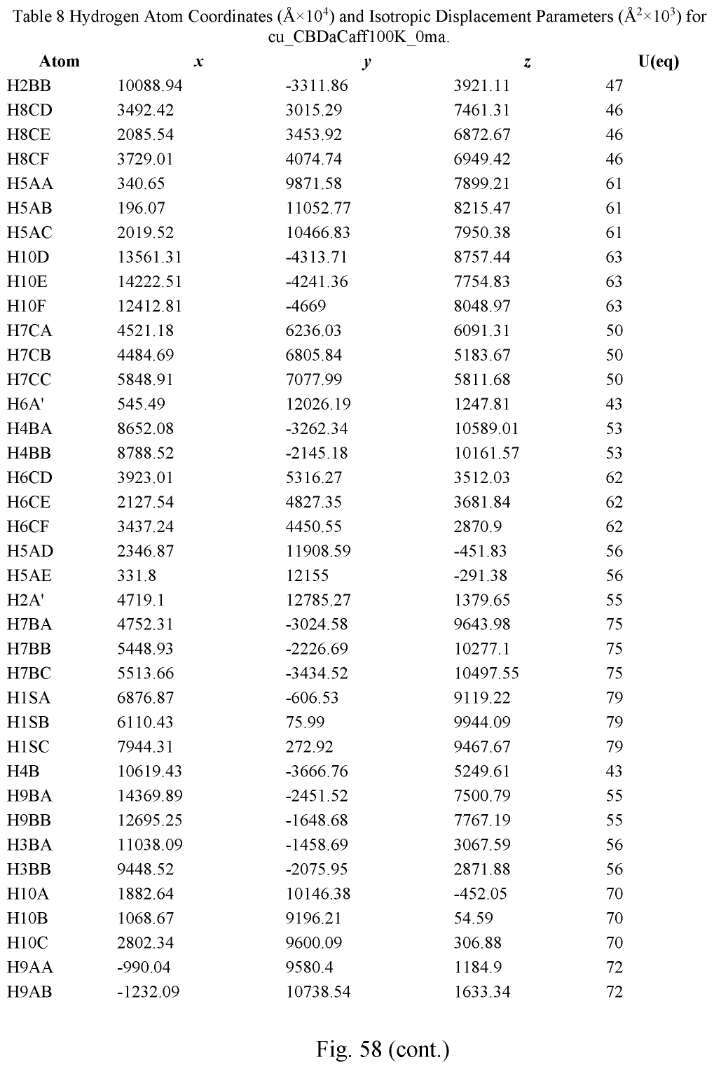

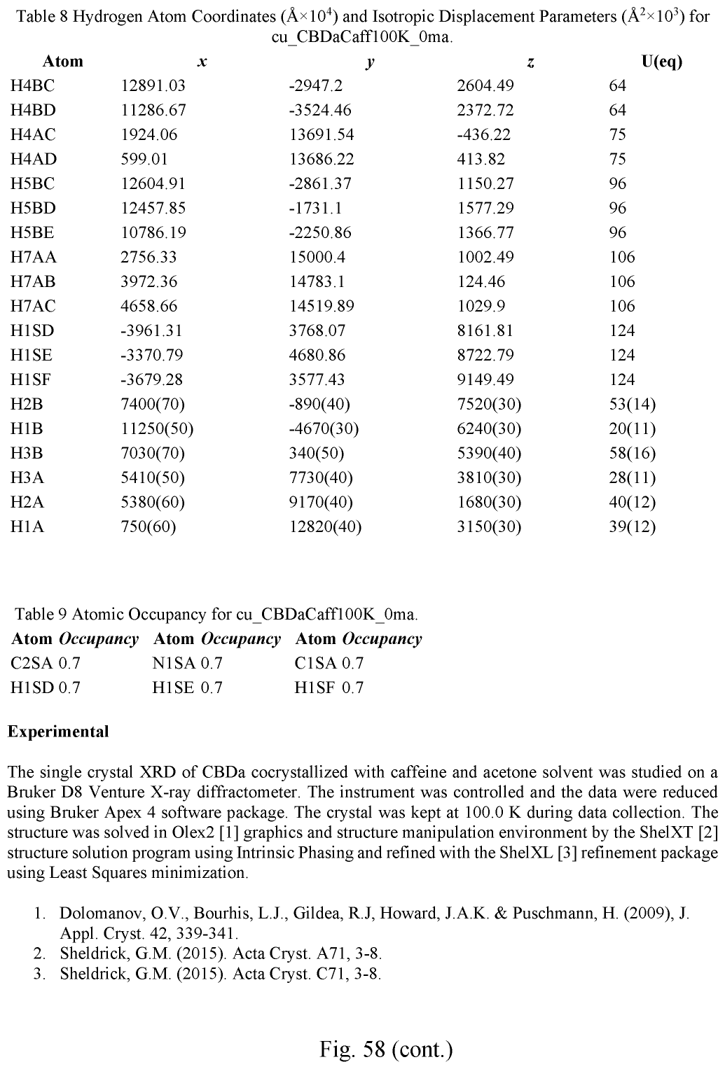

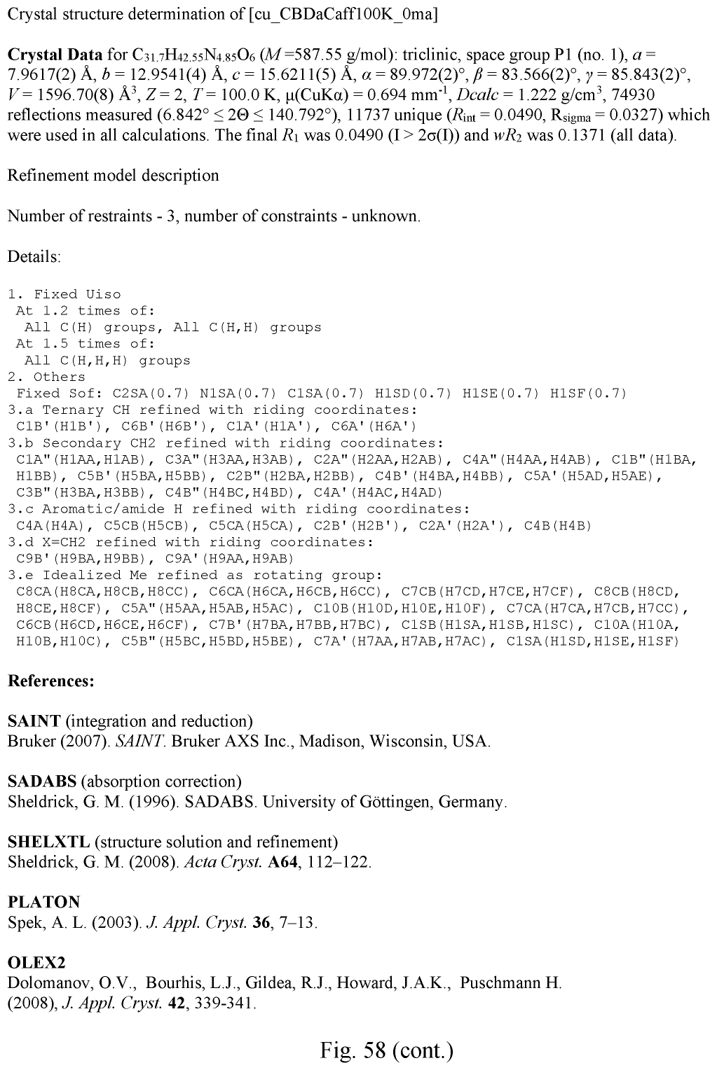

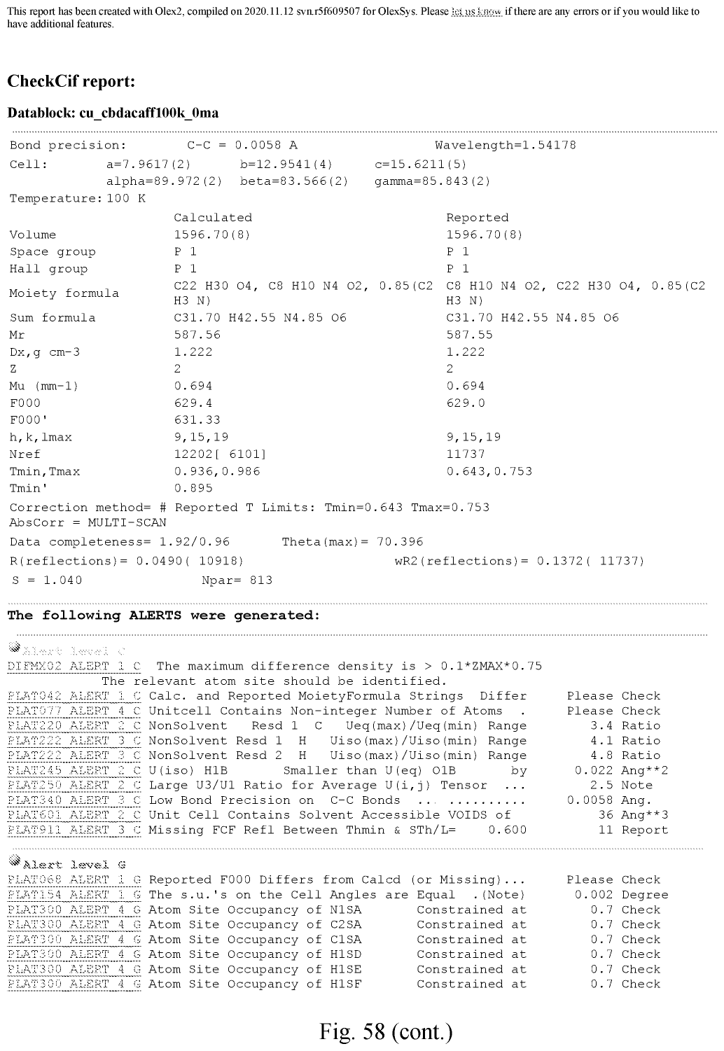

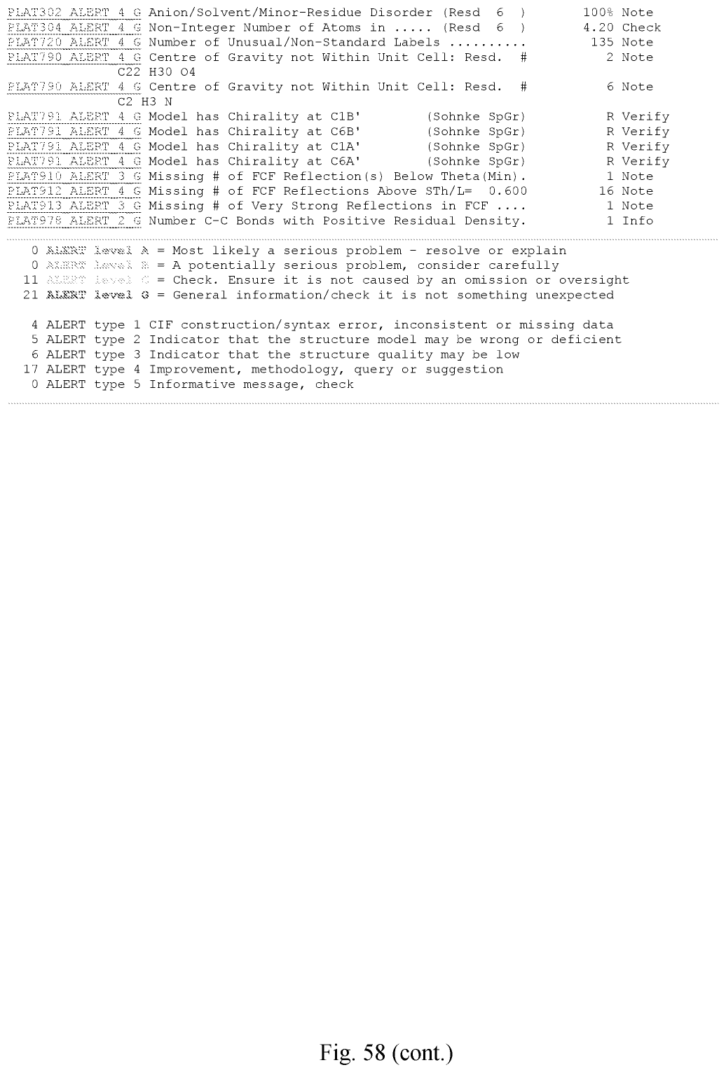

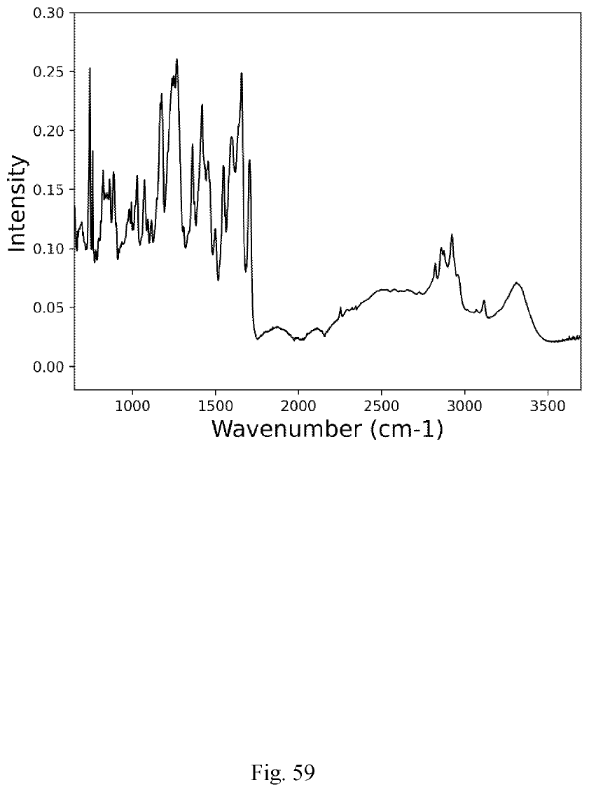

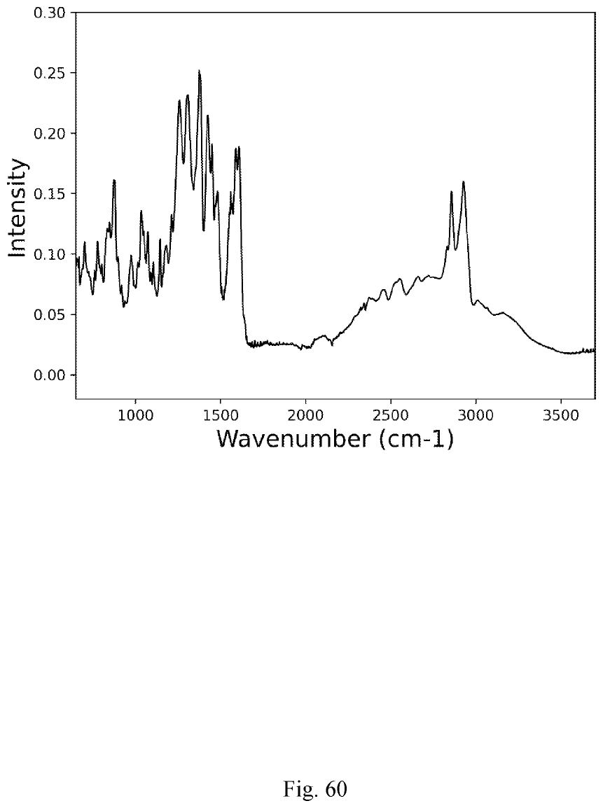

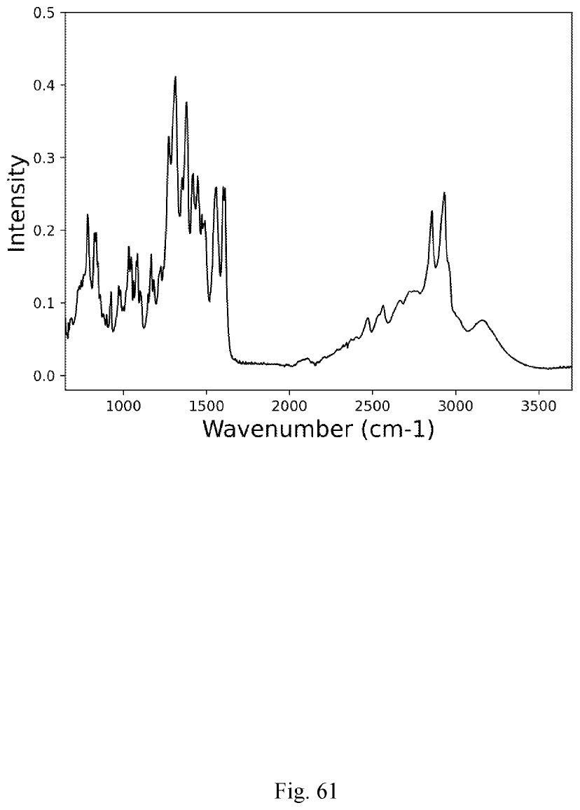

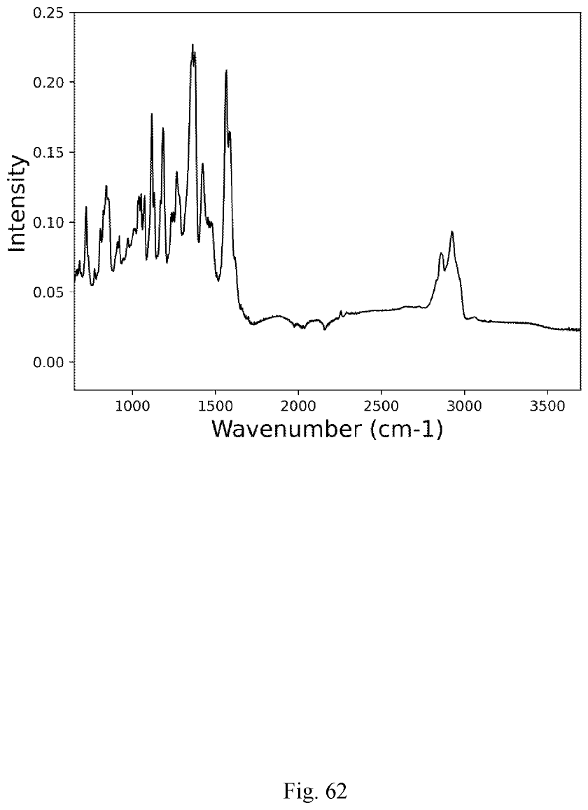

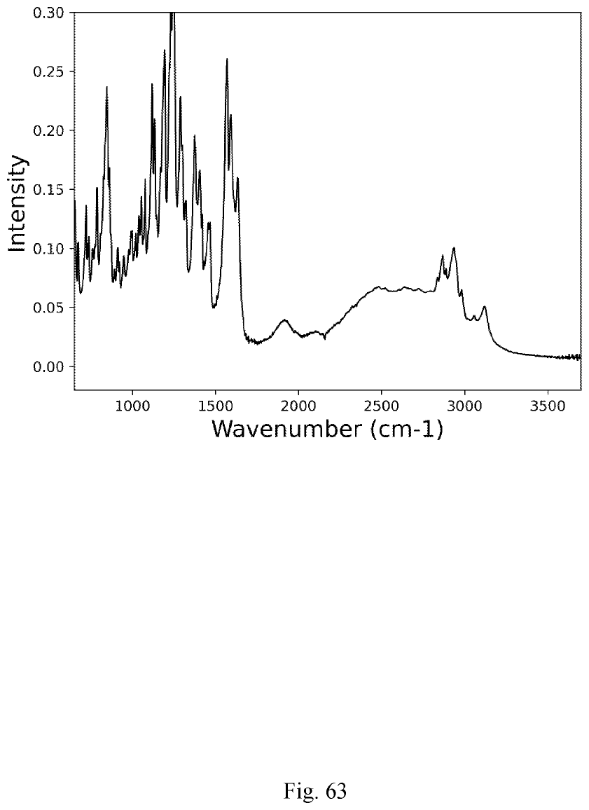

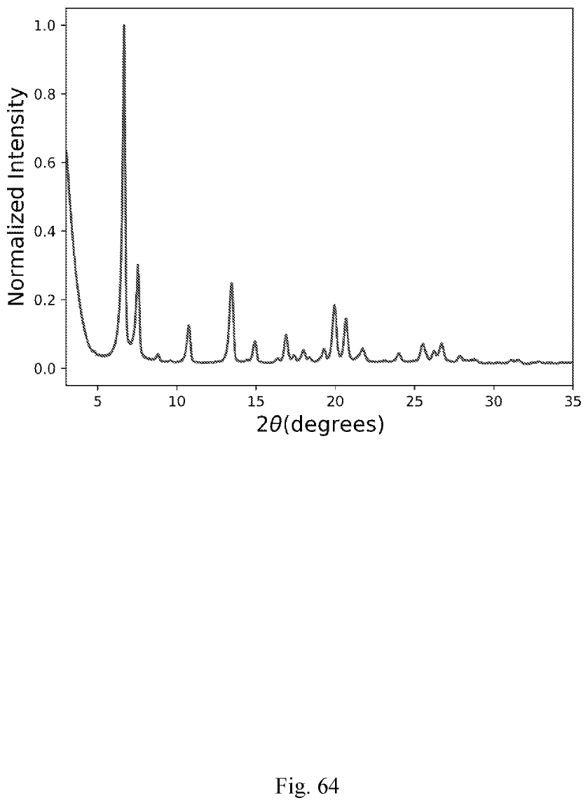

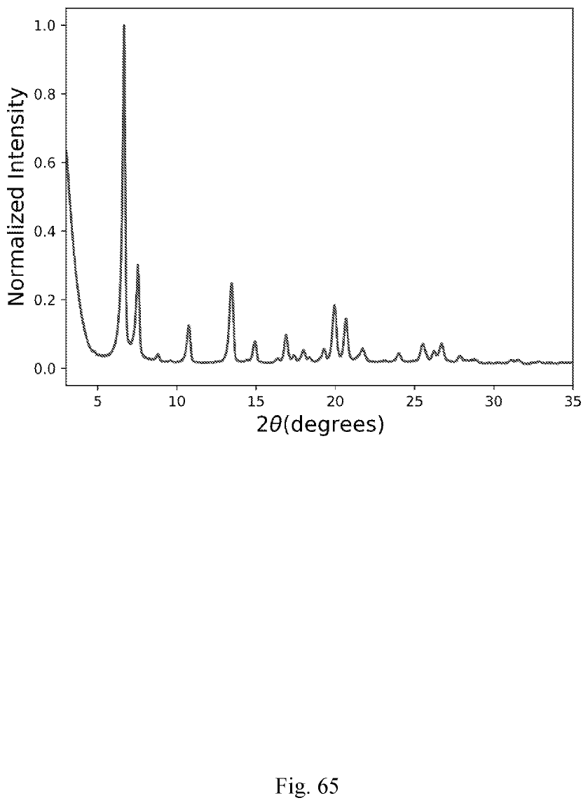

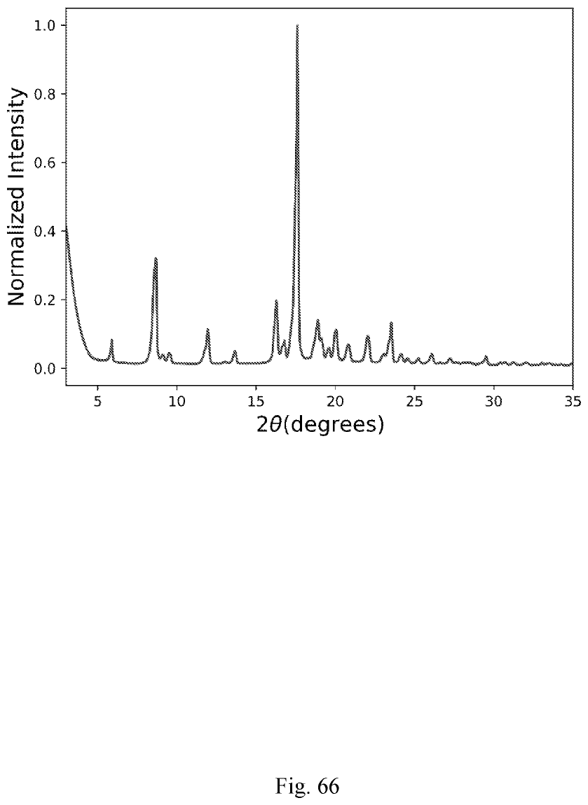

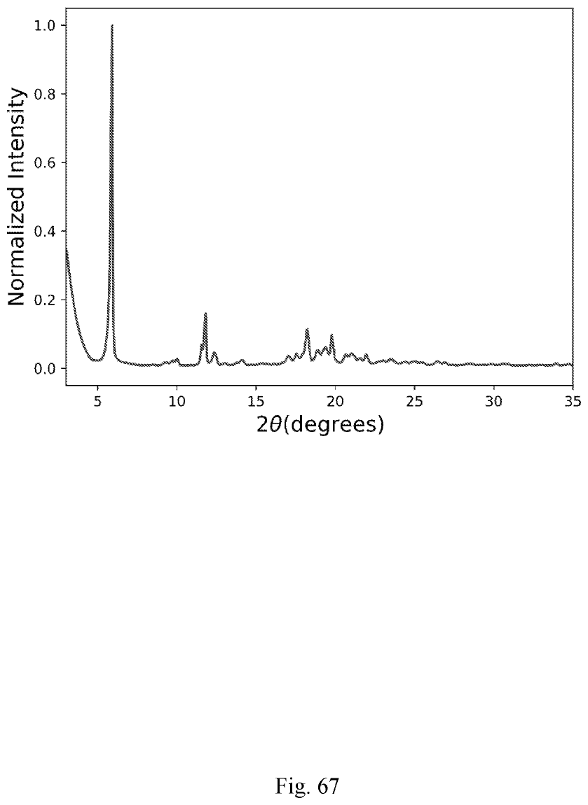

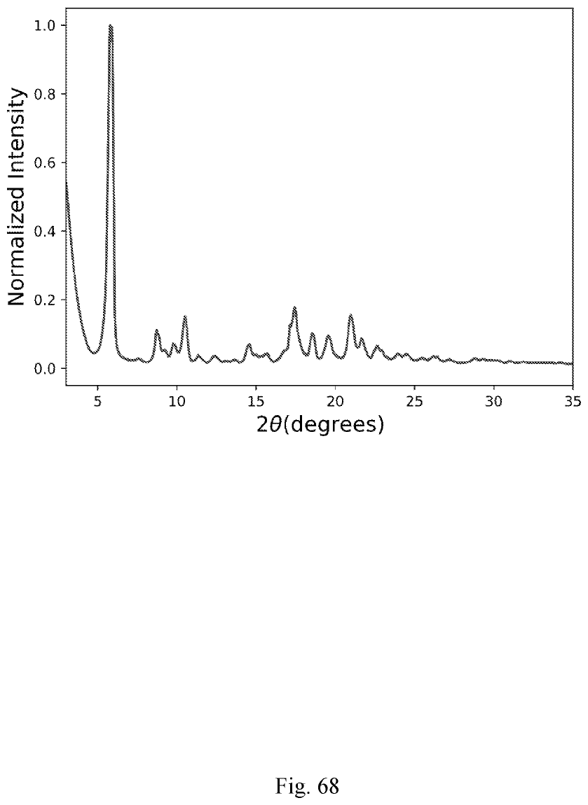

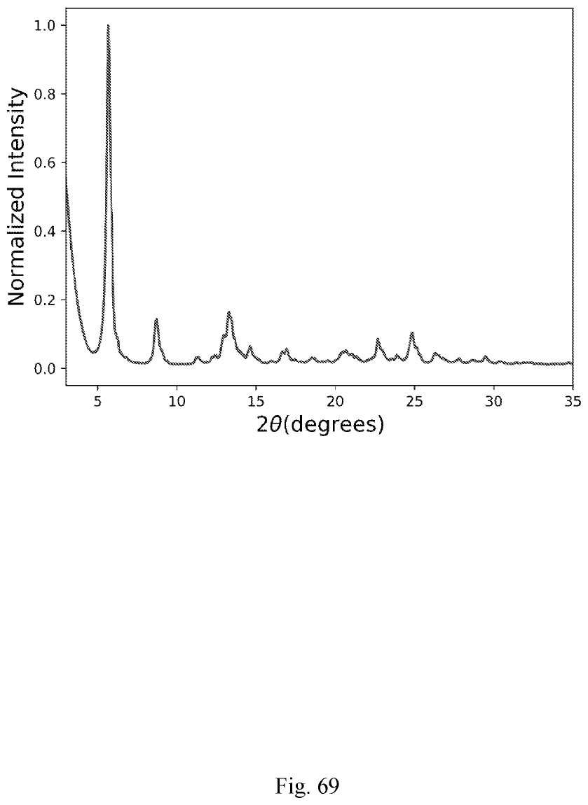

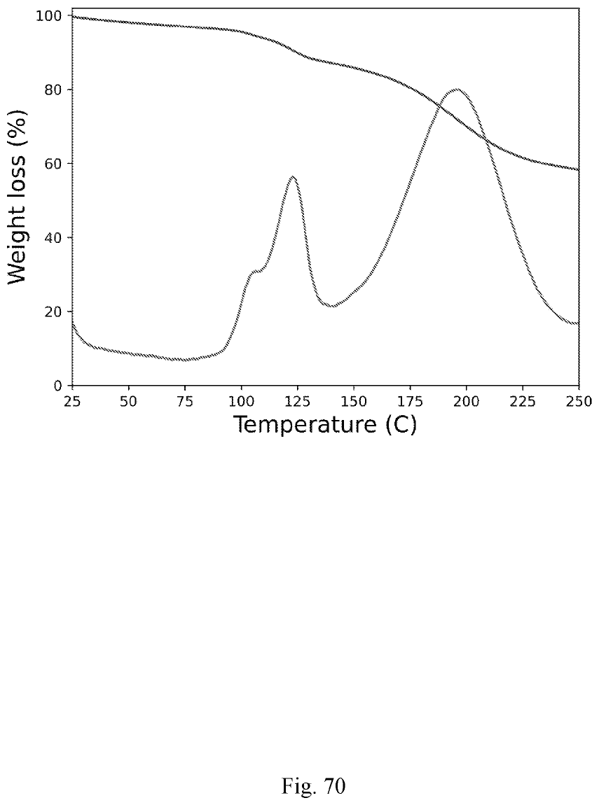

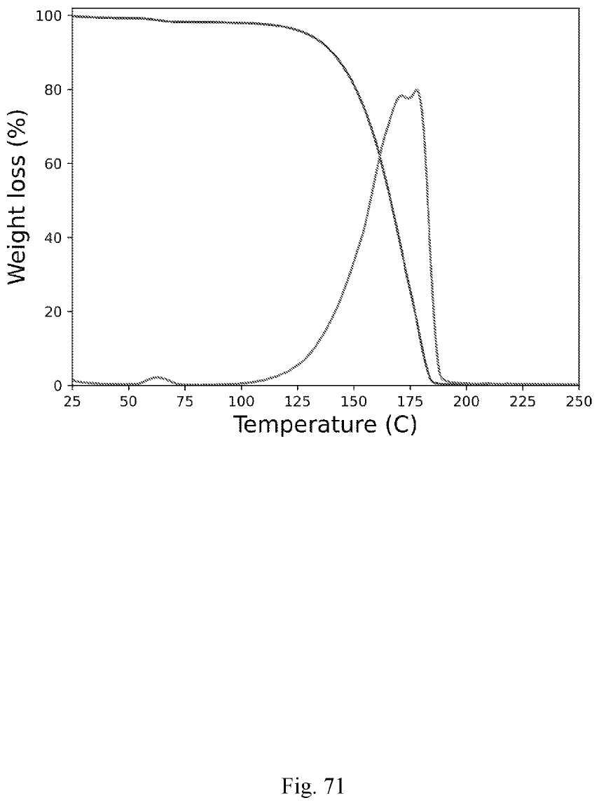

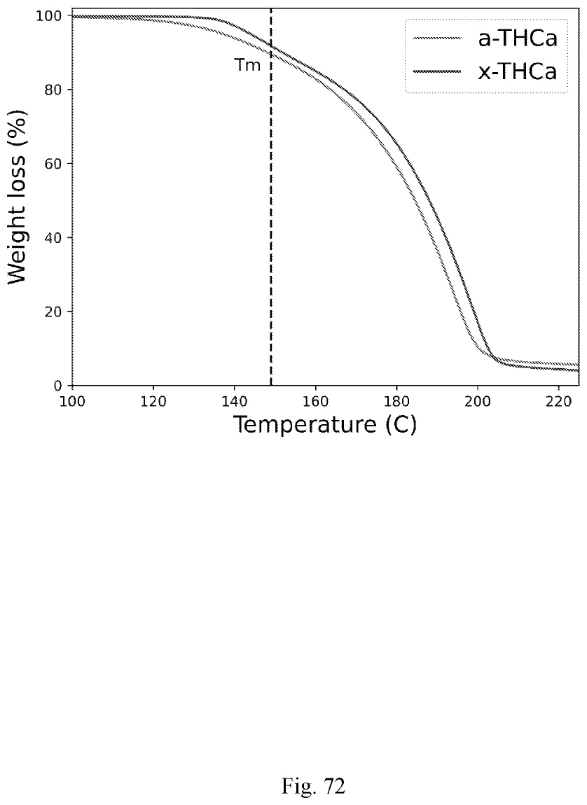

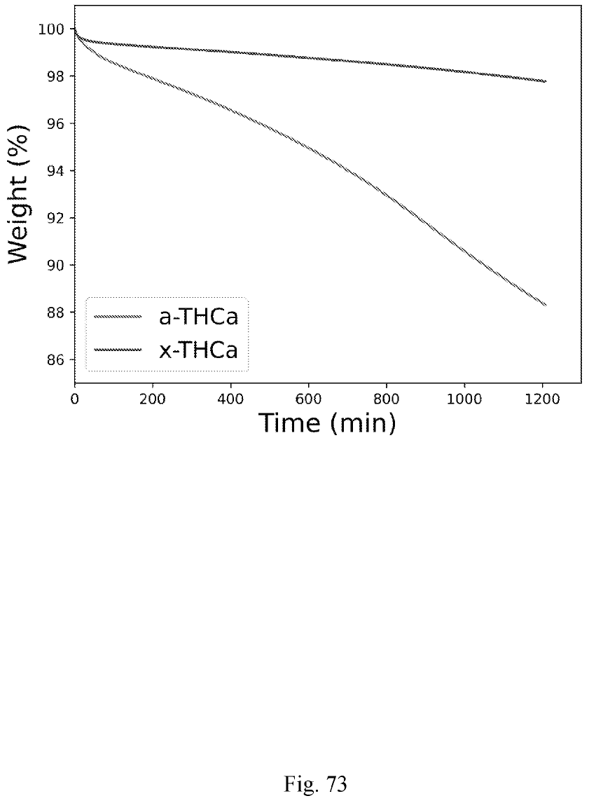

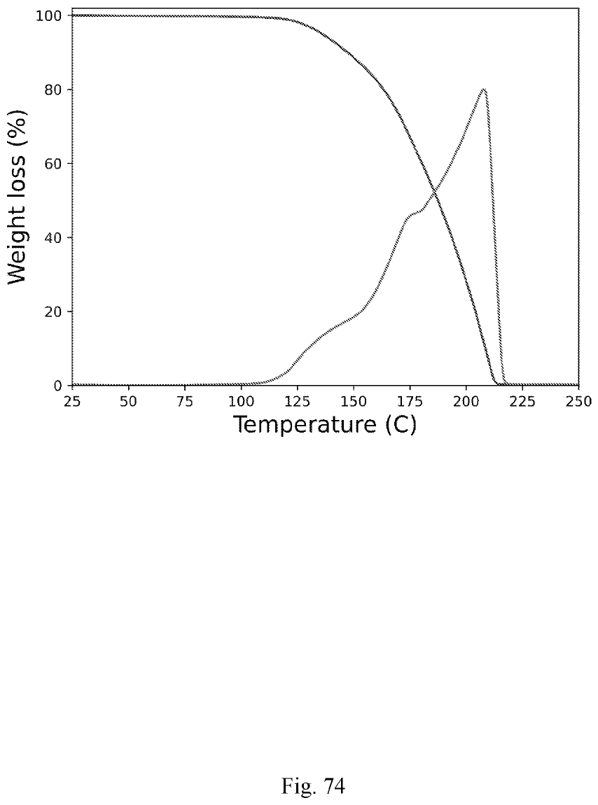

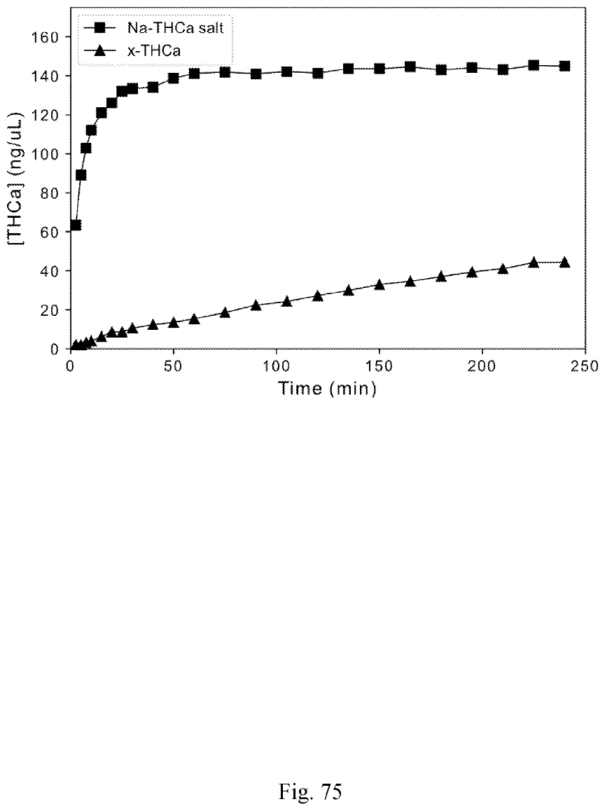

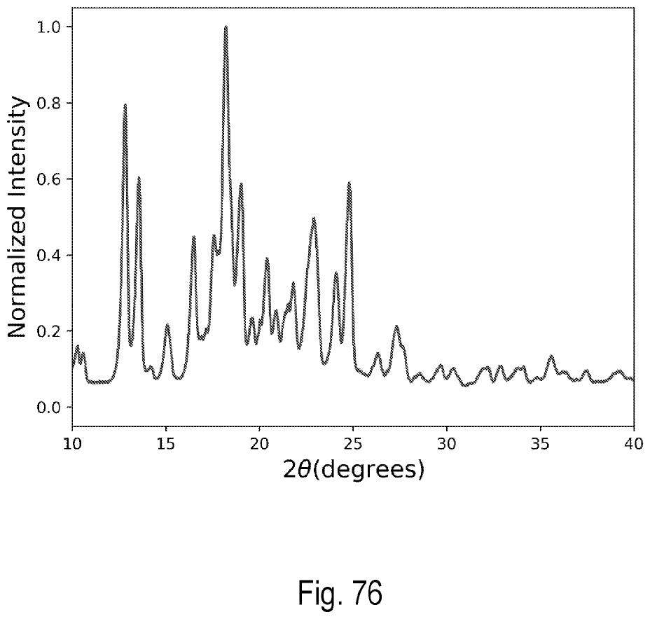

In embodiments the crystalline form of cannabidiolic acid (CBDa) can comprise an ¹H MAS NMR spectrum with at least one characteristic peak at about 1.2, 4.3, 5.3, 6.5, 11.2, 12.6 ppm. In embodiments, the crystalline form cannabidiolic acid (CBDa) can comprise an ¹H MAS NMR spectrum substantially as show in FIG. 54.

Solid State ¹H-¹³C Cross Polarization (CP) Magic Angle Spinning (MAS)

In embodiments solid state ¹H-¹³C cross polarization (CP) Magic Angle Spinning (MAS) NMR spectrum can be used to characterize crystalline forms of the compositions described herein. One of skill in the art will appreciate that peak shifts are not absolute and can vary based experimental conditions. In embodiments, one of skill in the art will recognize that peak shifts can vary by about +/−1 ppm.

In embodiments the crystalline form of tetrahydrocannabinolic acid (THCa) can comprise an ¹H-¹³C CP MAS NMR spectrum with at least one characteristic peak at about 13.8, 15.4, 17.9, 19.3, 20.7, 23.6, 24.2, 24.6, 25.1, 25.9, 26.6, 27.5, 28.5, 31.0, 32.3, 32.9, 35.3, 37.6, 44.1, 45.9, 77.8, 78.9, 101.5, 102.6, 109.6, 110.1, 102.6, 109.6, 110.1, 113.3, 123.1, 124.0, 132.3, 133.2, 146.6, 161.0, 162.0, 165.0, 165.6, 178.1, and 178.5 ppm. In embodiments, the crystalline form of tetrahydrocannabinolic acid (THCa) can comprise an ¹H-¹³C CP MAS NMR spectrum substantially as show in FIG. 7.

In embodiments the crystalline form of cannabigerolic acid (CBGa) can comprise an ¹H-¹³C CP MAS NMR spectrum with at least one characteristic peak at about 9.8, 11.3, 13.5, 16.7, 18.8, 21.8, 23.6, 26.3, 28.3, 33.8, 37.7, 98.9, 107.3, 109.0, 117.9, 120.9, 126.0, 132.3, 142.5, 153.5, 159.7, 171.8 ppm. In embodiments, the crystalline form of cannabigerolic acid (CBGa) can comprise an ¹H-¹³C CP MAS NMR spectrum substantially as show in FIG. 35.

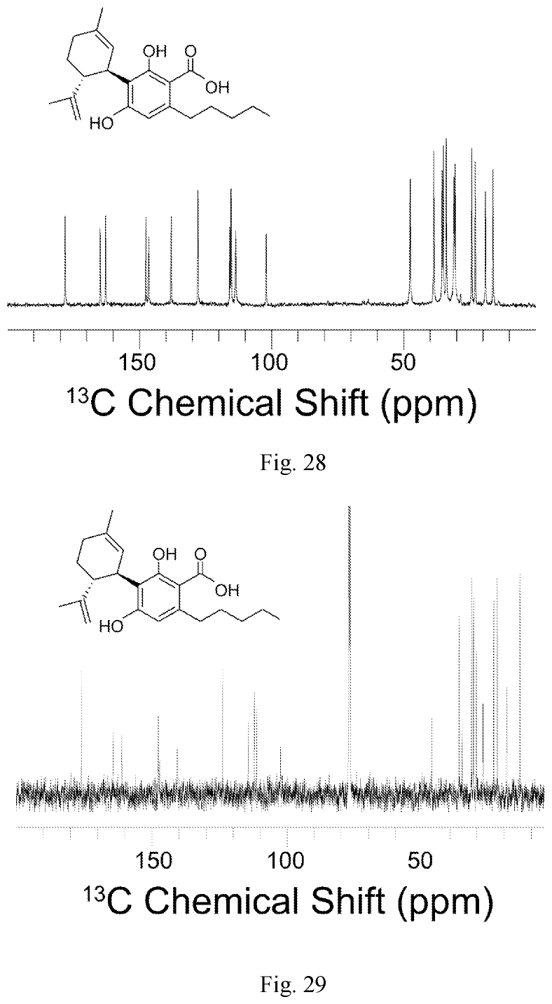

In embodiments the crystalline form of cannabidiolic acid (CBDa) can comprise an ¹H-¹³C CP MAS NMR spectrum with at least one characteristic peak at about 16.2, 19.0, 22.8, 24.2, 30.5, 31.0, 33.8, 34.94, 35.43, 38.6, 47.5, 102.0, 113.5, 115.3, 115.8, 127.8, 138.0, 146.5, 147.5, 162.8, 164.8, 178.1 ppm. In embodiments, the crystalline form cannabidiolic acid (CBDa) can comprise an ¹H-¹³C CP MAS NMR spectrum substantially as show in FIG. 28.

### Thermogravimetric Analysis (TGA) Preparation and Experimental Conditions

Thermal gravimetric analysis (TGA) was carried out using a TA instruments 2910 under dry nitrogen flow (30 mL/min for both the furnace and the balance). For a typical experiment, 5-10 mg of solid material was placed into the pan and was allowed to equilibrate at room temperature under nitrogen for approximately 5 minutes prior to the start of each experiment to ensure a stable baseline. A heating rate of 1° C./min or 5° C./min was utilized.

In embodiments the crystalline form of tetrahydrocannabinolic acid (THCa) can comprise an 20% w/w loss from about 125° C. to about 165° C. and an about 70% w/w/loss from about 165° C. to about 200° C. and a degradation onset at about 125° C. In embodiments, the crystalline form of tetrahydrocannabinolic acid (THCa) can comprise a TGA thermogram as show in FIG. 9.

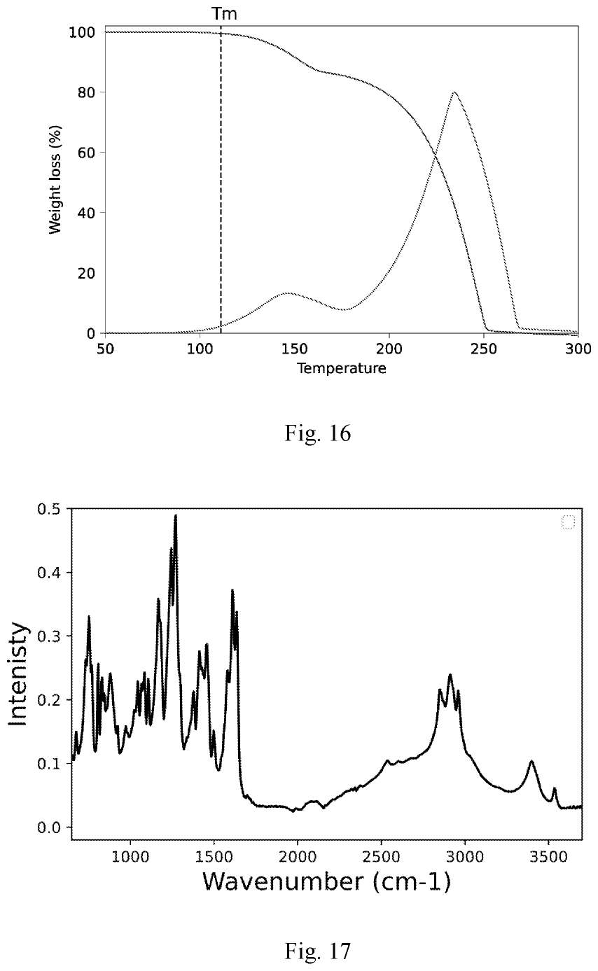

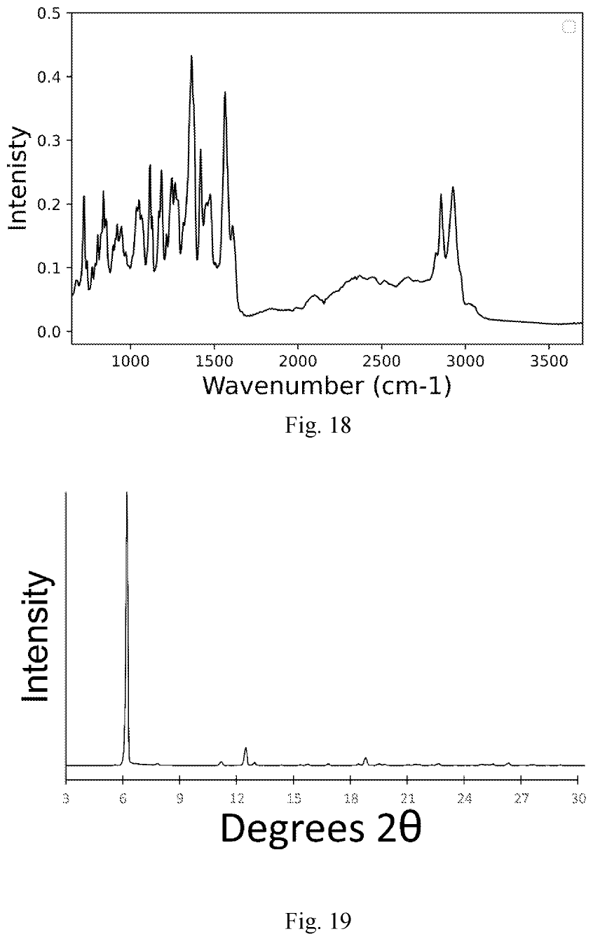

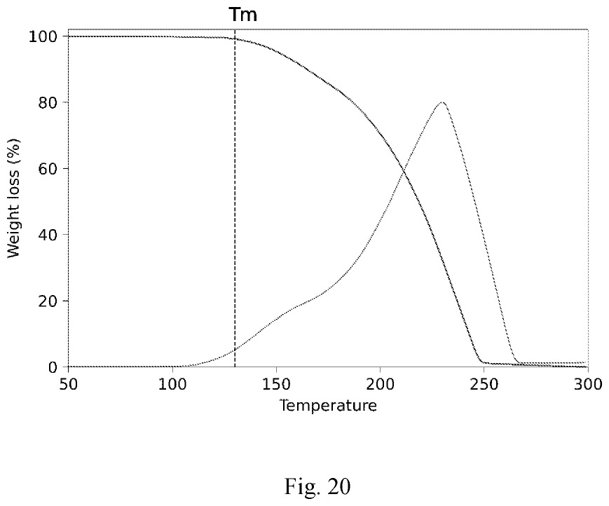

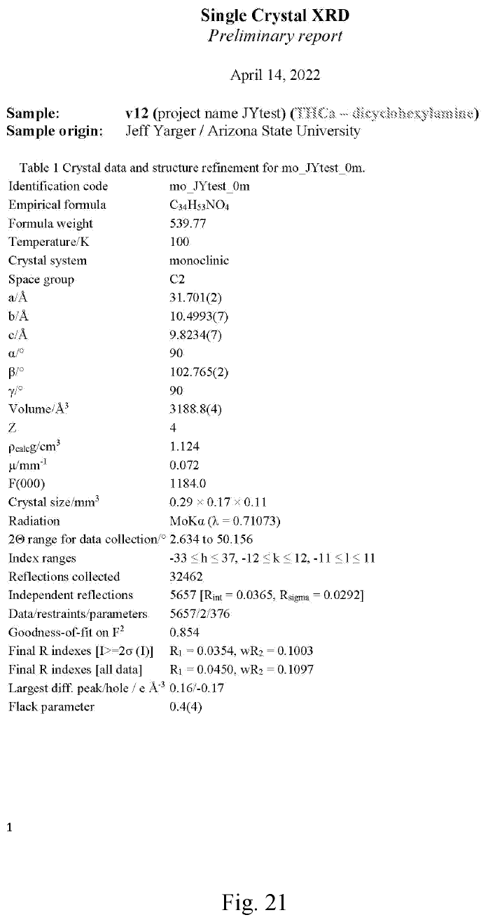

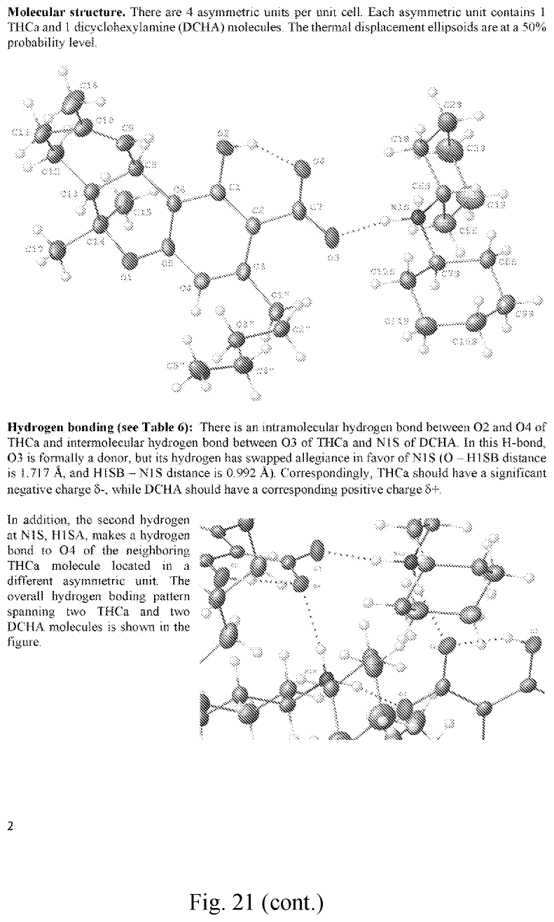

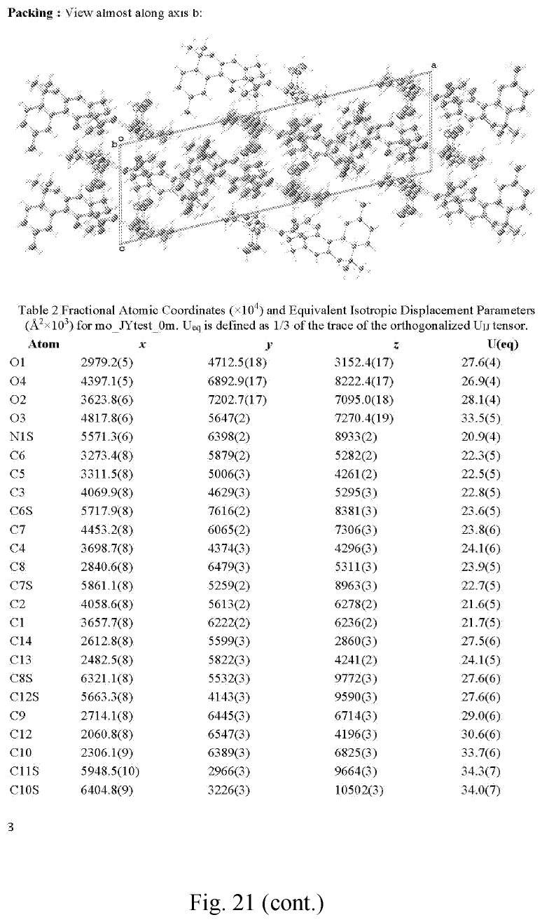

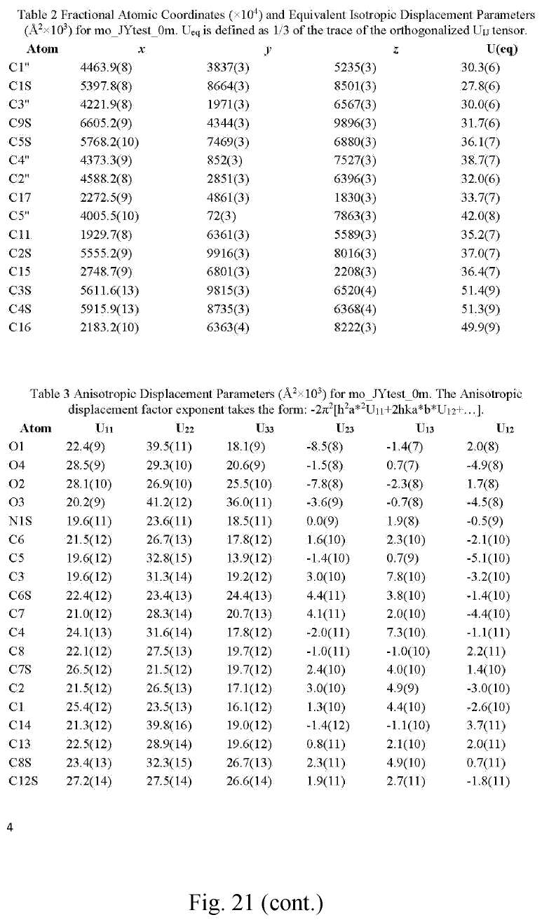

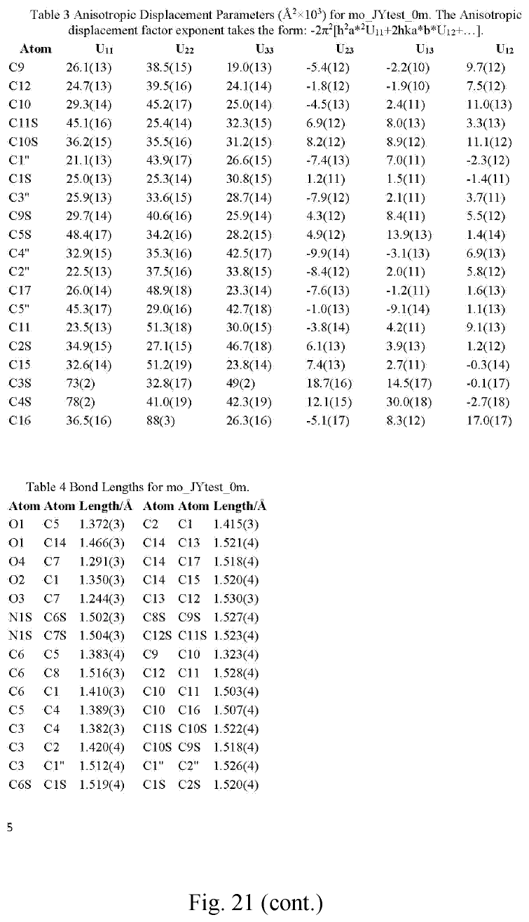

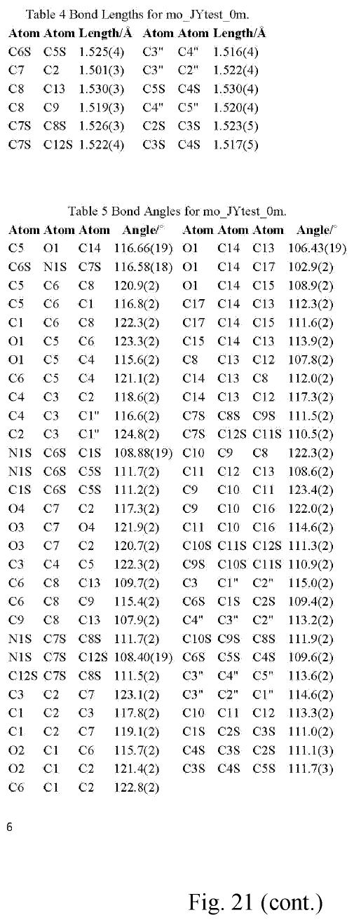

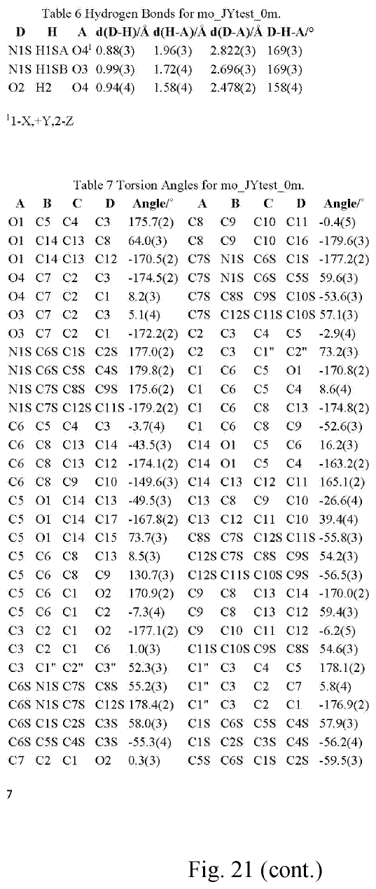

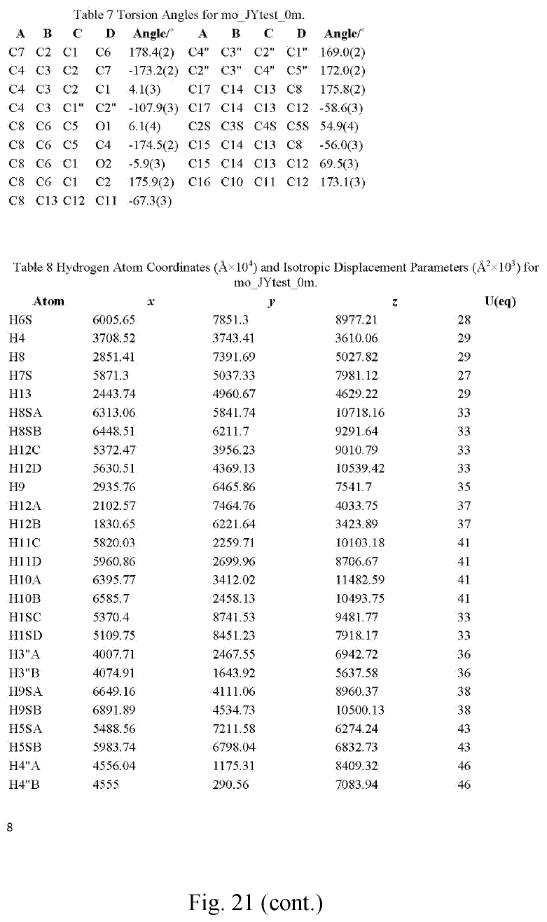

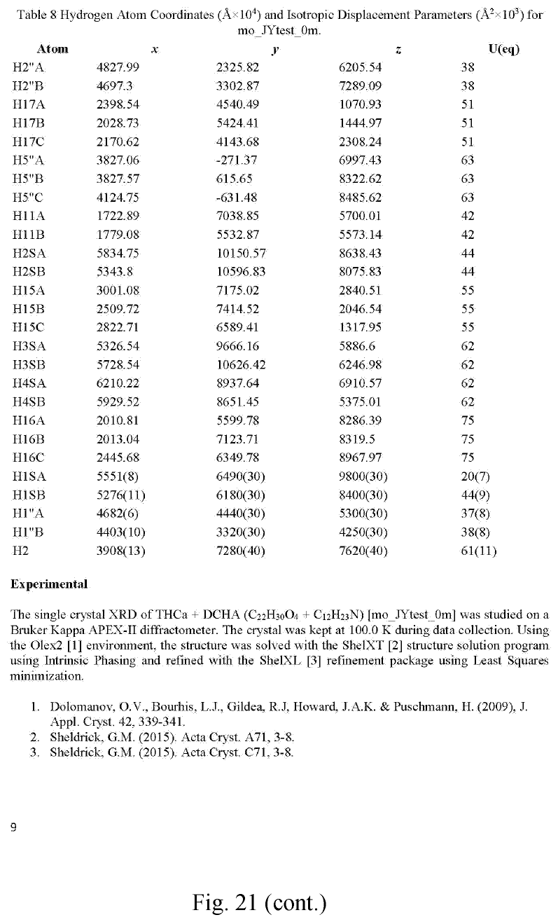

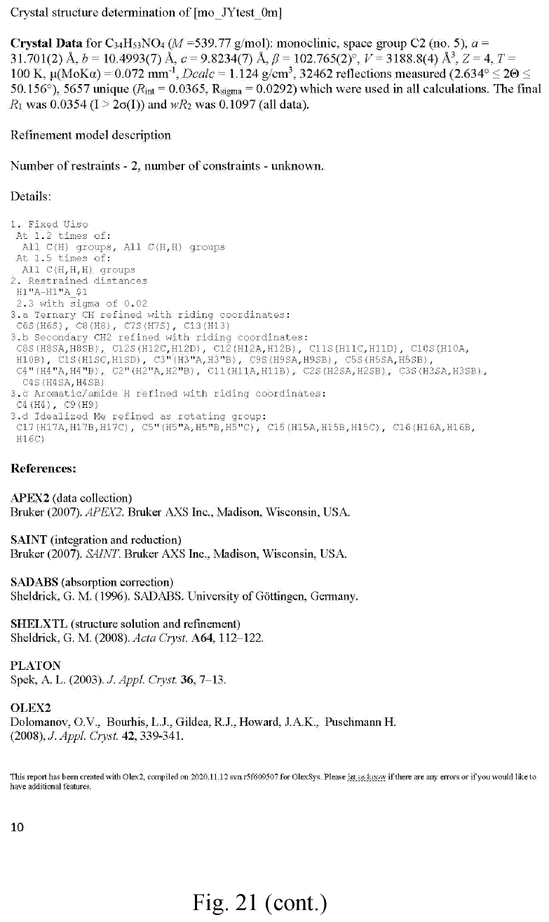

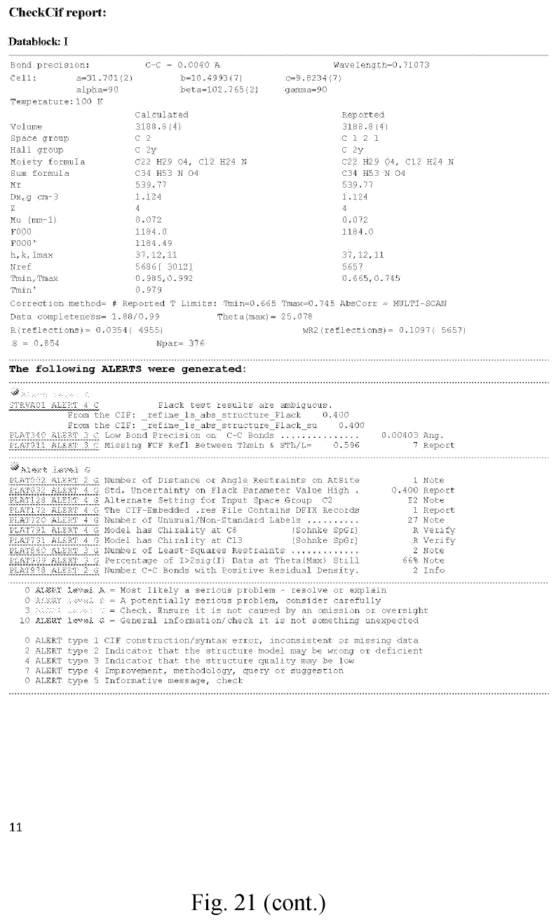

In embodiments the crystalline form of cannabigerolic acid (CBGa) can comprise an about 13% w/w loss from about 1150 to about 155° C. and 87% w/w loss from about 1550 to about 250° C. In embodiments, the crystalline form can comprise a degradation on set at about 115115°. In embodiments, the crystalline form of cannabigerolic acid (CBGa) can comprise a TGA thermogram as show in FIG. 16.

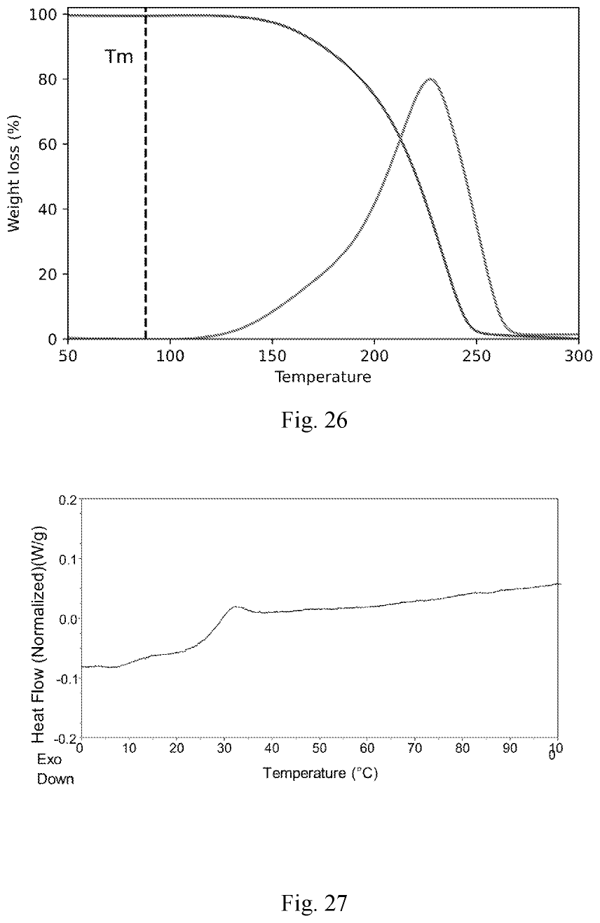

In embodiments the crystalline form of cannabidiolic acid (CBDa) can comprise an 100% w/w loss from about 150° to about 200° C. In embodiments, the crystalline form of CBDa can comprise a degradation onset of about 150° C. In embodiments, the crystalline form of cannabidiolic acid (CBDa) can comprise a TGA thermogram as show in FIG. 26.

### Differential Scanning Calorimetry (DSC) Preparation and Experimental Conditions

Differential Scanning Calorimetry (DSC) thermograms were collected using a TA Instruments Discovery 2500 DSC equipped with an autosampler. The purge gas used for the measurements was 50 mL/min, and the scan rates were about 10-20 C/min. Samples were hermetically sealed in aluminum Tzero pans prior to analysis.

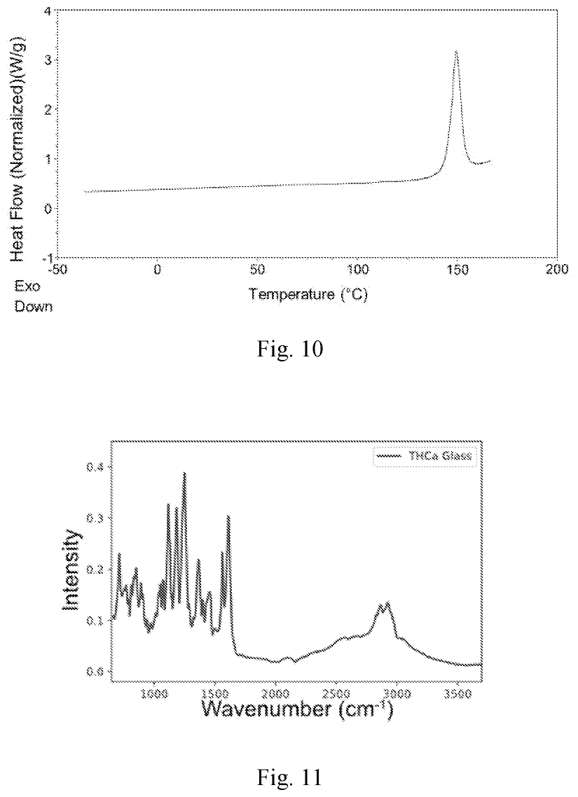

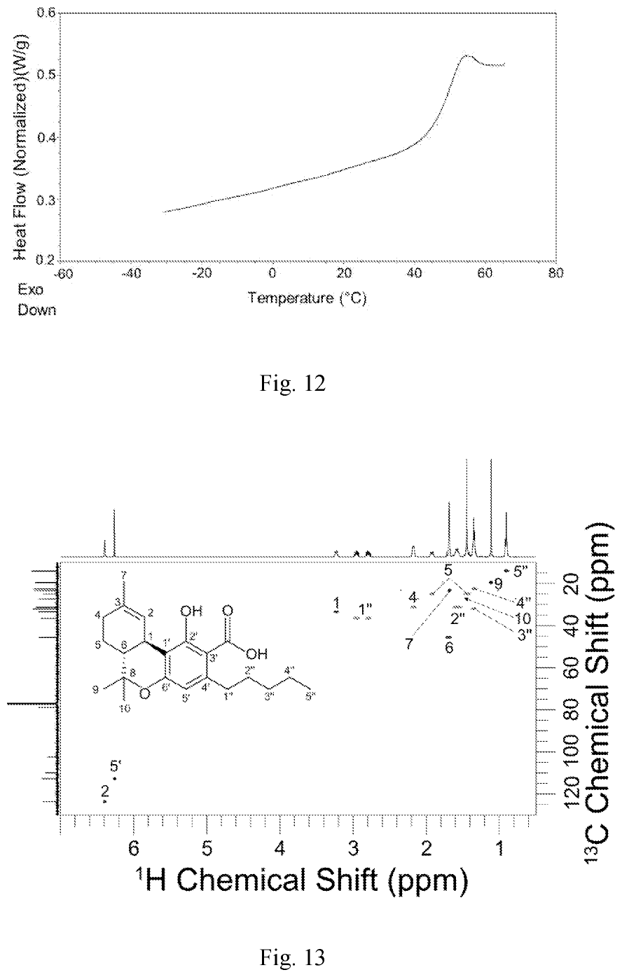

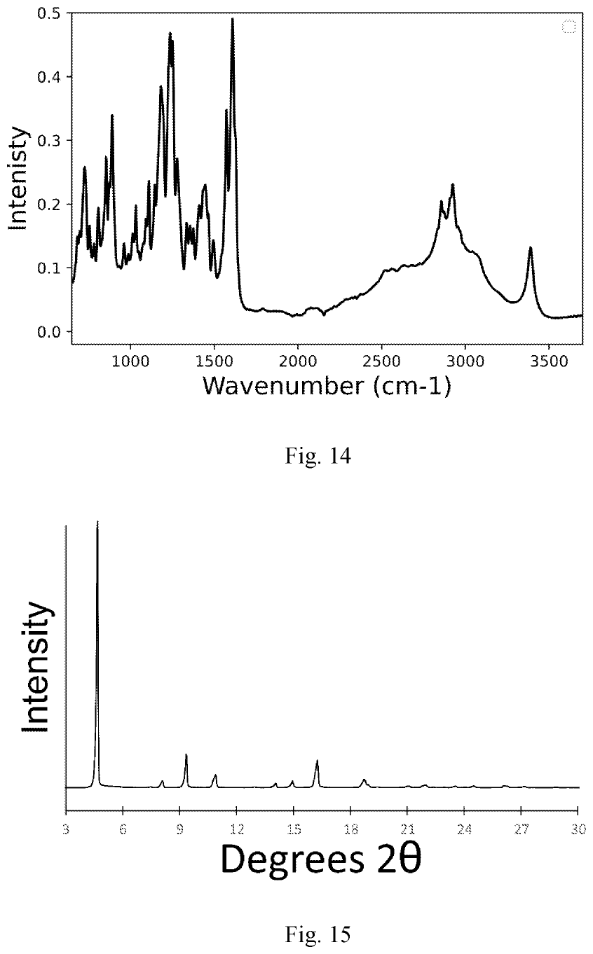

In embodiments the crystalline form of tetrahydrocannabinolic acid (THCa) can comprise an endothermic onset at about 149° C. and a degradation onset at about 125° C. In embodiments, the crystalline form of THCa comprises an endothermic event in the range of about 140° C. to about 149° C. In embodiments, the crystalline form of tetrahydrocannabinolic acid (THCa) characterized by the differential scanning calorimetry (DSC) thermogram substantially as shown in FIG. 10.

In embodiments the crystalline form of cannabigerolic acid (CBGa) can comprise an endothermic onset at about 115° C. In embodiments, the crystalline form can further comprise an endothermic peak at about 61° C. In embodiments, the crystalline form of cannabigerolic acid (CBGa) can be characterized by the differential scanning calorimetry (DSC) thermogram substantially as shown in FIG. 32.

In embodiments the crystalline form of cannabidiolic acid (CBDa) can comprise an endothermic onset at about 85.3° C. In embodiments, the crystalline form of cannabidiolic acid (CBDa) can be characterized by the differential scanning calorimetry (DSC) thermogram substantially as shown in FIG. 25.

### Methods of Crystallization

In embodiments, crystalline cannabinoid acids can be obtained by suspending material which has reasonably high purity in a hydrocarbon or polar aprotic solvent at an elevated temperature and allowing it to slowly cool to −15 C. Alternatively, slowly allowing the solvent to evaporate at room temperature can also result in the formation of crystalline cannabinoid acids.

Pure single crystals and polycrystalline solids of THCa were generated by suspending a high THCa whole plant extract in the minimum amount of warm liquid hydrocarbon (e.g., heptane), and storing in a freezer at −15° C. until crystals formed in 12-24 hours.

The crystals of CBGa were obtained by extracting high CBGa hemp (10 grams) using 100 mL of cold (−15° C.) heptane with agitation for approximately 10 minutes followed by vacuum filtration

The filtrate was dried using a rotary evaporator to produce crude CBGa crystals. The crude CBGa crystals were recrystallized by dissolving in the minimum amount of warm heptane and then allowing to cool 0° C. in a freezer.

The CBDa crystals were obtained by dissolving a high CBDa plant extract (20 grams) in cold acetonitrile (100 mL) and shaken for approximately 5 minutes followed by vacuum filtration.

Approximately 50 mL of acetonitrile was removed under a flow of nitrogen to concentrate the sample and the sample was placed at −15° C. until crystals were observed.

### Dissolution Testing

Dissolution tests were carried out using the paddle method according to USP 29 Apparatus 2 guidelines. The Medium Simulating Fed State Duodenum mimic (MSFeSDM) was prepared using a previously reported solution [Dressman 1998]. It consisted of 15 mM sodium taurocholate, 3.75 mM L-alpha-lecithin, 144 mM glacial acetic acid, 200 mM sodium chloride, and sodium hydroxide to buffer the solution to pH 5 (100 mM) in 1 L of reverse osmosis water. This solution was heated to 37.0° C.±0.5° C. in an RC-1 Dissolution tester (Sinopham, China) with a consistent paddle speed of 100 rotations per minute. Briefly, tablets were made by hand pressing equivalent amounts of API (50 mg) with a microcrystalline cellulose excipient to create a total tablet weight of 400 mg. These tablets were then placed in 250 mL of solution and sampled by withdrawing approximately 1 mL at predetermined time points over the course of four hours. This sample was filtered through a 0.45 μm syringe filter and sampled directly via HPLC.

### Pharmaceutical Compositions

In some aspects, provided herein are compositions, such as pharmaceutical compositions, comprising the crystalline compounds described herein. While it is possible to administer a compound employed in the disclosed methods directly without any formulation, the compounds are usually administered in the form of pharmaceutical compositions.

“Pharmaceutical compositions” are compositions that include the disclosed compound(s) together in an amount (for example, in a unit dosage form) with a pharmaceutically acceptable carrier, diluent, or excipient. Some embodiments will not have a single carrier, diluent, or excipient alone, but will include multiple carriers, diluents, and/or excipients. Compositions can be prepared by standard pharmaceutical formulation techniques such as disclosed in, e.g., Remington: The Science & Practice of Pharmacy (2020) 23rd ed., Academic Press., Cambridge, Mass.

“Pharmaceutically acceptable” used in connection with an excipient, carrier, diluent, or other ingredient can refer to the ingredient is generally safe and, within the scope of sound medical judgment, suitable for use in contact with cells of humans and animals without undue toxicity, irritation, allergic response, or complication, commensurate with a reasonable risk/benefit ratio.

In some embodiments, pharmaceutical compositions comprising a disclosed compound can be administered by a variety of routes including oral, mucosal (e.g., buccal, sublingual), rectal, transdermal, subcutaneous, intravenous, intramuscular, inhaled, intraocular, topical, and intranasal. In some embodiments, the compounds employed in the methods of this invention are effective as oral, mucosal (e.g., buccal, sublingual), rectal, transdermal, subcutaneous, intravenous, intramuscular, inhaled, topical, and intranasal compositions. Such compositions are prepared in a manner well known in the pharmaceutical art and comprise at least one active compound. (See, e.g., Remington, 2020.)

In making the compositions employed in the invention the active ingredient is usually mixed with an excipient, diluted by an excipient, or enclosed within such a carrier which can be in the form of a capsule, sachet, paper or other container. When the excipient serves as a diluent, it can be a solid, semi-solid, or liquid material, which acts as a vehicle, carrier, or medium for the active ingredient. Thus, the compositions can be in the form of tablets (including orally disintegrating, swallowable, sublingual, buccal, and chewable tablets), pills, powders, lozenges, troches, oral films, thin strips, sachets, cachets, elixirs, suspensions, emulsions, microemulsions, liposomal dispersions, aqueous and non-aqueous solutions, slurries, syrups, aerosols (as a solid or in a liquid medium), ointments containing for example up to 10% by weight of the active compound, soft and hard gelatin capsules, suppositories, topical preparations, transdermal patches, sterile injectable solutions, and sterile packaged powders. Compositions can be formulated as immediate release, controlled release, sustained (extended) release or modified release formulations. In some embodiments, the composition is prepared as a dry powder for inhalation or a liquid preparation for vaporization and inhalation, and is administered, e.g., using an electronic cigarette or other vaping device, a nebulizer, a pressurized metered dose inhaler (pMDI), a dry powder inhaler (DPI), or the like.

In other embodiments are disclosed multiple variations in the pharmaceutical dosages of disclosed compositions as further outlined below. Another embodiment of the invention includes various forms of preparations including using solids, liquids, immediate or delayed or extended-release forms. Many types of variations are possible as known to those of skill.

In other embodiments are disclosed multiple routes of administration, which can differ in different patients according to their preference, comorbidities, side effect profile, pharmacokinetic and pharmacodynamic considerations, and other factors (IV, PO, transdermal, etc.). In other embodiments are disclosed the presence of other substances with the active drugs, known to those of skill, such as fillers, carriers, gels, skin patches, lozenges, or other modifications in the preparation to facilitate absorption through various routes (such as gastrointestinal, transdermal, intraocular, etc.) and/or to extend the effect of the drugs, and/or to attain higher or more stable serum levels or to enhance the therapeutic effect of the disclosed compounds.

In preparing a formulation, it can be necessary to mill a disclosed compound to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

Non-limiting examples of suitable excipients comprise lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, methyl cellulose, stearic acid, silica, magnesium stearate, povidone, povidone vinyl acetate, Hypromellose (hydroxypropyl methylcellulose), polyethylene glycol, and polysorbate 80, polysorbate 20. Formulations can additionally include: lubricating agents such as talc, magnesium stearate, and mineral oil; wetting agents; emulsifying and suspending agents; preserving agents such as methyl- and propylhydroxybenzoates; sweetening agents; and flavoring agents. The disclosed compositions can be formulated so as to provide quick, sustained or delayed release of the active agent(s) after administration to the patient by employing procedures known in the art.

The disclosed compositions can be formulated in a unit dosage form, each dosage containing a therapeutically effective amount of the active ingredients, for example in the dosage amounts disclosed herein. The term “unit dosage form” can refer to a physically discrete unit suited as unitary dosages for the subject to be treated, each unit containing a predetermined quantity of active material calculated to produce the desired therapeutic effect(s), in association with a suitable pharmaceutical carrier, diluent, or excipient. Unit dosage forms can be used for ease of administration and uniformity of dosage. Unit dosage forms can contain a single or individual dose or unit, a sub-dose, or an appropriate fraction thereof (e.g., one half a “full” dose for a “booster” dose as described below), of the pharmaceutical composition administered.

Unit dosage forms comprise capsules, troches, cachets, lozenges, tablets, ampules and vials, which can comprise a composition in a freeze-dried or lyophilized state; a sterile liquid carrier, for example, can be added prior to administration or delivery in vivo. Unit dosage forms also comprise ampules and vials with liquid compositions disposed therein. Unit dosage forms further comprise compositions for transdermal administration, such as “patches” that contact the epidermis (including the mucosa) of a subject for an extended or brief period of time.

In some embodiments, the disclosed compositions are formulated in a pharmaceutically acceptable oral dosage form. Oral dosage forms comprise oral liquid dosage forms (such as tinctures, drops, emulsions, syrups, elixirs, suspensions, and solutions, and the like) and oral solid dosage forms. The disclosed pharmaceutical compositions can also be prepared as formulations suitable for intramuscular, subcutaneous, intraperitoneal, or intravenous injection, comprising physiologically acceptable sterile aqueous or non-aqueous solutions, dispersions, suspensions or emulsions, liposomes, and sterile powders for reconstitution into sterile injectable solutions or dispersions.

Oral solid dosage forms can comprise but are not limited to, lozenges, troches, tablets, capsules, caplets, powders, pellets, multiparticulates, beads, spheres, and/or any combinations thereof. Oral solid dosage forms can be formulated as immediate release, controlled release, sustained release, extended release, or modified release formulations. Accordingly, in some embodiments, the disclosed oral solid dosage forms can be in the form of a tablet (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation can be in the form of a powder. In still other embodiments, the pharmaceutical formulation can be in the form of a tablet, including a fast-melt tablet. Additionally, pharmaceutical formulations can be administered as a single capsule or in multiple capsule dosage form. In some embodiments, the pharmaceutical formulation is administered in two, three, four, or more capsules or tablets.

Oral solid dosage forms can contain pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof. Oral solid dosage forms also can comprise one or more pharmaceutically acceptable additives such as a compatible carrier, complexing agent, ionic dispersion modulator, disintegrating agent, surfactant, lubricant, colorant, moistening agent, plasticizer, stabilizer, penetration enhancer, wetting agent, anti-foaming agent, alone or in combination, as well as supplementary active compound(s).

Supplementary active compounds include preservatives, antioxidants, antimicrobial agents including biocides and biostats such as antibacterial, antiviral and antifungal agents. Preservatives can be used to inhibit microbial growth or increase stability of the active ingredient thereby prolonging the shelf life of the formulation. Suitable preservatives are known in the art and include EDTA, EGTA, benzalkonium chloride or benzoic acid or benzoates, such as sodium benzoate. Antioxidants include vitamin A, vitamin C (ascorbic acid), vitamin E, tocopherols, other vitamins or provitamins, and compounds such as alpha lipoic acid.

Using standard coating procedures, a film coating can be provided around the disclosed compounds (see Remington, supra). In one embodiment, some or all of the disclosed compounds are coated. In another embodiment, some or all of the disclosed compounds are microencapsulated. In yet another embodiment, some or all of the disclosed compounds is amorphous material coated and/or microencapsulated with inert excipients. In still another embodiment, the disclosed compounds are not microencapsulated and are uncoated.

Suitable carriers for use in oral solid dosage forms can comprise acacia, gelatin, colloidal silicon dioxide, calcium glycerophosphate, calcium lactate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose (HPMC), hydroxypropylmethylcellulose acetate stearate (HPMCAS), sucrose, microcrystalline cellulose, lactose, and mannitol.

Suitable filling agents for use in oral solid dosage forms can comprise lactose, calcium carbonate, calcium phosphate, dibasic calcium phosphate, calcium sulfate, microcrystalline cellulose, cellulose powder, dextrose, dextrates, dextrose, dextran, starches, pregelatinized starch, HPMC, HPMCAS, hydroxypropylmethylcellulose phthalate, sucrose, xylitol, lactitol, mannitol, sorbitol, sodium chloride, and PEG.

Suitable disintegrants for use in oral solid dosage forms can comprise those disclosed herein for oral liquid aqueous suspensions and dispersions.

Suitable binders impart cohesiveness to solid oral dosage form formulations. For powder-filled capsules, they aid in plug formation that can be filled into soft or hard shell capsules. For tablets, they ensure that the tablet remains intact after compression and help assure blend uniformity prior to a compression or fill step. Materials suitable for use as binders in the solid dosage forms described herein comprise celluloses, microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar (e.g., sucrose, glucose, dextrose, molasses, mannitol, sorbitol, xylitol, lactose), a natural or synthetic gum (e.g., acacia, tragacanth, ghatti gum, mucilage of isapol husks), starch, PVP, larch arabinogalactan, Veegum®, PEG, waxes, and sodium alginate.

Binder levels of 20-70% can be used in powder-filled gelatin capsule formulations. Binder usage level in tablet formulations is a function of whether direct compression, wet granulation, roller compaction, or usage of other excipients such as fillers which itself can act as moderate binders are used. Formulators skilled in the art can determine binder level for formulations, but binder usage of up to 70% in tablet formulations is common.

Suitable lubricants or glidants for use in oral solid dosage forms comprise stearic acid, calcium hydroxide, talc, corn starch, sodium stearyl fumarate, alkali-metal and alkaline earth metal salts, stearic acid, sodium stearates, magnesium stearate, zinc stearate, waxes, Stearowet®, boric acid, sodium benzoate, sodium acetate, sodium chloride, leucine, PEG, methoxy-polyethylene glycol, propylene glycol, sodium oleate, glyceryl behenate, glyceryl palmitostearate, glyceryl benzoate, and magnesium or sodium lauryl sulfate.

Suitable diluents for use in oral solid dosage forms comprise sugars (including lactose, sucrose, and dextrose), polysaccharides (including dextrates and maltodextrin), polyols (including mannitol, xylitol, and sorbitol), and cyclodextrins. Non-water-soluble diluents are compounds typically used in the formulation of pharmaceuticals, such as calcium phosphate, calcium sulfate, starches, modified starches and microcrystalline cellulose, and micro cellulose (e.g., having a density of about 0.45 g/cm3, e.g., Avicel, powdered cellulose), and talc.

Suitable wetting agents for use in oral solid dosage forms can comprise oleic acid, triethanolamine oleate, glyceryl monostearate, sorbitan monooleate, sorbitan monolaurate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, and vitamin E TPGS. Wetting agents comprise surfactants.

Suitable surfactants for use in the solid dosage forms described herein comprise docusate and its pharmaceutically acceptable salts, sodium lauryl sulfate, sorbitan monooleate, poly-oxyethylene sorbitan monooleate, polysorbates, poloxamers, bile salts, glyceryl monostearate, copolymers of ethylene oxide and propylene oxide, e.g., Pluronic® (BASF), and the like.

Suitable suspending agents for use in oral solid dosage forms comprise polyvinylpyrrolidone, PEG (having a molecular weight of about 300 to about 6000, or about 3350 to about 4000, or about 7000 to about 18000), vinylpyrrolidone/vinyl acetate copolymer (S630), sodium alginate, gums (e.g., gum tragacanth and gum acacia, guar gum, xanthans, including xanthan gum), sugars, celluloses, polysorbate-80, polyethoxylated sorbitan monolaurate, polyethoxylated sorbitan monolaurate, and povidone.

Suitable antioxidants for use in oral solid dosage forms comprise butylated hydroxytoluene (BHT), butyl hydroxyanisole (BHA), sodium ascorbate, Vitamin E TPGS, ascorbic acid, sorbic acid, and tocopherol.

Immediate-release formulations can be prepared by combining a superdisintegrant such as croscarmellose sodium and different grades of microcrystalline cellulose in different ratios. To aid disintegration, sodium starch glycolate can be added.

In cases where different agents included in the disclosed fixed-dose combinations are incompatible, cross-contamination can be avoided by incorporation of the agents in different layers in the oral dosage form with the inclusion of barrier layer(s) between the different layers, wherein the barrier layer(s) comprise inert and non-functional material(s).

The above-listed additives are exemplary types of additives that can be included in the disclosed solid dosage forms of the present invention. The amounts of such additives can be readily determined by one skilled in the art, according to the particular properties desired.

Tablets of the invention can be prepared by methods well known in the art. Various methods for the preparation of the immediate release, modified release, controlled release, and extended-release dosage forms (e.g., as matrix tablets having one or more modified, controlled, or extended-release layers) and the vehicles therein are well known in the art. For example, a tablet can be made by compression or molding. Compressed tablets can be prepared by compressing, in a suitable machine, an active ingredient in a free-flowing form such as a powder or granules, optionally mixed with a binder, lubricant, inert diluent, preservative, surface-active or dispersing agent. Molded tablets can be produced by molding, in a suitable apparatus, a mixture of powdered compound moistened with an inert liquid diluent. The tablets can be coated or scored and can be formulated so as to provide a slow or controlled release of the active ingredient therein. Generally recognized compendia of methods include: Remington (2020).

In embodiments, the oral liquid dosage forms of the disclosure comprise tinctures, drops, emulsions, syrups, elixirs, suspensions, and solutions, and the like. These oral liquid dosage forms can be formulated with any pharmaceutically acceptable excipient known to those of skill in the art for the preparation of liquid dosage forms, and with solvents, diluents, carriers, excipients, and the like chosen as appropriate to the solubility and other properties of the active agents and other ingredients. Solvents can be, for example, water, glycerin, simple syrup, alcohol, medium chain triglycerides (MCT), and combinations thereof.

Liquid dosage forms for oral administration can be in the form of pharmaceutically acceptable emulsions, syrups, elixirs, suspensions, and solutions, which can contain an inactive diluent, such as water. Pharmaceutical formulations can be prepared as liquid suspensions or solutions using a sterile liquid, such as but not limited to, an oil, water, an alcohol, and combinations of these pharmaceutically suitable surfactants, suspending agents, emulsifying agents, can be added for oral or parenteral administration. Liquid formulations also can be prepared as single dose or multi-dose beverages. Suspensions can include oils. Such oils include peanut oil, sesame oil, cottonseed oil, corn oil, and olive oil. Suitable oils also include carrier oils such as MCT and long chain triglyceride (LCT) oils. Suspension preparation can also contain esters of fatty acids such as ethyl oleate, isopropyl myristate, fatty acid glycerides, and acetylated fatty acid glycerides. Suspension formulations can comprise alcohols, (such as ethanol, isopropyl alcohol, hexadecyl alcohol), glycerol, and propylene glycol. Ethers, such as poly(ethylene glycol), petroleum hydrocarbons such as mineral oil and petrolatum, and water can also be used in suspension formulations. Suspension can thus include an aqueous liquid or a non-aqueous liquid, an oil-in-water liquid emulsion, or a water-in-oil emulsion.

In some embodiments, formulations are provided comprising the disclosed compositions and at least one dispersing agent or suspending agent for oral administration to a subject. The formulation can be a powder and/or granules for suspension, and upon admixture with water, a substantially uniform suspension is obtained. The aqueous dispersion can comprise amorphous and non-amorphous particles consisting of multiple effective particle sizes such that a drug is absorbed in a controlled manner over time.

Dosage forms for oral administration can be aqueous suspensions selected from the group including pharmaceutically acceptable aqueous oral dispersions, emulsions, solutions, and syrups. In addition to the disclosed compounds, the liquid dosage forms can comprise additives, such as one or more (a) disintegrating agents, (b) dispersing agents, (c) wetting agents, (d) preservatives, (e) viscosity enhancing agents, (f) sweetening agents, or (g) flavoring agents.

Examples of disintegrating agents for use in the aqueous suspensions and dispersions include a starch, e.g., a natural starch such as corn starch or potato starch, a pregelatinized starch, or sodium starch glycolate; a cellulose such as a wood product, microcrystalline cellulose, methylcellulose, croscarmellose, or a cross-linked cellulose, such as cross-linked sodium carboxymethylcellulose, cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a clay; a gum such as agar, guar, locust bean, Karaya, pectin, or tragacanth; sodium starch glycolate; bentonite; a natural sponge; a surfactant; a resin such as a cation-exchange resin; citrus pulp; and sodium lauryl sulfate.

Examples of dispersing agents suitable for the aqueous suspensions and dispersions comprise hydrophilic polymers, electrolytes, Tween® 60 or 80, polyethylene glycol (PEG), polyvinylpyrrolidone (PVP), carbohydrate-based dispersing agents, noncrystalline cellulose, magnesium aluminum silicate, triethanolamine, polyvinyl alcohol (PVA), polyvinylpyrrolidone/vinyl acetate copolymer, poloxamers, and poloxamines.

Examples of wetting agents (including surfactants) suitable for the aqueous suspensions and dispersions comprise acetyl alcohol, glycerol monostearate, polyoxyethylene sorbitan fatty acid esters, PEG, oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, and phosphatidylcholine.

Examples of preservatives suitable for aqueous suspensions or dispersions comprise potassium sorbate, parabens (e.g., methylparaben and propylparaben) and their salts, benzoic acid and its salts, other esters of para hydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Examples of viscosity enhancing agents suitable for aqueous suspensions or dispersions comprise methyl cellulose, xanthan gum, carboxymethylcellulose, hydroxypropyl cellulose, hydroxypropylmethyl cellulose, Plasdone® 5-630, carbomer, polyvinyl alcohol, alginates, acacia, chitosans, and combinations thereof. The concentration of the viscosity-enhancing agent will depend upon the agent selected and the viscosity desired.

In addition to the additives listed above, the disclosed liquid formulations can also comprise inert diluents commonly used in the art, such as water or other solvents, solubilizing agents, emulsifiers, flavoring agents and/or sweeteners. Co-solvents and adjuvants also can be added to a formulation. Non-limiting examples of co-solvents contain hydroxyl groups or other polar groups, for example, alcohols, glycols, glycerol, polyoxyethylene alcohols, and polyoxyethylene fatty acid esters. Adjuvants include surfactants such as soy lecithin and oleic acid, sorbitan esters such as sorbitan trioleate, and PVP.

In other embodiments, disclosed pharmaceutical compositions can be formulated into a topical dosage form. Topical dosage forms include transmucosal and transdermal formulations, such as aerosols, emulsions, sprays, ointments, salves, gels, pastes, lotions, liniments, oils, and creams. For such formulations, penetrants and carriers can be included in the pharmaceutical composition. Penetrants are known in the art, and include, for transmucosal administration, detergents, bile salts, and fusidic acid derivatives. For transdermal administration, carriers which can be used include Vaseline®, lanolin, PEG, alcohols, transdermal enhancers, and combinations thereof.

An exemplary topical delivery system is a transdermal delivery device (“patch”) containing the active agents. Such transdermal patches can be used to provide continuous or discontinuous infusion of the disclosed compounds in controlled amounts. Such patches can be constructed for continuous, gradual, pulsatile, or on demand delivery of pharmaceutical agents. In one embodiment, a “patch” can be a medicated adhesive patch, i.e., a patch impregnated with a disclosed composition for application onto the skin. Thus, a patch can be a single-layer or multi-layer drug-in-adhesive patch, wherein the one or more adhesive layers also contain the active agents.

A patch can also be a “matrix” (or “monolithic”) patch, wherein the adhesive layer surrounds and overlays the drug layer (wherein a solution or suspension of the active agents is in a semisolid matrix). A “reservoir” patch can also be used, comprising a drug layer, typically as a solution or suspension of the active agents in a liquid compartment (i.e., the reservoir), separate from an adhesive layer. For example, the reservoir can be totally encapsulated in a shallow compartment molded from a drug-impermeable metallic plastic laminate, with a rate-controlling membrane made of vinyl acetate or a like polymer on one surface. A patch also can be part of a delivery system, for instance used with an electronic device communicatively coupled to the mobile device of a user, and coupled with a mobile application (e.g., to control the delivery rate from the reservoir, and optionally to provide information about delivery back to the application or user). Various transdermal patch technologies can be accordingly utilized.

One such transdermal patch technology as herein contemplated comprises a self-contained module including a built-in battery that produces a low-level electric current to heat the skin and deliver a prescribed dose of a composition of the invention, wherein a therapeutically effective amount of the composition crosses the skin and enters the underlying tissue, so as to produce a therapeutic effect. Such a transdermal delivery device can, for example, comprise an adhesive layer, a protective film, a drug-containing reservoir (for the disclosed pharmaceutical compositions), a heating coil, a battery, a hardware board, optionally all within a device holder, and optionally, functionally coupled to a device which is able to control drug delivery (e.g., a mobile device such as a smartphone) using a downloadable application. Such devices can, for instance, additionally shut off drug delivery automatically when a prescribed dose has been administered or can shut off automatically upon reaching a certain temperature or defined time. Such transdermal devices can be reusable or disposable.

Disclosed compositions also can be prepared as formulations suitable for intramuscular, subcutaneous, intraperitoneal, or intravenous injection, comprising physiologically acceptable sterile aqueous or non-aqueous solutions, dispersions, suspensions or emulsions, liposomes, and sterile powders for reconstitution into sterile injectable solutions or dispersions.

Examples of suitable aqueous and non-aqueous carriers, diluents, solvents, or vehicles include water, ethanol, polyols, suitable mixtures thereof, vegetable oils, and injectable organic esters such as ethyl oleate. Additionally, the disclosed compositions can be dissolved at concentrations of >1 mg/ml using water-soluble beta cyclodextrins (e.g., beta-sulfobutyl-cyclodextrin and 2-hydroxypropyl-betacyclodextrin. Proper fluidity can be maintained, for example, by the use of a coating such as a lecithin, by the maintenance of the required particle size in the case of dispersions, and by the use of surfactants.

Formulations suitable for subcutaneous injection also can contain additives such as preserving, wetting, emulsifying, and dispensing agents. Prevention of the growth of microorganisms can be ensured by various antibacterial and antifungal agents, such as parabens, benzoic acid, benzyl alcohol, chlorobutanol, phenol, and sorbic acid. Isotonic agents, such as sugars and sodium chloride can be used. Prolonged drug absorption of an injectable form can be brought about by use of agents delaying absorption, e.g., aluminum monostearate or gelatin.

Disclosed compositions also can be prepared as suspension formulations designed for extended-release via subcutaneous or intramuscular injection. Such formulations avoid first-pass metabolism, and lower dosages of the active agents will be necessary to maintain equivalent plasma levels when compared to oral formulations. In such formulations, the mean particle size of the active agents and the range of total particle sizes can be used to control the release of those agents by controlling the rate of dissolution in fat or muscle. The compositions also can be prepared for microinjection or injection cannula.

### Dose and Dosage, Additional Agents, and Kits

In some embodiments, pharmaceutical compositions comprise a therapeutically effective amount or an effective amount of a disclosed compound, such as for administration to a subject. Administration of pharmaceutical compositions in a “therapeutically effective amount,” or an “effective amount” to a subject can refer to administration of an amount of composition sufficient to achieve the desired effect. When an “effective amount” can be an amount effective in treating the stated disorder or symptoms in a subject, “therapeutic effect” can refer the responses(s) in a mammal after treatment that are judged to be desirable and beneficial. Hence, depending on the disorder to be treated, and depending on the particular constituent(s) in the disclosed compositions under consideration, those responses can differ, but can be readily understood by those of ordinary skill, through an understanding of the disclosure herein and the general knowledge of the art.

In embodiments, the pharmaceutical compositions disclosed herein comprise therapeutic amounts of substituted tryptamines and in some embodiments other active or inactive ingredients. Dosage amounts will be understood by reference to all of the teachings herein as well as the general knowledge in the art, but certain exemplary dosage amounts, known to be useful in the practice of the invention, are listed below for ease of reference.

In some embodiments, where a pharmaceutical composition comprises a disclosed composition, it can be present in an amount so that a single dose is (in a milligram dosage amount calculated based on the kilogram weight of the patient), e.g., 0.25 mg/kg or less (including a dose of 0.10 mg/kg or less, 0.05 mg/kg or less, 0.01 mg/kg or less, and 0.005 mg/kg or less), at least 0.50 mg/kg, at least 0.55 mg/kg, at least 0.60 mg/kg, at least 0.65 mg/kg, at least 0.70 mg/kg, at least 0.75 mg/kg, at least 0.80 mg/kg, at least 0.85 mg/kg, at least 0.90 mg/kg, at least 0.95 mg/kg, at least 1.0 mg/kg, at least 1.1 mg/kg, at least 1.2 mg/kg, at least 1.3 mg/kg, or at least 1.4 mg/kg, at least 1.5 mg/kg, at least 1.6 mg/kg, at least 1.7 mg/kg, at least 1.8 mg/kg, at least 1.9 mg/kg, at least 2.0 mg/kg, at least 2.1 mg/kg, at least 2.2 mg/kg, at least 2.3 mg/kg, at least 2.4 mg/kg, at least 2.5 mg/kg, at least 2.6 mg/kg, at least 2.7 mg/kg, at least 2.8 mg/kg, at least 2.9 mg/kg, or at least 3.0 mg/kg, as well as amounts within these ranges.

In some embodiments, where a pharmaceutical composition comprises a disclosed compound, it can be present in an amount so that a single dose is (in a milligram dosage amount calculated based on the kilogram weight of the patient) less than about 0.01 mg/kg, between about 0.01 mg/kg and 0.1 mg/kg, such as about 0.01 mg/kg, about 0.02 mg/kg, about 0.03 mg/kg, about 0.04 mg/kg, about 0.05 mg/kg, about 0.06 mg/kg, about 0.07 mg/kg about 0.08 mg/kg about 0.09 mg/kg, and about 0.1 mg/kg, as well as ranges between these values. In some embodiments, a single dose is between about 0.1 mg/kg and 1.0 mg/kg, such as about 0.1 mg/kg, about 0.2 mg/kg, about 0.3 mg/kg, about 0.4 mg/kg, about 0.5 mg/kg, about 0.6 mg/kg, about 0.7 mg/kg about 0.8 mg/kg about 0.9 mg/kg, and about 1.0 mg/kg, as well as ranges between these values.

In some embodiments, where a pharmaceutical composition comprises a composition described herein, it can be present in an amount so that a single dose is (whether or not such dose is present in a unit dosage form), e.g., 0.1 mg or less (including a dose of 0.05 mg or less, 0.025 mg or less, 0.001 mg or less, and 0.0005 mg or less), at least 0.25 mg, at least 0.5 mg, at least 1 mg, at least 2.5 mg, at least 5 mg, at least 10 mg, at least 20, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 105 mg, at least 110 mg, at least 115 mg, at least 120 mg, at least 125 mg, at least 130 mg, at least 135 mg, at least 140 mg, at least 145 mg, at least 150 mg, at least 155 mg, at least 160 mg, at least 165 mg, at least 170 mg, at least 175 mg, at least 180 mg, at least 185 mg, at least 190 mg, at least 195 mg, at least 200 mg, at least 225 mg, least 250 mg, at least 275 mg, at least 300 mg, at least 325 mg, at least 350 mg, at least 375 mg, at least 400 mg, at least 425 mg, at least 450 mg, at least 475 mg, at least 500 mg, at least 525 mg, at least 550 mg, at least 575 mg, at least 600 mg, at least 625 mg, at least 650 mg, at least 675 mg, at least 700 mg, at least 725 mg, at least 750 mg, at least 775 mg, at least 800 mg, at least 825 mg, at least 850 mg, at least 875 mg, at least 900 mg, at least 925 mg, at least 950 mg, at least 975 mg, at least 1000 mg, at least 1025 mg, at least 1050 mg, at least 1175 mg, at least 1200 mg, at least 1225 mg, at least 1250 mg, at least 1275 mg, at least 1300 mg, at least 1400 mg, at least 1500 mg, at least 1600 mg, at least 1700 mg, at least 1800 mg, at least 1900, and at least 2000 mg, as well as amounts within these ranges.

In some embodiments, where a pharmaceutical composition includes a disclosed compound, it can be present in an amount so that a single dose is (whether or not such dose is present in a unit dosage form) between about 0.1 mg and 1.0 mg, such as about 0.1 mg, about 0.2 mg, about 0.3 mg, about 0.4 mg, about 0.5 mg, about 0.6 mg, about 0.7 mg, about 0.8 mg, about 0.9 mg, and about 1.0 mg, as well as ranges between these values. In some embodiments, a single dose is between about 1 mg and 10 mg, such as about 1 mg, about 2 mg, about 3 mg, about 4 mg, about 5 mg, about 6 mg, about 7 mg, about 8 mg, about 9 mg, and about 10 mg, as well as ranges between these values. In some embodiments, a single dose is between about 10 mg and 100 mg.

In some embodiments, where a pharmaceutical composition comprises an additional active compound, for instance where the additional active compound is a NSAID, it can be present in an amount so that a single dose is (in a milligram dosage amount calculated based on the kilogram weight of the patient), e.g., 0.25 mg/kg or less (including a dose of 0.10 mg/kg or less, 0.05 mg/kg or less, 0.01 mg/kg or less, and 0.005 mg/kg or less), at least 0.50 mg/kg, at least 0.55 mg/kg, at least 0.60 mg/kg, at least 0.65 mg/kg, at least 0.70 mg/kg, at least 0.75 mg/kg, at least 0.80 mg/kg, at least 0.85 mg/kg, at least 0.90 mg/kg, at least 0.95 mg/kg, at least 1.0 mg/kg, at least 1.1 mg/kg, at least 1.2 mg/kg, at least 1.3 mg/kg, or at least 1.4 mg/kg, at least 1.5 mg/kg, at least 1.6 mg/kg, at least 1.7 mg/kg, at least 1.8 mg/kg, at least 1.9 mg/kg, at least 2.0 mg/kg, at least 2.1 mg/kg, at least 2.2 mg/kg, at least 2.3 mg/kg, at least 2.4 mg/kg, at least 2.5 mg/kg, at least 2.6 mg/kg, at least 2.7 mg/kg, at least 2.8 mg/kg, at least 2.9 mg/kg, or at least 3.0 mg/kg, as well as amounts within these ranges.

In some embodiments, where a pharmaceutical composition comprises an additional active compound, for instance where the additional active compound is a NSAID, it can be present in an amount so that a single dose is (whether or not such dose is present in a unit dosage form), e.g., 25 mg or less (including a dose of 10 mg or less, 5 mg or less, 1 mg or less, and 0.5 mg or less), at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 105 mg, at least 110 mg, at least 115 mg, at least 120 mg, at least 125 mg, at least 130 mg, at least 135 mg, at least 140 mg, at least 145 mg, at least 150 mg, at least 155 mg, at least 160 mg, at least 165 mg, at least 170 mg, at least 175 mg, at least 180 mg, at least 185 mg, at least 190 mg, at least 195 mg, at least 200 mg, at least 225 mg, or at least 250 mg, as well as amounts within these ranges.

Dosages can vary depending upon whether the treatment is therapeutic or prophylactic, the onset, progression, severity, frequency, duration, probability of or susceptibility of the symptom to which treatment is directed, clinical endpoint desired, previous, simultaneous or subsequent treatments, general health, age, gender, and race of the subject, bioavailability, potential adverse systemic, regional or local side effects, the presence of other disorders or diseases in the subject, and other factors that will be appreciated by the skilled artisan (e.g., medical or familial history).

Dose amount, frequency or duration can be increased or reduced, as indicated by the clinical outcome desired, status of the pathology or symptom, any adverse side effects of the treatment or therapy, or concomitant medications. The skilled artisan with the teaching of this disclosure in hand will appreciate the factors that can influence the dosage, frequency, and timing required to provide an amount sufficient or effective for providing a therapeutic effect or benefit, and to do so depending on the type of therapeutic effect desired, as well as to avoid or minimize adverse effects.

In some embodiments, the dose actually administered will be determined by a physician, in light of the relevant circumstances, including the disorder to be treated, the chosen route of administration, the actual composition or formulation administered, the age, weight, and response of the individual patient, and the severity of the patient's symptoms, and therefore any dosage ranges disclosed herein are not intended to limit the scope of the invention. In some instances, dosage levels below the lower limit of a disclosed range can be more than adequate, while in other cases doses above a range can be employed without causing any harmful side effects, provided for instance that such larger doses also can be divided into several smaller doses for administration, either taken together or separately.

In these embodiments, the disclosed pharmaceutical compositions can be administered and dosed in accordance with good medical practice, taking into account the method and scheduling of administration, prior and concomitant medications and medical supplements, the clinical condition of the individual patient and the severity of the underlying disease, the patient's age, sex, body weight, and other such factors relevant to medical practitioners, and knowledge of the particular compound(s) used. Starting and maintenance dosage levels thus can differ from patient to patient, for individual patients across time, and for different pharmaceutical compositions and formulations, but shall be able to be determined with ordinary skill.

In embodiments, e.g., when the disclosed compositions are taken without the direct intervention or guidance of a medical professional, appropriate dosages to achieve a therapeutic effect, including the upper and lower bounds of any dose ranges, can be determined by an individual by reference to available public information and knowledge, and reference to subjective considerations regarding desired outcomes and effects.

Determination of appropriate dosing can comprise not only the determination of single dosage amounts, but also the determination of the number and timing of doses, e.g., administration of a particular dosage amount once per day, twice per day, or more than twice per day, and the time(s) of day or time(s) during a therapy session preferable for their administration.

In some embodiments, e.g., where a formulation is prepared in single unit dosage form, such as a capsule, tablet, or lozenge, suggested dosage amounts shall be known by reference to the format of the preparation itself. In other embodiments, where a formulation is prepared in multiple dosage form, for instance liquid suspensions and topical preparations, suggested dosage amounts can be known by reference to the means of administration or by reference to the packaging and labeling, package insert(s), marketing materials, training materials, or other information and knowledge available to those of skill or the public.

Accordingly, another aspect of this disclosure provides pharmaceutical kits containing a pharmaceutical composition or formulation of the invention, suggested administration guidelines or prescribing information therefor, and a suitable container. Individual unit dosage forms can be included in multi-dose kits or containers. pharmaceutical formulations also can be packaged in single or multiple unit dosage forms for uniformity of dosage and ease of administration.

In an exemplary pharmaceutical kit, capsules, tablets, caplets, or other unit dosage forms are packaged in blister packs. “Blister pack” refers to any of several types of pre-formed container, especially plastic packaging, that contains separate receptacles (e.g., cavities or pockets) for single unit doses, where such separate receptacles are individually sealed and can be opened individually. Blister packs thus include such pharmaceutical blister packs known to those of ordinary skill, including Aclar® Rx160, Rx20e, SupRx, and UltRx 2000, 3000, 4000, and 6000 (Honeywell). Within the definition of multi-dose containers, and also often referred to as blister packs, are blister trays, blister cards, strip packs, push-through packs, and the like.

In embodiments, information pertaining to dosing and proper administration (if needed) will be printed onto a multi-dose kit directly (e.g., on a blister pack or other interior packaging holding the compositions or formulations of the invention); however, kits of the invention can further contain package inserts and other printed instructions (e.g., on exterior packaging) for administering the disclosed compositions and for their appropriate therapeutic use.

In some embodiments, a patient will have the option of using online software such as a website, or downloadable software such as a mobile application, to assist with compliance or to provide data relating to treatment. Such software can be used to, e.g., keep track of last dose taken and total doses taken, provide reminders and alerts for upcoming doses, provide feedback to discourage taking doses outside of set schedules, and allow for recording of specific subjective effects, or provide means for unstructured journaling. Such data collection can assist with individual patient compliance, can be used to improve or tailor individual patient care plans, and can be anonymized, aggregated, and analyzed (including by AI or natural language processing means) to allow research into the effects of various methods of treatment.

The disclosed compositions are not limited to combinations of a single compound, or (when formulated as a pharmaceutical composition) limited to a single carrier, diluent, and/or excipient alone, but can also include combinations of multiple compounds (including additional active compounds), and/or multiple carriers, diluents, and excipients. Pharmaceutical compositions of this invention thus can comprise a compound of described herein together with one or more other active agents (or their derivatives and analogs) in combination, together with one or more pharmaceutically-acceptable carriers, diluents, and/or excipients, and additionally with one or more other active compounds.

In some embodiments, a formulation of the invention will be prepared so as to increase an existing therapeutic effect, provide an additional therapeutic effect, increase a desired property such as stability or shelf-life, decrease an unwanted effect or property, alter a property in a desirable way (such as pharmacokinetics or pharmacodynamics), modulate a desired system or pathway (e.g., a neurotransmitter system), or provide synergistic effects.

“Therapeutic effects” that can be increased or added in embodiments of the invention include, but are not limited to, antioxidant, anti-inflammatory, analgesic, antineuropathic, antinociceptive, antimigraine, anxiolytic, antidepressant, antipsychotic, anti-PTSD, dissociative, immunostimulant, anti-cancer, antiemetic, anti-epileptic, orexigenic, antiulcer, anti-IBS, antihistamine, antihypertensive, anticonvulsant, antiepileptic, bronchodilator, neuroprotective, sedative, sedative-hypnotic, anti-epileptic, anti-convulsant, anti-viral, and stimulant effects.

“Synergistic effects” can refer to increases in potency, bioactivity, bioaccessibility, bioavailability, or therapeutic effect, that are greater than the additive contributions of the components acting alone. Numerous methods known to those of skill in the art exist to determine whether there is synergy as to a particular effect, i.e., whether, when two or more components are mixed together, the effect is greater than the sum of the effects of the individual components when applied alone, thereby producing “1+1 >2.” The goal of increasing an existing therapeutic effect, providing an additional therapeutic effect, increasing a desired property such as stability or shelf-life, decreasing an unwanted effect or property, altering a property in a desirable way (such as pharmacokinetics or pharmacodynamics), modulating a desired system or pathway (e.g, a neurotransmitter system), or otherwise inducing synergy, in some embodiments is achieved by the inclusion of an additional active compound.

Such additional active compounds can comprise amino acids, anti-virals, antioxidants, anti-inflammatory agents, analgesics, antineuropathic and antinociceptive agents, antimigraine agents, anxiolytics, antidepressants, antipsychotics, anti-PTSD agents, NSAIDs, dissociatives, immunostimulants, anti-cancer agents, antiemetics, orexigenics, antiulcer agents, antihistamines, antihypertensives, anticonvulsants, antiepileptics, bronchodilators, neuroprotectants, nootropics, empathogens, psychedelics, monoamine oxidase inhibitors, sedatives, stimulants, and vitamins. These ingredients can be in ion, freebase, or salt form, and can be isomers, prodrugs, derivatives (preferably physiologically functional derivatives), or analogs.

### Methods of Use

Aspects of the invention are drawn towards methods of using the crystalline forms described herein. In embodiments, the crystalline forms described herein can be used as active pharmacological ingredients (APIs). In embodiments, the crystalline forms described herein can be administered to a subject. Non-limiting, exemplary methods of use, formulations, and dosing of the crystalline forms described herein can encompass those used in other cannabinoid compositions in the art (e.g., Campos et al., A Systemic Review of Medical Cannabinoids dosing in Human, Clinical Therapeutics, Vol. 44, No. 12, 2022).

As used herein, the terms “subject,” “user,” “patient,” and “individual” are used interchangeably, and can refer to any mammal, including murines, simians, mammalian farm animals, mammalian sport animals, and mammalian pets, such as canines and felines, although preferably humans. Such terms will be understood to include one who has an indication for which a compound, composition, or method described herein can be efficacious, or who otherwise can benefit by the invention. In general, all of the compounds, compositions, and disclosed methods will be appreciated to work for all individuals, although individual variation is to be expected, and will be understood. The disclosed methods of treatment also can be modified to treat multiple patients at once, including couples or families. Hence, these terms can also refer to two or more individuals.

In some embodiments, the disclosed crystalline forms are used to treat a condition, such as a disease or a disorder. In some embodiments, described herein are disclosed compounds for use in treating a condition, such as a disease or a disorder. In some embodiments, the disclosed compounds are used in the manufacture of a medicament to treat a condition, such as a disease or disorder. In some embodiments, described are methods of administering disclosed compounds to a subject having a condition, such as a disease or disorder, thereby treating said condition.

In some embodiments, disclosed compounds or pharmaceutical compositions comprising the disclosed crystalline forms are administered to a subject by one or more routes of administration, including, e.g., oral, mucosal, rectal, subcutaneous, intravenous, intramuscular, intranasal, inhaled, intraocular, topical, and transdermal routes. When administered through one or more of such routes, the compound(s) of the invention and the disclosed compositions and formulations comprising them are useful in methods for treating a patient in need of such treatment.

As used herein, “an effective amount”, “a therapeutically effective amount”, or “a pharmacologically effective amount” can be used interchangeably. As used herein, “a therapeutically effective amount” can refer to an amount of an active agent that is sufficient to provide the desired therapeutic effect. The effective amount will vary depending upon the subject and the disease condition being treated or health benefit sought, the weight and age of the subject, the severity of the disease condition or degree of health benefit sought, the manner of administration, and the like, all of which can readily be determined by one of ordinary skill in the art.

Herein, “therapeutic effect” or “therapeutic efficacy” refers to the responses(s) in a mammal, and preferably a human, after treatment that are judged to be desirable and beneficial. Depending on the disorder to be treated, or improvement in mental health or functioning sought, and depending on the particular constituent(s) in the disclosed compositions under consideration, those responses can therefore differ, but can be readily understood by those of ordinary skill.

Measures of therapeutic effect includes any outcome measure, endpoint, effect measure, or measure of effect within clinical or medical practice or research which is used to assess the effect, both positive and negative, of an intervention or treatment, whether patient-reported (e.g., questionnaires), based on other patient data (e.g., patient monitoring), gathered through laboratory tests such as blood work, urine samples, etc., through medical examination by a doctor or other medical professional, or by digital tools or means, e.g., electronic tools such as online tools, smartphones, wireless devices, biosensors, or health apps.

In some embodiments, measures of therapeutic effect will include an assessment. “Assessment” can refer to any means or method used with a patient, whether before, during, after, or unrelated in time to a specific treatment protocol, to measure, estimate, or evaluate a nature, ability, symptom, disorder, or other characteristic of the patient, whether qualitatively or quantitatively, and whether performed by the therapist or other clinician (e.g., an interview), by the patient his or herself (e.g., a self-reported questionnaire), by a third-party or by a computer, including a medical device (e.g., as such as defined by the FDA or other regulatory body) or other device (e.g., a medical sensor or biosensor, a watch or fitness tracker, or a “wearable”), and whether graded by a human decision-maker or an artificial intelligence, machine learning, or computer algorithm.

In some embodiments, disclosed crystalline forms are used to modulate neurotransmission. In some embodiments, disclosed crystalline forms are used to treat a condition, such as a disease, disorder, or a symptom thereof. As used herein, the term “condition” can be used interchangeably with the terms “disease”, “disorder”, or “symptom”. In some embodiments, disclosed crystalline forms are used in the manufacture of a medicament for the therapeutic and/or the prophylactic treatment of a condition, such as a disease disorder. In some embodiments, disclosed crystalline forms are administered in a therapeutically effective amount to a subject having a condition, such as a disease or a disorder. In some embodiments, the condition is a mental health disorder. In some embodiments, the condition is a neurodegenerative disorder. In some embodiments, the condition is an inflammatory disorder. In some embodiments, the condition is pain and/or inflammation. In some embodiments, the condition is epilepsy. In some embodiments, the condition is osteoarthritis. In some embodiments, the condition is nausea. In some embodiments, the condition is anxiety. In some embodiments, the condition is migraine. In some embodiments, the condition is headache. In some embodiments, the condition is sleep disruptions. In some embodiments, the condition is insomnia. In some embodiments, the condition is attention deficit/hyperactivity disorder (ADHD). In some embodiments, the condition is post-traumatic stress disorder (PTSD). In some embodiments, the condition is fibromyalgia. In some embodiments, the condition is glaucoma. In some embodiments, the condition is inflammatory bowel disease (IBD) and/or inflammatory bowel syndrome (IBS). In some embodiments, the condition is a viral infection. For example, the viral infection comprises an infection with a coronavirus. For example, the coronavirus comprises severe acute respiratory syndrome coronavirus-2 (SARS-CoV-2), see e.g., van Breemen et al., Cannabinoids Block Cellular Entry of SARS-CoV-2 and the Emerging Variants, J. Nat. Prod. 2022, 85, 176-184. In some embodiments, the condition is spasticity. In some embodiments, the condition is vomiting. In some embodiments, the condition is dementia. In some embodiments, the condition is Parkinson disease. In some embodiments, the condition is schizophrenia. In some embodiments, the condition is reduced appetite. In some embodiments, the condition is Tourette syndrome. In some embodiments, the condition is addiction. For example, the addiction can comprise an opioid addiction. In some embodiments, the condition can comprise cancer. For example, the cancer can comprise glioma. In some embodiments, the condition can comprise cancer-associated anorexia. In some embodiments, the condition can comprise anorexia nervosa. In some embodiments, the condition can comprise cachexia syndrome. In some embodiments, the condition can comprise symptoms of amyotrophic lateral sclerosis. In embodiments, the condition can comprise neuropsychiatric symptoms. In some embodiments, disclosed compounds are administered to a subject that is healthy.

In some embodiments, disclosed compounds or compositions thereof are orally, mucosally, rectally, subcutaneously, intravenously, intramuscularly, intranasally, by inhalation or transdermally administered to a subject. In some embodiments, when administered through one or more such routes, the disclosed compounds and the disclosed compositions and formulations comprising them are useful in methods for treating a patient in need of such treatment.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1—Example 1 Cannabinoid Acid Crystal Forms Cannabinoid Acids, Salts, and Cocrystals

Described herein provides crystalline solid forms of cannabinoid acids that can be applicable to a wide range of products, including pharmacological active ingredients.

Cannabinoid acids are a class of natural products which can be isolated from the plant C. sativa. Tetrahydrocannabinolic acid (THCa), cannabidiolic acid (CBDa), cannabigerolic acid (CBGa) are all examples of isomeric cannabinoid acids found in C. sativa., with a molecular weight of about 358 Da and a molecular formula of C₂₂H₃₀O₄.

The best known and most widely used cannabinoids (e.g., THC, CBD, CBG and CBN) are the result of decarboxylation of the plant synthesized (natural products) cannabinoid acids (e.g., THCa, CBDa, CBGa and CBNa). Purified forms of cannabinoids extracted from herbal cannabis (i.e., C. sativa) or synthetically produced cannabinoids are both potentially useful as active pharmaceutical agents. For example, THC (known as dronabinol) is FDA approved for the treatment of anorexia associated with weight loss in AIDS patients and shows potential pharmacological activity in the treatment of nausea, anxiety, glaucoma and migraines. More recently, the FDA approved Epidiolex, which contains a purified form of the drug substance cannabidiol (CBD) for the treatment of seizures associated with several medical conditions. Cannabinoid acids have only recently been characterized and analyzed for their potential as pharmacological active ingredients in several medical conditions and symptoms. For example, cannabinoid acids have been shown to be COX-1 and COX-2 inhibitors, making them candidates for nonsteroidal anti-inflammatory drugs (NSAIDs). Additionally, CBDa and CBGa have both been found to bind to the SARS-CoV-2 spike protein S1 C-terminal domain, which can inhibit the virus' ability to enter new host cells. This can lead to their evaluation as anti-viral drugs for use against SARS-CoV-2. As cannabinoid acids become a larger focus of study, additional applications are sure to be found for these important natural product molecules.

Cannabinoid acids are produced by the C. sativa plant via a biosynthetic pathway resulting in olivetolic acid and divarinolic acid. These acid substrates are then converted to either cannabigerovarinic acid (CBGVa) or cannabigerolic acid (CBGa) by the geranyl transferase enzyme. CBGa is further enzymatically converted to CBDa, THCa or CBCa by the corresponding synthase. Additionally, CBGVa can be further enzymatically converted to CBDVa, THCVa and CBCVa, by the corresponding CBDa, THCa or CBCa synthase. Heat, light and oxidizers can act on all 6 of these cannabinoids to produce a range of other cannabinoids via isomerizations, oxidations or degradation (e.g., decarboxylation). For example, photo irradiation can cause CBCa to isomerize to cannabicyclolic acid (CBLa) or cannabicitranic acid (CBTa). Furthermore, oxidation of THCa or CBDa can lead to cannabinonalic acid (CBNa) or cannabinodiolic acid (CBNDa), respectively. The examples above are only a small subset of the possible isomerizations and additional cannabinoids, which can be obtained from the plant-produced cannabinoid acids.

Cannabinoid acids tend to degrade over time leading to shelf life stability concerns. One of the main mechanisms of decomposition involves the decarboxylation of the acid group over time to form the associated unacidified cannabinoids. Which changes their structure in a way that affects their binding affinity to receptors and in turn their efficacy for a desired application. For example, cannabinoid acids have much different effects on the COX-1 and COX-2 enzymes than their decarboxylated counterparts. Decomposition occurs slowly over time and is accelerated by the presence of heat and light, leading to storage challenges. Some cannabinoids are more susceptible to decarboxylation than others, for example CBDa decarboxylates at a faster rate than THCa under similar conditions. Therefore, it is important to mitigate decarboxylation related decomposition for some cannabinoids which are more susceptible to this process. By purifying and solidifying cannabinoid acids in specific crystal forms with controlled and molecularly understood intermolecular hydrogen bonding and molecular lattice packing, we can use molecular engineering and molecular interactions to predict and control solid cannabinoid acids that are more resistant to chemical degradation (e.g., decarboxylation) or reaction (e.g., oxidation).

Herein provides molecular structural elucidation of new crystalline states of cannabinoid acids which have improved stability and resistance to chemical reaction and degradation, when compared to their liquid, amorphous or molecular mixture and solution states, which are the common naturally occurring forms of cannabinoid acids. Additionally, the methods for producing the crystalline states of the cannabinoid acids are described herein.

Pure single crystals and polycrystalline solids of THCa were generated by suspending a high THCa whole plant extract in the minimum amount of warm liquid hydrocarbon (e.g., heptane), and storing in a freezer at −15° C. until crystals formed overnight. Alternatively, the liquid hydrocarbon can be allowed to evaporate at controlled higher temperatures until crystallization occurred. Repeating the process of recrystallization provides a method of producing highly molecularly purified cannabinoid acid compounds. Prior to molecular elucidation of these new solid forms, material was recrystallized three times to ensure high molecular purity. The crystals of CBGa were obtained by extracting high CBGa hemp (10 grams) using 100 mL of cold (−15° C.) heptane with agitation for approximately 10 minutes followed by vacuum filtration. The filtrate was dried using a rotary evaporator to produce crude CBGa crystals. The crude CBGa crystals were recrystallized by dissolving in the minimum amount of warm heptane and then allowing to cool 0° C. in a freezer. Alternatively, the heptane was allowed to slowly evaporate until crystals were observed. This process was repeated 3 times to obtain higher purity crystals. The CBDa crystals were obtained by dissolving a high CBDa plant extract (20 grams) in cold acetonitrile (100 mL) and shaken for approximately 5 minutes followed by vacuum filtration. Approximately 50 mL of acetonitrile was removed under a flow of nitrogen to concentrate the sample. This sample was then placed into a freezer at −15° C. for an extended period of time until crystals were observed.

### Characterization of Cannabinoid Acid Crystals

The cannabinoid acid crystals were characterized using a range of molecular techniques including, powder X-ray diffraction (pXRD), single crystal x-ray diffraction (SCXRD), Fourier transform infrared (FTIR) spectroscopy, solid-state NMR, liquid-state NMR, thermogravimetric analysis (TGA), and differential scanning calorimetry (DSC).

Molecular elucidation and engineering of solids with enhanced intermolecular interactions to increase the molecular stability of cannabinoid acids that otherwise in their isolated state have a significantly higher propensity for chemical degradation or reaction.

This disclosure provides crystalline forms of cannabinoid acids with increased molecular stability.

Improved molecular stability over current non-crystalline solid and liquid forms. This can, for example, increase shelf life and reduce the cost associated with cannabinoid acid API degradation. It can also improve the safety of cannabinoid acid products due to the reduction in decomposition to degradants with unknown biological activity.

### References Cited in this Example

(1) Gigopulu, O.; Geskovski, N.; Stefkov, G.; Stoilkovska Gjorgievska, V.; Slaveska Spirevska, I.; Huck, C. W.; Makreski, P. A Unique Approach for In-Situ Monitoring of the THCA Decarboxylation Reaction in Solid State.
Spectrochimica Acta Part A: Molecular and Biomolecular Spectroscopy 2022, 267, 120471. //doi.org/10.1016/j.saa.2021.120471.
(2)van Breemen, R. B.; Muchiri, R. N.; Bates, T. A.; Weinstein, J. B.; Leier, H. C.; Farley, S.; Tafesse, F. G. Cannabinoids Block Cellular Entry of SARS-CoV-2 and the Emerging Variants. J. Nat. Prod. 2022, 85 (1), 176-184. //doi.org/10.1021/acs.jnatprod.1c00946.
(3) Mahmud, M. S.; Hossain, M. S.; Faiz Ahmed, A. T. M.; Islam, M. Z.; Sarker, M. E.; Islam, M. R. Antimicrobial and Antiviral (SARS-CoV-2) Potential of Cannabinoids and Cannabis Sativa: A Comprehensive Review. Molecules 2021, 26 (23). //doi.org/10.3390/molecules26237216.
(4) Punyamurthula, N. S.; Hingorani, T.; Adelli, G.; Gul, W.; ElSohly, M. A.; Repka, M. A.; Majumdar, S. Controlled Release Tablet Formulation Containing Natural A9-Tetrahydrocannabinol. Drug Development and Industrial Pharmacy 2016, 42 (7), 1158-1164. //doi.org/10.3109/03639045.2015.1118490.
(5) Cuadari, A.; Pollastro, F.; Unciti-Broceta, J. D.; Caprioglio, D.; Minassi, A.; Lopatriello, A.; Munoz, E.; Taglialatela-Scafati, O.; Appendino, G. The Dimerization of A9-Tetrahydrocannabinolic Acid A (THCA-A).
Acta Pharmaceutica Sinica B 2019, 9 (5), 1078-1083. //doi.org/10.1016/j.apsb.2019.06.007.
(6) Abraham, O. R.; Waddell Smith, R. Optical and Spectroscopic Characterization of Crystalline Structures in Cannabis Extracts. Journal of Forensic Sciences 2021. //doi.org/10.1111/1556-4029.14940.

### Example 2—Cannabinoid Acid Crystal Forms

Cannabinoid acids are a class of natural products which can be isolated from the plant C. sativa. Purified forms of cannabinoids extracted from herbal cannabis or synthetically produced cannabinoids can be useful as active pharmaceutical agents. For example, THC is FDA approved for the treatment of anorexia associated with weight loss in AIDS patients and shows potential pharmacological activity in the treatment of nausea, anxiety, glaucoma and migraines. However, cannabinoid acids tend to degrade over time leading to shelf-life stability concerns. This degradation changes their structure in a way that affects their binding affinity to receptors and in turn their efficacy for a desired application. Herein described provides crystalline solid forms of cannabinoid acids that can be applicable to a wide range of products, including pharmacological active ingredients.

The molecular structural of crystalline states of the proposed cannabinoid acids have improved stability and resistance to chemical reaction and degradation.

Pure single crystals and polycrystalline solids of THCa were generated by suspending a high THCa whole plant extract in the minimum amount of warm liquid hydrocarbon (e.g., heptane), and storing in a freezer at −15° C. until crystals formed overnight

The crystals of CBGa were obtained by extracting high CBGa hemp (10 grams) using 100 mL of cold (−15° C.) heptane with agitation for approximately 10 minutes followed by vacuum filtration

The filtrate was dried using a rotary evaporator to produce crude CBGa crystals. The crude CBGa crystals were recrystallized by dissolving in the minimum amount of warm heptane and then allowing to cool 0° C. in a freezer.

The CBDa crystals were obtained by dissolving a high CBDa plant extract (20 grams) in cold acetonitrile (100 mL) and shaken for approximately 5 minutes followed by vacuum filtration.

Approximately 50 mL of acetonitrile was removed under a flow of nitrogen to concentrate the sample and the sample was placed at −15° C. until crystals were observed.

The method increases the molecular stability of cannabinoid acids.

The method increases shelf life and reduces the cost associated with cannabinoid acid API degradation.

The method also improves the safety of cannabinoid acid products due to the reduction in decomposition to degradants with unknown biological activity.

### Example 3—Crystalline Solids of Cannabinoid Acids, Salts, and Cocrystals

### Screening for Additional Co-Crystals and Salts:

Aspects of the invention are drawn towards the generation of cocrystals and salts of cannabinoids. Without wishing to be bound by theory, these salts and cocrystals can have increased stability over their non-crystalline forms.

To screen for additional cocrystals and salts, the cannabinoid acids and the selected coformer, will be dispersed in a solution of methanol, methanol/water, acetonitrile, or THF, in a 1-to-1 molar ratio. The solvent will be evaporated via nitrogen gas flow and dried under vacuum. The resultant solid will be redispersed in one of the following solvents: Acetonitrile, methanol, toluene, heptane, ethyl acetate, tetrahydrofuran. The temperature will be increased to 50 C and then slowly cooled to 1° C. or −15 C to precipitate crystals of the salt or cocrystal. Alternatively, the solvent will be allowed to slowly evaporate to generate co-crystals or salts. Alternatively, the cannabinoid acid and the conformer will be ground together using a motor and pestle or ball mill or similar equipment, with and without the presence of a small amount of solvent. The potential salts/cocrystals will be characterized by analysis with FT-IR, NMR, and powder XRD.

### Stability Studies:

Stability studies will be carried out to determine the expected shelf-life stability of the crystalline forms of the cannabinoid acids compared to their amorphous/viscous liquid forms commonly used. This study will be carried out by storing cannabinoid acid crystals and amorphous cannabinoid samples in similar conditions of temperature and humidity and periodically sampling them via HPLC and/or liquid-state NMR to determine the extent to which the cannabinoid acid has decomposed.

### Bioavailability Studies:

For select salts and cocrystals where an increase in bioavailability might be expected, the dissolution kinetics will be measured and compared to that of the crystalline cannabinoid acid. Bioavailability studies will be carried out in a USP-2 paddle type apparatus operating at 37 C with a rotation speed of approximately 150 RPM. The solution will be comprised of a fed state simulated intestinal fluid (FeSSIF), or similar. The cannabinoid acids or cannabinoid acid cocrystal/salt will be pressed into a pellet along with a mixture of standard pharmaceutical excipients (microcrystalline cellulose, magnesium stearate, etc.). The pellets will be placed into the dissolution tester apparatus and samples will be collected periodically. The collected samples will be analyzed quantitatively on an HPLC.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1.-17. (canceled)

18. A crystalline form of tetrahydrocannabinolic acid (THCa) of the following structure:

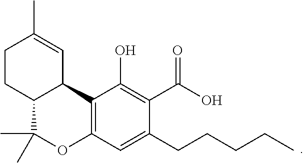 [SMILES: CCCCCc1cc2c(c(O)c1C(=O)O)[C@@H]1C=C(C)CC[C@H]1C(C)(C)O2]

19. The crystalline form of claim 18, wherein the crystalline form is selected from a single component crystalline form or a multicomponent crystalline form.

20. The crystalline form of claim 19, wherein the multicomponent crystalline form comprises a co-crystal, a salt, or a combination thereof.

21. The crystalline form of claim 19, wherein the single component crystalline form comprises at least one of the following features (i) to (ix):
(i) a powder x-ray diffraction (PXRD) pattern comprising at least one characteristic peak at 2θ angles of about 6.5, 8.8, 9.6, 10.1, 12.7, 13.1, 14.1, 15.1, 16.4, 17.0, 18.7, 19.4, 19.8, 20.3, 21.3, 21.7, 22.7, 23.6, 25.3, 27.1, and 29.4;
(ii) a single crystal x-ray diffraction (SCXRD) pattern comprising at least one characteristic peak at 2θ angles described herein;
(iii) a Fourier transform infrared spectroscopy spectrum comprising at least one characteristic peak at about 742, 820, 887, 973, 1029, 1070, 1107, 1167, 1234, 1360, 1428, 1547, 1644, 1700, 2860, 2926, 2957 cm⁻¹;
(iv) a liquid state ¹H nuclear magnetic resonance (NMR) spectrum comprising at least one characteristic peak at about 0.903, 1.11, 1.35(m), 1.44, 1.58(m), 1.68, 1.92(m), 2.17, 2.79(m), 2.95(m), 3.23, 6.26, 6.39, 11.70, 12.16 ppm in CDCl₃;
(v) a liquid state ¹³C (¹H decoupled) nuclear magnetic resonance (NMR) spectrum comprising at least one characteristic peak at about 14.1, 19.5, 22.5, 23.4, 25.0, 27.4, 31.2, 31.3, 32.0, 33.5, 36.5, 45.6, 78.9, 102.3, 109.9, 112.7, 123.6, 133.9, 147.0, 159.8, 164.7, 176.5 ppm in CDCl₃;
(vi) a solid state ¹H NMR spectrum comprising at least one characteristic peak at about 1.5, 6.2, 11.2, 11.8, 12.7 ppm;
(vii) a solid state ¹H-¹³C cross polarization (CP) magic angle spinning (MAS) NMR spectrum comprising at least one characteristic peak at about 13.8, 15.4, 17.9, 19.3, 20.7, 23.6, 24.2, 24.6, 25.1, 25.9, 26.6, 27.5, 28.5, 31.0, 32.3, 32.9, 35.3, 37.6, 44.1, 45.9, 77.8, 78.9, 101.5, 102.6, 109.6, 110.1, 102.6, 109.6, 110.1, 113.3, 123.1, 124.0, 132.3, 133.2, 146.6, 161.0, 162.0, 165.0, 165.6, 178.1, and 178.5 ppm;
(viii) a thermogravimetric analysis (TGA) thermogram comprising a 20% w/w loss from about 125° C. to about 165° C.; and 70% w/w loss from about 165° C. to 200° C.; or
(ix) a differential scanning calorimetry (DSC) thermogram comprising an endothermic onset at about 149° C.

22. The crystalline form of claim 21, wherein the single crystal belongs to the orthorhombic system, P2₁2₁2₁ space group, and is characterized by the following single-crystal lattice parameters: a=11.4635(8)Å, b=18.0506(13)Å, c=19.2347(14)Å, α=90 degrees, β=90 degrees, and γ=90 degrees.

23. The crystalline form of claim 21, wherein the single crystal x-ray diffraction (SCXRD) pattern is characterized by an Oak Ridge Thermal Ellipsoid Plot substantially as shown in FIG. 4.

24. (canceled)

25. The crystalline form of claim 21, characterized by a powder x-ray diffraction (PXRD) pattern substantially as shown in FIG. 5.

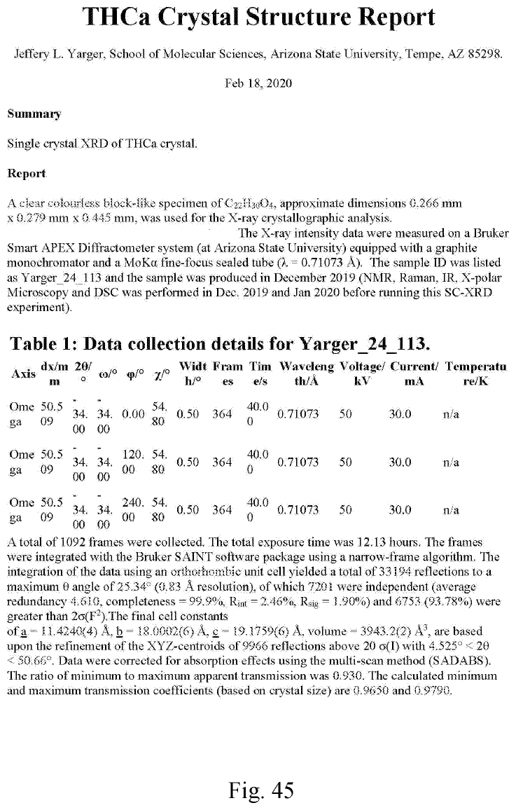

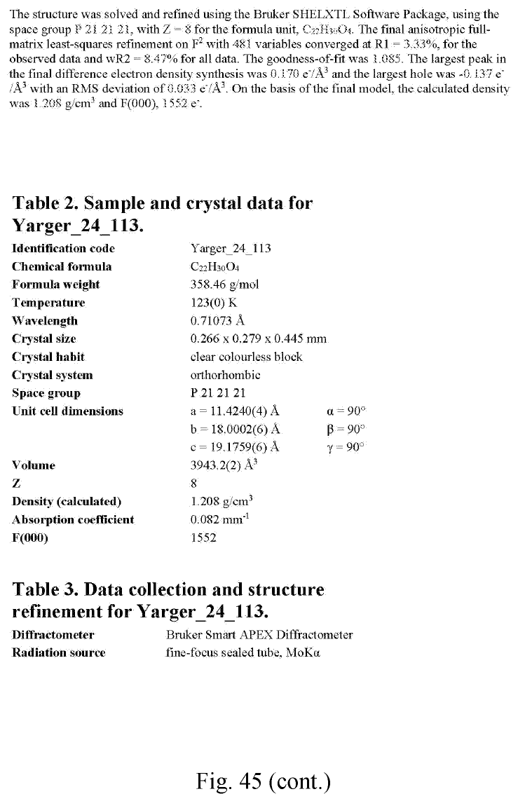

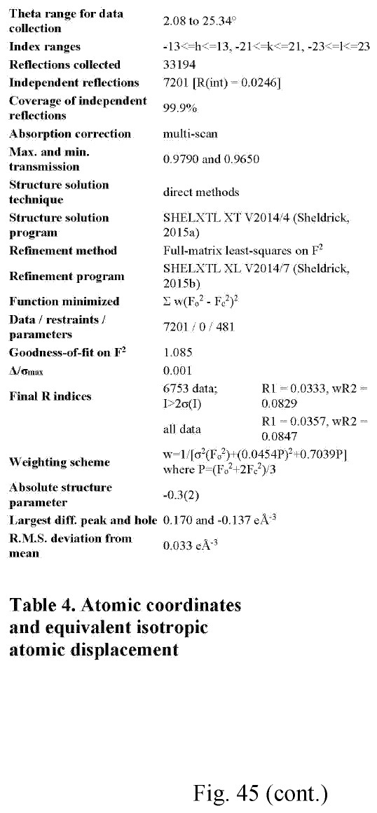

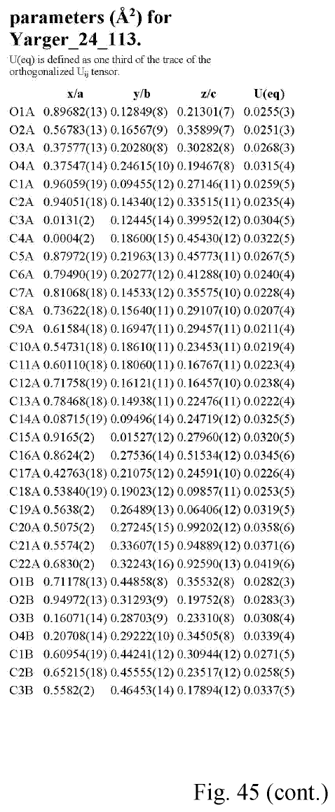

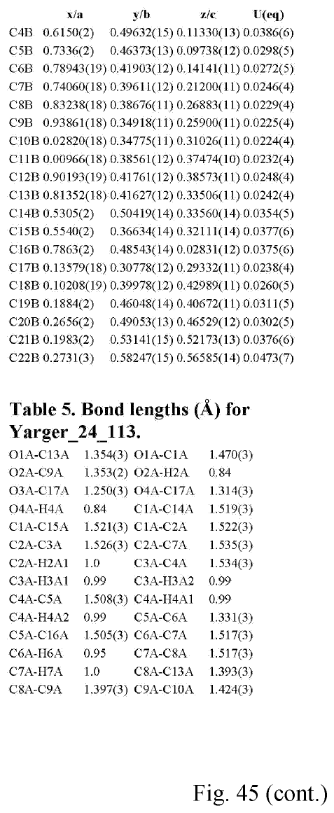

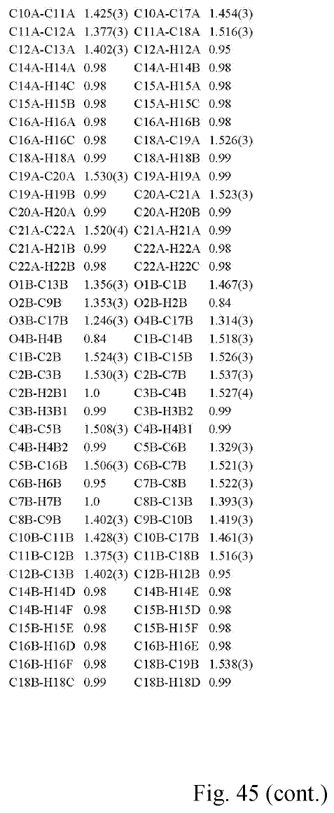

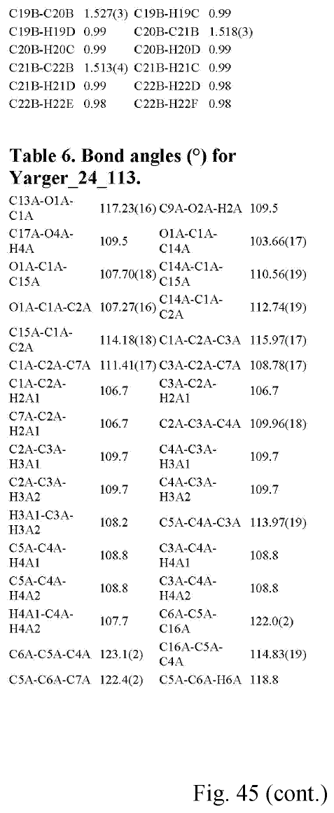

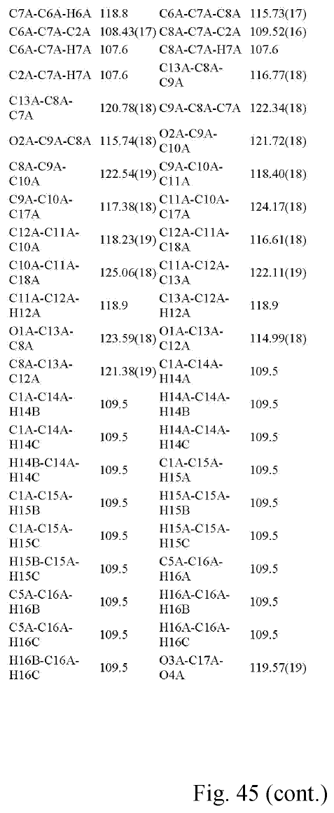

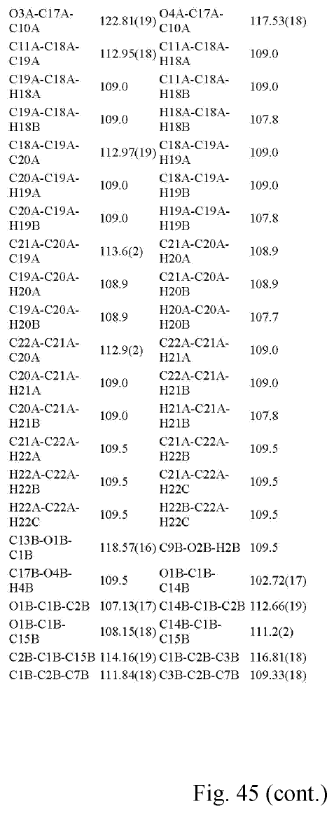

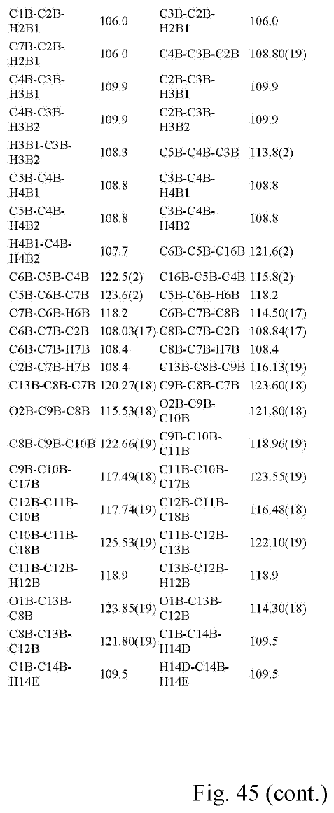

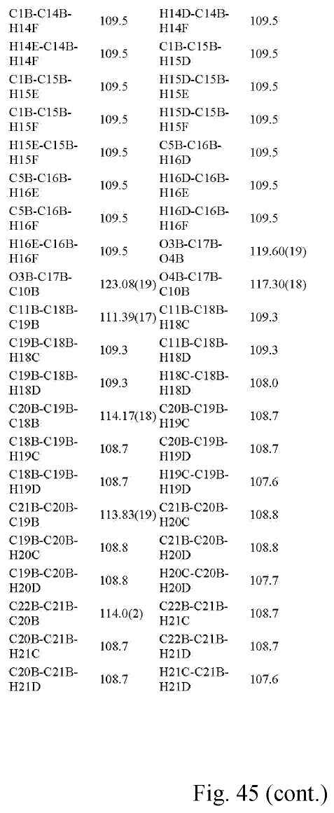

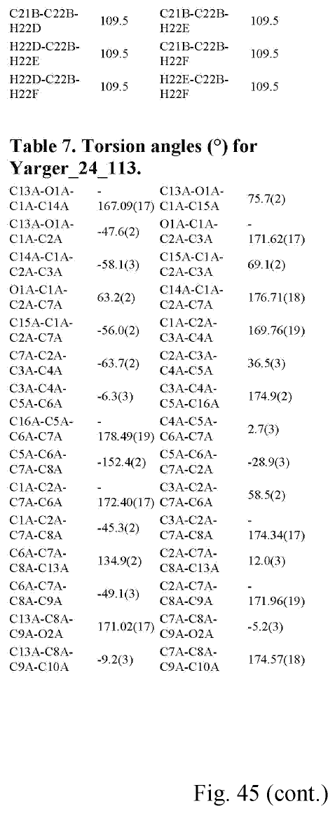

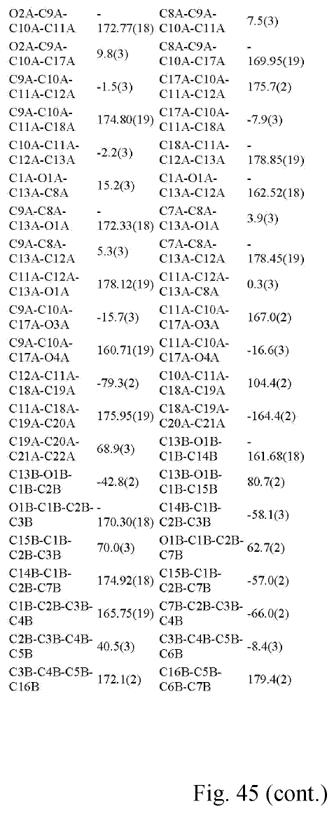

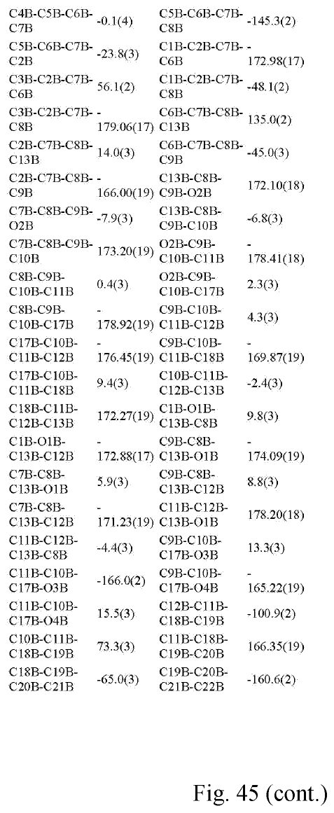

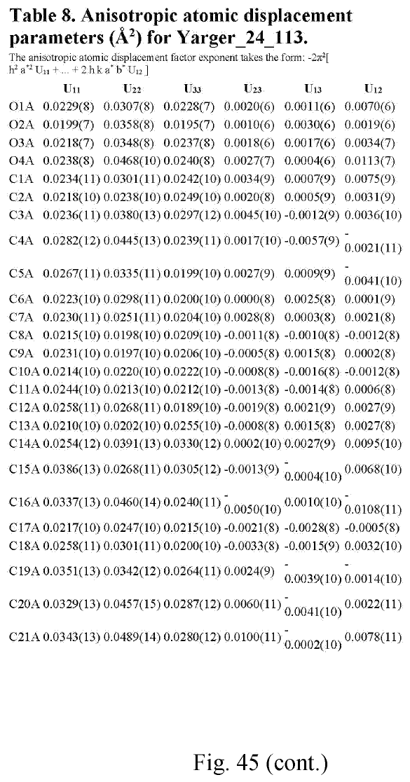

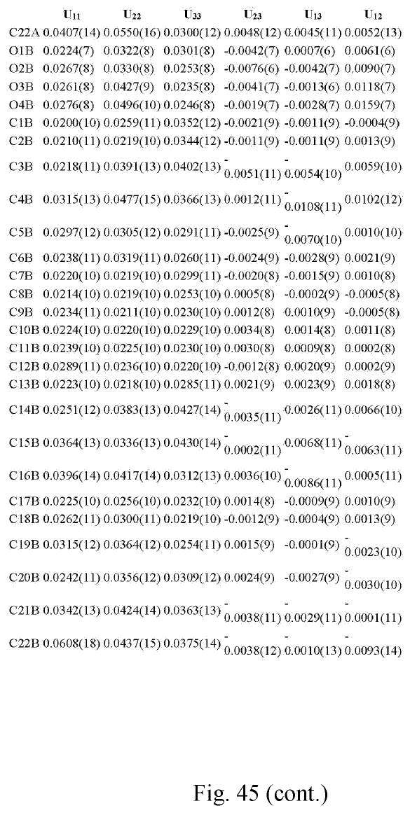

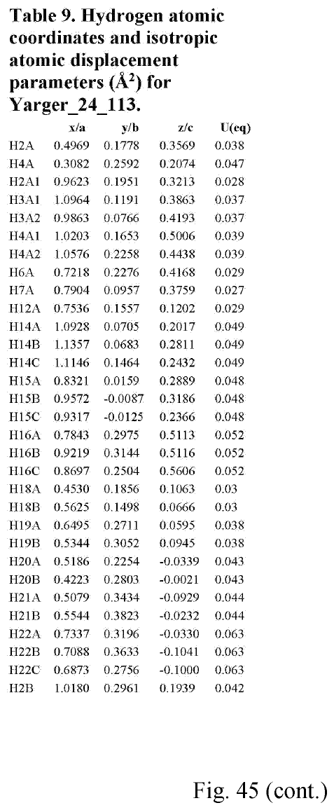

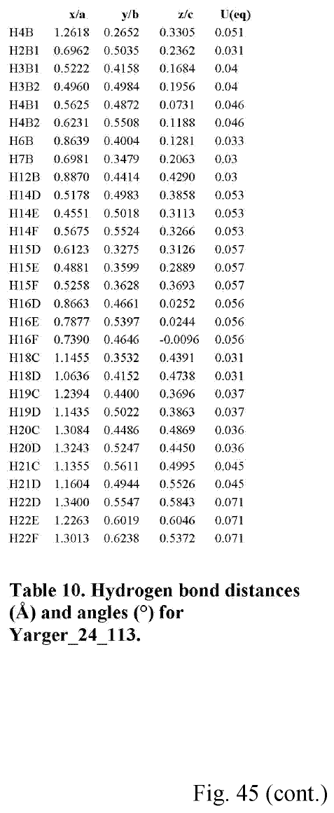

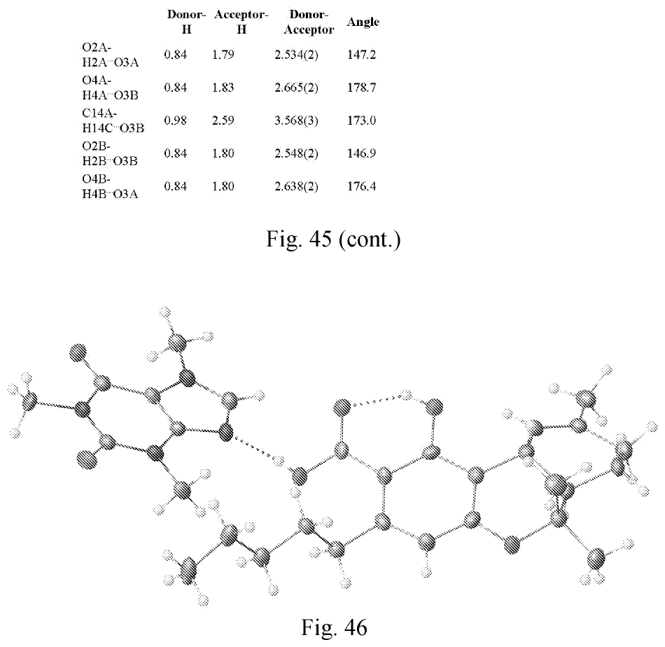

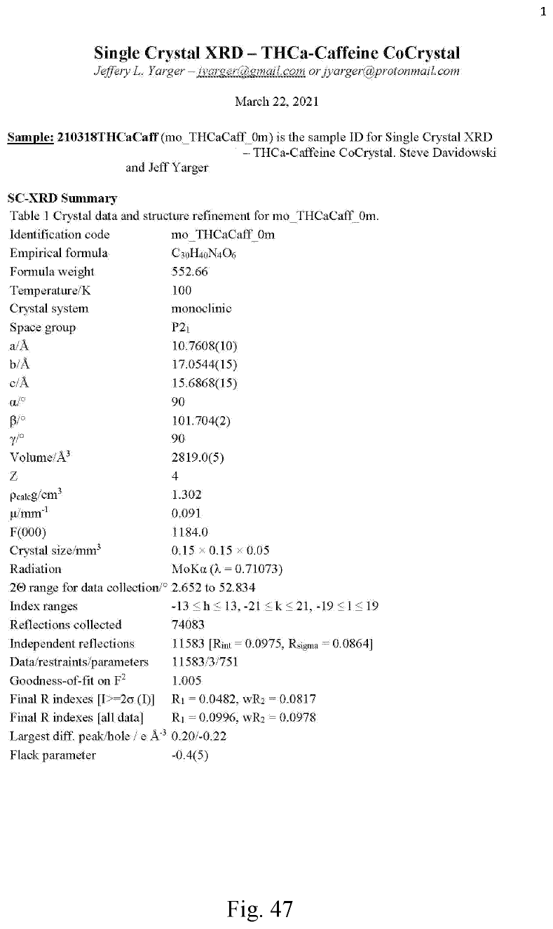

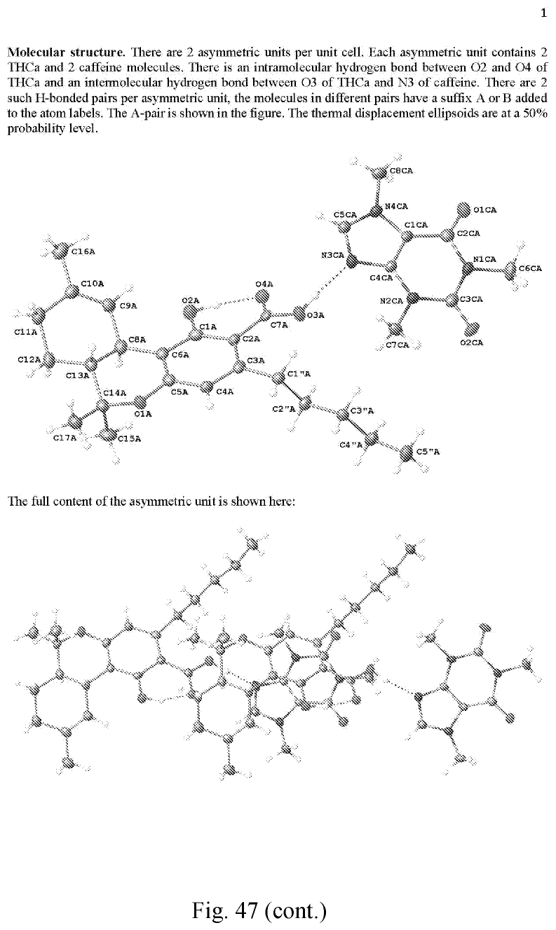

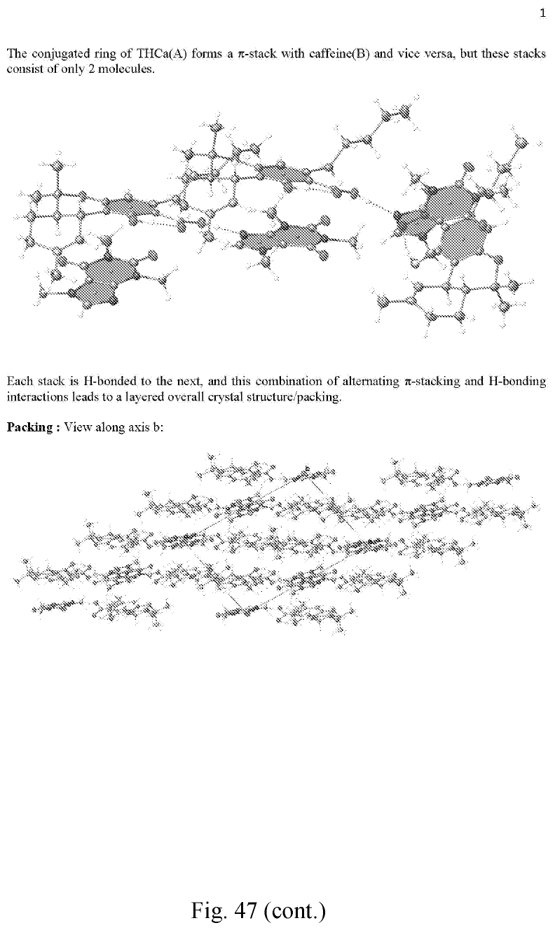

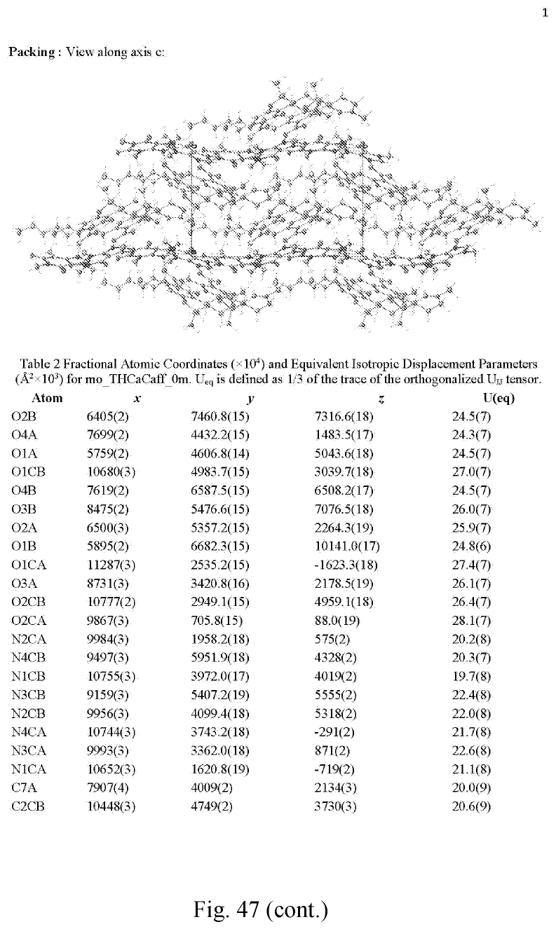

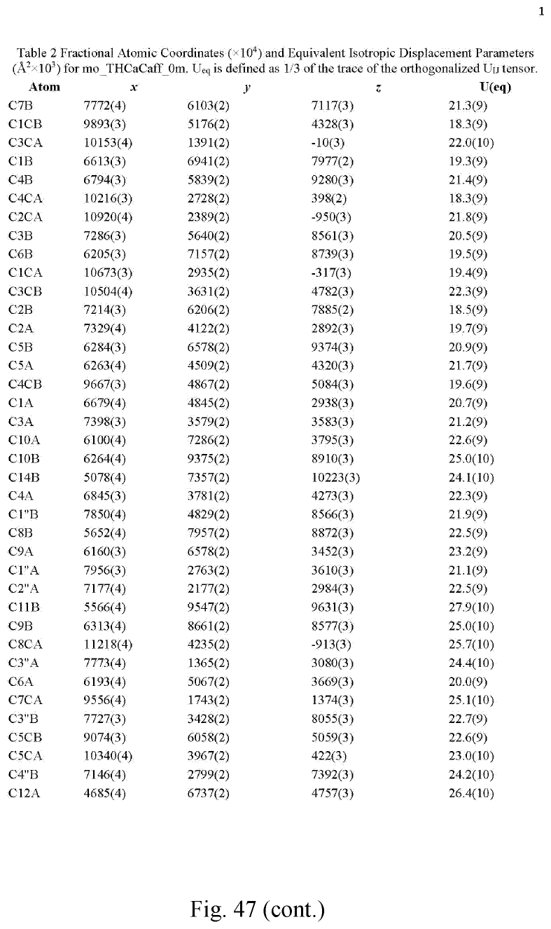

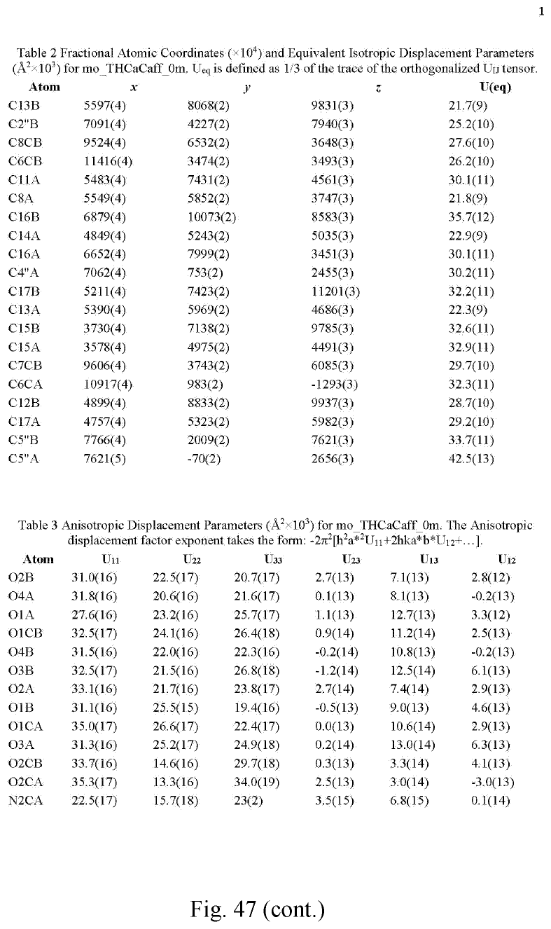

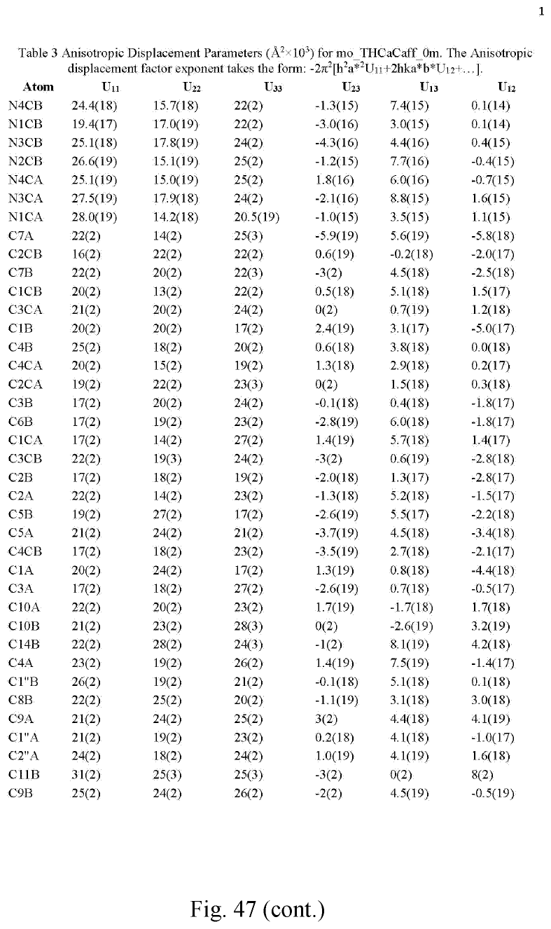

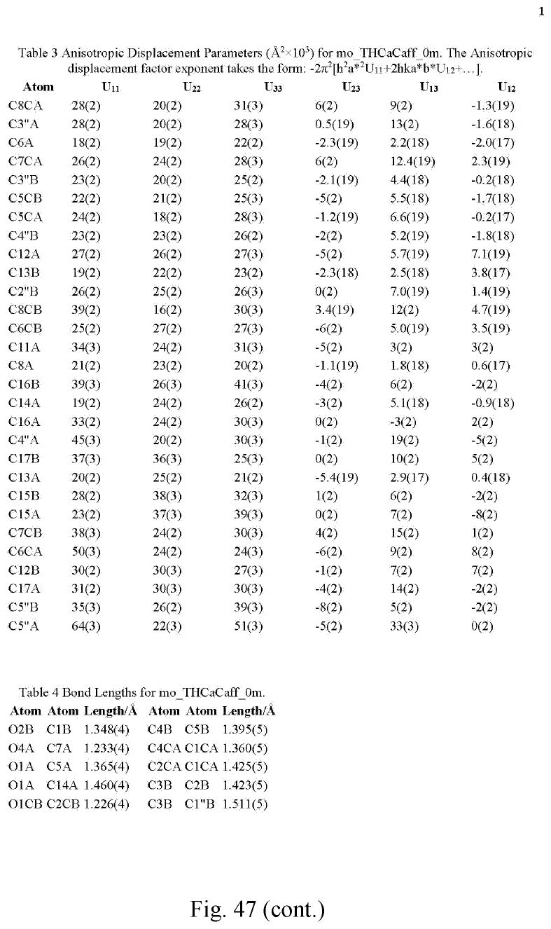

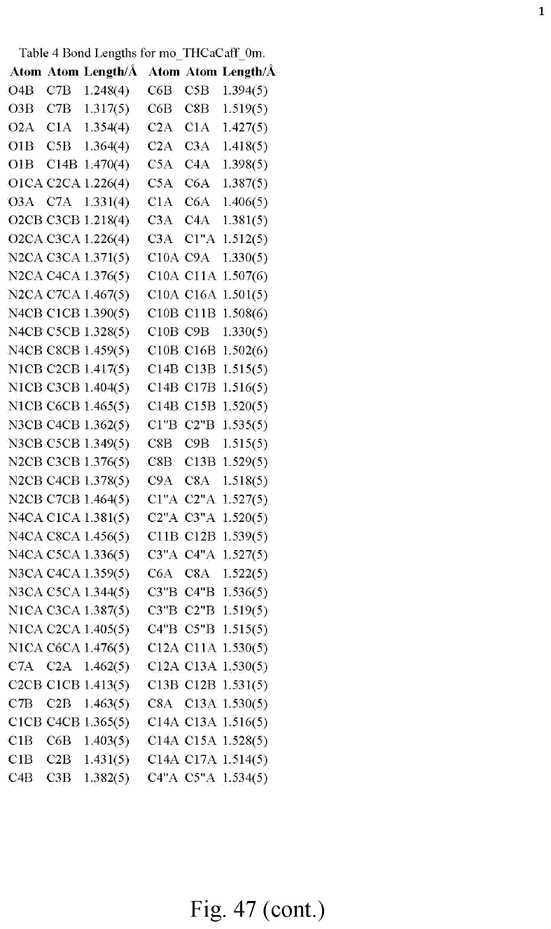

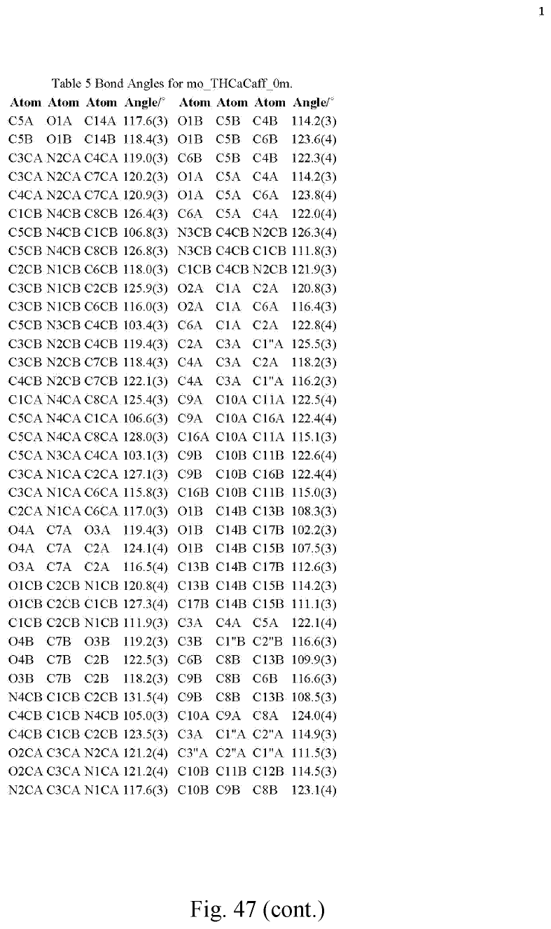

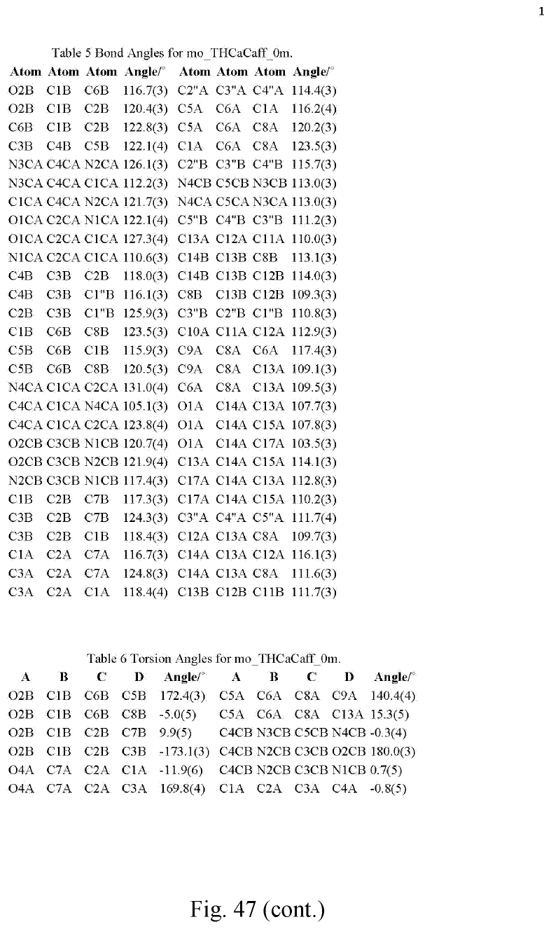

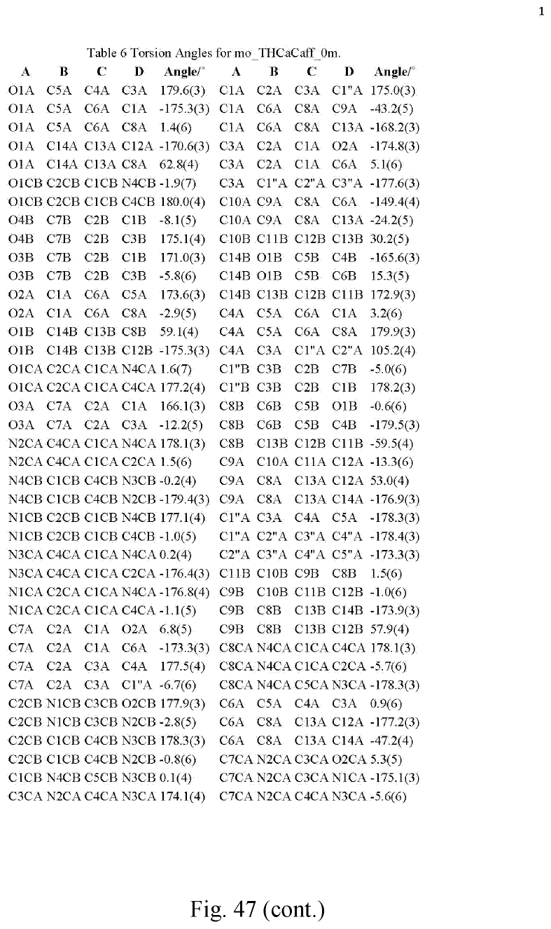

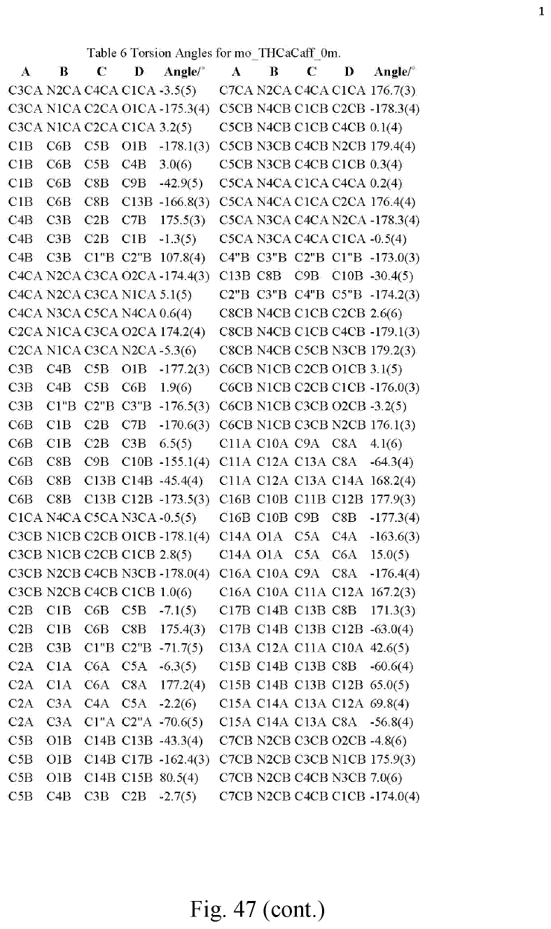

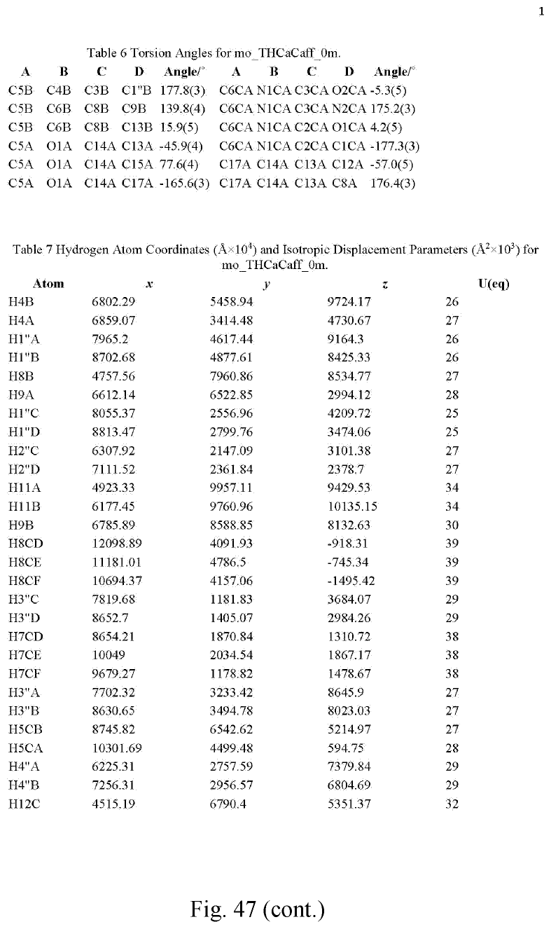

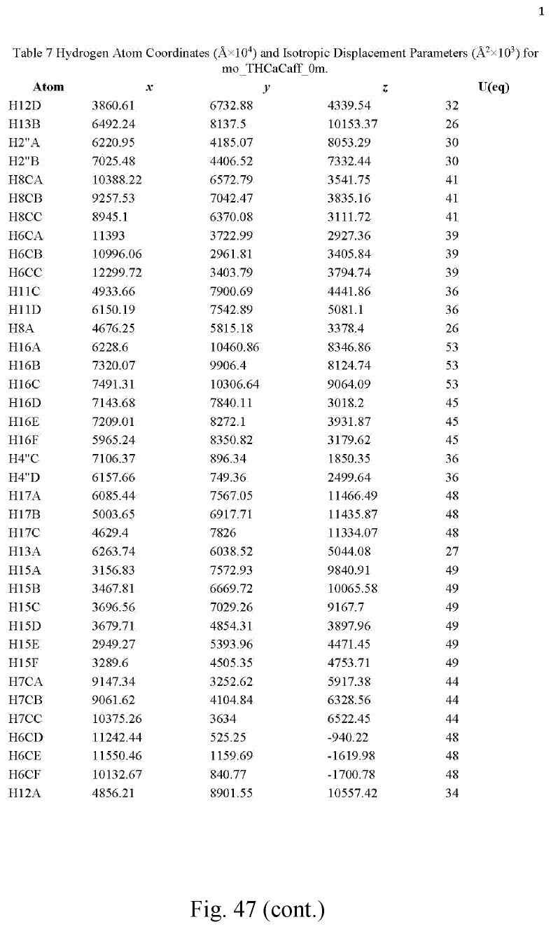

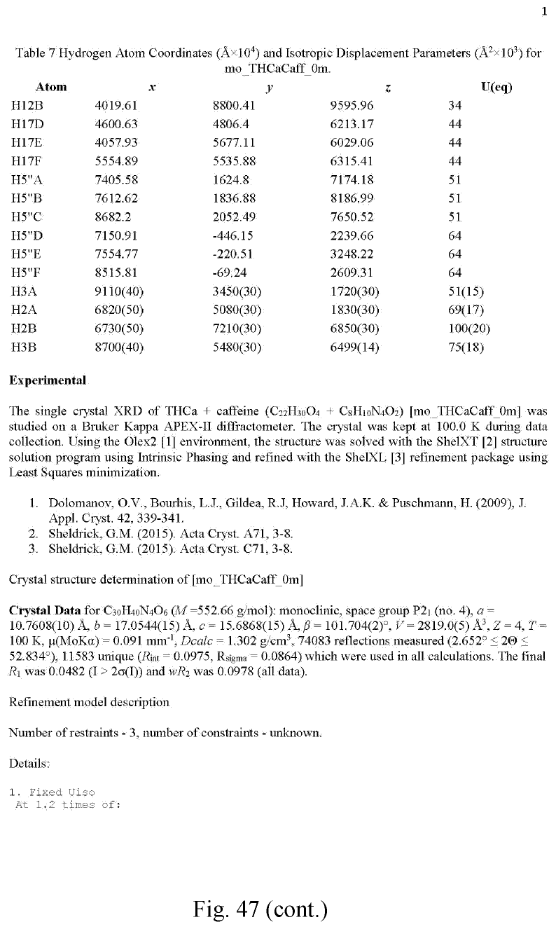

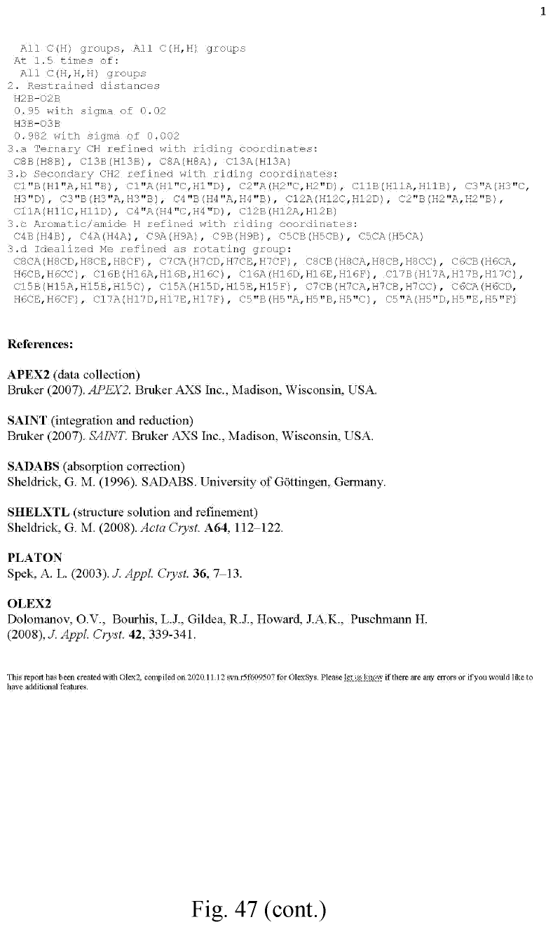

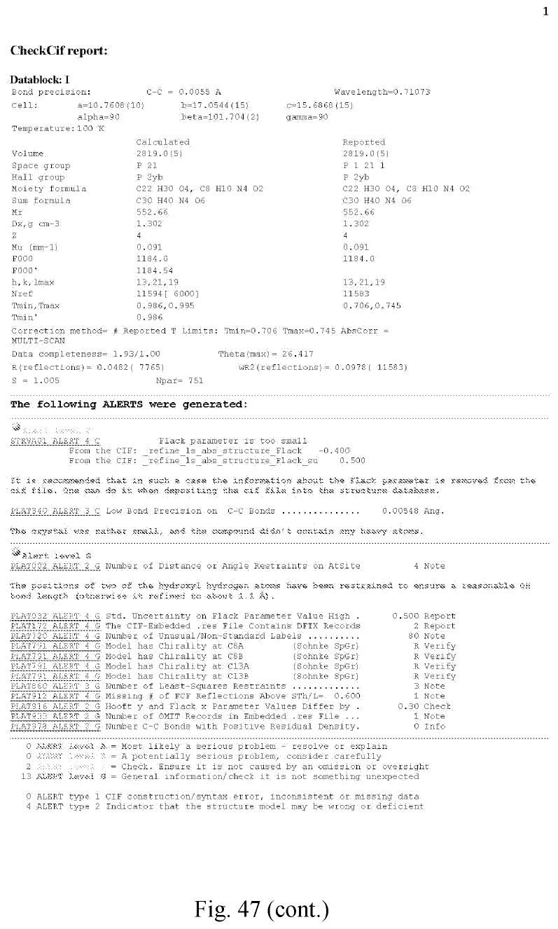

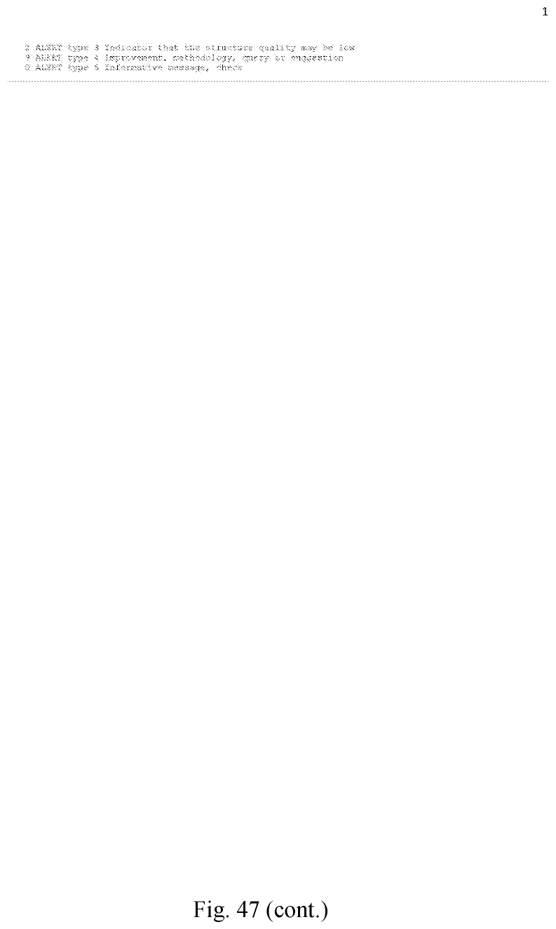

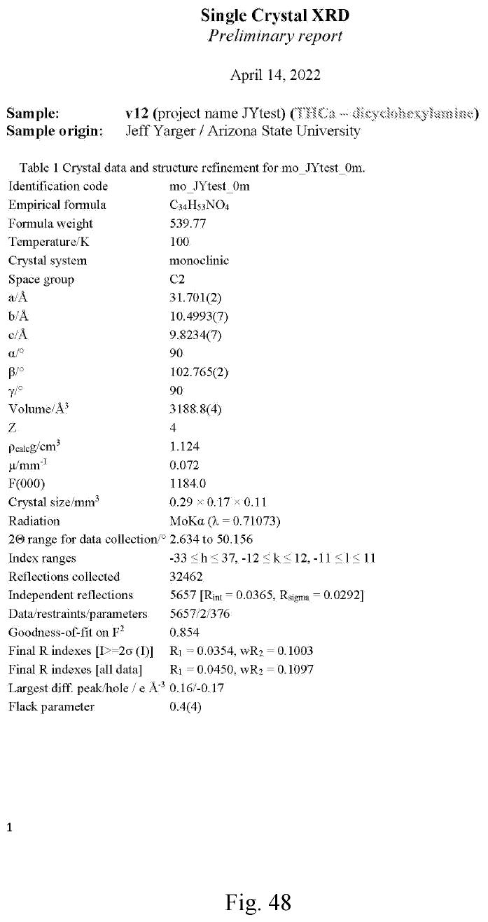

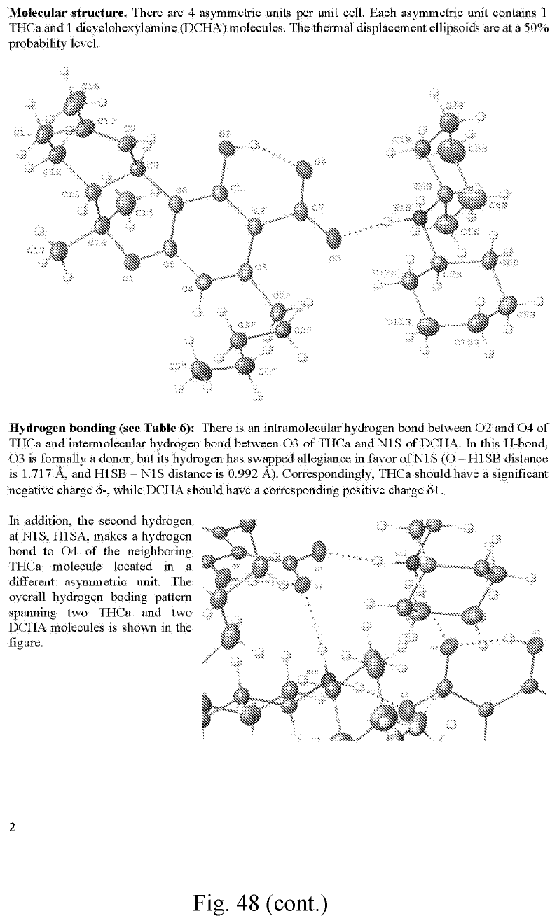

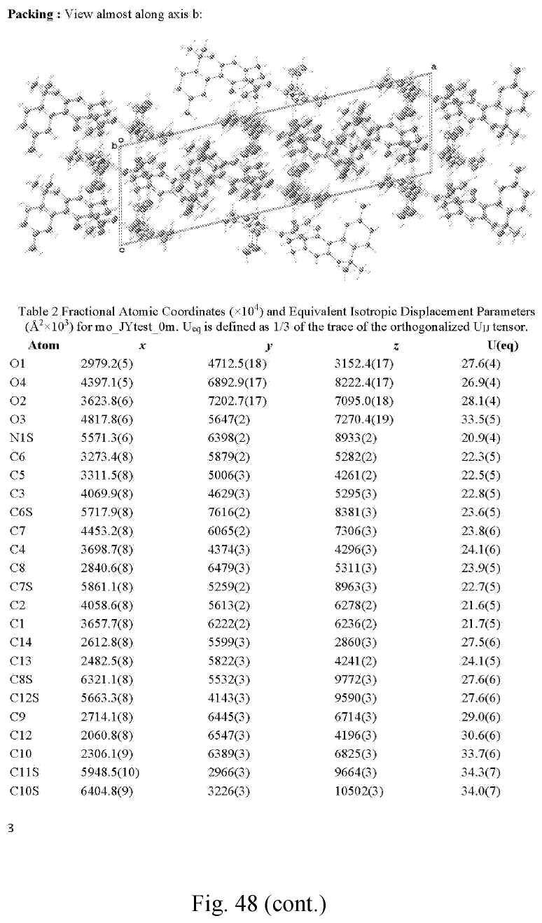

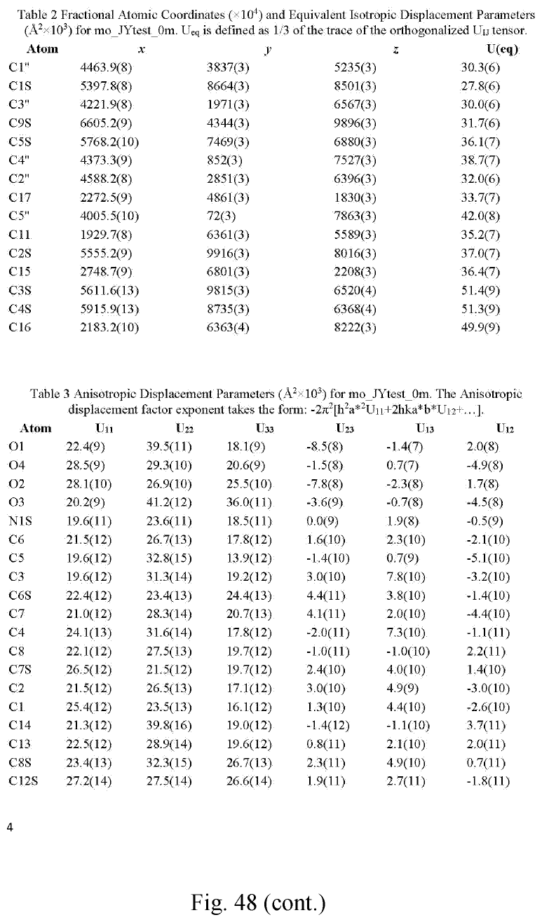

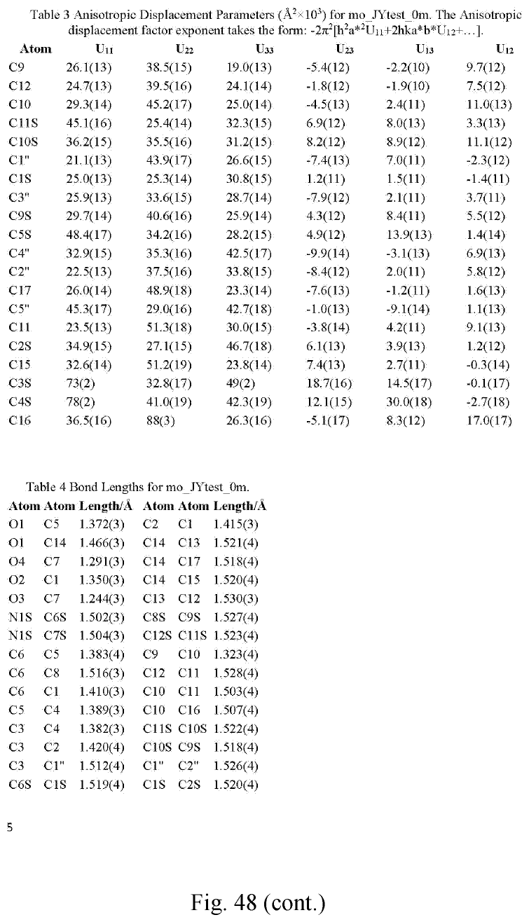

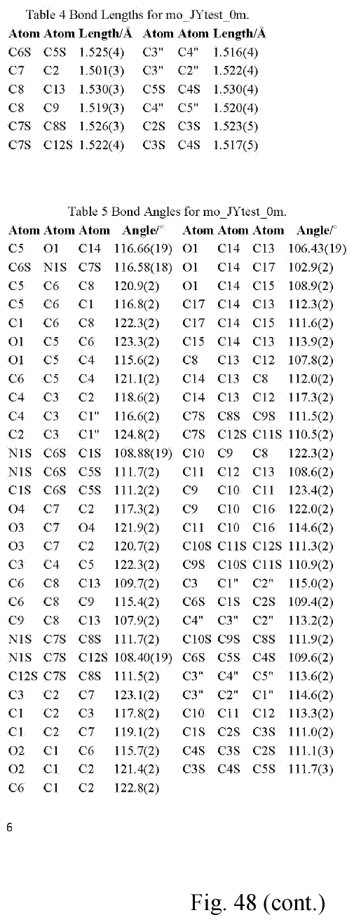

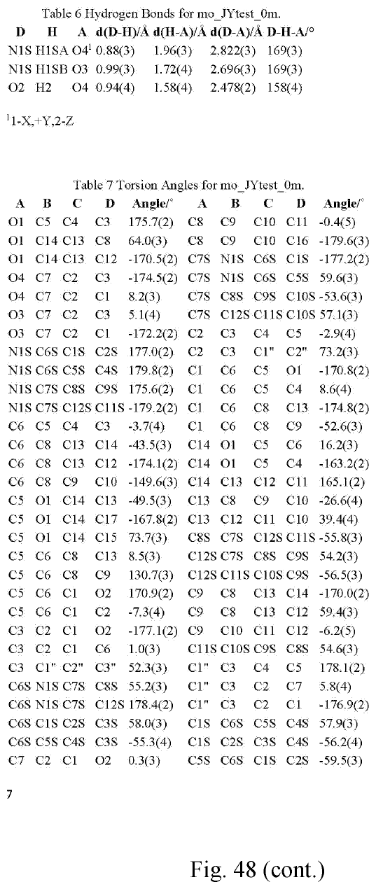

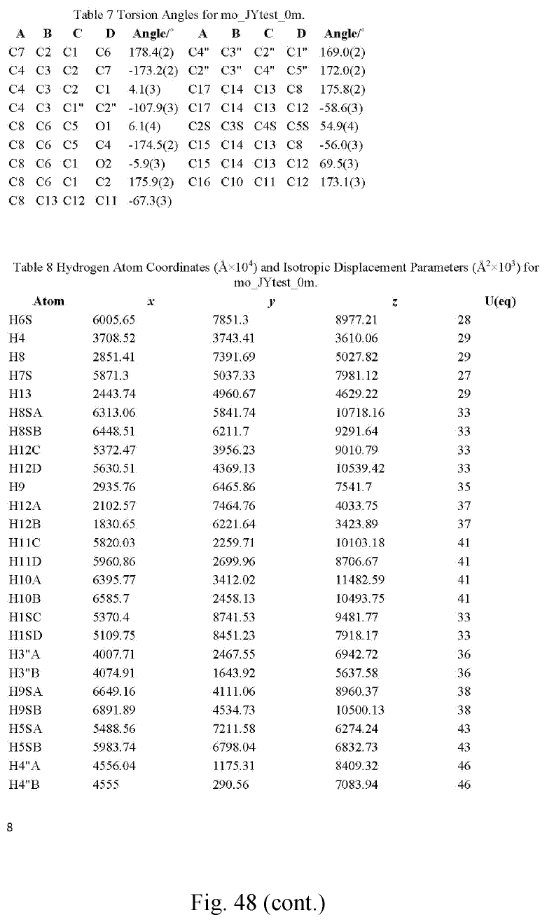

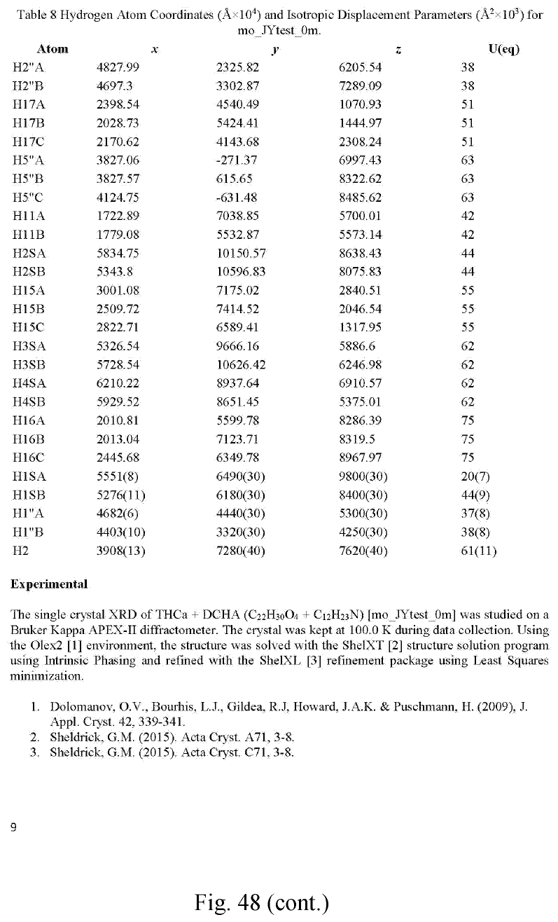

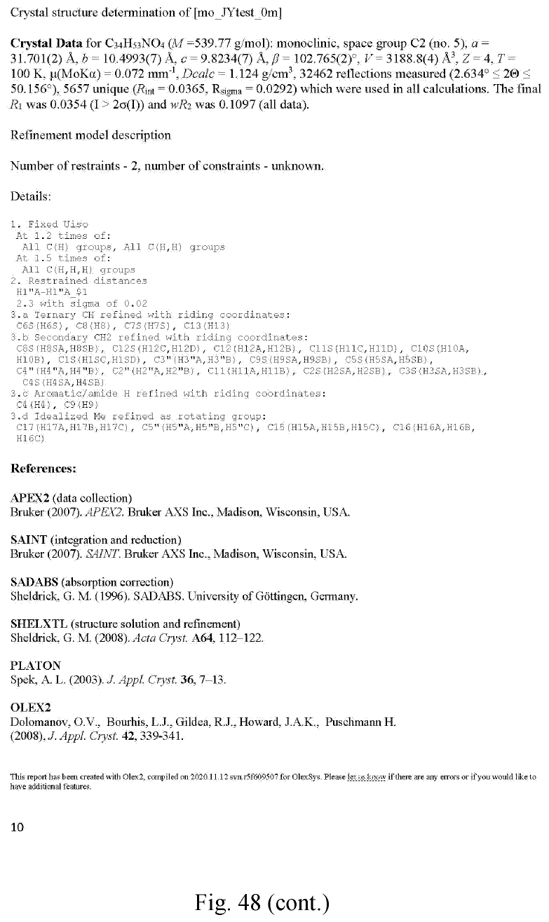

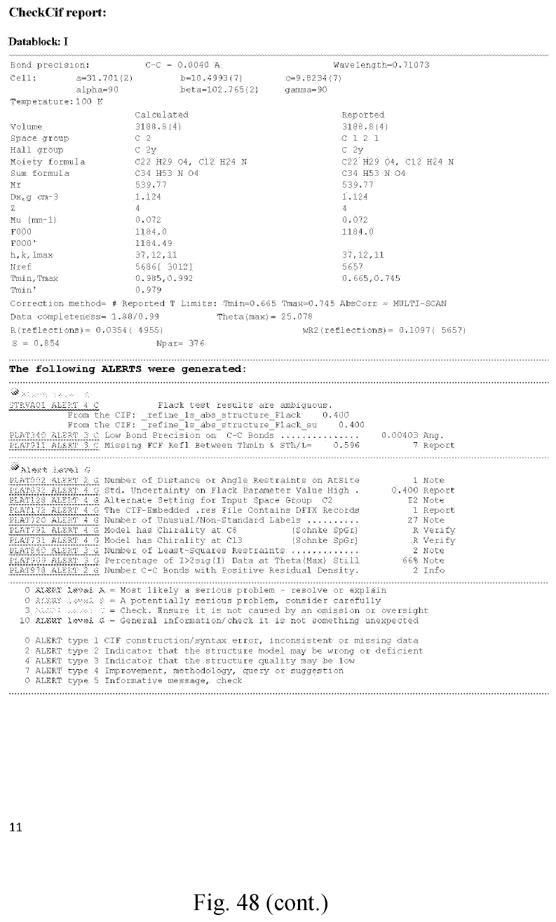

26. The crystalline form of claim 21, characterized by a single crystal x-ray diffraction (SCXRD) pattern substantially as shown in FIGS. 44 and 45.

27. The crystalline form of claim 21, characterized by the Fourier transform infrared spectroscopy spectrum substantially as shown in FIG. 8.

28. The crystalline form of claim 21, characterized by the liquid state ¹H nuclear magnetic resonance (NMR) spectrum substantially as shown in FIG. 2.

29. The crystalline form of claim 21, characterized by the ¹H-¹³C cross polarization (CP) magic-angle spinning (MAS) NMR spectrum substantially as shown in FIG. 7.

30. The crystalline form of claim 21, further characterized by the solid state ¹H NMR spectrum substantially as shown in FIG. 6.

31. The crystalline form of claim 21, characterized by the liquid state ¹³C (¹H decoupled) nuclear magnetic resonance (NMR) spectrum substantially as shown in FIG. 3.

32. The crystalline form of claim 21, wherein the TGA thermogram comprises a degradation onset of about 125° C.

33. The crystalline form of claim 21, characterized by the thermogravimetric analysis (TGA) thermogram substantially as shown in FIG. 9.

34. The crystalline form of claim 21, wherein the crystalline form is further characterized by a differential scanning calorimetry thermogram comprising an endothermic event in the range of about 140° C. to 149° C.

35. The crystalline form of claim 21, characterized by the differential scanning calorimetry (DSC) thermogram substantially as shown in FIG. 10.

36. A method of preparing a single component crystalline form or a multicomponent crystalline form of tetrahydrocannabinolic acid (THCa) comprising:
suspending a plant extract comprising THCa in minimum amount (10-50% w/w) of a solvent at a temperature of about 25° C. to about 80° C.;
storing the suspension in at about −15° C. for about 6 to 48 hrs; and
repeating the process until obtaining a purified crystal.

37. (canceled)

38. A method of preparing a single component or multicomponent crystalline form of tetrahydrocannabinolic acid (THCa) comprising:
suspending a plant extract comprising THCa in a solvent at a temperature of about −15° C. to about 25° C. and
allowing the solvent to evaporate.

39.-61. (canceled)

62. The composition of claim 18, wherein the crystalline form further comprises a pharmaceutically acceptable carrier or an edible carrier.

63. (canceled)
